(19) United States
(12) Patent Application Publication
(10) Pub. No.: US 2017/0198040 A1
(43) Pub. Date: Jul. 13, 2017

(54) ANTIBODIES TARGETING CD32b AND METHODS OF USE THEREOF

(71) Applicant: Novartis AG, Basel (CH)

(72) Inventors: Nicole BALKE, Basel (CH); Thomas CALZASCIA, Binningen (CH); Stefan EWERT, Allschwil (CH); Alan HARRIS, Cambridge, MA (US); Heather Adkins HUET, Arlington, MA (US); Isabelle ISNARDI, Basel (CH); Haihui LU, Cambridge, MA (US); Matthew John MEYER, Framingham, MA (US); Nicholas WILSON, Somerville, MA (US); Fangmin WU, Cambridge, MA (US)

(73) Assignee: Novartis AG, Basel (CH)

(21) Appl. No.: 15/382,251

(22) Filed: Dec. 16, 2016

Related U.S. Application Data
(60) Provisional application No. 62/269,444, filed on Dec. 18, 2015.
(60) Provisional application No. 62/334,747, filed on May 11, 2016.

(51) Int. Cl. C07K 16/28; A61K 45/06

(52) U.S. Cl. CPC C07K 16/2803; C07K 16/2887; A61K 45/06; C07K 2317/41; C07K 2317/56; C07K 2317/72; C07K 2317/732; C07K 2317/76; C07K 2317/35; C07K 2317/31; A61K 2039/507

(57) ABSTRACT

The present invention relates to isolated antibodies and antigen-binding fragments thereof which selectively bind human CD32b. Also provided herein are compositions comprising the antibodies or antigen-binding fragments thereof, methods of using the antibodies or antigen-binding fragments thereof, and methods of making the antibodies or antigen-binding fragments thereof.

### SEQUENCE LISTING

The instant application contains a Sequence Listing which has been filed electronically in ASCII format and is hereby incorporated by reference in its entirety. Said ASCII copy, created on Dec. 15, 2016, is named PAT057036-WO-PCT_SL.txt and is 467,216 bytes in size.

### FIELD OF THE INVENTION

The present invention relates to antibodies and antigen-binding fragments thereof which bind human CD32b, and compositions and methods of use thereof.

### BACKGROUND OF THE INVENTION

Fc gamma receptors (FcγR) bind IgG and they are expressed by many immune cells, enabling them to serve as the link between innate and humoral immunity. Activatory FcγR contain immune-receptor tyrosine-based activating motifs (ITAMs) either directly in their intracellular portion or in the cytoplasmic domain of associated signaling units such as the homodimeric common γ chain. These ITAM motifs become phosphorylated when the receptors are cross-linked by antigen-antibody complexes. Activatory FcγR contain or are associated with immune-receptor tyrosine-based activating motifs (ITAMs) which become phosphorylated when the receptors are cross-linked by antigen-antibody complexes. Upon activation, these receptors mediate immune responses including phagocytosis and antibody dependent cellular cytotoxicity (ADCC) (Nimmerjahn and Ravetch, Nature Rev. Immunol. 2008: 8(1) 34-47). CD32b is the sole inhibitory FcγR and contains an intracellular immune-receptor tyrosine-based inhibitory mofit (ITIM). CD32b is expressed by immune cells including dendritic cells and macrophages (Nimmerjahn and Ravetch, Nature Rev. Immunol. 2008: 8(1) 34-47) and is the only FcγR expressed on B cells (Amigorena et al., Eur. J. Immunol. 1989:19(8) 1379-1385). Activation of CD32b and ITIM phosphorylation results in inhibition of activatory FcγR functions (Smith and Clatworthy, Nat. Rev. Immunol. 2010: (5) 328-343) or, when cross-linked to the B cell receptor, reduced B cell function (Horton et al., J. Immunol. 2011: 186(7):4223-4233). Consistent with its inhibitory role, therapeutic antibodies with Fc dependent activity/ADCC mode of action have a more robust anti-tumor response in CD32b knockout mice than in WT mice (Clynes et al., Nat. Med. 2000: 6(4):443-6). Additionally, polymorphisms that impair CD32b function are associated with development of autoimmunity (Floto et al., Nat. Med. 2005: 11(10) 1056-1058). CD32b is expressed as two splice variants, CD32b1 and CD32b2, which have similar extracellular domains but different intracellular domains that dictate their propensity for internalization. The full length variant, CD32b1 (UniProtKB P31944-1), is expressed on lymphoid cells and has an intracellular signal sequence that prevents internalization. CD32b2 (UniProtKB P31944-2), which is expressed on myloid cells, lacks this signal sequence and is therefore more susceptible to internalization (Brooks et al., J. Exp. Med. 1989: 170(4) 1369-1385).

In addition to being expressed throughout B cell maturation, CD32b is found highly expressed on the malignant counter parts of these cells. Specifically, CD32b is found expressed on B cell lymphomas including CLL, NHL, multiple myeloma, and CD32b has been proposed as a therapeutic target for these indications (e.g. Rankin et al., Blood 2006: 108(7) 2384-2391) and others including systemic light-chain amyloidosis (Zhou et al., Blood 2008: 111(7) 3403-3406).

Expression of CD32b on tumor cells has been shown to correlate with reduced clinical benefit from rituximab containing treatment regimens (Lim et al., Blood 2011: 118(9) 2530-2540). Furthermore, CD32b expression was found to be increased in a B cell leukemia model upon developing resistance to alemtuzumab in vivo and knockdown of CD32b re-sensitized the leukemic cells to alemtuzumab mediated ADCC activity (Pallasch et al., Cell 2014: 156(3) 590-602). Taken together, these data support a role for CD32b as a mechanism of resistance to antibodies with Fc dependent (e.g. ADCC mediated) anti-tumor activity. This mechanism is not well understood but several hypotheses exist. Lim et al. (Blood 2011: 118(9) 2530-2540) and Vaughan et al. (Blood 2014: 123(5) 669-677) demonstrated with lymphoma cells that CD32b binds the Fc of CD20 bound rituximab causing the tripartite complex to internalize and ultimately resulting in reduced CD20 bound rituximab coating the lymphoma cell surface. It has also been proposed that CD32b on lymphoma cells engage the Fc region of, for example, CD20 bound rituximab in cis effectively masking the rituximab Fc. The anticipated consequence of the rituximab Fc masking is a reduced opportunity to engage the activatory FcγR on effector cells in trans (Vaughan et al. Blood 2014: 123(5) 669-677). Evidence that FcγR can function in this manner has been demonstrated during herpes simplex virus infection, where a virally encoded FcγR engages the Fc region of antibodies bound to viral antigens expressed by the infected cell thereby protecting it from antibody-dependent cellular cytotoxicity (Van Vliet et al., Immunology 1992: 77(1) 109-115). In both mechanisms outlined above, CD32b effectively reduces the interactions between a therapeutic mAb Fc, e.g. rituximab, and activatory FcγR on effector cells resulting in a diminished immune response/ADCC activity.

### SUMMARY OF THE INVENTION

The present invention provides an isolated antibody or antigen-binding fragment thereof, which comprises:

(a) A heavy chain variable region CDR1 comprising an amino acid sequence selected from any one of SEQ ID NOs: 1, 4, 7, 53, 56, 59, 105, 108, 111, 157, 160, 163, 209, 212, 215, 261, 264, 267, 313, 316, 319, 365, 368, 371, 417, 420, 423, 469, 472, 475, 521, 524, 527, 547, 550, 553, 573, 576, 579, 625, 628, and 631;
(b) a heavy chain variable region CDR2 comprising an amino acid sequence selected from any of SEQ ID NOs: 2, 5, 8, 54, 57, 60, 106, 109, 112, 158, 161, 164, 210, 213, 216, 262, 265, 268, 314, 317, 320, 366, 369, 372, 418, 421; 424, 470, 473, 476, 522, 525, 528, 548, 551, 554, 574, 577, 580, 626, 629, and 632;
(c) a heavy chain variable region CDR3 comprising an amino acid sequence selected from any of SEQ ID NOs: 3, 6, 9, 55, 58, 61, 107, 110, 113, 159, 162, 165, 211, 214, 217, 263, 266, 269, 315, 318, 321, 367, 370, 373, 419, 422, 425, 471, 474, 477, 523, 526, 529, 549, 552, 555, 575, 578, 581, 627, 630, and 633;
(d) a light chain variable region CDR1 comprising an amino acid sequence selected from any of SEQ ID NOs: 14, 17, 20, 66, 69, 72, 118, 121, 124, 170, 173, 176, 222, 225, 228, 274, 277, 280, 326, 329, 332, 378, 381, 384, 430, 433, 436, 482, 485, 488, 534, 537, 540, 560, 563, 566, 586, 589, 592, 638, 641, 644;
(e) a light chain variable region CDR2 comprising an amino acid sequence selected from any of SEQ ID NOs: 15, 18, 21, 67, 70, 73, 119, 122, 125, 171, 174, 177, 223, 226, 229, 275, 278, 281, 327, 330, 333, 379, 382, 385, 431, 434, 437, 483, 486, 489, 535, 538, 541, 561, 564, 567, 587, 590, 593, 639, 642, and 645; and
(f) a light chain variable region CDR3 comprising an amino acid sequence selected from any of SEQ ID NOs: 16, 19, 22, 68, 71, 74, 120, 123, 126, 172, 175, 178, 224, 227, 230, 276, 279, 282, 328, 331, 334, 380, 383, 386, 432, 435, 438, 484, 487, 490, 536, 539, 542, 562, 565, 568, 588, 591, 594, 640, 643, and 646;
wherein the antibody selectively binds human CD32b.

In another embodiment, this application discloses an antibody or antigen-binding fragment thereof, wherein the antibody comprises: a heavy chain variable region comprising an amino acid sequence selected from any of SEQ ID NOs: 10, 62, 114, 166, 218, 270, 322, 374, 426, 478, 530, 556, 582, and 634; and a light chain variable region comprising an amino acid sequence selected from any of SEQ ID NOs: 23, 75, 127, 179, 231, 283, 335, 387, 439, 491, 543, 569, 595, and 647, wherein the antibody selectively binds human CD32b.

In yet another embodiment, the present application discloses an antibody or antigen-binding fragment, wherein the antibody comprises: a heavy chain comprising an amino acid sequence selected from any of SEQ ID NOs: 12, 64, 116, 168, 220, 272, 324, 376, 428, 480, 584, and 636; and a light chain comprising an amino acid sequence selected from any of SEQ ID NOs: 25, 77, 129, 181, 233, 285, 337, 389, 441, 493, 597, and 649, wherein the antibody selectively binds human CD32b.

The present application further discloses an antibody or antigen-binding fragment thereof, wherein the antibody comprises: a heavy chain comprising an amino acid sequence selected from any of SEQ ID NOs: 38, 90, 142, 194, 246, 298, 350, 402, 454, 506, 532, 558, 610, and 662; and a light chain comprising an amino acid sequence selected from any of SEQ ID NOs: 51, 103, 155, 207, 259, 311, 363, 415, 467, 519, 545, 571, 623, and 675, wherein the antibody selectively binds human CD32b.

In a further embodiment, the present application discloses an antibody or antigen-binding fragment thereof, wherein the antibody comprises:

(a) HCDR1, HCDR2, and HCDR3 sequences of SEQ ID NOs: 1, 2, and 3, respectively, and LCDR1, LCDR2, and LCDR3 sequences of SEQ ID NOs: 14, 15, and 16, respectively;
(b) HCDR1, HCDR2, and HCDR3 sequences of SEQ ID NOs: 4, 5, and 6, respectively, and LCDR1, LCDR2, and LCDR3 sequences of SEQ ID NOs: 17, 18, and 19, respectively;
(c) HCDR1, HCDR2, and HCDR3 sequences of SEQ ID NOs: 7, 8, and 9, respectively, and LCDR1, LCDR2, and LCDR3 sequences of SEQ ID NOs: 20, 21, and 22, respectively;
(d) HCDR1, HCDR2, and HCDR3 sequences of SEQ ID NOs: 53, 54, and 55, respectively, and LCDR1, LCDR2, and LCDR3 sequences of SEQ ID NOs: 66, 67, and 68 respectively;
(e) HCDR1, HCDR2, and HCDR3 sequences of SEQ ID NOs: 56, 57, and 58, respectively, and LCDR1, LCDR2, and LCDR3 sequences of SEQ ID NOs: 69, 70, and 71 respectively;
(f) HCDR1, HCDR2, and HCDR3 sequences of SEQ ID NOs: 59, 60, and 61, respectively, and LCDR1, LCDR2, and LCDR3 sequences of SEQ ID NOs: 72, 73, and 74 respectively;
(g) HCDR1, HCDR2, and HCDR3 sequences of SEQ ID NOs: 105, 106, and 107 respectively, and LCDR1, LCDR2, and LCDR3 sequences of SEQ ID NOs: 118, 119, 120, respectively;
(h) HCDR1, HCDR2, and HCDR3 sequences of SEQ ID NOs: 108, 109, and 110 respectively, and LCDR1, LCDR2, and LCDR3 sequences of SEQ ID NOs: 121, 122, 123, respectively;
(i) HCDR1, HCDR2, and HCDR3 sequences of SEQ ID NOs: 111, 112, and 113 respectively, and LCDR1, LCDR2, and LCDR3 sequences of SEQ ID NOs: 124, 125, 126, respectively;
(j) HCDR1, HCDR2, and HCDR3 sequences of SEQ ID NOs: 157, 158, and 159, respectively, and LCDR1, LCDR2, and LCDR3 sequences of SEQ ID NOs: 170, 171, 172, respectively;
(k) HCDR1, HCDR2, and HCDR3 sequences of SEQ ID NOs: 160, 161, and 162, respectively, and LCDR1, LCDR2, and LCDR3 sequences of SEQ ID NOs: 173, 174, 175, respectively;
(l) HCDR1, HCDR2, and HCDR3 sequences of SEQ ID NOs: 163, 164, and 165, respectively, and LCDR1, LCDR2, and LCDR3 sequences of SEQ ID NOs: 176, 177, 178, respectively;
(m) HCDR1, HCDR2, and HCDR3 sequences of SEQ ID NOs: 209, 210, and 211, respectively, and LCDR1, LCDR2, and LCDR3 sequences of SEQ ID NOs: 222, 223, and 224, respectively;
(n) HCDR1, HCDR2, and HCDR3 sequences of SEQ ID NOs: 212, 213, and 214, respectively, and LCDR1, LCDR2, and LCDR3 sequences of SEQ ID NOs: 225, 226, and 227, respectively;
(o) HCDR1, HCDR2, and HCDR3 sequences of SEQ ID NOs: 215, 216, and 217 respectively, and LCDR1, LCDR2, and LCDR3 sequences of SEQ ID NOs: 228, 229, and 230, respectively;
(p) HCDR1, HCDR2, and HCDR3 sequences of SEQ ID NOs: 261, 262, and 263, respectively, and LCDR1, LCDR2, and LCDR3 sequences of SEQ ID NOs: 274, 275, and 276, respectively;
(q) HCDR1, HCDR2, and HCDR3 sequences of SEQ ID NOs: 264, 265, and 266, respectively, and LCDR1, LCDR2, and LCDR3 sequences of SEQ ID NOs: 277, 278, and 279, respectively;
(r) HCDR1, HCDR2, and HCDR3 sequences of SEQ ID NOs: 267, 268, and 269, respectively, and LCDR1, LCDR2, and LCDR3 sequences of SEQ ID NOs: 280, 281, and 282, respectively;
(s) HCDR1, HCDR2, and HCDR3 sequences of SEQ ID NOs: 313, 314, and 315, respectively, and LCDR1, LCDR2, and LCDR3 sequences of SEQ ID NOs: 326, 327, and 328, respectively;
(t) HCDR1, HCDR2, and HCDR3 sequences of SEQ ID NOs: 316, 317, and 318, respectively, and LCDR1, LCDR2, and LCDR3 sequences of SEQ ID NOs: 329, 330, and 331, respectively;
(u) HCDR1, HCDR2, and HCDR3 sequences of SEQ ID NOs: 319, 320, and 321, respectively, and LCDR1, LCDR2, and LCDR3 sequences of SEQ ID NOs: 332, 333, and 334, respectively;
(v) HCDR1, HCDR2, and HCDR3 sequences of SEQ ID NOs: 365, 366, and 367, respectively, and LCDR1, LCDR2, and LCDR3 sequences of SEQ ID NOs: 378, 379, and 380, respectively;
(w) HCDR1, HCDR2, and HCDR3 sequences of SEQ ID NOs: 368, 369, and 370, respectively, and LCDR1, LCDR2, and LCDR3 sequences of SEQ ID NOs: 381, 382, and 383, respectively;
(x) HCDR1, HCDR2, and HCDR3 sequences of SEQ ID NOs: 371, 372, and 373, respectively, and LCDR1, LCDR2, and LCDR3 sequences of SEQ ID NOs: 384, 385, and 386, respectively;
(y) HCDR1, HCDR2, and HCDR3 sequences of SEQ ID NOs: 417, 418, and 419, respectively, and LCDR1, LCDR2, and LCDR3 sequences of SEQ ID NOs: 430, 431, and 432, respectively;
(z) HCDR1, HCDR2, and HCDR3 sequences of SEQ ID NOs: 420, 421, and 422, respectively, and LCDR1, LCDR2, and LCDR3 sequences of SEQ ID NOs: 433, 434, and 435, respectively;
(aa) HCDR1, HCDR2, and HCDR3 sequences of SEQ ID NOs: 423, 424, and 425, respectively, and LCDR1, LCDR2, and LCDR3 sequences of SEQ ID NOs: 436, 437, and 438, respectively;
(bb) HCDR1, HCDR2, and HCDR3 sequences of SEQ ID NOs: 469, 470, and 471, respectively, and LCDR1, LCDR2, and LCDR3 sequences of SEQ ID NOs: 482, 483, and 484, respectively;
(cc) HCDR1, HCDR2, and HCDR3 sequences of SEQ ID NOs: 472, 473, and 474, respectively, and LCDR1, LCDR2, and LCDR3 sequences of SEQ ID NOs: 485, 486, and 487, respectively;
(dd) HCDR1, HCDR2, and HCDR3 sequences of SEQ ID NOs: 475, 476, and 477, respectively, and LCDR1, LCDR2, and LCDR3 sequences of SEQ ID NOs: 488, 489, and 490, respectively;
(ee) HCDR1, HCDR2, and HCDR3 sequences of SEQ ID NOs: 521, 522, and 523, respectively, and LCDR1, LCDR2, and LCDR3 sequences of SEQ ID NOs: 534, 535, and 536, respectively;
(ff) HCDR1, HCDR2, and HCDR3 sequences of SEQ ID NOs: 524, 525, and 526, respectively, and LCDR1, LCDR2, and LCDR3 sequences of SEQ ID NOs: 537, 538, and 539, respectively;
(gg) HCDR1, HCDR2, and HCDR3 sequences of SEQ ID NOs: 527, 528, and 529, respectively, and LCDR1, LCDR2, and LCDR3 sequences of SEQ ID NOs: 540, 541, and 542, respectively;
(hh) HCDR1, HCDR2, and HCDR3 sequences of SEQ ID NOs: 547, 548, and 549, respectively, and LCDR1, LCDR2, and LCDR3 sequences of SEQ ID NOs: 560, 561, and 562, respectively;
(ii) HCDR1, HCDR2, and HCDR3 sequences of SEQ ID NOs: 550, 551, and 552, respectively, and LCDR1, LCDR2, and LCDR3 sequences of SEQ ID NOs: 563, 564, and 565, respectively;
(jj) HCDR1, HCDR2, and HCDR3 sequences of SEQ ID NOs: 553, 554, and 555, respectively, and LCDR1, LCDR2, and LCDR3 sequences of SEQ ID NOs: 566, 567, and 568, respectively;
(kk) HCDR1, HCDR2, and HCDR3 sequences of SEQ ID NOs: 573, 574, and 575, respectively, and LCDR1, LCDR2, and LCDR3 sequences of SEQ ID NOs: 586, 587, and 588, respectively;
(ll) HCDR1, HCDR2, and HCDR3 sequences of SEQ ID NOs: 576, 577, and 578, respectively, and LCDR1, LCDR2, and LCDR3 sequences of SEQ ID NOs: 589, 590, and 591, respectively;
(mm) HCDR1, HCDR2, and HCDR3 sequences of SEQ ID NOs: 579, 580, and 581, respectively, and LCDR1, LCDR2, and LCDR3 sequences of SEQ ID NOs: 592, 593, and 594, respectively;
(nn) HCDR1, HCDR2, and HCDR3 sequences of SEQ ID NOs: 625, 626, and 627, respectively, and LCDR1, LCDR2, and LCDR3 sequences of SEQ ID NOs: 638, 639, and 640, respectively;
(oo) HCDR1, HCDR2, and HCDR3 sequences of SEQ ID NOs: 628, 629, and 630, respectively, and LCDR1, LCDR2, and LCDR3 sequences of SEQ ID NOs: 641, 642, and 643, respectively; or
(pp) HCDR1, HCDR2, and HCDR3 sequences of SEQ ID NOs: 631, 632, and 633, respectively, and LCDR1, LCDR2, and LCDR3 sequences of SEQ ID NOs: 644, 645, and 646, respectively.

In addition embodiments, the application discloses an isolated antibody or antigen-binding fragment thereof, comprising:

(a) A VH sequence of SEQ ID NO: 10 and a VL sequence of SEQ ID NO: 23;
(b) A VH sequence of SEQ ID NO: 62 and a VL sequence of SEQ ID NO: 75;
(c) A VH sequence of SEQ ID NO: 114 and VL sequence of SEQ ID NO: 127;
(d) A VH sequence of SEQ ID NO: 166 and a VL sequence of SEQ ID NO: 179;
(e) A VH sequence of SEQ ID NO: 218 and a VL sequence of SEQ ID NO: 231;
(f) A VH sequence of SEQ ID NO: 270 and a VL sequence of SEQ ID NO: 283;
(g) A VH sequence of SEQ ID NO: 322 and a VL sequence of SEQ ID NO: 335;
(h) A VH sequence of SEQ ID NO: 374 and VL sequence of SEQ ID NO: 387;
(i) A VH sequence of SEQ ID NO: 426 and a VL sequence of SEQ ID NO: 439;
(j) A VH sequence of SEQ ID NO: 478 and a VL sequence of SEQ ID NO: 491;
(k) A VH sequence of SEQ ID NO: 530 and a VL sequence of SEQ ID NO: 543;
(l) A VH sequence of SEQ ID NO: 556 and a VL sequence of SEQ ID NO: 569;
(m) A VH sequence of SEQ ID NO: 582 and a VL sequence of SEQ ID NO: 595; or
(n) A VH sequence of SEQ ID NO: 634 and a VL sequence of SEQ ID NO: 647.

In yet another embodiment, the present application discloses an isolated antibody or antigen-binding fragment thereof, comprising:

(a) A heavy chain sequence of SEQ ID NO: 12; and a light chain sequence of SEQ ID NO: 25;
(b) A heavy chain sequence of SEQ ID NO: 64; and a light chain sequence of SEQ ID NO: 77;
(c) A heavy chain sequence of SEQ ID NO: 116; and a light chain sequence of SEQ ID NO: 129;
(d) A heavy chain sequence of SEQ ID NO: 168; and a light chain sequence of SEQ ID NO: 181;
(e) A heavy chain sequence of SEQ ID NO: 220; and a light chain sequence of SEQ ID NO: 233;
(f) A heavy chain sequence of SEQ ID NO: 272; and a light chain sequence of SEQ ID NO: 285;
(g) A heavy chain sequence of SEQ ID NO: 324; and a light chain sequence of SEQ ID NO: 337;
(h) A heavy chain sequence of SEQ ID NO: 376; and a light chain sequence of SEQ ID NO: 389;
(i) A heavy chain sequence of SEQ ID NO: 428; and a light chain sequence of SEQ ID NO: 441;
(j) A heavy chain sequence of SEQ ID NO: 480; and a light chain sequence of SEQ ID NO: 493;
(k) A heavy chain sequence of SEQ ID NO: 584; and a light chain sequence of SEQ ID NO: 597; or
(l) A heavy chain sequence of SEQ ID NO: 636; and a light chain sequence of SEQ ID NO: 649.

In one embodiment, the application discloses an isolated antibody or antigen-binding fragment thereof, comprising:

(a) A heavy chain sequence of SEQ ID NO: 38; and a light chain sequence of SEQ ID NO: 51;
(b) A heavy chain sequence of SEQ ID NO: 90; and a light chain sequence of SEQ ID NO: 103;
(c) A heavy chain sequence of SEQ ID NO: 142; and a light chain sequence of SEQ ID NO: 155;
(d) A heavy chain sequence of SEQ ID NO: 194; and a light chain sequence of SEQ ID NO: 207;
(e) A heavy chain sequence of SEQ ID NO: 246; and a light chain sequence of SEQ ID NO: 259;
(f) A heavy chain sequence of SEQ ID NO: 298; and a light chain sequence of SEQ ID NO: 311;
(g) A heavy chain sequence of SEQ ID NO: 350; and a light chain sequence of SEQ ID NO: 363;
(h) A heavy chain sequence of SEQ ID NO: 402; and a light chain sequence of SEQ ID NO: 415;
(i) A heavy chain sequence of SEQ ID NO: 454; and a light chain sequence of SEQ ID NO: 467;
(j) A heavy chain sequence of SEQ ID NO: 506; and a light chain sequence of SEQ ID NO: 519;
(k) A heavy chain sequence of SEQ ID NO: 532; and a light chain sequence of SEQ ID NO: 545;
(l) A heavy chain sequence of SEQ ID NO: 558; and a light chain sequence of SEQ ID NO: 571;
(m) A heavy chain sequence of SEQ ID NO: 610; and a light chain sequence of SEQ ID NO: 623; or
(n) A heavy chain sequence of SEQ ID NO: 662; and a light chain sequence of SEQ ID NO: 675.

The present application also discloses an isolated antibody or antigen binding fragment thereof comprising:

(a) a HCDR1 comprising the amino acid sequence selected from SEQ ID NOs: 157, 160, or 163;
(b) a HCDR2 comprising the amino acid sequence selected from SEQ ID NOs: 158, 161, or 164;
(c) a HCDR3 comprising the amino acid sequence selected from SEQ ID NOs: 159, 315, 367, 419, 471, 523, 549, 575, or 627;
(d) a LCDR1 comprising the amino acid sequence selected from SEQ ID NOs: 170, 173, or 176;
(e) a LCDR2 comprising the amino acid sequence selected from SEQ ID NOs: 171, 174, or 177; and
(f) a LCDR3 comprising the amino acid sequence of SEQ ID NO: 172.

In a further embodiment, the present application provides an isolated antibody or antigen binding fragment thereof comprising:

(a) a HCDR1 comprising the amino acid sequence selected from SEQ ID NOs: 157, 160, or 163;
(b) a HCDR2 comprising the amino acid sequence selected from SEQ ID NOs: 158, 161, or 164;
(c) a HCDR3 comprising the amino acid sequence EQX₁PX₂X₃GX₄GGX₅PX₆EAMDV (SEQ ID NO: 683), wherein X₁ is D or S, X₂ is E or S, X₃ is Y, F, A, or S; X₄ is Y or F; X₅ is F or Y, and X₆ is Y or F;
(d) a LCDR1 comprising the amino acid sequence selected from SEQ ID NOs: 170, 173, or 176;
(e) a LCDR2 comprising the amino acid sequence selected from SEQ ID NOs: 171, 174, or 177; and
(f) a LCDR3 comprising the amino acid sequence of SEQ ID NO: 172.

In another embodiment, this application discloses an isolated antibody or antigen-binding fragment thereof, comprising:

(a) a HCDR1 comprising the amino acid sequence selected from SEQ ID NO: 157, 160, or 163;
(b) a HCDR2 comprising the amino acid sequence selected from SEQ ID NO: 158, 161, or 164;
(c) a HCDR3 comprising the amino acid sequence of SEQ ID NO: 159, 315, 367, or 419;
(d) a LCDR1 comprising the amino acid sequence selected from SEQ ID NOs: 170, 173, or 176;
(e) a LCDR2 comprising the amino acid sequence selected from SEQ ID NOs: 171, 174, or 177; and
(f) a LCDR3 comprising the amino acid sequence of SEQ ID NO: 172.

In yet another embodiment, the present application discloses an isolated antibody or antigen-binding fragment thereof, comprising:

(a) a HCDR1 comprising the amino acid sequence selected from SEQ ID NO: 417;
(b) a HCDR2 comprising the amino acid sequence selected from SEQ ID NO: 418;
(c) a HCDR3 comprising the amino acid sequence of SEQ ID NO: 419;
(d) a LCDR1 comprising the amino acid sequence selected from SEQ ID NOs: 430;
(e) a LCDR2 comprising the amino acid sequence selected from SEQ ID NOs: 431; and
(f) a LCDR3 comprising the amino acid sequence of SEQ ID NO: 432.

In one embodiment of the present application, there is provided an afucosylated antibody or antigen-binding fragment thereof comprising:

(a) a HCDR1 comprising the amino acid sequence selected from SEQ ID NO: 417;
(b) a HCDR2 comprising the amino acid sequence selected from SEQ ID NO: 418;
(c) a HCDR3 comprising the amino acid sequence of SEQ ID NO: 419;
(d) a LCDR1 comprising the amino acid sequence selected from SEQ ID NOs: 430;
(e) a LCDR2 comprising the amino acid sequence selected from SEQ ID NOs: 431; and
(f) a LCDR3 comprising the amino acid sequence of SEQ ID NO: 432.

In a further embodiment, the present application provides an afucosylated antibody or antigen-binding fragment thereof, comprising a variable heavy chain region comprising the amino acid sequence of SEQ ID NO: 426 and a light chain variable region comprising the amino acid sequence of SEQ ID NO: 441.

In another embodiment, the present application discloses an afucosylated antibody or antigen-binding fragment, comprising a heavy chain comprising the amino acid sequence of SEQ ID NO: 428 and a light chain comprising the amino acid sequence of SEQ ID NO: 441.

The present application also provides an antibody or antigen-binding fragment thereof, wherein the antibody or antigen-binding fragment thereof comprises a heavy chain variable region comprising an amino acid sequence that is at least 90% identical to the amino acid sequence selected from the group consisting of SEQ ID NOs: 10, 62, 114, 166, 218, 270, 322, 374, 426, 478, 530, 556, 582, and 634; and a light chain variable region comprising an amino acid sequence that is at least 90% identical to the amino acid sequence selected from the group consisting of SEQ ID NOs: 23, 75, 127, 179, 231, 283, 335, 387, 439, 491, 543, 569, 595, and 647; wherein the antibody specifically binds to human CD32b protein.

The present application further provides an isolated antibody or antigen-binding fragment thereof, wherein the antibody or antigen-binding fragment thereof comprises a heavy chain comprising an amino acid sequence that is at least 90% identical to the amino acid sequence selected from the group consisting of SEQ ID NOs: 12, 38, 64, 90, 116, 142, 168, 194, 220, 246, 272, 298, 324, 350, 376, 402, 428, 454, 480, 506, 532, 558, 584, 610, 636, and 662; and a light chain comprising an amino acid sequence that is at least 90% identical to the amino acid sequence selected from the group consisting of SEQ ID NOs: 25, 51, 77, 103, 129, 155, 181, 207, 233, 259, 285, 311, 337, 363, 389, 415, 441, 467, 493, 519, 545, 571, 597, 623, 649, and 675; wherein the antibody specifically binds to human CD32b protein.

The present application provides that in some of the embodiments of the isolated antibody or antigen-binding fragment thereof described above, the antibody is afucosylated. In other embodiments, the Fc portion of the antibody is modified to enhance ADCC activity.

In all of the embodiments described herein, the isolated antibody or antigen-binding fragment thereof selectively binds human CD32b over human CD32a.

In some embodiments disclosed in the present application, the isolated antibody or antigen-binding fragment thereof is an IgG selected from the group consisting of an IgG1, an IgG2, an IgG3 and an IgG4. In other embodiments, the isolated antibody or antigen-binding fragment is selected from the group consisting of: a monoclonal antibody, a chimeric antibody, a single chain antibody, a Fab and a scFv. In yet other embodiments, the isolated antibody or antigen-binding fragment thereof disclosed herein are chimeric, humanized or fully human.

In one embodiment, the antibody or antigen-binding fragment thereof disclosed in the present application inhibits binding of human CD32b to immunoglobulin Fc domains.

In a further embodiment, the isolated antibody or antigen-binding fragment thereof disclosed herein is a component of an immunoconjugate.

In some embodiments of the present application, a multivalent antibody comprises any of the isolated antibody or antigen-binding fragment thereof disclosed herein. In a further embodiment, the multivalent antibody is a bispecific antibody.

Also disclosed herein are compositions comprising the isolated antibody or antigen-binding fragment thereof or multivalent antibody disclosed herein, in combination with one or more additional antibodies that bind a cell surface antigen that is co-expressed with CD32b on a cell. The cell surface antigen and CD32b may be co-expressed on B cells. In some embodiments, the cell surface antigen is selected from the group consisting of CD20, CD38, CD52, CS1/SLAMF7, CD56, CD138, KiR, CD19, CD40, Thy-1, Ly-6, CD49, Fas, Cd95, APO-1, EGFR, HER2, CXCR4, HLA molecules, GM1, CD22, CD23, CD80, CD74, or DRD. In some embodiments, the additional antibody is selected from the group consisting of rituximab, elotuzumab, ofatumumab, obinutumumab, daratumumab, and alemtuzumab.

In yet another embodiment, the isolated antibody or antigen-binding fragment thereof or the multivalent antibody disclosed herein, or a composition comprising the isolated antibody or antigen-binding fragment thereof or the multivalent antibody disclosed herein may further comprise an additional therapeutic compound. In some embodiments, the additional therapeutic compound is an immunomodulator. In one embodiment, the immunomodulator is IL15. In another embodiment, the immunomodulator is an agonist of a costimulatory molecule selected from OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3, CD83 ligand, and STING. In another embodiment, the immunomodulator is an inhibitor molecule of a target selected from PD-1, PD-L1, PD-L2, CTLA-4, TIM-3, LAG-3, CEACAM-1, CEACAM-3, CEACAM-5, VISTA, BTLA, TIGIT, LAIR1, CD160, 2B4, TGFR beta, and IDO. In a further embodiment, the additional therapeutic compound is selected from ofatumumab, ibrutinib, belinostat, romidepsin, brentuximab vedotin, obinutuzumab, pralatrexate, pentostatin, dexamethasone, idelalisib, ixazomib, liposomal doxyrubicin, pomalidomide, panobinostat, elotuzumab, daratumumab, alemtuzumab, thalidomide, and lenalidomide.

The present application also provides pharmaceutical compositions comprising the isolated antibody or antigen-binding fragment thereof, the multivalent antibody, or compositions comprising the isolated antibody or antigen-binding fragment thereof or the multivalent antibody disclosed herein, and a pharmaceutically acceptable carrier.

In another embodiment, the present application discloses an isolated antibody or antigen binding fragment thereof that specifically binds to CD32b within the Fc binding domain of CD32b. In some embodiments, the antibody binds within amino acid residues 107-123 (VLRCHSWKDKPLVKVTF) of CD32b. In other embodiments, the antibody prevents or reduces CD32b binding to the immunoglobulin Fc domain of a second antibody that binds to a tumor antigen co-expressed with CD32b on a B-cell. In some embodiments, the second antibody binds to a tumor antigen selected from the group consisting of CD20, CD38, CD52, CS1/SLAMF7, CD56, CD138, KiR, CD19, CD40, Thy-1, Ly-6, CD49, Fas, Cd95, APO-1, EGFR, HER2, CXCR4, HLA molecules, GM1, CD22, CD23, CD80, CD74, or DRD. In particular embodiments, the second antibody binds to a tumor antigen selected from the group consisting of CD20, CD38, CS1/SLAMF7 and CD52. In further embodiments, the second antibody is selected from the group consisting of rituximab, elotuzumab, ofatumumab, obinutumumab, daratumumab, and alemtuzumab. In some embodiments, the isolated antibody or antigen binding fragments that specifically binds to CD32b within the Fc binding in domain of CD32b is an antibody as disclosed herein.

In yet another embodiment, the present application discloses an isolated antibody or antigen binding fragment thereof that specifically binds to CD32b and inhibits or reduces CD32b immunoreceptor tyrosine-based inhibition motif (ITIM) signaling mediated by a second antibody that binds to a tumor antigen co-expressed with CD32b on a B-cell. The B-cell can be a normal B-cell or malignant B-cell.

In a further embodiment, this application discloses a method of inhibiting or reducing CD32b ITIM signaling that is induced by administration of a therapeutic antibody that binds to a tumor antigen co-expressed with CD32b on a B-cell comprising administering an isolated antibody or antigen binding fragment thereof that specifically binds to the Fc binding domain of CD32b. The isolated antibody or antigen binding fragment thereof does not stimulate ITIM signaling. In some embodiments of this method, the therapeutic antibody binds to a tumor antigen selected from the group consisting of CD20, CD38, CD52, CS1/SLAMF7, CD56, CD138, KiR, CD19, CD40, Thy-1, Ly-6, CD49, Fas, Cd95, APO-1, EGFR, HER2, CXCR4, HLA molecules, GM1, CD22, CD23, CD80, CD74, or DRD. In other embodiments of the method, the therapeutic antibody is selected from the group consisting of rituximab, elotuzumab, ofatumumab, obinutumumab, daratumumab, and alemtuzumab.

This application also provides methods of treating a CD32b-related condition in a subject in need thereof comprising administering to the subject a therapeutically effective amount of the antibody or antigen-binding fragment thereof, the multivalent antibody, or compositions comprising the isolated antibody or antigen-binding fragment thereof or the multivalent antibody as disclosed herein. Also provided are the antibody or antigen-binding fragment thereof, the multivalent antibody, or compositions comprising the isolated antibody or antigen-binding fragment thereof or the multivalent antibody as disclosed herein, for use in treating a CD32b-related condition in a subject in need thereof. Further provided are uses of the antibody or antigen-binding fragment thereof, the multivalent antibody, or compositions comprising the isolated antibody or antigen-binding fragment thereof or the multivalent antibody as disclosed herein, to treat a CD32b-related condition in a subject in need thereof, or for the manufacture of a medicament for treatment of a CD32b-related condition, in a subject in need thereof. In some embodiments, the CD32b-related condition is selected from B cell malignancies, Hodgkins lymphoma, Non-Hodgkins lymphoma, multiple myeloma, diffuse large B cell lymphoma, acute lymphocytic leukemia, chronic lymphocytic leukemia, small lymphocytic lymphoma, diffuse small cleaved cell lymphoma, MALT lymphoma, mantel cell lymphoma, marginal zone lymphoma, follicular lymphoma, or systemic light chain amyloidosis.

The present application also discloses method of treating a patient who is resistant or refractory to treatment using an antibody that binds to a cell surface antigen that is co-expressed with CD32b on a cell, comprising co-administering the antibody with any one of the isolated anti-CD32b antibodies or an antigen-binding fragment thereof or the multivalent antibodies disclosed herein. This application also discloses use of any one of the isolated anti-CD32b antibodies or an antigen-binding fragment thereof or the multivalent antibodies disclosed herein for treatment of a patient who is resistant or refractory to treatment using an antibody that binds to a cell surface antigen that is co-expressed with CD32b on a cell, comprising co-administering the antibody with the anti-Cd32b antibodies or antigen-binding fragment thereof. This application further discloses the isolated anti-CD32b antibodies or an antigen-binding fragment thereof or the multivalent antibodies disclosed herein for treatment of a patient who is resistant or refractory to treatment using an antibody that binds to a cell surface antigen that is co-expressed with CD32b on a cell, comprising co-administering the antibody with the anti-Cd32b antibodies or antigen-binding fragment thereof.

The present application also provides nucleic acids encoding the antibody or antigen-binding fragment thereof disclosed herein, as well as a vector comprising the nucleic acid, and a host cell comprising the nucleic acid or the vector. Also provided are methods of producing the antibody or antigen-binding fragment thereof disclosed herein, the method comprising: culturing a host cell expressing a nucleic acid encoding the antibody; and collecting the antibody from the culture.

The present application also provides an isolated polynucleotide encoding an antibody or antigen-binding fragment thereof which selectively binds a human CD32b antibody comprising a CDR listed in Table 1.

### BRIEF DESCRIPTION OF THE FIGURES

FIG. 1 depicts an electropherogram of antibody NOV1216. Capillary zone electrophoresis (CZE) analysis of mammalian expressed NOV1216 in IgG revealed that the antibody existed as three predominant species, unmodified, +80 daltons, and +160 daltons.

FIGS. 2A-2H depict electropherograms of eight CD32b-binding CDR-H3 mutant antibodies by capillary zone electrophoresis.

FIG. 3 is a series of graphs depicting results from binding assays of a panel of CD32b-binding antibodies to CHO cells expressing CD32b or CD32a, as measured by flow cytometry.

FIG. 4 is a series of graphs depicting results from binding assays of a panel of CD32b-binding antibodies to CHO cells expressing variants of human CD16 and CD64, as measured by flow cytometry.

FIG. 5 is a series of graphs depicting results from binding assays of a panel of CD32b-binding antibodies to human B cells, as measured by flow cytometry.

FIG. 6 is a series of graphs depicting the results from binding assays of a panel of CD32b-binding antibodies to BJAB cells, as measured by flow cytometry.

FIG. 7a and FIG. 7b depict a series of 3D models of WT and mutant CD32b proteins designed to characterize the binding epitope of CD32b-binding antibodies.

FIG. 8a-FIG. 8c are a series of graphs depicting the binding characteristics of a panel of CD32b-binding antibodies, as measured by flow cytometry, to CHO cells expressing WT and mutant CD32b proteins designed to characterize the binding epitope of the antibodies.

FIG. 9 is a series of graphs depicting the binding characteristics of a panel of CD32b-binding antibodies to cell lines featuring a range of CD32b expression, CD32a expression, or no CD32b or CD32a expression.

FIG. 10 is a series of graphs depicting the binding characteristics of a panel of CDR-H3 mutant CD32b-binding antibodies to cell lines featuring a range of CD32b expression, CD32a expression, or no CD32b or CD32a expression.

FIG. 11a and FIG. 11b are a series of graphs depicting the activity of a panel of CD32b-binding antibodies having wild type Fc regions (Fc WT) in primary NK cell ADCC assays.

FIG. 12 is a graph depicting the in vivo antitumor activity of a panel of Fc WT CD32b-binding antibodies against established, disseminated mantle cell lymphoma Jeko1 xenografts in immunocompromised mice.

FIG. 13 is a series of graphs depicting the dose-responsive, in vivo antitumor activity of Fc WT CD32b-binding antibody NOV1216 against established Daudi xenografts in immunocompromised mice.

FIG. 14a-FIG. 14d are a series of graphs depicting the activity of Fc WT, enhanced ADCC (eADCC) Fc mutant, afucosylated, or N297A Fc mutant CD32b-binding antibodies in a primary NK cell ADCC assay and a CD16a activation reporter assay with Daudi and Jeko1 as target cells.

FIG. 15 is a series of graphs depicting the activity of Fc WT, eADCC Fc mutant, and N297A Fc mutant verions CD32b-binding antibodies in a primary NK cell ADCC assay with Jeko1 as the target cells.

FIG. 16 is a series of graphs depicting the activity of Fc WT, eADCC Fc mutant, and N297A Fc mutant versions of CD32b-binding antibody NOV1216 in CD16a reporter assays with target cells displaying a range of CD32b expression.

FIG. 17 is a series of graphs depicting the activity of of afucosylated CD32b-binding CDR-H3 mutant antibodies in a CD16a reporter assay with target cells displaying a range of CD32b expression.

FIG. 18 is a series of graphs depicting the activity of afucosylated CD32b-binding CDR-H3 mutant antibodies in primary NK cell ADCC assays.

FIG. 19 is a graph depicting the activity of afucosylated CD32b-binding CDR-H3 mutant antibodies in a primary NK cell ADCC assay.

FIG. 20 is a series of graphs depicting the in vivo antitumor activity of Fc WT, N297A, and eADCC Fc mutant versions of CD32b-binding antibody NOV1216 against established Daudi xenografts.

FIG. 21 is a graph depicting the in vivo antitumor activity of afucosylated CDR-H3 mutant CD32b-binding antibodies against established Daudi xenografts.

FIG. 22 is a series of graphs depicting the activity of rituximab and obinutuzumab when combined with Fc silent CD32b-binding antibody NOV1216 N297A in a CD16a activation assay.

FIG. 23 is a graph depicting improvement in rituximab activity when combined with Fc silent CD32b-binding CDR-H3 mutant antibodies in a CD16 activation assay.

FIG. 24 is a series of graphs depicting in vivo antitumor activity of rituximab or obinutuzumab combined with CD32b-binding antibody NOV1216 eADCC Fc mutant in mice bearing established Daudi xenografts.

FIG. 25 is a graph depicting improvement in daratumumab activity when combined with Fc silent CD32b-binding CDR-H3 mutant NOV2108 N297A in a CD16a activation assay.

FIG. 26 is a graph depicting the ability of wildtype and afucosylated NOV1216 and CDR-H3 mutant NOV2108, compared to wildtype clone 10 antibodies to mediate Daudi target cell killing by humanmacrophages.

FIG. 27 is a series of graphs depicting the impact of CD32b-binding antibodies 2B6 and NOV1216 on basal and crosslinked anti-IgM stimulated CD32b ITIM phosphorylation in primary human B cells.

FIG. 28 is a graph depicting the ability of afucosylated CD32b-binding antibody NOV1216 to modulate rituximab stimulated CD32b ITIM phosphorylation in primary human B cells, Daudi cells, and Karpas422 cells.

FIG. 29 is a graph depicting expression of CD32b on primary patient multiple myeloma samples, plasma B cells, and two established cell lines as assessed by flow cytometry.

FIG. 30 is a graph depicting the ability of Fc silent, Fc wildtype, and afucosylated versions of antibody NOV2108 compared to wildtype clone 10 antibody to mediate Daudi target cell killing by human NK cells.

FIG. 31 is a series of graphs depicting binding of NOV1216 and NOV2108 to WT huCD32b and huCD32b mutants.

FIG. 32 depicts a peptide coverage map for human CD32b construct (aa1-175) (SEQ ID NO: 682) as determined in deuterium exchange experiments to map putative binding site of CD32b antibody NOV2108. Each bar on the chart represents a peptide whose deuterium uptake was monitored.

FIG. 33 is a graph depicting differences in deuterium uptake for human CD32b and Ab NOV2108 Fab complex for amino acids 1 through 175.

FIG. 34 depicts the deuterium exchange protection site on human CD32b upon binding of Ab NOV2108 Fab mapped on the human CD32b crystal structure.

FIG. 35 is a graph depicting CDC activity of NOV2108 in an assay using KARPAS422 cells.

FIG. 36 is a series of graphs depicting cell surface CD32b expression analysis by flow cytometry.

FIG. 37 is a graph depicting sensitivity of Daudi cells compared to macrophages as target cells to NOV2108 Ab-mediated ADCC by NK cells.

FIG. 38 is a graph depicting quantification of cells phagocytosed by Cell tracker green labeled Macrophages over four hours. Replicate of 4 positions per well, per time frame were averaged.

FIG. 39a-FIG. 39c are a series of graphs depicting effect of Ab NOV2108 (WT and afucosylated) on B cells, monocytes, and granulocytes in a whole blood assay. Afucosylated NOV2108 enhances B-cell killing and retains viability of monocytes and granulocytes.

FIG. 40 is a graph depicting NOV2108 mediated lysis of multiple myeloma (MM) cell line Karpas620 by primary NK cells.

FIG. 41 is a graph depicting that Lenalidomide (LEN) treatment of PBMCs enhanced ADCC activity of NOV1216. Such enhancement was dramatically reduced when T cells were depleted from the PBMCs.

FIG. 42 is a graph depicting FACS assessment of CD32b expression on the KMS-12-BM multiple myeloma cell line.

FIG. 43 is a series of graphs depicting in vivo antitumor activity associated with combining an Fc enhanced anti-CD32b mAb and the HDAC inhibitor panobinostat in mice bearing CD32b low KMS-12-BM MM subcutaneous xenografts.

FIG. 44 is a graph depicting dose dependent anti-tumor activity of afucosylated NOV2108 administered intravenously to nude mice bearing subcutaneous Daudi xenografts.

FIG. 45 is a graph depicting antitumor activity of afucosylated NOV2108 in nude mice bearing subcutaneous xenografts of the KARPAS620 MM cell line.

FIG. 46 is a graph depicting the influence of intravenous eADCC Fc mutant NOV2108 administration on F4/80 positivity in Daudi xenografts subcutaneously engrafted in nude mice.

### DETAILED DESCRIPTION OF THE INVENTION

The present invention provides antibodies and antigen-binding fragments thereof that specifically bind to human CD32b protein, and pharmaceutical compositions, production methods, and methods of use of such antibodies and compositions.

### Definitions

Unless defined otherwise, all technical and scientific terms used herein have the same meaning as commonly understood by those of ordinary skill in the art to which this invention pertains.

“CD32A” or “CD32a”, as used herein, means human CD32a protein, also referred to as human FCγ Receptor 2A or FCγR2A or FCGR2a or FCGR2A. There are two variants known as H131 and R131 (when referenced without the signal sequence) or H167 and R167 (when referenced with the signal sequence). The amino acid sequence of the H167 variant is deposited under accession number UniProtKB P12318 and set forth below:

(SEQ ID NO: 677)
MTMETQMSQNVCPRNLWLLQPLTVLLLLASADSQAAAPPKAVLKLEPPWI
NVLQEDSVTLTCQGARSPESDSIQWFHNGNLIPTHTQPSYRFKANNNDSG
EYTCQTGQTSLSDPVHLTVLSEWLVLQTPHLEFQEGETIMLRCHSWKDKP
LVKVTFFQNGKSQKFSHLDPTFSIPQANHSHSGDYHCTGNIGYTLFSSKP
VTITVQVPSMGSSSPMGIIVAVVIATAVAAIVAAVVALIYCRKKRISANS
TDPVKAAQFEPPGRQMIAIRKRQLEETNNDYETADGGYMTLNPRAPTDDD
KNIYLTLPPNDHVNSNN.

“CD32B” or “CD32b”, as used herein, means human CD32b protein, also referred to as human FCγ Receptor 2B or FCγR2B or FCGR2b or FCGR2B. The amino acid sequence for CD32b variant 1 is deposited under accession number UniProtKB P31994-1 and set forth below:

(SEQ ID NO: 678)
MGILSFLPVLATESDWADCKSPQPWGHMLLWTAVLFLAPVAGTPAAPPKA
VLKLEPQWINVLQEDSVTLTCRGTHSPESDSIQWFHNGNLIPTHTQPSYR
FKANNNDSGEYTCQTGQTSLSDPVHLTVLSEWLVLQTPHLEFQEGETIVL
RCHSWKDKPLVKVTFFQNGKSKKFSRSDPNFSIPQANHSHSGDYHCTGNI
GYTLYSSKPVTITVQAPSSSPMGIIVAVVTGIAVAAIVAAVVALIYCRKK
RISALPGYPECREMGETLPEKPANPTNPDEADKVGAENTITYSLLMHPDA
LEEPDDQNRI.

The amino acid sequence for CD32b variant 2 is deposited under accession number UniProtKB P31994-2 and set forth below:

(SEQ ID NO: 679)
MGILSFLPVLATESDWADCKSPQPWGHMLLWTAVLFLAPVAGTPAAPPKA
VLKLEPQWINVLQEDSVTLTCRGTHSPESDSIQWFHNGNLIPTHTQPSYR
FKANNNDSGEYTCQTGQTSLSDPVHLTVLSEWLVLQTPHLEFQEGETIVL
RCHSWKDKPLVKVTFFQNGKSKKFSRSDPNFSIPQANHSHSGDYHCTGNI
GYTLYSSKPVTITVQAPSSSPMGIIVAVVTGIAVAAIVAAVVALIYCRKK
RISANPTNPDEADKVGAENTITYSLLMHPDALEEPDDQNRI.

As described herein, an antibody or antigen-binding fragment thereof which binds to CD32b binds to human CD32b protein. As used herein “huCD32b” refers to human CD32b protein or a fragment thereof.

The term “antibody” and the like, as used herein, include whole antibodies and any antigen-binding fragment (i.e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The terms “antigen-binding fragment”, “antigen-binding fragment thereof,” “antigen binding portion” of an antibody, and the like, as used herein, refer to one or more fragments of an intact antibody that retain the ability to specifically bind to a given antigen (e.g., CD32b). Antigen binding functions of an antibody can be performed by fragments of an intact antibody. Examples of binding fragments encompassed within the term “antigen binding portion” of an antibody include a Fab fragment, a monovalent fragment consisting of the VL, VH, CL and CH1 domains; a F (ab)₂ fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., 1989 Nature 341:544-546), which consists of a VH domain; and an isolated complementarity determining region (CDR).

Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more “antigen binding portions” of an antibody. These antibody fragments are obtained using conventional techniques known to those of skill in the art, and the fragments are screened for utility in the same manner as are intact antibodies.

Antigen binding portions can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies).

Antigen binding portions can be incorporated into single chain molecules comprising a pair of tandem Fv segments (VH-CH1-VH-CH1) which, together with complementary light chain polypeptides, form a pair of antigen binding regions (Zapata et al., 1995 Protein Eng. 8 (10):1057-1062; and U.S. Pat. No. 5,641,870).

As used herein, the term “Affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity.

As used herein, the term “Avidity” refers to an informative measure of the overall stability or strength of the antibody-antigen complex. It is controlled by three major factors: antibody epitope affinity; the valency of both the antigen and antibody; and the structural arrangement of the interacting parts. Ultimately these factors define the specificity of the antibody, that is, the likelihood that the particular antibody is binding to a precise antigen epitope.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “binding specificity” as used herein refers to the ability of an individual antibody combining site to react with one antigenic determinant and not with a different antigenic determinant. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody.

Specific binding between two entities means a binding with an equilibrium constant (KA or K_{A}) of at least 1×10⁷ M⁻¹, 10⁸ M⁻¹, 10⁹ M⁻¹, 10¹⁰ M⁻¹, 10¹¹ M⁻¹, 10¹² M⁻¹, 10¹³ M⁻¹, or 10¹⁴M⁻¹. The phrase “specifically (or selectively) binds” to an antigen (e.g., CD32b-binding antibody) refers to a binding reaction that is determinative of the presence of a cognate antigen (e.g., a human CD32b protein) in a heterogeneous population of proteins and other biologics. A CD32b-binding antibody of the invention binds to CD32b with a greater affinity than it does to a non-specific antigen (e.g., CD32a). The phrases “an antibody recognizing an antigen” and “an antibody specific for an antigen” are used interchangeably herein with the term “an antibody which binds specifically to an antigen”.

The term “chimeric antibody” (or antigen-binding fragment thereof) is an antibody molecule (or antigen-binding fragment thereof) in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In one embodiment, the term “conservative sequence modifications” are used to refer to amino acid modifications that do not significantly affect or alter the binding characteristics of the antibody containing the amino acid sequence.

The term “blocks” as used herein refers to stopping or preventing an interaction or a process, e.g., stopping ligand-dependent or ligand-independent signaling.

The term “recognize” as used herein refers to an antibody antigen-binding fragment thereof that finds and interacts (e.g., binds) with its conformational epitope.

The terms “cross-block”, “cross-blocked”, “cross-blocking”, “compete”, “cross compete” and related terms are used interchangeably herein to mean the ability of an antibody or other binding agent to interfere with the binding of other antibodies or binding agents to CD32b in a standard competitive binding assay.

The ability or extent to which an antibody or other binding agent is able to interfere with the binding of another antibody or binding molecule to CD32b, and therefore whether it can be said to cross-block according to the invention, can be determined using standard competition binding assays. One suitable assay involves the use of the Biacore technology (e.g. by using the BIAcore 3000 instrument (Biacore, Uppsala, Sweden)), which can measure the extent of interactions using surface plasmon resonance technology. Another assay for measuring cross-blocking uses an ELISA-based approach. Although the techniques are expected to produce substantially similar results, measurement by the Biacore technique is considered definitive.

The term “neutralizes” means that an antibody, upon binding to its target, reduces the activity, level or stability of the target; e.g., a CD32b antibody, upon binding to CD32b neutralizes CD32b by at least partially reducing an activity, level or stability of CD32b, such as its role in engaging Fc portions of antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “epitope” includes any protein determinant capable of specific binding to an immunoglobulin or otherwise interacting with a molecule. Epitopic determinants generally consist of chemically active surface groupings of molecules such as amino acids or carbohydrate or sugar side chains and can have specific three-dimensional structural characteristics, as well as specific charge characteristics. An epitope may be “linear” or “conformational.”

The term “linear epitope” refers to an epitope with all of the points of interaction between the protein and the interacting molecule (such as an antibody) occurring linearally along the primary amino acid sequence of the protein (continuous).

As used herein, the term “high affinity” for an IgG antibody refers to an antibody having a KD of 10⁻⁸M or less, 10⁻⁹ M or less, or 10⁻¹⁰ M, or 10⁻¹¹M or less for a target antigen, e.g., CD32b. However, “high affinity” binding can vary for other antibody isotypes. For example, “high affinity” binding for an IgM isotype refers to an antibody having a KD of 10⁻⁷ M or less, or 10⁻⁸M or less.

The term “human antibody” (or antigen-binding fragment thereof), as used herein, is intended to include antibodies (and antigen-binding fragments thereof) having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies and antigen-binding fragments thereof of the invention may include amino acid residues not encoded by human sequences (e.g., mutations introduced by random or site-specific mutagenesis in vitro or by somatic mutation in vivo).

The phrases “monoclonal antibody” or “monoclonal antibody composition” (or antigen-binding fragment thereof) as used herein refers to polypeptides, including antibodies, antibody fragments, bispecific antibodies, etc. that have substantially identical to amino acid sequence or are derived from the same genetic source. This term also includes preparations of antibody molecules of single molecular composition. A monoclonal antibody composition displays a single binding specificity and affinity for a particular epitope.

The term “human monoclonal antibody” (or antigen-binding fragment thereof) refers to antibodies (and antigen-binding fragments thereof) displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The phrase “recombinant human antibody” (or antigen-binding fragment thereof), as used herein, includes all human antibodies (and antigen-binding fragments thereof) that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In one embodiment, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “humanized” antibody (or antigen-binding fragment thereof), as used herein, is an antibody (or antigen-binding fragment thereof) that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlan, Molec. Immun, 28:489-498, 1991; and Padlan, Molec. Immun, 31:169-217, 1994. Other examples of human engineering technology include, but is not limited to Xoma technology disclosed in U.S. Pat. No. 5,766,886.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, 96%, 97%, 98% or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length. Optionally, the identity exists over a region that is at least 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, John Wiley & Sons, Inc. (ringbou ed., 2003)).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (N) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “isolated antibody” (or antigen-binding fragment thereof), as used herein, refers to an antibody (or antigen-binding fragment thereof) that is substantially free of other antibodies having different antigenic specificities (e.g., an isolated antibody that specifically binds CD32b is substantially free of antibodies that specifically bind antigens other than CD32b). Moreover, an isolated antibody may be substantially free of other cellular material and/or chemicals.

The term “isotype” refers to the antibody class (e.g., IgM, IgE, IgG such as IgG1 or IgG4) that is provided by the heavy chain constant region genes. Isotype also includes modified versions of one of these classes, where modifications have been made to after the Fc function, for example, to enhance or reduce effector functions or binding to Fc receptors.

The term “Kassoc”, “Ka” or “Kₒₙ”, as used herein, is intended to refer to the association rate of a particular antibody-antigen interaction, whereas the term “Kdis”, “Kd,” or “K_{off}”, as used herein, is intended to refer to the dissociation rate of a particular antibody-antigen interaction. In one embodiment, the term “KD”, as used herein, is intended to refer to the dissociation constant, which is obtained from the ratio of Kd to Ka (i.e. Kd/Ka) and is expressed as a molar concentration (M). KD values for antibodies can be determined using methods well established in the art. A method for determining the KD of an antibody is by using surface plasmon resonance, or using a biosensor system such as a Biacore® system. Where the dissociation constant is less than about 10⁻⁹ M, solution equilibrium kinetic exclusion KD measurement (MSD-SET) is a preferred method for determining the KD of an antibody (see, e.g., Friquet, B., Chaffotte, A. F., Djavadi-Ohaniance, L., and Goldberg, M. E. (1985). Measurements of the true affinity constant in solution of antigen-antibody complexes by enzyme-linked immunosorbent assay J Immnunol Meth 77, 305-319; herein incorporated by reference).

The term “IC50,” as used herein, refers to the concentration of an antibody or an antigen-binding fragment thereof, which induces an inhibitory response, either in an in vitro or an in vivo assay, which is 50% of the maximal response, i.e., halfway between the maximal response and the baseline.

The terms “monoclonal antibody” (or antigen-binding fragment thereof) or “monoclonal antibody (or antigen-binding fragment thereof) composition” as used herein refer to a preparation of an antibody molecule (or antigen-binding fragment thereof) of single molecular composition. A monoclonal antibody composition displays a single binding specificity and affinity for a particular epitope.

The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonisation and lysis of cell pathogens. The activation of complement also stimulates the inflammatory response and may also be involved in autoimmune hypersensitivity. Effector function also includes Fc receptor (FcR)-mediated effector function, which may be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody may be altered by altering, e.g., enhancing or reducing, the affinity of the antibody for an effector molecule such as an Fc receptor or a complement component. Binding affinity will generally be varied by modifying the effector molecule binding site, and in this case it is appropriate to locate the site of interest and modify at least part of the site in a suitable way. It is also envisaged that an alteration in the binding site on the antibody for the effector molecule need not alter significantly the overall binding affinity but may alter the geometry of the interaction rendering the effector mechanism ineffective as in non-productive binding. It is further envisaged that an effector function may also be altered by modifying a site not directly involved in effector molecule binding, but otherwise involved in performance of the effector function.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “recombinant human antibody” (or antigen-binding fragment thereof), as used herein, includes all human antibodies (and antigen-binding fragments thereof) that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In one embodiment, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “treat,” “treated,” “treating,” and “treatment,” include the administration of compositions or antibodies to prevent or delay the onset of the symptoms, complications, or biochemical indicia of a disease, alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease. Treatment can be measured by the therapeutic measures described herein. The methods of “treatment” of the present invention include administration of a CD32b antibody or antigen binding fragment thereof to a subject in order to cure, reduce the severity of, or ameliorate one or more symptoms of a fibrotic disease or condition, in order to prolong the health or survival of a subject beyond that expected in the absence of such treatment. For example, “treatment” includes the alleviation of a disease symptom in a subject by at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or more.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### Cd32B Antibodies and Antigen-Binding Fragments Thereof

The present invention provides antibodies and antigen-binding fragments thereof that specifically bind to human CD32b.

In one embodiment, the present invention provides isolated antibodies or antigen-binding fragments thereof that bind with a higher affinity for human CD32b protein, than to human CD32a protein. Selectivity for CD32b over CD32a is desired to ensure selective binding to CD32b positive B-cell malignancies and B-cells while lacking binding to CD32a positive immune cells, including monocytes and neutrophils.

Antibodies of the invention include, but are not limited to, the human and humanized monoclonal antibodies isolated as described herein, including in the Examples.

Examples of such anti-human CD32b antibodies are antibodies NOV0281, NOV0308, NOV0563, NOV1216, NOV1218, NOV1219, NOV2106, NOV2107, NOV2108, NOV2109, NOV2110, NOV2111, NOV2112, and NOV2113 (including antibodies with wild type Fc regions or containing the N297A mutation in the Fc region) whose sequences are listed in Table 1. Additional details regarding the generation and characterization of the antibodies described herein are provided in the Examples.

The present invention provides antibodies that specifically bind CD32b (e.g., human CD32b protein), said antibodies comprising a VH domain listed in Table 1. The present invention also provides antibodies that specifically bind to CD32b protein, said antibodies comprising a VH CDR having an amino acid sequence of any one of the VH CDRs listed in Table 1. In particular, the invention provides antibodies that specifically bind to CD32b protein, said antibodies comprising (or alternatively, consisting of) one, two, three, four, five or more VH CDRs having an amino acid sequence of any of the VH CDRs listed in Table 1.

The invention also provides antibodies and antigen-binding fragments thereof that specifically bind to CD32b, said antibodies or antigen-binding fragments thereof comprising (or alternatively, consisting of) a VH amino acid sequence listed in Table 1, wherein no more than about 10 amino acids in a framework sequence (for example, a sequence which is not a CDR) have been mutated (wherein a mutation is, as various non-limiting examples, an addition, substitution or deletion). The invention also provides antibodies and antigen-binding fragments thereof that specifically bind to CD32b, said antibodies or antigen-binding fragments thereof comprising (or alternatively, consisting of) a VH amino acid sequence listed in Table 1, wherein no more than 10 amino acids in a framework sequence (for example, a sequence which is not a CDR) have been mutated (wherein a mutation is, as various non-limiting examples, an addition, substitution or deletion).

The invention also provides antibodies and antigen-binding fragments thereof that specifically bind to CD32b, said antibodies or antigen-binding fragments thereof comprising (or alternatively, consisting of) a VH amino acid sequence listed in Table 1, wherein no more than about 20 amino acids in a framework sequence (for example, a sequence which is not a CDR) have been mutated (wherein a mutation is, as various non-limiting examples, an addition, substitution or deletion). The invention also provides antibodies and antigen-binding fragments thereof that specifically bind to CD32b, said antibodies or antigen-binding fragments thereof comprising (or alternatively, consisting of) a VH amino acid sequence listed in Table 1, wherein no more than 20 amino acids in a framework sequence (for example, a sequence which is not a CDR) have been mutated (wherein a mutation is, as various non-limiting examples, an addition, substitution or deletion).

The present invention provides antibodies and antigen-binding fragments thereof that specifically bind to CD32b protein, said antibodies or antigen-binding fragments thereof comprising a VL domain listed in Table 1. The present invention also provides antibodies and antigen-binding fragments thereof that specifically bind to CD32b protein, said antibodies or antigen-binding fragments thereof comprising a VL CDR having an amino acid sequence of any one of the VL CDRs listed in Table 1. In particular, the invention provides antibodies and antigen-binding fragments thereof that specifically bind to CD32b protein, said antibodies or antigen-binding fragments thereof comprising (or alternatively, consisting of) one, two, three or more VL CDRs having an amino acid sequence of any of the VL CDRs listed in Table 1.

The invention also provides antibodies and antigen-binding fragments thereof that specifically bind to CD32b, said antibodies or antigen-binding fragments thereof comprising (or alternatively, consisting of) a VL amino acid sequence listed in Table 1, wherein no more than about 10 amino acids in a framework sequence (for example, a sequence which is not a CDR) have been mutated (wherein a mutation is, as various non-limiting examples, an addition, substitution or deletion). The invention also provides antibodies and antigen-binding fragments thereof that specifically bind to CD32b, said antibodies or antigen-binding fragments thereof comprising (or alternatively, consisting of) a VL amino acid sequence listed in Table 1, wherein no more than 10 amino acids in a framework sequence (for example, a sequence which is not a CDR) have been mutated (wherein a mutation is, as various non-limiting examples, an addition, substitution or deletion).

The invention also provides antibodies and antigen-binding fragments thereof that specifically bind to CD32b, said antibodies or antigen-binding fragments thereof comprising (or alternatively, consisting of) a VL amino acid sequence listed in Table 1, wherein no more than about 20 amino acids in a framework sequence (for example, a sequence which is not a CDR) have been mutated (wherein a mutation is, as various non-limiting examples, an addition, substitution or deletion). The invention also provides antibodies and antigen-binding fragments thereof that specifically bind to CD32b, said antibodies or antigen-binding fragments thereof comprising (or alternatively, consisting of) a VL amino acid sequence listed in Table 1, wherein no more than 20 amino acids in a framework sequence (for example, a sequence which is not a CDR) have been mutated (wherein a mutation is, as various non-limiting examples, an addition, substitution or deletion).

Other antibodies and antigen-binding fragments thereof of the invention include amino acids that have been mutated, yet have at least 60, 70, 80, 90, 91, 92, 93, 94, 95, 96, 97, 98 or 99 percent identity in the CDR regions with the CDR regions depicted in the sequences described in Table 1. In one aspect, other antibodies and antigen-binding fragments thereof of the invention includes mutant amino acid sequences wherein no more than 1, 2, 3, 4 or 5 amino acids have been mutated in the CDR regions when compared with the CDR regions depicted in the sequence described in Table 1.

The present invention also provides nucleic acid sequences that encode VH, VL, the full length heavy chain, and the full length light chain of the antibodies and antigen-binding fragments thereof that specifically bind to CD32b protein. Such nucleic acid sequences can be optimized for expression in mammalian cells (for example, Table 1 shows example nucleic acid sequences for the heavy chain (including sequences for antibodies having a wild type Fc region or containing the N297A mutation in the Fc region) and light chain of Antibodies NOV0281, NOV0308, NOV0563, NOV1216, NOV1218, NOV1219, NOV2106, NOV2107, NOV2108, NOV2109, NOV2110, NOV2111, NOV2112, and NOV2113).

Throughout the text of this application, should there be a discrepancy between the text of the specification (e.g., Table 1) and the sequence listing, the text of the specification shall prevail.

TABLE 1
Examples of CD32bAntibodies of the Present Invention

| | | |
|---|---|---|
| SEQ ID | | |
| NO: | Description | Sequence |

NOV0281

| | | |
|---|---|---|
| 1 | HCDR1 | GGTFSDYAIS |
| | (Combined) | |
| 2 | HCDR2 | GIIPISGTANYAQKFQG |
| | (Combined) | |
| 3 | HCDR3 | DHSSSSYDYQYGLAV |
| | (Combined) | |
| 4 | HCDR1 | DYAIS |
| | (Kabat) | |
| 5 | HCDR2 | GIIPISGTANYAQKFQG |
| | (Kabat) | |
| 6 | HCDR3 | DHSSSSYDYQYGLAV |
| | (Kabat) | |
| 7 | HCDR1 | GGTFSDY |
| | (Chothia) | |
| 8 | HCDR2 | IPISGT |
| | (Chothia) | |
| 9 | HCDR3 | DHSSSSYDYQYGLAV |
| | (Chothia) | |
| 10 | VH | QVQLVQSGAEVKKPGSSVKVSCKASGGTFSDYAISVWRQAPGQ |
| | | GLEWMGGIIPISGTANYAQKFQGRVTITADESTSTAYMELSSLRS |
| | | EDTAVYYCARDHSSSSYDYQYGLAVWGQGTLVTVSS |
| 11 | DNA VH | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGCGACTACGCTATTAGCTGGGTCAGACAGGCCCCAGG |
| | | TCAGGGCCTGGAGTGGATGGGCGGAATTATCCCTATTAGCGG |
| | | CACCGCTAACTACGCTCAGAAATTTCAGGGTAGAGTGACTATC |
| | | ACCGCCGACGAGTCTACTAGCACCGCCTATATGGAACTGTCT |
| | | AGCCTGAGATCAGAGGACACCGCCGTCTACTACTGCGCTAGA |
| | | GATCACTCTAGCTCTAGCTACGACTATCAGTACGGCCTGGCC |
| | | GTGTGGGGTCAGGGCACCCTGGTCACCGTGTCTAGC |
| 12 | Heavy Chain | QVQLVQSGAEVKKPGSSVKVSCKASGGTFSDYAISVWRQAPGQ |
| | | GLEWMGGIIPISGTANYAQKFQGRVTITADESTSTAYMELSSLRS |
| | | EDTAVYYCARDHSSSSYDYQYGLAVWGQGTLVTVSSASTKGPS |
| | | VFPLAPSSKSTSGGTAALGCLVKDYFPEPVTVSWNSGALTSGVH |
| | | TFPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVDVSHEDPEVKFNWYVDGVEVHNAKTKPREEQYNSTYR |
| | | VVSVLTVLHQDWLNGKEYKCKVSNKALPAPIEKTISKAKGQPREP |
| | | QVYTLPPSREEMTKNQVSLTCLVKGFYPSDIAVEWESNGQPENN |
| | | YKTTPPVLDSDGSFFLYSKLTVDKSRWGQGNVFSCSVMHEALH |
| | | NHYTQKSLSLSPGK |
| 13 | DNA Heavy | CAGGTGCAGCTGGTGCAGTCAGGCGCCGAAGTGAAGAAACC |
| | Chain | CGGCTCTAGCGTGAAAGTCAGCTGTAAAGCTAGTGGCGGCAC |
| | | CTTTAGCGACTACGCTATTAGCTGGGTCAGACAGGCCCCAGG |
| | | TCAGGGCCTGGAGTGGATGGGCGGAATTATCCCTATTAGCGG |
| | | CACCGCTAACTACGCTCAGAAATTTCAGGGTAGAGTGACTATC |
| | | ACCGCCGACGAGTCTACTAGCACCGCCTATATGGAACTGTCT |
| | | AGCCTGAGATCAGAGGACACCGCCGTCTACTACTGCGCTAGA |
| | | GATCACTCTAGCTCTAGCTACGACTATCAGTACGGCCTGGCC |
| | | GTGTGGGGTCAGGGCACCCTGGTCACCGTGTCTAGCGCTAG |
| | | CACTAAGGGCCCAAGTGTGTTTCCCCTGGCCCCCAGCAGCAA |
| | | GTCTACTTCCGGCGGAACTGCTGCCCTGGGTTGCCTGGTGAA |
| | | GGACTACTTCCCCGAGCCCGTGACAGTGTCCTGGAACTCTGG |
| | | GGCTCTGACTTCCGGCGTGCACACCTTCCCCGCCGTGCTGCA |
| | | GAGCAGCGGCCTGTACAGCCTGAGCAGCGTGGTGACAGTGC |
| | | CCTCCAGCTCTCTGGGAACCCAGACCTATATCTGCAACGTGA |
| | | ACCACAAGCCCAGCAACACCAAGGTGGACAAGAGAGTGGAG |
| | | CCCAAGAGCTGCGACAAGACCCACACCTGCCCCCCCTGCCC |
| | | AGCTCCAGAACTGCTGGGAGGGCCTTCCGTGTTCCTGTTCCC |
| | | CCCCAAGCCCAAGGACACCCTGATGATCAGCAGGACCCCCG |
| | | AGGTGACCTGCGTGGTGGTGGACGTGTCCCACGAGGACCCA |
| | | GAGGTGAAGTTCAACTGGTACGTGGACGGCGTGGAGGTGCA |
| | | CAACGCCAAGACCAAGCCCAGAGAGGAGCAGTACAACAGCA |
| | | CCTACAGGGTGGTGTCCGTGCTGACCGTGCTGCACCAGGACT |
| | | GGCTGAACGGCAAAGAATACAAGTGCAAAGTCTCCAACAAGG |
| | | CCCTGCCAGCCCCAATCGAAAAGACAATCAGCAAGGCCAAGG |
| | | GCCAGCCACGGGAGCCCCAGGTGTACACCCTGCCCCCCAGC |
| | | CGGGAGGAGATGACCAAGAACCAGGTGTCCCTGACCTGTCTG |
| | | GTGAAGGGCTTCTACCCCAGCGATATCGCCGTGGAGTGGGA |
| | | GAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTC |
| | | AGCTGCAGCGTGATGCACGAGGCCCTGCACAACCACTACACC |
| | | CAGAAGTCCCTGAGCCTGAGCCCCGGCAAG |
| 14 | LCDR1 | SGDKLGDYYVH |
| | (Combined) | |
| 15 | LCDR2 | QDSKRPS |
| | (Combined) | |
| 16 | LCDR3 | GATDLSPWSIV |
| | (Combined) | |
| 17 | LCDR1 | SGDKLGDYYVH |
| | (Kabat) | |
| 18 | LCDR2 | QDSKRPS |
| | (Kabat} | |
| 19 | LCDR3 | GATDLSPWSIV |
| | (Kabat) | |
| 20 | LCDR1 | DKLGDYY |
| | (Chothia) | |
| 21 | LCDR2 | QDS |
| | (Chothia) | |
| 22 | LCDR3 | TDLSPWSI |
| | (Chothia) | |
| 23 | VL | DIELTQPPSVSVSPGETASITCSGDKLGDYYVHWYQQKPGGAPV |
| | | LVIYQDSKRPSGIPERFSGSNSGNTATLTISGTQAEDEADYYCGA |
| | | TDLSPWSIVFGGGTKLTVL |
| 24 | DNA VL | GATATCGAGCTGACTCAGCCCCCTAGCGTCAGCGTCAGCCCT |
| | | GGCGAGACAGCCTCTATCACCTGTAGCGGCGATAAGCTGGG |
| | | CGACTACTACGTGCACTGGTATCAGCAGAAGCCCGGTCAGGC |
| | | CCCCGTGCTGGTGATCTATCAGGACTCTAAGCGGCCTAGCGG |
| | | AATCCCCGAGCGGTTTAGCGGCTCTAATAGCGGTAACACCGC |
| | | TACCCTGACTATTAGCGGCACTCAGGCCGAGGACGAGGCCG |
| | | ACTACTACTGCGGCGCTACCGACCTGAGCCCCTGGTCTATCG |
| | | TGTTCGGCGGAGGCACTAAGCTGACCGTGCTG |
| 25 | Light Chain | DIELTQPPSVSVSPGETASITCSGDKLGDYYVHWYQQKPGQAPV |
| | | LVIYQDSKRPSGIPERFSGSNSGNTATLTISGTQAEDEADYYCGA |
| | | TDLSPWSIVFGGGTKLTVLGQPKAAPSVTLFPPSSEELQANKATL |
| | | VCLISDFYPGAVTVAWKADSSPVKAGVETTTPSKQSNNKYAASS |
| | | YLSLTPEQWKSHRSYSCQVTHEGSTVEKTVAPTECS |
| 26 | DNA Light | GATATCGAGCTGACTCAGCCCCCTAGCGTCAGCGTCAGCCCT |
| | Chain | GGCGAGACAGCCTCTATCACCTGTAGCGGCGATAAGCTGGG |
| | | CGACTACTACGTGCACTGGTATCAGCAGAAGCCCGGTCAGGC |
| | | CCCCGTGCTGGTGATCTATCAGGACTCTAAGCGGCCTAGCGG |
| | | AATCCCCGAGCGGTTTAGCGGCTCTAATAGCGGTAACACCGC |
| | | TACCCTGACTATTAGCGGCACTCAGGCCGAGGACGAGGCCG |
| | | ACTACTACTGCGGCGCTACCGACCTGAGCCCCTGGTCTATCG |
| | | TGTTCGGCGGAGGCACTAAGCTGACCGTGCTGGGTCAGCCTA |
| | | AGGCTGCCCCCAGCGTGACCCTGTTCCCCCCCAGCAGCGAG |
| | | GAGCTGCAGGCCAACAAGGCCACCCTGGTGTGCCTGATCAG |
| | | CGACTTCTACCCAGGCGCCGTGACCGTGGCCTGGAAGGCCG |
| | | ACAGCAGCCCCGTGAAGGCCGGCGTGGAGACCACCACCCCC |
| | | AGCAAGCAGAGCAACAACAAGTACGCCGCCAGCAGCTACCTG |
| | | AGCCTGACCCCCGAGCAGTGGAAGAGCCACAGGTCCTACAG |
| | | CTGCCAGGTGACCCACGAGGGCAGCACCGTGGAAAAGACCG |
| | | TGGCCCCAACCGAGTGCAGC |

NOV0281_N297A

| | | |
|---|---|---|
| 27 | HCDR1 | GGTFSDYAIS |
| | (Combined) | |
| 28 | HCDR2 | GIIPISGTANYAQKFQG |
| | (Combined) | |
| 29 | HCDR3 | DHSSSSYDYQYGLAV |
| | (Combined) | |
| 30 | HCDR1 | DYAIS |
| | (Kabat) | |
| 31 | HCDR2 | GIIPISGTANYAQKFQG |
| | (Kabat) | |
| 32 | HCDR3 | DHSSSSYDYQYGLAV |
| | (Kabat) | |
| 33 | HCDR1 | GGTFSDY |
| | (Chothia) | |
| 34 | HCDR2 | IPISGT |
| | (Chothia) | |
| 35 | HCDR3 | DHSSSSYDYQYGLAV |
| | (Chothia) | |
| 36 | VH | QVQLVQSGAEVKKPGSSVKVSCKASGGTFSDYAISWVRQAPGQ |
| | | GLEWMGGIIPISGTANYAQKFQGRVTITADESTSTAYMELSSLRS |
| | | EDTAVYYCARDHSSSSYDYQYGLAVWGQGTLVTVSS |
| 37 | DNA VH | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTTCTGACTACGCTATCTCTTGGGTGCGCCAGGCCCCGGG |
| | | CCAGGGCCTCGAGTGGATGGGCGGTATCATCCCGATCTCTG |
| | | GCACTGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGACCATTCTTCTTCTTCTTACGACTACCAGTACGGTCTGGC |
| | | TGTTTGGGGCCAAGGCACCCTGGTGACTGTTAGCTCA |
| 38 | Heavy | QVQLVGSGAEVKKPGSSVKVSCKASGGTFSDYAISVWRQAPGQ |
| | Chain | GLEWMGGIIPISGTANYAQKFQGRVTITADESTSTAYMELSSLRS |
| | | EDTAVYYCARDHSSSSYDYQYGLAVWGQGTLVTVSSASTKGPS |
| | | VFPLAPSSKSTSGGTAALGCLVKDYFPEPVTVSWNSGALTSGVH |
| | | TFPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVDVSHEDPEVKFNWYVDGVEVHNAKTKPREEQYASTYR |
| | | VVSVLTVLHQDWLNGKEYKCKVSNKALPAPIEKTISKAKGQPREP |
| | | QVYTLPPSREEMTKNQVSLTCLVKGFYPSDIAVEWESNGQPENN |
| | | YKTTPPVLDSDGSFFLYSKLTVDKSRWQQGNVFSCSVMHEALH |
| | | NHYTQKSLSLSPGK |
| 39 | DNA Heavy | CAGGTGCAATTGGTGCAGAGCGGTGCCGAAGTGAAAAAACCG |
| | Chain | GGCAGCAGCGTGAAAGTTAGCTGCAAAGCATCCGGAGGGAC |
| | | GTTTTCTGACTACGCTATCTCTTGGGTGCGCCAGGCCCCGGG |
| | | CCAGGGCCTCGAGTGGATGGGCGGTATCATCCCGATCTCTG |
| | | GCACTGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGACCATTCTTCTTCTTCTTACGACTACCAGTACGGTCTGGC |
| | | TGTTTGGGGCCAAGGCACCCTGGTGACTGTTAGCTCAGCCTC |
| | | CACCAAGGGTCCATCGGTCTTCCCCCTGGCACCCTCCTCCAA |
| | | GAGCACCTCTGGGGGCACAGCGGCCCTGGGCTGCCTGGTCA |
| | | AGGACTACTTCCCCGAACCGGTGACGGTGTCGTGGAACTCAG |
| | | GCGCCCTGACCAGCGGCGTGCACACCTTCCCGGCTGTCCTA |
| | | CAGTCCTCAGGACTCTACTCCCTCAGCAGCGTGGTGACCGTG |
| | | CCCTCCAGCAGCTTGGGCACCCAGACCTACATCTGCAACGTG |
| | | AATCACAAGCCCAGCAACACCAAGGTGGACAAGAGAGTTGAG |
| | | CCCAAATCTTGTGACAAAACTCACACATGCCCACCGTGCCCA |
| | | GCACCTGAACTCCTGGGGGGACCGTCAGTCTTCCTCTTCCCC |
| | | CCAAAACCCAAGGACACCCTCATGATCTCCCGGACCCCTGAG |
| | | GTCACATGCGTGGTGGTGGACGTGAGCCACGAAGACCCTGA |
| | | GGTCAAGTTCAACTGGTACGTGGACGGCGTGGAGGTGCATAA |
| | | TGCCAAGACAAAGCCGCGGGAGGAGCAGTACGCCAGCACGT |
| | | ACCGGGTGGTCAGCGTCCTCACCGTCCTGCACCAGGACTGG |
| | | CTGAATGGCAAGGAGTACAAGTGCAAGGTCTCCAACAAAGCC |
| | | CTCCCAGCCCCCATCGAGAAAACCATCTCCAAAGCCAAAGGG |
| | | CAGCCCCGAGAACCACAGGTGTACACCCTGCCCCCATCCCG |
| | | GGAGGAGATGACCAAGAACCAGGTCAGCCTGACCTGCCTGG |
| | | TCAAAGGCTTCTATCCCAGCGACATCGCCGTGGAGTGGGAGA |
| | | GCAATGGGCAGCCGGAGAACAACTACAAGACCACGCCTCCC |
| | | GTGCTGGACTCCGACGGCTCCTTCTTCCTCTACAGCAAGCTC |
| | | ACCGTGGACAAGAGCAGGTGGCAGCAGGGGAACGTCTTCTC |
| | | ATGCTCCGTGATGCATGAGGCTCTGCACAACCACTACACGCA |
| | | GAAGAGCCTCTCCCTGTCTCCGGGTAAA |
| 40 | LCDR1 | SGDKLGDYYVH |
| | (Combined) | |
| 41 | LCDR2 | QDSKRPS |
| | (Combined) | |
| 42 | LCDR3 | GATDLSPWSIV |
| | (Combined) | |
| 43 | LCDR1 | SGDKLGDYYVH |
| | (Kabat) | |
| 44 | LCDR2 | QDSKRPS |
| | (Kabat) | |
| 45 | LCDR3 | GATDLSPWSIV |
| | (Kabat) | |
| 46 | LCDR1 | DKLGDYY |
| | (Chothia) | |
| 47 | LCDR2 | QDS |
| | (Chothia) | |
| 48 | LCDR3 | TDLSPWSI |
| | (Chothia) | |
| 49 | VL | DIELTQPPSVSVSPGETASITCSGDKLGDYYVHVWQQKPGQAPV |
| | | LVIYQDSKRPSGIPERFSGSNSGNTATLTISGTQAEDEADYYCGA |
| | | TDLSPWSIVFGGGTKLTVL |
| 50 | DNA VL | GATATCGAACTGACCCAGCCGCCGAGCGTGAGCGTGAGCCC |
| | | GGGCGAGACCGCGAGCATTACCTGTAGCGGCGATAAACTGG |
| | | GTGACTACTACGTTCATTGGTACCAGCAGAAACCGGGCCAGG |
| | | CGCCGGTGCTGGTGATCTACCAGGACTCTAAACGTCCGAGCG |
| | | GCATCCCGGAACGTTTTAGCGGATCCAACAGCGGCAACACCG |
| | | CGACCCTGACCATTAGCGGCACCCAGGCGGAAGACGAAGCG |
| | | GATTATTACTGCGGTGCTACTGACCTGTCTCCGTGGTCTATCG |
| | | TGTTTGGCGGCGGCACGAAGTTAACCGTCCTA |
| 51 | Light Chain | DIELTQPPSVSVSPGETASITCSGDKLGDYYVHWYQQKPGQAPV |
| | | LVIYQDSKRPSGIPERFSGSNSGNTATLTISGTQAEDEADYYCGA |
| | | TDLSPWSIVFGGGTKLTVLGQPKAAPSVTLFPPSSEELQANKATL |
| | | VCLISDFYPGAVTVAWKADSSPVKAGVETTTPSKQSNNKYAASS |
| | | YLSLTPEQWKSMRSYSCQVTHEGSTVEKTVAPTECS |
| 52 | DNA Light | GATATCGAACTGACCCAGCCGCCGAGCGTGAGCGTGAGCCC |
| | Chain | GGGCGAGACCGCGAGCATTACCTGTAGCGGCGATAAACTGG |
| | | GTGACTACTACGTTCATTGGTACCAGCAGAAACCGGGCCAGG |
| | | CGCCGGTGCTGGTGATCTACCAGGACTCTAAACGTCCGAGCG |
| | | GCATCCCGGAACGTTTTAGCGGATCCAACAGCGGCAACACCG |
| | | CGACCCTGACCATTAGCGGCACCCAGGCGGAAGACGAAGCG |
| | | GATTATTACTGCGGTGCTACTGACCTGTCTCCGTGGTCTATCG |
| | | TGTTTGGCGGCGGCACGAAGTTAACCGTCCTAGGTCAGCCCA |
| | | AGGCTGCCCCCTCGGTCACTCTGTTCCCGCCCTCCTCTGAGG |
| | | AGCTTCAAGCCAACAAGGCCACACTGGTGTGTCTCATAAGTG |
| | | ACTTCTACCCGGGAGCCGTGACAGTGGCCTGGAAGGCAGATA |
| | | GCAGCCCCGTCAAGGCGGGAGTGGAGACCACCACACCCTCC |
| | | AAACAAAGCAACAACAAGTACGCGGCCAGCAGCTATCTGAGC |
| | | CTGACGCCTGAGCAGTGGAAGTCCCACAGAAGCTACAGCTGC |
| | | CAGGTCACGCATGAAGGGAGCACCGTGGAGAAGACAGTGGC |
| | | CCCTACAGAATGTTCA |

NOV0308

| | | |
|---|---|---|
| 53 | HCDR1 | GGTFSSYAIS |
| | (Combined) | |
| 54 | HCDR2 | GIIPVLGTANYAQKFQG |
| | (Combined) | |
| 55 | HCDR3 | VPTDYFDY |
| | (Combined) | |
| 56 | HCDR1 | SYAIS |
| | (Kabat) | |
| 57 | HCDP2 | GIIPVLGTANYAQKFQG |
| | (Kabat) | |
| 58 | HCDR3 | VPTDYFDY |
| | (Kabat) | |
| 59 | HCDR1 | GGTFSSY |
| | (Chothia) | |
| 60 | HCDR2 | IPVLGT |
| | (Chothia) | |
| 61 | HCDR3 | VPTDYFDY |
| | (Chothia) | |
| 62 | VH | QVQLVQSGAEVKKPGSSVKVSCKASGGTFSSYAISVWRQAPGQ |
| | | GLEWMGGIIPVLGTANYAQKFQGRVTITADESTSTAYMELSSLRS |
| | | EDTAVYYCARVPTDYFDYWGQGTLVTVSS |
| 63 | DNA VH | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTCTCTAGCTACGCTATTAGCTGGGTCAGACAGGCCCCAGG |
| | | TCAGGGCCTGGAGTGGATGGGCGGAATTATCCCCGTGCTGG |
| | | GCACCGCTAACTACGCTCAGAAATTTCAGGGTAGAGTGACTAT |
| | | CACCGCCGACGAGTCTACTAGCACCGCCTATATGGAACTGTC |
| | | TAGCCTGAGATCAGAGGACACCGCCGTCTACTACTGCGCTAG |
| | | AGTGCCTACCGACTACTTCGACTACTGGGGTCAGGGCACCCT |
| | | GGTCACCGTGTCTAGC |
| 64 | Heavy Chain | QVQLVQSGAEVKKPGSSVKVSCKASGGTFSSYAISWVRQAPGQ |
| | | GLEWMGGIIPVLGTANYAQKFQGRVTITADESTSTAYMELSSLRS |
| | | EDTAVYYCARVPTDYFDYWGQGTLVTVSSASTKGPSVFPLAPSS |
| | | KSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTVLH |
| | | QDWLNGKEYKCKVSNKALPAPIEKTISKAKGQPREPQVYTLPPSR |
| | | EEMTKNGVSLTCLVKGFYPSDIAVEWESNGQPENNYKTTPPVLD |
| | | SDGSFFLYSKLTVDKSRWQQGNVFSCSVMHEALHNHYTQKSLS |
| | | LSPGK |
| 65 | DNA Heavy | CAGGTGCAGCTGGTGCAGTCAGGCGCCGAAGTGAAGAAACC |
| | Chain | CGGCTCTAGCGTGAAAGTCAGCTGTAAAGCTAGTGGCGGCAC |
| | | CTTCTCTAGCTACGCTATTAGCTGGGTCAGACAGGCCCCAGG |
| | | TCAGGGCCTGGAGTGGATGGGCGGAATTATCCCCGTGCTGG |
| | | GCACCGCTAACTACGCTCAGAAATTTCAGGGTAGAGTGACTAT |
| | | CACCGCCGACGAGTCTACTAGCACCGCCTATATGGAACTGTC |
| | | TAGCCTGAGATCAGAGGACACCGCCGTCTACTACTGCGCTAG |
| | | AGTGCCTACCGACTACTTCGACTACTGGGGTCAGGGCACCCT |
| | | GGTCACCGTGTCTAGCGCTAGCACTAAGGGCCCAAGTGTGTT |
| | | TCCCCTGGCCCCCAGCAGCAAGTCTACTTCCGGCGGAACTGC |
| | | TGCCCTGGGTTGCCTGGTGAAGGACTACTTCCCCGAGCCCGT |
| | | GACAGTGTCCTGGAACTCTGGGGCTCTGACTTCCGGCGTGCA |
| | | CACCTTCCCCGCCGTGCTGCAGAGCAGCGGCCTGTACAGCCT |
| | | GAGCAGCGTGGTGACAGTGCCCTCCAGCTCTCTGGGAACCCA |
| | | GACCTATATCTGCAACGTGAACCACAAGCCCAGCAACACCAA |
| | | GGTGGACAAGAGAGTGGAGCCCAAGAGCTGCGACAAGACCC |
| | | ACACCTGCCCCCCCTGCCCAGCTCCAGAACTGCTGGGAGGG |
| | | CCTTCCGTGTTCCTGTTCCCCCCCAAGCCCAAGGACACCCTG |
| | | ATGATCAGCAGGACCCCCGAGGTGACCTGCGTGGTGGTGGA |
| | | CGTGTCCCACGAGGACCCAGAGGTGAAGTTCAACTGGTACGT |
| | | GGACGGCGTGGAGGTGCACAACGCCAAGACCAAGCCCAGAG |
| | | AGGAGCAGTACAACAGCACCTACAGGGTGGTGTCCGTGCTGA |
| | | CCGTGCTGCACCAGGACTGGCTGAACGGCAAAGAATACAAGT |
| | | GCAAAGTCTCCAACAAGGCCCTGCCAGCCCCAATCGAAAAGA |
| | | CAATCAGCAAGGCCAAGGGCCAGCCACGGGAGCCCCAGGTG |
| | | TACACCCTGCCCCCCAGCCGGGAGGAGATGACCAAGAACCAG |
| | | GTGTCCCTGACCTGTCTGGTGAAGGGCTTCTACCCCAGCGAT |
| | | ATCGCCGTGGAGTGGGAGAGCAACGGCCAGCCCGAGAACAA |
| | | CTACAAGACCACCCCCCCAGTGCTGGACAGCGACGGCAGCTT |
| | | CTTCCTGTACAGCAAGCTGACCGTGGACAAGTCCAGGTGGCA |
| | | GCAGGGCAACGTGTTCAGCTGCAGCGTGATGCACGAGGCCCT |
| | | GCACAACCACTACACCCAGAAGTCCCTGAGCCTGAGCCCCGG |
| | | CAAG |
| 66 | LCDR1 | SGDNLGSKYVH |
| | (Combined) | |
| 67 | LCDR2 | DDNKRPS |
| | (Combined) | |
| 68 | LCDR3 | QSWTLGNWV |
| | (Combined) | |
| 69 | LCDR1 | SGDNLGSKYVH |
| | (Kabat) | |
| 70 | LCDR2 | DDNKRPS |
| | (Kabat) | |
| 71 | LCDR3 | QSWTLGNWV |
| | (Kabat) | |
| 72 | LCDR1 | DNLGSKY |
| | (Chothia) | |
| 73 | LCDR2 | DDN |
| | (Chothia) | |
| 74 | LCDR3 | WTLGNW |
| | (Chothia) | |
| 75 | VL | DIELTQPPSVSVSPGQTASITCSGDNLGSKYVHWYGQKPGQAPV |
| | | LVIYDDNKRPSGIPERFSGSNSGNTATLTISGTQAEDEADYYCQS |
| | | WTLGNVWFGGGTKLTVL |
| 76 | DNA VL | GATATCGAGCTGACTCAGCCCCCTAGCGTCAGCGTCAGCCCT |
| | | GGTCAGACCGCCTCTATCACCTGTAGCGGCGATAACCTGGGC |
| | | TCTAAATACGTGCACTGGTATCAGCAGAAGCCCGGTCAGGCC |
| | | CCCGTGCTGGTGATCTACGACGATAACAAGCGGCCTAGCGGA |
| | | ATCCCCGAGCGGTTTAGCGGCTCTAATAGCGGTAACACCGCT |
| | | ACCCTGACTATTAGCGGCACTCAGGCCGAGGACGAGGCCGAC |
| | | TACTACTGTCAGTCCTGGACCCTGGGCAACTGGGTGTTCGGC |
| | | GGAGGCACTAAGCTGACCGTGCTG |
| 77 | Light Chain | DIELTQPPSVSVSPGQTASITCSGDNLGSKYVHWVQQKPGGAPV |
| | | LVIYDDNKRPSGIPERFSGSNSGNTATLTISGTQAEDEADYYCGS |
| | | WTLGNWVFGGGTKLTVLGQPKAAPSVTLFPPSSEELQANKATLV |
| | | CLISDFYPGAVTVAWKADSSPVKAGVETTTPSKQSNNKYAASSY |
| | | LSLTPEQWKSHRSYSCQVTHEGSTVEKTVAPTECS |
| 78 | DNA Light | GATATCGAGCTGACTCAGCCCCCTAGCGTCAGCGTCAGCCCT |
| | Chain | GGTCAGACCGCCTCTATCACCTGTAGCGGCGATAACCTGGGC |
| | | TCTAAATACGTGCACTGGTATCAGCAGAAGCCCGGTCAGGCC |
| | | CCCGTGCTGGTGATCTACGACGATAACAAGCGGCCTAGCGGA |
| | | ATCCCCGAGCGGTTTAGCGGCTCTAATAGCGGTAACACCGCT |
| | | ACCCTGACTATTAGCGGCACTCAGGCCGAGGACGAGGCCGAC |
| | | TACTACTGTCAGTCCTGGACCCTGGGCAACTGGGTGTTCGGC |
| | | GGAGGCACTAAGCTGACCGTGCTGGGTCAGCCTAAGGCTGCC |
| | | CCCAGCGTGACCCTGTTCCCCCCCAGCAGCGAGGAGCTGCA |
| | | GGCCAACAAGGCCACCCTGGTGTGCCTGATCAGCGACTTCTA |
| | | CCCAGGCGCCGTGACCGTGGCCTGGAAGGCCGACAGCAGCC |
| | | CCGTGAAGGCCGGCGTGGAGACCACCACCCCCAGCAAGCAG |
| | | AGCAACAACAAGTACGCCGCCAGCAGCTACCTGAGCCTGACC |
| | | CCCGAGCAGTGGAAGAGCCACAGGTCCTACAGCTGCCAGGT |
| | | GACCCACGAGGGCAGCACCGTGGAAAAGACCGTGGCCCCAA |
| | | CCGAGTGCAGC |

NOV0308_N297A

| | | |
|---|---|---|
| 79 | HCDR1 | GGTFSSYAIS |
| | (Combined) | |
| 80 | HCDR2 | GIIPVLGTANYAQKFQG |
| | (Combined) | |
| 81 | HCDR3 | VPTDYFDY |
| | (Combined) | |
| 82 | HCDR1 | SYAIS |
| | (Kabat) | |
| 83 | HCDR2 | GIIPVLGTANYAQKFQG |
| | (Kabat) | |
| 84 | HCDR3 | VPTDYFDY |
| | (Kabat) | |
| 85 | HCDR1 | GGTFSSY |
| | (Chothia) | |
| 86 | HCDR2 | IPVLGT |
| | (Chothia) | |
| 87 | HCDR3 | VPTDYFDY |
| | (Chothia) | |
| 88 | VH | QVQLVQSGAEVKKPGSSVKVSCKASGGTFSSYAISWVRQAPGQ |
| | | GLEWMGGIIPVLGTANYAQKFQGRVTITADESTSTAYMELSSLRS |
| | | EDTAVYYCARVPTDYFDYWGQGTLVTVSS |
| 89 | DNA VH | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTTCTTCTTACGCTATCTCTTGGGTGCGCCAGGCCCCGGG |
| | | CCAGGGCCTCGAGTGGATGGGCGGTATCATCCCGGTTCTGG |
| | | GCACTGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGTTCCGACTGACTACTTCGATTACTGGGGCCAAGGCACCC |
| | | TGGTGACTGTTAGCTCA |
| 90 | Heavy Chain | QVQLVQSGAEVKKPGSSVKVSCKASGGTFSSYAISWVRQAPGQ |
| | | GLEWMGGIIPVLGTANYAQKFQGRVTITADESTSTAYMELSSLRS |
| | | EDTAVYYCARVPTDYFDYWGQGTLVTVSSASTKGPSVFPLAPSS |
| | | KSTSGGTAALGCLVKDYFPEPVTVSWNSGALTSGVHTFPAVLQS |
| | | SGLYSLSSWTVPSSSLGTQTYICNVNHKPSNTKVDKRVEPKSCD |
| | | KTHTCPPCPAPELLGGPSVFLFPPKPKDTLMISRTPEVTCVVVDV |
| | | SHEDPEVKFNWYVDGVEVHNAKTKPREEQYASTYRVVSVLTVLH |
| | | QDWLNGKEYKCKVSNKALPAPIEKTISKAKGQPREPQVYTLPPSR |
| | | EEMTKNQVSLTCLVKGFYPSDIAVEWESNGQPENNYKTTPPVLD |
| | | SDGSFFLYSKLTVDKSRWQQGNVFSCSVMHEALHNHYTQKSLS |
| | | LSPGK |
| 91 | DNA Heavy | CAGGTGCAATTGGTGCAGAGCGGTGCCGAAGTGAAAAAACCG |
| | Chain | GGCAGCAGCGTGAAAGTTAGCTGCAAAGCATCCGGAGGGAC |
| | | GTTTTCTTCTTACGCTATCTCTTGGGTGCGCCAGGCCCCGGG |
| | | CCAGGGCCTCGAGTGGATGGGCGGTATCATCCCGGTTCTGG |
| | | GCACTGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGTTCCGACTGACTACTTCGATTACTGGGGCCAAGGCACCC |
| | | TGGTGACTGTTAGCTCAGCCTCCACCAAGGGTCCATCGGTCTT |
| | | CCCCCTGGCACCCTCCTCCAAGAGCACCTCTGGGGGCACAGC |
| | | GGCCCTGGGCTGCCTGGTCAAGGACTACTTCCCCGAACCGGT |
| | | GACGGTGTCGTGGAACTCAGGCGCCCTGACCAGCGGCGTGC |
| | | ACACCTTCCCGGCTGTCCTACAGTCCTCAGGACTCTACTCCCT |
| | | CAGCAGCGTGGTGACCGTGCCCTCCAGCAGCTTGGGCACCC |
| | | AGACCTACATCTGCAACGTGAATCACAAGCCCAGCAACACCAA |
| | | GGTGGACAAGAGAGTTGAGCCCAAATCTTGTGACAAAACTCAC |
| | | ACATGCCCACCGTGCCCAGCACCTGAACTCCTGGGGGGACC |
| | | GTCAGTCTTCCTCTTCCCCCCAAAACCCAAGGACACCCTCATG |
| | | ATCTCCCGGACCCCTGAGGTCACATGCGTGGTGGTGGACGTG |
| | | AGCCACGAAGACCCTGAGGTCAAGTTCAACTGGTACGTGGAC |
| | | GGCGTGGAGGTGCATAATGCCAAGACAAAGCCGCGGGAGGA |
| | | GCAGTACGCCAGCACGTACCGGGTGGTCAGCGTCCTCACCGT |
| | | CCTGCACCAGGACTGGCTGAATGGCAAGGAGTACAAGTGCAA |
| | | GGTCTCCAACAAAGCCCTCCCAGCCCCCATCGAGAAAACCAT |
| | | CTCCAAAGCCAAAGGGCAGCCCCGAGAACCACAGGTGTACAC |
| | | CCTGCCCCCATCCCGGGAGGAGATGACCAAGAACCAGGTCAG |
| | | CCTGACCTGCCTGGTCAAAGGCTTCTATCCCAGCGACATCGC |
| | | CGTGGAGTGGGAGAGCAATGGGCAGCCGGAGAACAACTACA |
| | | AGACCACGCCTCCCGTGCTGGACTCCGACGGCTCCTTCTTCC |
| | | TCTACAGCAAGCTCACCGTGGACAAGAGCAGGTGGCAGCAGG |
| | | GGAACGTCTTCTCATGCTCCGTGATGCATGAGGCTCTGCACAA |
| | | CCACTACACGCAGAAGAGCCTCTCCCTGTCTCCGGGTAAA |
| 92 | LCDR1 | SGDNLGSKYVH |
| | (Combined) | |
| 93 | LCDR2 | DDNKRPS |
| | (Combined) | |
| 94 | LCDR3 | QSWTLGNWV |
| | (Combined) | |
| 95 | LCDR1 | SGDNLGSKYVH |
| | (Kabat) | |
| 96 | LCDR2 | DDNKRPS |
| | (Kabat) | |
| 97 | LCDR3 | QSWTLGNWV |
| | (Kabat) | |
| 98 | LCDR1 | DNLGSKY |
| | (Chothia) | |
| 99 | LCDR2 | DDN |
| | (Chothia) | |
| 100 | LCDR3 | WTLGNW |
| | (Chothia) | |
| 101 | VL | DIELTQPPSVSVSPGQTASITCSGDNLGSKYVHWYQQKPGQAPV |
| | | LVIYDDNKRPSGIPERFSGSNSGNTATLTISGTQAEDEADYYCQS |
| | | WTLGNWVFGGGTKLTVL |
| 102 | DNA VL | GATATCGAACTGACCCAGCCGCCGAGCGTGAGCGTGAGCCC |
| | | GGGCCAGACCGCGAGCATTACCTGTAGCGGCGATAACCTGG |
| | | GTTCTAAATACGTTCATTGGTACCAGCAGAAACCGGGCCAGG |
| | | CGCCGGTGCTGGTGATCTACGACGACAACAAACGTCCGAGCG |
| | | GCATCCCGGAACGTTTTAGCGGATCCAACAGCGGCAACACCG |
| | | CGACCCTGACCATTAGCGGCACCCAGGCGGAAGACGAAGCG |
| | | GATTATTACTGCCAGTCTTGGACTCTGGGTAACTGGGTGTTTG |
| | | GCGGCGGCACGAAGTTAACCGTCCTA |
| 103 | Light Chain | DIELTQPPSVSVSPGQTASITCSGDNLGSKYVHWYQQKPGQAPV |
| | | LVIYDDNKRPSGIPERFSGSNSGNTATLTISGTQAEDEADYYCQS |
| | | WTLGNWVFGGGTKLTVLGQPKAAPSVTLFPPSSEELQANKATLV |
| | | CLISDFYPGAVTVAWKADSSPVKAGVETTTPSKQSNNKYAASSY |
| | | LSLTPEQWKSHRSYSCQVTHEGSTVEKTVAPTECS |
| 104 | DNA Light | GATATCGAACTGACCCAGCCGCCGAGCGTGAGCGTGAGCCC |
| | Chain | GGGCCAGACCGCGAGCATTACCTGTAGCGGCGATAACCTGG |
| | | GTTCTAAATACGTTCATTGGTACCAGCAGAAACCGGGCCAGG |
| | | CGCCGGTGCTGGTGATCTACGACGACAACAAACGTCCGAGCG |
| | | GCATCCCGGAACGTTTTAGCGGATCCAACAGCGGCAACACCG |
| | | CGACCCTGACCATTAGCGGCACCCAGGCGGAAGACGAAGCG |
| | | GATTATTACTGCCAGTCTTGGACTCTGGGTAACTGGGTGTTTG |
| | | GCGGCGGCACGAAGTTAACCGTCCTAGGTCAGCCCAAGGCTG |
| | | CCCCCTCGGTCACTCTGTTCCCGCCCTCCTCTGAGGAGCTTC |
| | | AAGCCAACAAGGCCACACTGGTGTGTCTCATAAGTGACTTCTA |
| | | CCCGGGAGCCGTGACAGTGGCCTGGAAGGCAGATAGCAGCC |
| | | CCGTCAAGGCGGGAGTGGAGACCACCACACCCTCCAAACAAA |
| | | GCAACAACAAGTACGCGGCCAGCAGCTATCTGAGCCTGACGC |
| | | CTGAGCAGTGGAAGTCCCACAGAAGCTACAGCTGCCAGGTCA |
| | | CGCATGAAGGGAGCACCGTGGAGAAGACAGTGGCCCCTACA |
| | | GAATGTTCA |

NOV0563

| | | |
|---|---|---|
| 105 | HCDR1 | GGTFSDNAIS |
| | (Combined) | |
| 106 | HCDR2 | GINPDFGWANYAQKFQG |
| | (Combined) | |
| 107 | HCDR3 | DSSGMGY |
| | (Combined) | |
| 108 | HCDR1 | DNAIS |
| | (Kabat) | |
| 109 | HCDR2 | GINPDFGWANYAQKFQG |
| | (Kabat) | |
| 110 | HCDR3 | DSSGMGY |
| | (Kabat} | |
| 111 | HCDR1 | GGTFSDN |
| | (Chothia) | |
| 112 | HCDR2 | NPDFGW |
| | (Chothia) | |
| 113 | HCDR3 | DSSGMGY |
| | (Chothia) | |
| 114 | VH | QVQLVQSGAEVKKPGSSVKVSCKASGGTFSDNAISWVRQAPGQ |
| | | GLEWMGGINPDFGWANYAQKFQGRVTITADESTSTAYMELSSLR |
| | | SEDTAVYYCARDSSGMGYWGQGTLVTVSS |
| 115 | DNA VH | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGCGATAACGCTATTAGCTGGGTCAGACAGGCCCCAGG |
| | | TCAGGGCCTGGAGTGGATGGGCGGGATTAACCCCGACTTCG |
| | | GCTGGGCTAACTACGCTCAGAAATTTCAGGGTAGAGTGACTAT |
| | | CACCGCCGACGAGTCTACTAGCACCGCCTATATGGAACTGTC |
| | | TAGCCTGAGATCAGAGGACACCGCCGTCTACTACTGCGCTAG |
| | | GGACTCTAGCGGAATGGGCTACTGGGGTCAGGGCACCCTGG |
| | | TCACCGTGTCTAGC |
| 116 | Heavy Chain | QVQLVQSGAEVKKPGSSVKVSCKASGGTFSDNAISVWRQAPGQ |
| | | GLEWMGGINPDFGWANYAQKFGGRVTITADESTSTAYMELSSLR |
| | | SEDTAVYYCARDSSGMGYWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVD |
| | | VSHEDPEVKFNWYVDGVEVHNAKTKPREEQYNSTYRVVSVLTVL |
| | | HQDWLNGKEYKCKVSNKALPAPIEKTISKAKGQPREPQVYTLPPS |
| | | REEMTKNQVSLTCLVKGFYPSDIAVEWESNGQPENNYKTTPPVL |
| | | DSDGSFFLYSKLTVDKSRWQQGNVFSCSVMHEALHNHYTQKSL |
| | | SLSPGK |
| 117 | DNA Heavy | CAGGTGCAGCTGGTGCAGTCAGGCGCCGAAGTGAAGAAACC |
| | Chain | CGGCTCTAGCGTGAAAGTCAGCTGTAAAGCTAGTGGCGGCAC |
| | | CTTTAGCGATAACGCTATTAGCTGGGTCAGACAGGCCCCAGG |
| | | TCAGGGCCTGGAGTGGATGGGCGGGATTAACCCCGACTTCG |
| | | GCTGGGCTAACTACGCTCAGAAATTTCAGGGTAGAGTGACTAT |
| | | CACCGCCGACGAGTCTACTAGCACCGCCTATATGGAACTGTC |
| | | TAGCCTGAGATCAGAGGACACCGCCGTCTACTACTGCGCTAG |
| | | GGACTCTAGCGGAATGGGCTACTGGGGTCAGGGCACCCTGG |
| | | TCACCGTGTCTAGCGCTAGCACTAAGGGCCCAAGTGTGTTTC |
| | | CCCTGGCCCCCAGCAGCAAGTCTACTTCCGGCGGAACTGCTG |
| | | CCCTGGGTTGCCTGGTGAAGGACTACTTCCCCGAGCCCGTGA |
| | | CAGTGTCCTGGAACTCTGGGGCTCTGACTTCCGGCGTGCACA |
| | | CCTTCCCCGCCGTGCTGCAGAGCAGCGGCCTGTACAGCCTGA |
| | | GCAGCGTGGTGACAGTGCCCTCCAGCTCTCTGGGAACCCAGA |
| | | CCTATATCTGCAACGTGAACCACAAGCCCAGCAACACCAAGGT |
| | | GGACAAGAGAGTGGAGCCCAAGAGCTGCGACAAGACCCACA |
| | | CCTGCCCCCCCTGCCCAGCTCCAGAACTGCTGGGAGGGCCTT |
| | | CCGTGTTCCTGTTCCCCCCCAAGCCCAAGGACACCCTGATGA |
| | | TCAGCAGGACCCCCGAGGTGACCTGCGTGGTGGTGGACGTG |
| | | TCCCACGAGGACCCAGAGGTGAAGTTCAACTGGTACGTGGAC |
| | | GGCGTGGAGGTGCACAACGCCAAGACCAAGCCCAGAGAGGA |
| | | GCAGTACAACAGCACCTACAGGGTGGTGTCCGTGCTGACCGT |
| | | GCTGCACCAGGACTGGCTGAACGGCAAAGAATACAAGTGCAA |
| | | AGTCTCCAACAAGGCCCTGCCAGCCCCAATCGAAAAGACAAT |
| | | CAGCAAGGCCAAGGGCCAGCCACGGGAGCCCCAGGTGTACA |
| | | CCCTGCCCCCCAGCCGGGAGGAGATGACCAAGAACCAGGTG |
| | | TCCCTGACCTGTCTGGTGAAGGGCTTCTACCCCAGCGATATC |
| | | GCCGTGGAGTGGGAGAGCAACGGCCAGCCCGAGAACAACTA |
| | | CAAGACCACCCCCCCAGTGCTGGACAGCGACGGCAGCTTCTT |
| | | CCTGTACAGCAAGCTGACCGTGGACAAGTCCAGGTGGCAGCA |
| | | GGGCAACGTGTTCAGCTGCAGCGTGATGCACGAGGCCCTGCA |
| | | CAACCACTACACCCAGAAGTCCCTGAGCCTGAGCCCCGGCAA |
| | | G |
| 118 | LCDR1 | RASQDISSYLN |
| | (Combined) | |
| 119 | LCDR2 | DASTLQS |
| | (Combined) | |
| 120 | LCDR3 | QQSGHWLSKT |
| | (Combined) | |
| 121 | LCDR1 | RASQDISSYLN |
| | (Kabat) | |
| 122 | LCDR2 | DASTLQS |
| | (Kabat) | |
| 123 | LCDR3 | QQSGHWLSKT |
| | (Kabat) | |
| 124 | LCDR1 | SQDISSY |
| | (Chothia) | |
| 125 | LCDR2 | DAS |
| | (Chothia) | |
| 126 | LCDR3 | SGHWLSK |
| | (Chothia) | |
| 127 | VL | DIQMTGSPSSLSASVGDRVTITCRASQDISSYLNWYQQKPGKAP |
| | | KLLIYDASTLQSGVPSRFSGSGSGTDFTLTISSLQPEDFATYYCQ |
| | | QSGHWLSKTFGQGTKVEIK |
| 128 | DNA VL | GATATTCAGATGACTCAGTCACCTAGTAGCCTGAGCGCTAGTG |
| | | TGGGCGATAGAGTGACTATCACCTGTAGAGCCTCTCAGGATAT |
| | | CTCTAGCTACCTGAACTGGTATCAGCAGAAGCCCGGTAAAGC |
| | | CCCTAAGCTGCTGATCTACGACGCCTCTACCCTGCAGTCAGG |
| | | CGTGCCCTCTAGGTTTAGCGGTAGCGGTAGTGGCACCGACTT |
| | | CACCCTGACTATCTCTAGCCTGCAGCCCGAGGACTTCGCTAC |
| | | CTACTACTGTCAGCAGTCAGGCCACTGGCTGTCTAAGACCTTC |
| | | GGTCAGGGCACTAAGGTCGAGATTAAG |
| 129 | Light Chain | DIQMTQSPSSLSASVGDRVTITCRASQDISSYLNWYQQKPGKAP |
| | | KLLIYDASTLQSGVPSRFSGSGSGTDFTLTISSLQPEDFATYYCQ |
| | | QSGHWLSKTFGQGTKVEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 130 | DNA Light | GATATTCAGATGACTCAGTCACCTAGTAGCCTGAGCGCTAGTG |
| | Chain | TGGGCGATAGAGTGACTATCACCTGTAGAGCCTCTCAGGATAT |
| | | CTCTAGCTACCTGAACTGGTATCAGCAGAAGCCCGGTAAAGC |
| | | CCCTAAGCTGCTGATCTACGACGCCTCTACCCTGCAGTCAGG |
| | | CGTGCCCTCTAGGTTTAGCGGTAGCGGTAGTGGCACCGACTT |
| | | CACCCTGACTATCTCTAGCCTGCAGCCCGAGGACTTCGCTAC |
| | | CTACTACTGTCAGCAGTCAGGCCACTGGCTGTCTAAGACCTTC |
| | | GGTCAGGGCACTAAGGTCGAGATTAAGCGTACGGTGGCCGCT |
| | | CCCAGCGTGTTCATCTTCCCCCCCAGCGACGAGCAGCTGAAG |
| | | AGCGGCACCGCCAGCGTGGTGTGCCTGCTGAACAACTTCTAC |
| | | CCCCGGGAGGCCAAGGTGCAGTGGAAGGTGGACAACGCCCT |
| | | GCAGAGCGGCAACAGCCAGGAGAGCGTCACCGAGCAGGACA |
| | | GCAAGGACTCCACCTACAGCCTGAGCAGCACCCTGACCCTGA |
| | | GCAAGGCCGACTACGAGAAGCATAAGGTGTACGCCTGCGAGG |
| | | TGACCCACCAGGGCCTGTCCAGCCCCGTGACCAAGAGCTTCA |
| | | ACAGGGGCGAGTGC |

NOV0563_N297A

| | | |
|---|---|---|
| 131 | HCDR1 | GGTFGDNAIS |
| | (Combined) | |
| 132 | HCDR2 | GINPDFGWANYAQKFQG |
| | (Combined) | |
| 133 | HCDR3 | DSSGMGY |
| | (Combined) | |
| 134 | HCDR1 | DNAIS |
| | (Kabat) | |
| 135 | HCDR2 | GINPDFGWANYAQKFQG |
| | (Kabat) | |
| 136 | HCDR3 | DSSGMGY |
| | (Kabat) | |
| 137 | HCDR1 | GGTFSDN |
| | (Chothia) | |
| 138 | HCDR2 | NPDFGW |
| | (Chothia) | |
| 139 | HCDR3 | DSSGMGY |
| | (Chothia) | |
| 140 | VH | QVQLVQSGAEVKKPGSSVKVSCKASGGTFSDNAISVWRQAPGQ |
| | | GLEWMGGINPDFGWANYAQKFQGRVTITADESTSTAYMELSSLR |
| | | SEDTAVYYCARDSSGMGYWGQGTLVTVSS |
| 141 | DNA VH | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTTCTGACAACGCTATCTCTTGGGTGCGCCAGGCCCCGGG |
| | | CCAGGGCCTCGAGTGGATGGGCGGTATCAACCCGGACTTCG |
| | | GCTGGGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGACTCTTCTGGTATGGGTTACTGGGGCCAAGGCACCCTGG |
| | | TGACTGTTAGCTCA |
| 142 | Heavy Chain | QVQLVQSGAEVKKPGSSVKVSCKASGGTFSDNAISWVRQAPGQ |
| | | GLEWMGGINPDFGWANYAQKFQGRVTITADESTSTAYMELSSLR |
| | | SEDTAVYYCARDSSGMGYWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTGTYICNVNHKPSNTKVDKRVEPKSC |
| | | DKTHTCPPCPAPELLGGPSVFLFPPKPKDTLMISRTPEVTCVVVD |
| | | VSHEDPEVKFNWYVDGVEVHNAKTKPREEQYASTYRVVSVLTVL |
| | | HQDWLNGKEYKCKVSNKALPAPIEKTISKAKGQPPEPQVYTLPPS |
| | | REEMTKNQVSLTCLVKGFYPSDIAVEWESNGQPENNYKTTPPVL |
| | | DSDGSFFLYSKLTVDKSRWQQGNVFSCSVMHEALHNHYTQKSL |
| | | SLSPGK |
| 143 | DNA Heavy | CAGGTGCAATTGGTGCAGAGCGGTGCCGAAGTGAAAAAACCG |
| | Chain | GGCAGCAGCGTGAAAGTTAGCTGCAAAGCATCCGGAGGGAC |
| | | GTTTTCTGACAACGCTATCTCTTGGGTGCGCCAGGCCCCGGG |
| | | CCAGGGCCTCGAGTGGATGGGCGGTATCAACCCGGACTTCG |
| | | GCTGGGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGACTCTTCTGGTATGGGTTACTGGGGCCAAGGCACCCTGG |
| | | TGACTGTTAGCTCAGCCTCCACCAAGGGTCCATCGGTCTTCCC |
| | | CCTGGCACCCTCCTCCAAGAGCACCTCTGGGGGCACAGCGG |
| | | CCCTGGGCTGCCTGGTCAAGGACTACTTCCCCGAACCGGTGA |
| | | CGGTGTCGTGGAACTCAGGCGCCCTGACCAGCGGCGTGCAC |
| | | ACCTTCCCGGCTGTCCTACAGTCCTCAGGACTCTACTCCCTCA |
| | | GCAGCGTGGTGACCGTGCCCTCCAGCAGCTTGGGCACCCAG |
| | | ACCTACATCTGCAACGTGAATCACAAGCCCAGCAACACCAAG |
| | | GTGGACAAGAGAGTTGAGCCCAAATCTTGTGACAAAACTCACA |
| | | CATGCCCACCGTGCCCAGCACCTGAACTCCTGGGGGGACCGT |
| | | CAGTCTTCCTCTTCCCCCCAAAACCCAAGGACACCCTCATGAT |
| | | CTCCCGGACCCCTGAGGTCACATGCGTGGTGGTGGACGTGA |
| | | GCCACGAAGACCCTGAGGTCAAGTTCAACTGGTACGTGGACG |
| | | GCGTGGAGGTGCATAATGCCAAGACAAAGCCGCGGGAGGAG |
| | | CAGTACGCCAGCACGTACCGGGTGGTCAGCGTCCTCACCGTC |
| | | CTGCACCAGGACTGGCTGAATGGCAAGGAGTACAAGTGCAAG |
| | | GTCTCCAACAAAGCCCTCCCAGCCCCCATCGAGAAAACCATCT |
| | | CCAAAGCCAAAGGGCAGCCCCGAGAACCACAGGTGTACACCC |
| | | TGCCCCCATCCCGGGAGGAGATGACCAAGAACCAGGTCAGCC |
| | | TGACCTGCCTGGTCAAAGGCTTCTATCCCAGCGACATCGCCG |
| | | TGGAGTGGGAGAGCAATGGGCAGCCGGAGAACAACTACAAGA |
| | | CCACGCCTCCCGTGCTGGACTCCGACGGCTCCTTCTTCCTCT |
| | | ACAGCAAGCTCACCGTGGACAAGAGCAGGTGGCAGCAGGGG |
| | | AACGTCTTCTCATGCTCCGTGATGCATGAGGCTCTGCACAACC |
| | | ACTACACGCAGAAGAGCCTCTCCCTGTCTCCGGGTAAA |
| 144 | LCDR1 | RASQDISSYLN |
| | (Combined) | |
| 145 | LCDR2 | DASTLQS |
| | (Combined) | |
| 146 | LCDR3 | QQSGHWLSKT |
| | (Combined) | |
| 147 | LCDR1 | RASQDISSYLN |
| | (Kabat) | |
| 148 | LCDR2 | DASTLQS |
| | (Kabat) | |
| 149 | LCDR3 | QQSGHWLSKT |
| | (Kabat) | |
| 150 | LCDR1 | SQDISSY |
| | (Chothia) | |
| 151 | LCDR2 | DAS |
| | (Chothia) | |
| 152 | LCDR3 | SGHWLSK |
| | (Chothia) | |
| 153 | VL | DIQMTQSPSSLSASVGDRVTITCRASQDISSYLNWYQQKPGKAP |
| | | KLLIYDASTLQSGVPSRFSGSGSGTDFTLTISSLQPEDFATYYCQ |
| | | QSGHWLSKTFGQGTKVEIK |
| 154 | DNA VL | GATATCCAGATGACCCAGAGCCCGAGCAGCCTGAGCGCCAGC |
| | | GTGGGCGATCGCGTGACCATTACCTGCAGAGCCAGCCAGGAC |
| | | ATTTCTTCTTACCTGAACTGGTACCAGCAGAAACCGGGCAAAG |
| | | CGCCGAAACTATTAATCTACGACGCTTCTACTCTGCAAAGCGG |
| | | CGTGCCGAGCCGCTTTAGCGGCAGCGGATCCGGCACCGATTT |
| | | CACCCTGACCATTAGCTCTCTGCAACCGGAAGACTTTGCGACC |
| | | TATTATTGCCAGCAGTCTGGTCATTGGCTGTCTAAAACCTTTG |
| | | GCCAGGGCACGAAAGTTGAAATTAAA |
| 155 | Light Chain | DIQMTQSPSSLSASVGDRVTITCRASQDISSYLNWYQQKPGKAP |
| | | KLLIYDASTLQSGVPSRFSGSGSGTDFTLTISSLQPEDFATYYCQ |
| | | QSGHWLSKTFGQGTKVEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 156 | DNA Light | GATATCCAGATGACCCAGAGCCCGAGCAGCCTGAGCGCCAGC |
| | Chain | GTGGGCGATCGCGTGACCATTACCTGCAGAGCCAGCCAGGAC |
| | | ATTTCTTCTTACCTGAACTGGTACCAGCAGAAACCGGGCAAAG |
| | | CGCCGAAACTATTAATCTACGACGCTTCTACTCTGCAAAGCGG |
| | | CGTGCCGAGCCGCTTTAGCGGCAGCGGATCCGGCACCGATTT |
| | | CACCCTGACCATTAGCTCTCTGCAACCGGAAGACTTTGCGACC |
| | | TATTATTGCCAGCAGTCTGGTCATTGGCTGTCTAAAACCTTTG |
| | | GCCAGGGCACGAAAGTTGAAATTAAACGTACGGTGGCCGCTC |
| | | CCAGCGTGTTCATCTTCCCCCCCAGCGACGAGCAGCTGAAGA |
| | | GCGGCACCGCCAGCGTGGTGTGCCTGCTGAACAACTTCTACC |
| | | CCCGGGAGGCCAAGGTGCAGTGGAAGGTGGACAACGCCCTG |
| | | CAGAGCGGCAACAGCCAGGAAAGCGTCACCGAGCAGGACAG |
| | | CAAGGACTCCACCTACAGCCTGAGCAGCACCCTGACCCTGAG |
| | | CAAGGCCGACTACGAGAAGCACAAGGTGTACGCCTGCGAGGT |
| | | GACCCACCAGGGCCTGTCCAGCCCCGTGACCAAGAGCTTCAA |
| | | CCGGGGCGAGTGT |

NOV1216

| | | |
|---|---|---|
| 157 | HCDR1 | GGTFRDYAIS |
| | (Combined) | |
| 158 | HCDR2 | GIIPAFGTANYAQKFQG |
| | (Combined) | |
| 159 | HCDR3 | EQDPEYGYGGYPYEAMDV |
| | (Combined) | |
| 160 | HCDR1 | DYAIS |
| | (Kabat) | |
| 161 | HCDR2 | GIIPAFGTANYAQKFQG |
| | (Kabat) | |
| 162 | HCDR3 | EQDPEYGYGGYPYEAMDV |
| | (Kabat) | |
| 163 | HCDR1 | GGTFRDY |
| | (Chothia) | |
| 164 | HCDR2 | IPAFGT |
| | (Chothia) | |
| 165 | HCDR3 | EQDPEYGYGGYPYEAMDV |
| | (Chothia) | |
| 166 | VH | QVQLVQSGAEVKKPGSSVKVSCKASGGTFRDYAISVWRQAP |
| | | GQGLEWMGGIIPAFGTANYAQKFQGRVTITADESTSTAYMELS |
| | | SLRSEDTAVYYCAREQDPEYGYGGYPYEAMDVWGQGTLVTV |
| | | SS |
| 167 | DNA VH | CAGGTGCAGCTGGTGCAGTCAGGCGCCGAAGTGAAGAAAC |
| | | CCGGCTCTAGCGTGAAAGTCAGCTGTAAAGCTAGTGGCGG |
| | | CACCTTTAGAGACTACGCTATTAGCTGGGTCAGACAGGCCC |
| | | CAGGTCAGGGCCTGGAGTGGATGGGCGGAATTATCCCCGC |
| | | CTTCGGCACCGCTAACTACGCTCAGAAATTTCAGGGTAGAG |
| | | TGACTATCACCGCCGACGAGTCTACTAGCACCGCCTATATG |
| | | GAACTGTCTAGCCTGAGATCAGAGGACACCGCCGTCTACTA |
| | | CTGCGCTAGAGAGCAGGACCCCGAGTACGGCTACGGCGG |
| | | CTACCCCTACGAGGCTATGGACGTGTGGGGTCAGGGCACC |
| | | CTGGTCACCGTGTCTAGC |
| 168 | Heavy Chain | QVQLVQSGAEVKKPGSSVKVSCKASGGTFRDYAISWVRQAP |
| | | GQGLEWMGGIIPAFGTANYAQKFQGRVTITADESTSTAYMELS |
| | | SLRSEDTAVYYCAREQDPEYGYGGYPYEAMDVWGQGTLVTV |
| | | SSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 169 | DNA Heavy | CAGGTGCAGCTGGTGCAGTCAGGCGCCGAAGTGAAGAAAC |
| | Chain | CCGGCTCTAGCGTGAAAGTCAGCTGTAAAGCTAGTGGCGG |
| | | CACCTTTAGAGACTACGCTATTAGCTGGGTCAGACAGGCCC |
| | | CAGGTCAGGGCCTGGAGTGGATGGGCGGAATTATCCCCGC |
| | | CTTCGGCACCGCTAACTACGCTCAGAAATTTCAGGGTAGAG |
| | | TGACTATCACCGCCGACGAGTCTACTAGCACCGCCTATATG |
| | | GAACTGTCTAGCCTGAGATCAGAGGACACCGCCGTCTACTA |
| | | CTGCGCTAGAGAGCAGGACCCCGAGTACGGCTACGGCGG |
| | | CTACCCCTACGAGGCTATGGACGTGTGGGGTCAGGGCACC |
| | | CTGGTCACCGTGTCTAGCGCTAGCACTAAGGGCCCAAGTG |
| | | TGTTTCCCCTGGCCCCCAGCAGCAAGTCTACTTCCGGCGG |
| | | AACTGCTGCCCTGGGTTGCCTGGTGAAGGACTACTTCCCC |
| | | GAGCCCGTGACAGTGTCCTGGAACTCTGGGGCTCTGACTT |
| | | CCGGCGTGCACACCTTCCCCGCCGTGCTGCAGAGCAGCGG |
| | | CCTGTACAGCCTGAGCAGCGTGGTGACAGTGCCCTCCAGC |
| | | TCTCTGGGAACCCAGACCTATATCTGCAACGTGAACCACAA |
| | | GCCCAGCAACACCAAGGTGGACAAGAGAGTGGAGCCCAAG |
| | | AGCTGCGACAAGACCCACACCTGCCCCCCCTGCCCAGCTC |
| | | CAGAACTGCTGGGAGGGCCTTCCGTGTTCCTGTTCCCCCC |
| | | CAAGCCCAAGGACACCCTGATGATCAGCAGGACCCCCGAG |
| | | GTGACCTGCGTGGTGGTGGACGTGTCCCACGAGGACCCAG |
| | | AGGTGAAGTTCAACTGGTACGTGGACGGCGTGGAGGTGCA |
| | | CAACGCCAAGACCAAGCCCAGAGAGGAGCAGTACAACAGC |
| | | ACCTACAGGGTGGTGTCCGTGCTGACCGTGCTGCACCAGG |
| | | ACTGGCTGAACGGCAAAGAATACAAGTGCAAAGTCTCCAAC |
| | | AAGGCCCTGCCAGCCCCAATCGAAAAGACAATCAGCAAGG |
| | | CCAAGGGCCAGCCACGGGAGCCCCAGGTGTACACCCTGCC |
| | | CCCCAGCCGGGAGGAGATGACCAAGAACCAGGTGTCCCTG |
| | | ACCTGTCTGGTGAAGGGCTTCTACCCCAGCGATATCGCCGT |
| | | GGAGTGGGAGAGCAACGGCCAGCCCGAGAACAACTACAAG |
| | | ACCACCCCCCCAGTGCTGGACAGCGACGGCAGCTTCTTCC |
| | | TGTACAGCAAGCTGACCGTGGACAAGTCCAGGTGGCAGCA |
| | | GGGCAACGTGTTCAGCTGCAGCGTGATGCACGAGGCCCTG |
| | | CACAACCACTACACCCAGAAGTCCCTGAGCCTGAGCCCCG |
| | | GCAAG |
| 170 | LCDR1 | SGDNIPQHSVH |
| | (Combined) | |
| 171 | LCDR2 | DDTERPS |
| | (Combined) | |
| 172 | LCDR3 | SSWDSSMDSVV |
| | (Combined) | |
| 173 | LCDR1 | SGDNIPQHSVH |
| | (Kabat) | |
| 174 | LCDR2 | DDTERPS |
| | (Kabat) | |
| 175 | LCDR3 | SSWDSSMDSVV |
| | (Kabat) | |
| 176 | LCDR1 | DNIPQHS |
| | (Chothia) | |
| 177 | LCDR2 | DDT |
| | (Chothia) | |
| 178 | LCDR3 | WDSSMDSV |
| | (Chothia) | |
| 179 | VL | SYELTQPLSVSVALGQTARITCSGDNIPQHSVHWYQQKPGQA |
| | | PVLVIYDDTERPSGIPERFSGSNSGNTATLTISRAQAGDEADYY |
| | | CSSWDSSMDSWFGGGTKLTVL |
| 180 | DNA VL | AGCTACGAGCTGACTCAGCCCCTGAGCGTCAGCGTGGCCC |
| | | TGGGTCAGACCGCTAGAATCACCTGTAGCGGCGATAATATC |
| | | CCTCAGCACTCAGTGCACTGGTATCAGCAGAAGCCCGGTC |
| | | AGGCCCCCGTGCTGGTGATCTACGACGACACCGAGCGGCC |
| | | TAGCGGAATCCCCGAGCGGTTTAGCGGCTCTAATAGCGGT |
| | | AACACCGCTACCCTGACTATCTCTAGGGCTCAGGCCGGCG |
| | | ACGAGGCCGACTACTACTGCTCTAGCTGGGATAGCTCTATG |
| | | GATAGCGTGGTGTTCGGCGGAGGCACTAAGCTGACCGTGC |
| | | TG |
| 181 | Light Chain | SYELTQPLSVSVALGQTARITCSGDNIPQHSVHWYQQKPGQA |
| | | PVLVIYDDTERPSGIPERFSGSNSGNTATLTISRAQAGDEADYY |
| | | CSSWDSSMDSVVFGGGTKLTVLGQPKAAPSVTLFPPSSEELQ |
| | | ANKATLVCLISDFYPGAVTVAWKADSSPVKAGVETTTPSKQSN |
| | | NKYAASSYLSLTPEQWKSHRSYSCQVTHEGSTVEKTVAPTEC |
| | | S |
| 182 | DNA Light | AGCTACGAGCTGACTCAGCCCCTGAGCGTCAGCGTGGCCC |
| | Chain | TGGGTCAGACCGCTAGAATCACCTGTAGCGGCGATAATATC |
| | | CCTCAGCACTCAGTGCACTGGTATCAGCAGAAGCCCGGTC |
| | | AGGCCCCCGTGCTGGTGATCTACGACGACACCGAGCGGCC |
| | | TAGCGGAATCCCCGAGCGGTTTAGCGGCTCTAATAGCGGT |
| | | AACACCGCTACCCTGACTATCTCTAGGGCTCAGGCCGGCG |
| | | ACGAGGCCGACTACTACTGCTCTAGCTGGGATAGCTCTATG |
| | | GATAGCGTGGTGTTCGGCGGAGGCACTAAGCTGACCGTGC |
| | | TGGGTCAGCCTAAGGCTGCCCCCAGCGTGACCCTGTTCCC |
| | | CCCCAGCAGCGAGGAGCTGCAGGCCAACAAGGCCACCCTG |
| | | GTGTGCCTGATCAGCGACTTCTACCCAGGCGCCGTGACCG |
| | | TGGCCTGGAAGGCCGACAGCAGCCCCGTGAAGGCCGGCG |
| | | TGGAGACCACCACCCCCAGCAAGCAGAGCAACAACAAGTA |
| | | CGCCGCCAGCAGCTACCTGAGCCTGACCCCCGAGCAGTGG |
| | | AAGAGCCACAGGTCCTACAGCTGCCAGGTGACCCACGAGG |
| | | GCAGCACCGTGGAAAAGACCGTGGCCCCAACCGAGTGCAG |
| | | C |

NOV1216_N297A

| | | |
|---|---|---|
| 183 | HCDR1 | GGTFRDYAIS |
| | (Combined) | |
| 184 | HCDR2 | GIIPAFGTANYAQKFQG |
| | (Combined) | |
| 185 | HCDR3 | EQDPEYGYGGYPYEAMDV |
| | (Combined) | |
| 186 | HCDR1 | DYAIS |
| | (Kabat) | |
| 187 | HCDR2 | GIIPAFGTANYAQKFQG |
| | (Kabat) | |
| 188 | HCDR3 | EQDPEYGYGGYPYEAMDV |
| | (Kabat) | |
| 189 | HCDR1 | GGTFRDY |
| | (Chothia) | |
| 190 | HCDR2 | IPAFGT |
| | (Chothia) | |
| 191 | HCDR3 | EQDPEYGYGGYPYEAMDV |
| | (Chothia) | |
| 192 | VH | QVQLVQSGAEVKKPGSSVKVSCKASGGTFRDYAISWVRQAP |
| | | GQGLEWMGGIIPAFGTANYAQKFQGRVTITADESTSTAYMELS |
| | | SLRSEDTAVYYCAREQDPEYGYGGYPYEAMDVWGQGTLVTV |
| | | SS |
| 193 | DNA VH | CAGGTGCAATTGGTGCAGAGCGGTGCCGAAGTGAAAAAAC |
| | | CGGGCAGCAGCGTGAAAGTTAGCTGCAAAGCATCCGGAGG |
| | | GACGTTTCGTGACTACGCTATCTCTTGGGTGCGCCAGGCCC |
| | | CGGGCCAGGGCCTCGAGTGGATGGGCGGTATCATCCCGG |
| | | CTTTCGGCACTGCGAACTACGCCCAGAAATTTCAGGGCCG |
| | | GGTGACCATTACCGCCGATGAAAGCACCAGCACCGCCTATA |
| | | TGGAACTGAGCAGCCTGCGCAGCGAAGATACGGCCGTGTA |
| | | TTATTGCGCGCGTGAACAGGACCCGGAATACGGTTACGGT |
| | | GGTTACCCGTATGAAGCTATGGATGTTTGGGGCCAAGGCAC |
| | | CCTGGTGACTGTTAGCTCA |
| 194 | Heavy Chain | QVQLVQSGAEVKKPGSSVKVSCKASGGTFRDYAISWVRQAP |
| | | GQGLEWMGGIIPAFGTANYAQKFQGRVTITADESTSTAYMELS |
| | | SLRSEDTAVYYCAREQDPEYGYGGYPYEAMDVWGQGTLVTV |
| | | SSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYASTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 195 | DNA Heavy | CAGGTGCAATTGGTGCAGAGCGGTGCCGAAGTGAAAAAAC |
| | Chain | CGGGCAGCAGCGTGAAAGTTAGCTGCAAAGCATCCGGAGG |
| | | GACGTTTCGTGACTACGCTATCTCTTGGGTGCGCCAGGCCC |
| | | CGGGCCAGGGCCTCGAGTGGATGGGCGGTATCATCCCGG |
| | | CTTTCGGCACTGCGAACTACGCCCAGAAATTTCAGGGCCG |
| | | GGTGACCATTACCGCCGATGAAAGCACCAGCACCGCCTATA |
| | | TGGAACTGAGCAGCCTGCGCAGCGAAGATACGGCCGTGTA |
| | | TTATTGCGCGCGTGAACAGGACCCGGAATACGGTTACGGT |
| | | GGTTACCCGTATGAAGCTATGGATGTTTGGGGCCAAGGCAC |
| | | CCTGGTGACTGTTAGCTCAGCCTCCACCAAGGGTCCATCG |
| | | GTCTTCCCCCTGGCACCCTCCTCCAAGAGCACCTCTGGGG |
| | | GCACAGCGGCCCTGGGCTGCCTGGTCAAGGACTACTTCCC |
| | | CGAACCGGTGACGGTGTCGTGGAACTCAGGCGCCCTGACC |
| | | AGCGGCGTGCACACCTTCCCGGCTGTCCTACAGTCCTCAG |
| | | GACTCTACTCCCTCAGCAGCGTGGTGACCGTGCCCTCCAG |
| | | CAGCTTGGGCACCCAGACCTACATCTGCAACGTGAATCACA |
| | | AGCCCAGCAACACCAAGGTGGACAAGAGAGTTGAGCCCAA |
| | | ATCTTGTGACAAAACTCACACATGCCCACCGTGCCCAGCAC |
| | | CTGAACTCCTGGGGGGACCGTCAGTCTTCCTCTTCCCCCCA |
| | | AAACCCAAGGACACCCTCATGATCTCCCGGACCCCTGAGGT |
| | | CACATGCGTGGTGGTGGACGTGAGCCACGAAGACCCTGAG |
| | | GTCAAGTTCAACTGGTACGTGGACGGCGTGGAGGTGCATA |
| | | ATGCCAAGACAAAGCCGCGGGAGGAGCAGTACGCCAGCAC |
| | | GTACCGGGTGGTCAGCGTCCTCACCGTCCTGCACCAGGAC |
| | | TGGCTGAATGGCAAGGAGTACAAGTGCAAGGTCTCCAACAA |
| | | AGCCCTCCCAGCCCCCATCGAGAAAACCATCTCCAAAGCCA |
| | | AAGGGCAGCCCCGAGAACCACAGGTGTACACCCTGCCCCC |
| | | ATCCCGGGAGGAGATGACCAAGAACCAGGTCAGCCTGACC |
| | | TGCCTGGTCAAAGGCTTCTATCCCAGCGACATCGCCGTGGA |
| | | GTGGGAGAGCAATGGGCAGCCGGAGAACAACTACAAGACC |
| | | ACGCCTCCCGTGCTGGACTCCGACGGCTCCTTCTTCCTCTA |
| | | CAGCAAGCTCACCGTGGACAAGAGCAGGTGGCAGCAGGG |
| | | GAACGTCTTCTCATGCTCCGTGATGCATGAGGCTCTGCACA |
| | | ACCACTACACGCAGAAGAGCCTCTCCCTGTCTCCGGGTAAA |
| 196 | LCDR1 | SGDNIPQHSVH |
| | (Combined) | |
| 197 | LCDR2 | DDTERPS |
| | (Combined) | |
| 198 | LCDR3 | SSWDSSMDSVV |
| | (Combined) | |
| 199 | LCDR1 | SGDNIPQHSVH |
| | (Kabat) | |
| 200 | LCDR2 | DDTERPS |
| | (Kabat) | |
| 201 | LCDR3 | SSWDSSMDSVV |
| | (Kabat) | |
| 202 | LCDR1 | DNIPQHS |
| | (Chothia) | |
| 203 | LCDR2 | DDT |
| | (Chothia) | |
| 204 | LCDR3 | WDSSMDSV |
| | (Chothia) | |
| 205 | VL | SYELTQPLSVSVALGQTARITCSGDNIPQHSVHWYQQKPGQA |
| | | PVLVIYDDTERPSGIPERFSGSNSGNTATLTISRAQAGDEADYY |
| | | CSSWDSSMPSWFGGGTKLTVL |
| 206 | DNA VL | AGCTACGAACTGACCCAGCCGCTGAGCGTGAGCGTGGCCC |
| | | TGGGCCAGACCGCGAGGATTACCTGTAGCGGCGATAACAT |
| | | CCCGCAGCATTCTGTTCATTGGTACCAGCAGAAACCGGGCC |
| | | AGGCGCCGGTGCTGGTGATCTACGACGACACTGAACGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTTGGGACTCTTCTATGG |
| | | ACTCTGTTGTGTTTGGCGGCGGCACGAAGTTAACCGTCCTA |
| 207 | Light Chain | SYELTQPLSVSVALGQTARITCSGDNIPQHSVHWYQQKPGQA |
| | | PVLVIYDDTERPSGIPERFSGSNSGNTATLTISRAQAGDEADYY |
| | | CSSWDSSMDSVVFGGGTKLTVLGQPKAAPSVTLFPPSSEELQ |
| | | ANKATLVCLISDFYPGAVTVAWKADSSPVKAGVETTTPSKQSN |
| | | NKYAASSYLSLTPEQWKSHRSYSCQVTHEGSTVEKTVAPTEC |
| | | S |
| 208 | DNA Light | AGCTACGAACTGACCCAGCCGCTGAGCGTGAGCGTGGCCC |
| | Chain | TGGGCCAGACCGCGAGGATTACCTGTAGCGGCGATAACAT |
| | | CCCGCAGCATTCTGTTCATTGGTACCAGCAGAAACCGGGCC |
| | | AGGCGCCGGTGCTGGTGATCTACGACGACACTGAACGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTTGGGACTCTTCTATGG |
| | | ACTCTGTTGTGTTTGGCGGCGGCACGAAGTTAACCGTCCTA |
| | | GGTCAGCCCAAGGCTGCCCCCTCGGTCACTCTGTTCCCGC |
| | | CCTCCTCTGAGGAGCTTCAAGCCAACAAGGCCACACTGGT |
| | | GTGTCTCATAAGTGACTTCTACCCGGGAGCCGTGACAGTGG |
| | | CCTGGAAGGCAGATAGCAGCCCCGTCAAGGCGGGAGTGGA |
| | | GACCACCACACCCTCCAAACAAAGCAACAACAAGTACGCGG |
| | | CCAGCAGCTATCTGAGCCTGACGCCTGAGCAGTGGAAGTC |
| | | CCACAGAAGCTACAGCTGCCAGGTCACGCATGAAGGGAGC |
| | | ACCGTGGAGAAGACAGTGGCCCCTACAGAATGTTCA |

NOV1218

| | | |
|---|---|---|
| 209 | HCDR1 | GFTFPTHGLH |
| | (Combined) | |
| 210 | HCDR2 | AISYDASETNYADSVKG |
| | (Combined) | |
| 211 | HCDR3 | ESIGGYFDY |
| | (Combined) | |
| 212 | HCDR1 | THGLH |
| | (Kabat) | |
| 213 | HCDR2 | AISYDASETNYADSVKG |
| | (Kabat) | |
| 214 | HCDR3 | ESIGGYFDY |
| | (Kabat) | |
| 215 | HCDR1 | GFTFPTH |
| | (Chothia) | |
| 216 | HCDR2 | SYDASE |
| | (Chothia) | |
| 217 | HCDR3 | ESIGGYFDY |
| | (Chothia) | |
| 218 | VH | QVQLLESGGGLVQPGGSLRLSCAASGFTFPTHGLHVWRQAP |
| | | GKGLEVWSAISYDASETNYADSVKGRFTISRDNSKNTLYLQMN |
| | | SLRAEDTAVYYCARESIGGYFDYWGQGTLVTVSS |
| 219 | DNA VH | CAGGTGCAGCTGCTGGAATCAGGCGGCGGACTGGTGCAGC |
| | | CTGGCGGTAGCCTGAGACTGAGCTGCGCTGCTAGTGGCTT |
| | | CACCTTCCCTACTCACGGCCTGCACTGGGTCAGACAGGCC |
| | | CCTGGTAAAGGCCTGGAGTGGGTCAGCGCTATTAGCTACG |
| | | ACGCTAGTGAAACTAACTACGCCGATAGCGTGAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCTAGAGAGTCTATCGGCGGCTACTTCGACTACTG |
| | | GGGTCAGGGCACCCTGGTCACCGTGTCTAGC |
| 220 | Heavy Chain | QVQLLESGGGLVQPGGSLRLSCAASGFTFPTHGLHWVRQAP |
| | | GKGLEWVSAISYDASETNYADSVKGRFTISRDNSKNTLYLQMN |
| | | SLRAEDTAVYYCARESIGGYFDYWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKRVEPKSCDKTHTCPPCPAPELLGGPSVFLFPPKPKDTLMI |
| | | SRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 221 | DNA Heavy | CAGGTGCAGCTGCTGGAATCAGGCGGCGGACTGGTGCAGC |
| | Chain | CTGGCGGTAGCCTGAGACTGAGCTGCGCTGCTAGTGGCTT |
| | | CACCTTCCCTACTCACGGCCTGCACTGGGTCAGACAGGCC |
| | | CCTGGTAAAGGCCTGGAGTGGGTCAGCGCTATTAGCTACG |
| | | ACGCTAGTGAAACTAACTACGCCGATAGCGTGAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCTAGAGAGTCTATCGGCGGCTACTTCGACTACTG |
| | | GGGTCAGGGCACCCTGGTCACCGTGTCTAGCGCTAGCACT |
| | | AAGGGCCCAAGTGTGTTTCCCCTGGCCCCCAGCAGCAAGT |
| | | CTACTTCCGGCGGAACTGCTGCCCTGGGTTGCCTGGTGAA |
| | | GGACTACTTCCCCGAGCCCGTGACAGTGTCCTGGAACTCT |
| | | GGGGCTCTGACTTCCGGCGTGCACACCTTCCCCGCCGTGC |
| | | TGCAGAGCAGCGGCCTGTACAGCCTGAGCAGCGTGGTGAC |
| | | AGTGCCCTCCAGCTCTCTGGGAACCCAGACCTATATCTGCA |
| | | ACGTGAACCACAAGCCCAGCAACACCAAGGTGGACAAGAG |
| | | AGTGGAGCCCAAGAGCTGCGACAAGACCCACACCTGCCCC |
| | | CCCTGCCCAGCTCCAGAACTGCTGGGAGGGCCTTCCGTGT |
| | | TCCTGTTCCCCCCCAAGCCCAAGGACACCCTGATGATCAGC |
| | | AGGACCCCCGAGGTGACCTGCGTGGTGGTGGACGTGTCCC |
| | | ACGAGGACCCAGAGGTGAAGTTCAACTGGTACGTGGACGG |
| | | CGTGGAGGTGCACAACGCCAAGACCAAGCCCAGAGAGGAG |
| | | CAGTACAACAGCACCTACAGGGTGGTGTCCGTGCTGACCG |
| | | TGCTGCACCAGGACTGGCTGAACGGCAAAGAATACAAGTG |
| | | CAAAGTCTCCAACAAGGCCCTGCCAGCCCCAATCGAAAAGA |
| | | CAATCAGCAAGGCCAAGGGCCAGCCACGGGAGCCCCAGGT |
| | | GTACACCCTGCCCCCCAGCCGGGAGGAGATGACCAAGAAC |
| | | CAGGTGTCCCTGACCTGTCTGGTGAAGGGCTTCTACCCCA |
| | | GCGATATCGCCGTGGAGTGGGAGAGCAACGGCCAGCCCG |
| | | AGAACAACTACAAGACCACCCCCCCAGTGCTGGACAGCGA |
| | | CGGCAGCTTCTTCCTGTACAGCAAGCTGACCGTGGACAAGT |
| | | CCAGGTGGCAGCAGGGCAACGTGTTCAGCTGCAGCGTGAT |
| | | GCACGAGGCCCTGCACAACCACTACACCCAGAAGTCCCTG |
| | | AGCCTGAGCCCCGGCAAG |
| 222 | LCDR1 | SGDALGKNTVS |
| | (Combined) | |
| 223 | LCDR2 | DDTDRPS |
| | (Combined) | |
| 224 | LCDR3 | SSTDLSTVV |
| | (Combined) | |
| 225 | LCDR1 | SGDALGKNTVS |
| | (Kabat) | |
| 226 | LCDR2 | DDTDRPS |
| | (Kabat) | |
| 227 | LCDR3 | SSTDLSTVV |
| | (Kabat) | |
| 228 | LCDR1 | DALGKNT |
| | (Chothia) | |
| 229 | LCDR2 | DDT |
| | (Chothia) | |
| 230 | LCDR3 | TDLSTV |
| | (Chothia) | |
| 231 | VL | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTVVFGGGTKLTVL |
| 232 | DNA VL | AGCTACGAGCTGACTCAGCCCCTGAGCGTCAGCGTGGCCC |
| | | TGGGTCAGACCGCTAGAATCACCTGTAGCGGCGACGCCCT |
| | | GGGTAAAAACACCGTCAGCTGGTATCAGCAGAAGCCCGGT |
| | | CAGGCCCCCGTGCTGGTGATCTACGACGACACCGATAGAC |
| | | CTAGCGGAATCCCCGAGCGGTTTAGCGGCTCTAATAGCGG |
| | | TAACACCGCTACCCTGACTATCTCTAGGGCTCAGGCCGGC |
| | | GACGAGGCCGACTACTACTGCTCTAGCACCGACCTGAGCA |
| | | CCGTGGTGTTCGGCGGAGGCACTAAGCTGACCGTGCTG |
| 233 | Light Chain | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTVVFGGGTKLTVLGQPKAAPSVTLFPPSSEELQANK |
| | | ATLVCLISDFYPGAVTVAWKADSSPVKAGVETTTPSKQSNNKY |
| | | AASSYLSLTPEQWKSHRSYSCQVTHEGSTVEKTVAPTECS |
| 234 | DNA Light | AGCTACGAGCTGACTCAGCCCCTGAGCGTCAGCGTGGCCC |
| | Chain | TGGGTCAGACCGCTAGAATCACCTGTAGCGGCGACGCCCT |
| | | GGGTAAAAACACCGTCAGCTGGTATCAGCAGAAGCCCGGT |
| | | CAGGCCCCCGTGCTGGTGATCTACGACGACACCGATAGAC |
| | | CTAGCGGAATCCCCGAGCGGTTTAGCGGCTCTAATAGCGG |
| | | TAACACCGCTACCCTGACTATCTCTAGGGCTCAGGCCGGC |
| | | GACGAGGCCGACTACTACTGCTCTAGCACCGACCTGAGCA |
| | | CCGTGGTGTTCGGCGGAGGCACTAAGCTGACCGTGCTGGG |
| | | TCAGCCTAAGGCTGCCCCCAGCGTGACCCTGTTCCCCCCC |
| | | AGCAGCGAGGAGCTGCAGGCCAACAAGGCCACCCTGGTGT |
| | | GCCTGATCAGCGACTTCTACCCAGGCGCCGTGACCGTGGC |
| | | CTGGAAGGCCGACAGCAGCCCCGTGAAGGCCGGCGTGGA |
| | | GACCACCACCCCCAGCAAGCAGAGCAACAACAAGTACGCC |
| | | GCCAGCAGCTACCTGAGCCTGACCCCCGAGCAGTGGAAGA |
| | | GCCACAGGTCCTACAGCTGCCAGGTGACCCACGAGGGCAG |
| | | CACCGTGGAAAAGACCGTGGCCCCAACCGAGTGCAGC |

NOV1218_N297A

| | | |
|---|---|---|
| 235 | HCDR1 | GFTFPTHGLH |
| | (Combined) | |
| 236 | HCDR2 | AISYDASETNYADSVKG |
| | (Combined) | |
| 237 | HCDR3 | ESIGGYFDY |
| | (Combined) | |
| 238 | HCDR1 | THGLH |
| | (Kabat) | |
| 239 | HCDR2 | AISYDASETNYADSVKG |
| | (Kabat) | |
| 240 | HCDR3 | ESIGGYFDY |
| | (Kabat) | |
| 241 | HCDR1 | GFTFPTH |
| | (Chothia) | |
| 242 | HCDR2 | SYDASE |
| | (Chothia) | |
| 243 | HCDR3 | ESIGGYFDY |
| | (Chothia) | |
| 244 | VH | QVQLLESGGGLVQPGGSLRLSCAASGFTFPTHGLHVWRQAP |
| | | GKGLEWVSAISYDASETNYADSVKGRFTISRDNSKNTLYLQMN |
| | | SLRAEDTAVYYCARESIGGYFDYWGQGTLVTVSS |
| 245 | DNA VH | CAGGTGCAATTGCTGGAAAGCGGCGGTGGCCTGGTGCAGC |
| | | CGGGTGGCAGCCTGCGTCTGAGCTGCGCGGCGTCCGGATT |
| | | CACCTTTCCTACTCATGGTCTGCATTGGGTGCGCCAGGCCC |
| | | CGGGCAAAGGTCTCGAGTGGGTTTCCGCTATCTCTTACGAC |
| | | GCCTCTGAAACCAACTATGCGGATAGCGTGAAAGGCCGCTT |
| | | TACCATCAGCCGCGATAATTCGAAAAACACCCTGTATCTGC |
| | | AAATGAACAGCCTGCGTGCGGAAGATACGGCCGTGTATTAT |
| | | TGCGCGCGTGAATCTATCGGTGGTTACTTCGATTACTGGGG |
| | | CCAAGGCACCCTGGTGACTGTTAGCTCA |
| 246 | Heavy Chain | QVQLLESGGGLVQPGGSLRLSCAASGFTFPTHGLHVWRQAP |
| | | GKGLEWVSAISYDASETNYADSVKGRFTISRDNSKNTLYLQMN |
| | | SLRAEDTAVYYCARESIGGYFDYWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKRVEPKSCDKTHTCPPCPAPELLGGPSVFLFPPKPKDTLMI |
| | | SRTPEVTCVVVDVSHEDPEVKFNWYVDGVEVHNAKTKPREE |
| | | QYASTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 247 | DNA Heavy | CAGGTGCAATTGCTGGAAAGCGGCGGTGGCCTGGTGCAGC |
| | Chain | CGGGTGGCAGCCTGCGTCTGAGCTGCGCGGCGTCCGGATT |
| | | CACCTTTCCTACTCATGGTCTGCATTGGGTGCGCCAGGCCC |
| | | CGGGCAAAGGTCTCGAGTGGGTTTCCGCTATCTCTTACGAC |
| | | GCCTCTGAAACCAACTATGCGGATAGCGTGAAAGGCCGCTT |
| | | TACCATCAGCCGCGATAATTCGAAAAACACCCTGTATCTGC |
| | | AAATGAACAGCCTGCGTGCGGAAGATACGGCCGTGTATTAT |
| | | TGCGCGCGTGAATCTATCGGTGGTTACTTCGATTACTGGGG |
| | | CCAAGGCACCCTGGTGACTGTTAGCTCAGCCTCCACCAAG |
| | | GGTCCATCGGTCTTCCCCCTGGCACCCTCCTCCAAGAGCA |
| | | CCTCTGGGGGCACAGCGGCCCTGGGCTGCCTGGTCAAGG |
| | | ACTACTTCCCCGAACCGGTGACGGTGTCGTGGAACTCAGG |
| | | CGCCCTGACCAGCGGCGTGCACACCTTCCCGGCTGTCCTA |
| | | CAGTCCTCAGGACTCTACTCCCTCAGCAGCGTGGTGACCGT |
| | | GCCCTCCAGCAGCTTGGGCACCCAGACCTACATCTGCAAC |
| | | GTGAATCACAAGCCCAGCAACACCAAGGTGGACAAGAGAG |
| | | TTGAGCCCAAATCTTGTGACAAAACTCACACATGCCCACCG |
| | | TGCCCAGCACCTGAACTCCTGGGGGGACCGTCAGTCTTCC |
| | | TCTTCCCCCCAAAACCCAAGGACACCCTCATGATCTCCCGG |
| | | ACCCCTGAGGTCACATGCGTGGTGGTGGACGTGAGCCACG |
| | | AAGACCCTGAGGTCAAGTTCAACTGGTACGTGGACGGCGT |
| | | GGAGGTGCATAATGCCAAGACAAAGCCGCGGGAGGAGCAG |
| | | TACGCCAGCACGTACCGGGTGGTCAGCGTCCTCACCGTCC |
| | | TGCACCAGGACTGGCTGAATGGCAAGGAGTACAAGTGCAA |
| | | GGTCTCCAACAAAGCCCTCCCAGCCCCCATCGAGAAAACCA |
| | | TCTCCAAAGCCAAAGGGCAGCCCCGAGAACCACAGGTGTA |
| | | CACCCTGCCCCCATCCCGGGAGGAGATGACCAAGAACCAG |
| | | GTCAGCCTGACCTGCCTGGTCAAAGGCTTCTATCCCAGCGA |
| | | CATCGCCGTGGAGTGGGAGAGCAATGGGCAGCCGGAGAA |
| | | CAACTACAAGACCACGCCTCCCGTGCTGGACTCCGACGGC |
| | | TCCTTCTTCCTCTACAGCAAGCTCACCGTGGACAAGAGCAG |
| | | GTGGCAGCAGGGGAACGTCTTCTCATGCTCCGTGATGCAT |
| | | GAGGCTCTGCACAACCACTACACGCAGAAGAGCCTCTCCCT |
| | | GTCTCCGGGTAAA |
| 248 | LCDR1 | SGDALGKNTVS |
| | (Combined) | |
| 249 | LCDR2 | DDTDRPS |
| | (Combined) | |
| 250 | LCDR3 | SSTDLSTVV |
| | (Combined) | |
| 251 | LCDR1 | SGDALGKNTVS |
| | (Kabat) | |
| 252 | LCDR2 | DDTDRPS |
| | (Kabat) | |
| 253 | LCDR3 | SSTDLSTVV |
| | (Kabat) | |
| 254 | LCDR1 | DALGKNT |
| | (Chothia) | |
| 255 | LCDR2 | DDT |
| | (Chothia) | |
| 256 | LCDR3 | TDLSTV |
| | (Chothia) | |
| 257 | VL | SYELTQPLSVSVALGQTARITCSGDALGKNTVSWYQQKPGQA |
| | | PVLVIYQDTDRPSGIPERFSGSNSGNTATLTISRAQAGDEADYY |
| | | CSSTDLSTVVFGGGTKLTVL |
| 258 | DNA VL | AGCTACGAACTGACCCAGCCGCTGAGCGTGAGCGTGGCCC |
| | | TGGGCCAGACCGCGAGGATTACCTGTAGCGGCGATGCTCT |
| | | GGGTAAAAACACTGTTTCTTGGTACCAGCAGAAACCGGGCC |
| | | AGGCGCCGGTGCTGGTGATCTACGACGACACTGACCGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTACTGACCTGTCTACTG |
| | | TTGTGTTTGGCGGCGGCACGAAGTTAACCGTCCTA |
| 259 | Light Chain | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTWFGGGTKLTVLGQPKAAPSVTLFPPSSEELQANK |
| | | ATLVCLISDFYPGAVTVAWKADSSPVKAGVETTTPSKQSNNKY |
| | | AASSYLSLTPEQWKSHRSYSCQVTHEGSTVEKTVAPTECS |
| 260 | DNA Light | AGCTACGAACTGACCCAGCCGCTGAGCGTGAGCGTGGCCC |
| | Chain | TGGGCCAGACCGCGAGGATTACCTGTAGCGGCGATGCTCT |
| | | GGGTAAAAACACTGTTTCTTGGTACCAGCAGAAACCGGGCC |
| | | AGGCGCCGGTGCTGGTGATCTACGACGACACTGACCGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTACTGACCTGTCTACTG |
| | | TTGTGTTTGGCGGCGGCACGAAGTTAACCGTCCTAGGTCAG |
| | | CCCAAGGCTGCCCCCTCGGTCACTCTGTTCCCGCCCTCCT |
| | | CTGAGGAGCTTCAAGCCAACAAGGCCACACTGGTGTGTCTC |
| | | ATAAGTGACTTCTACCCGGGAGCCGTGACAGTGGCCTGGA |
| | | AGGCAGATAGCAGCCCCGTCAAGGCGGGAGTGGAGACCAC |
| | | CACACCCTCCAAACAAAGCAACAACAAGTACGCGGCCAGCA |
| | | GCTATCTGAGCCTGACGCCTGAGCAGTGGAAGTCCCACAG |
| | | AAGCTACAGCTGCCAGGTCACGCATGAAGGGAGCACCGTG |
| | | GAGAAGACAGTGGCCCCTACAGAATGTTCA |

NOV1219

| | | |
|---|---|---|
| 261 | HCDR1 | GFTFPTHGLH |
| | (Combined) | |
| 262 | HCDR2 | AISYEGSETNYADSVKG |
| | (Combined) | |
| 263 | HCDR3 | ESIGGYFDY |
| | (Combined) | |
| 264 | HCDR1 | THGLH |
| | (Kabat) | |
| 265 | HCDR2 | AISYEGSETNYADSVKG |
| | (Kabat) | |
| 266 | HCDR3 | ESIGGYFDY |
| | (Kabat) | |
| 267 | HCDR1 | GFTFPTH |
| | (Chothia) | |
| 268 | HCDR2 | SYEGSE |
| | (Chothia) | |
| 269 | HCDR3 | ESIGGYFDY |
| | (Chothia) | |
| 270 | VH | QVQLLESGGGLVQPGGSLRLSCAASGFTFPTHGLHVWRQAP |
| | | GKGLEWVSAISYEGSETNYADSVKGRFTISRDNSKNTLYLQMN |
| | | SLRAEDTAVYYCARESIGGYFDYWGQGTLVTVSS |
| 271 | DNA VH | CAGGTGCAGCTGCTGGAATCAGGCGGCGGACTGGTGCAGC |
| | | CTGGCGGTAGCCTGAGACTGAGCTGCGCTGCTAGTGGCTT |
| | | CACCTTCCCTACTCACGGCCTGCACTGGGTCAGACAGGCC |
| | | CCTGGTAAAGGCCTGGAGTGGGTCAGCGCTATTAGCTACG |
| | | AGGGTAGCGAGACTAACTACGCCGATAGCGTGAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCTAGAGAGTCTATCGGCGGCTACTTCGACTACTG |
| | | GGGTCAGGGCACCCTGGTCACCGTGTCTAGC |
| 272 | Heavy Chain | QVGLLESGGGLVQPGGSLRLSCAASGFTFPTHGLHVWRQAP |
| | | GKGLEVWSAISYEGSETNYADSVKGRFTISRDNSKNTLYLQMN |
| | | SLRAEDTAVYYCARESIGGYFDYWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKRVEPKSCDKTHTCPPCPAPELLGGPSVFLFPPKPKDTLMI |
| | | SRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 273 | DNA Heavy | CAGGTGCAGCTGCTGGAATCAGGCGGCGGACTGGTGCAGC |
| | Chain | CTGGCGGTAGCCTGAGACTGAGCTGCGCTGCTAGTGGCTT |
| | | CACCTTCCCTACTCACGGCCTGCACTGGGTCAGACAGGCC |
| | | CCTGGTAAAGGCCTGGAGTGGGTCAGCGCTATTAGCTACG |
| | | AGGGTAGCGAGACTAACTACGCCGATAGCGTGAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCTAGAGAGTCTATCGGCGGCTACTTCGACTACTG |
| | | GGGTCAGGGCACCCTGGTCACCGTGTCTAGCGCTAGCACT |
| | | AAGGGCCCAAGTGTGTTTCCCCTGGCCCCCAGCAGCAAGT |
| | | CTACTTCCGGCGGAACTGCTGCCCTGGGTTGCCTGGTGAA |
| | | GGACTACTTCCCCGAGCCCGTGACAGTGTCCTGGAACTCT |
| | | GGGGCTCTGACTTCCGGCGTGCACACCTTCCCCGCCGTGC |
| | | TGCAGAGCAGCGGCCTGTACAGCCTGAGCAGCGTGGTGAC |
| | | AGTGCCCTCCAGCTCTCTGGGAACCCAGACCTATATCTGCA |
| | | ACGTGAACCACAAGCCCAGCAACACCAAGGTGGACAAGAG |
| | | AGTGGAGCCCAAGAGCTGCGACAAGACCCACACCTGCCCC |
| | | CCCTGCCCAGCTCCAGAACTGCTGGGAGGGCCTTCCGTGT |
| | | TCCTGTTCCCCCCCAAGCCCAAGGACACCCTGATGATCAGC |
| | | AGGACCCCCGAGGTGACCTGCGTGGTGGTGGACGTGTCCC |
| | | ACGAGGACCCAGAGGTGAAGTTCAACTGGTACGTGGACGG |
| | | CGTGGAGGTGCACAACGCCAAGACCAAGCCCAGAGAGGAG |
| | | CAGTACAACAGCACCTACAGGGTGGTGTCCGTGCTGACCG |
| | | TGCTGCACCAGGACTGGCTGAACGGCAAAGAATACAAGTG |
| | | CAAAGTCTCCAACAAGGCCCTGCCAGCCCCAATCGAAAAGA |
| | | CAATCAGCAAGGCCAAGGGCCAGCCACGGGAGCCCCAGGT |
| | | GTACACCCTGCCCCCCAGCCGGGAGGAGATGACCAAGAAC |
| | | CAGGTGTCCCTGACCTGTCTGGTGAAGGGCTTCTACCCCA |
| | | GCGATATCGCCGTGGAGTGGGAGAGCAACGGCCAGCCCG |
| | | AGAACAACTACAAGACCACCCCCCCAGTGCTGGACAGCGA |
| | | CGGCAGCTTCTTCCTGTACAGCAAGCTGACCGTGGACAAGT |
| | | CCAGGTGGCAGCAGGGCAACGTGTTCAGCTGCAGCGTGAT |
| | | GCACGAGGCCCTGCACAACCACTACACCCAGAAGTCCCTG |
| | | AGCCTGAGCCCCGGCAAG |
| 274 | LCDR1 | SGDALGKNTVS |
| | (Combined) | |
| 275 | LCDR2 | DDTDRPS |
| | (Combined) | |
| 276 | LCDR3 | SSTDLSTVV |
| | (Combined) | |
| 277 | LCDR1 | SGDALGKNTVS |
| | (Kabat) | |
| 278 | LCDR2 | DDTDRPS |
| | (Kabat) | |
| 279 | LCDR3 | SSTDLSTVV |
| | (Kabat) | |
| 280 | LCDR1 | DALGKNT |
| | (Chothia) | |
| 281 | LCDR2 | DDT |
| | (Chothia) | |
| 282 | LCDR3 | TDLSTV |
| | (Chothia) | |
| 283 | VL | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTWFGGGTKLTVL |
| 284 | DNA VL | AGCTACGAGCTGACTCAGCCCCTGAGCGTCAGCGTGGCCC |
| | | TGGGTCAGACCGCTAGAATCACCTGTAGCGGCGACGCCCT |
| | | GGGTAAAAACACCGTCAGCTGGTATCAGCAGAAGCCCGGT |
| | | CAGGCCCCCGTGCTGGTGATCTACGACGACACCGATAGAC |
| | | CTAGCGGAATCCCCGAGCGGTTTAGCGGCTCTAATAGCGG |
| | | TAACACCGCTACCCTGACTATCTCTAGGGCTCAGGCCGGC |
| | | GACGAGGCCGACTACTACTGCTCTAGCACCGACCTGAGCA |
| | | CCGTGGTGTTCGGCGGAGGCACTAAGCTGACCGTGCTG |
| 285 | Light Chain | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTVVFGGGTKLTVLGQPKAAPSVTLFPPSSEELQANK |
| | | ATLVCLISDFYPGAVTVAWKADSSPVKAGVETTTPSKQSNNKY |
| | | AASSYLSLTPEQWKSHRSYSCQVTHEGSTVEKTVAPTECS |
| 286 | DNA Light | AGCTACGAGCTGACTCAGCCCCTGAGCGTCAGCGTGGCCC |
| | Chain | TGGGTCAGACCGCTAGAATCACCTGTAGCGGCGACGCCCT |
| | | GGGTAAAAACACCGTCAGCTGGTATCAGCAGAAGCCCGGT |
| | | CAGGCCCCCGTGCTGGTGATCTACGACGACACCGATAGAC |
| | | CTAGCGGAATCCCCGAGCGGTTTAGCGGCTCTAATAGCGG |
| | | TAACACCGCTACCCTGACTATCTCTAGGGCTCAGGCCGGC |
| | | GACGAGGCCGACTACTACTGCTCTAGCACCGACCTGAGCA |
| | | CCGTGGTGTTCGGCGGAGGCACTAAGCTGACCGTGCTGGG |
| | | TCAGCCTAAGGCTGCCCCCAGCGTGACCCTGTTCCCCCCC |
| | | AGCAGCGAGGAGCTGCAGGCCAACAAGGCCACCCTGGTGT |
| | | GCCTGATCAGCGACTTCTACCCAGGCGCCGTGACCGTGGC |
| | | CTGGAAGGCCGACAGCAGCCCCGTGAAGGCCGGCGTGGA |
| | | GACCACCACCCCCAGCAAGCAGAGCAACAACAAGTACGCC |
| | | GCCAGCAGCTACCTGAGCCTGACCCCCGAGCAGTGGAAGA |
| | | GCCACAGGTCCTACAGCTGCCAGGTGACCCACGAGGGCAG |
| | | CACCGTGGAAAAGACCGTGGCCCCAACCGAGTGCAGC |

NOV1219_N297A

| | | |
|---|---|---|
| 287 | HCDR1 | GFTFPTHGLH |
| | (Combined) | |
| 288 | HCDR2 | AISYEGSETNYADSVKG |
| | (Combined) | |
| 289 | HCDR3 | ESIGGYFDY |
| | (Combined) | |
| 290 | HCDR1 | THGLH |
| | (Kabat) | |
| 291 | HCDR2 | AISYEGSETNYADSVKG |
| | (Kabat) | |
| 292 | HCDR3 | ESIGGYFDY |
| | (Kabat) | |
| 293 | HCDR1 | GFTFPTH |
| | (Chothia) | |
| 294 | HCDR2 | SYEGSE |
| | (Chothia) | |
| 295 | HCDR3 | ESIGGYFDY |
| | (Chothia) | |
| 296 | VH | QVQLLESGGGLVQPGGSLRLSCAASGFTFPTHGLHVWRQAP |
| | | GKGLEWVSAISYEGSETNYADSVKGRFTISRDNSKNTLYLQMN |
| | | SLRAEDTAVYYCARESIGGYFDYWGQGTLVTVSS |
| 297 | DNA VH | CAGGTGCAATTGCTGGAAAGCGGCGGTGGCCTGGTGCAGC |
| | | CGGGTGGCAGCCTGCGTCTGAGCTGCGCGGCGTCCGGATT |
| | | CACCTTTCCTACTCATGGTCTGCATTGGGTGCGCCAGGCCC |
| | | CGGGCAAAGGTCTCGAGTGGGTTTCCGCTATCTCTTACGAG |
| | | GGTTCTGAAACCAACTATGCGGATAGCGTGAAAGGCCGCTT |
| | | TACCATCAGCCGCGATAATTCGAAAAACACCCTGTATCTGC |
| | | AAATGAACAGCCTGCGTGCGGAAGATACGGCCGTGTATTAT |
| | | TGCGCGCGTGAATCTATCGGTGGTTACTTCGATTACTGGGG |
| | | CCAAGGCACCCTGGTGACTGTTAGCTCA |
| 298 | Heavy Chain | QVQLLESGGGLVQPGGSLRLSCAASGFTFPTHGLHVWRQAP |
| | | GKGLEWVSAISYEGSETNYADSVKGRFTISRDNSKNTLYLQMN |
| | | SLRAEDTAVYYCARESIGGYFDYWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKRVEPKSCDKTHTCPPCPAPELLGGPSVFLFPPKPKDTLMI |
| | | SRTPEVTCVVVDVSHEDPEVKFNWYVDGVEVHNAKTKPREE |
| | | QYASTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 299 | DNA Heavy | CAGGTGCAATTGCTGGAAAGCGGCGGTGGCCTGGTGCAGC |
| | Chain | CGGGTGGCAGCCTGCGTCTGAGCTGCGCGGCGTCCGGATT |
| | | CACCTTTCCTACTCATGGTCTGCATTGGGTGCGCCAGGCCC |
| | | CGGGCAAAGGTCTCGAGTGGGTTTCCGCTATCTCTTACGAG |
| | | GGTTCTGAAACCAACTATGCGGATAGCGTGAAAGGCCGCTT |
| | | TACCATCAGCCGCGATAATTCGAAAAACACCCTGTATCTGC |
| | | AAATGAACAGCCTGCGTGCGGAAGATACGGCCGTGTATTAT |
| | | TGCGCGCGTGAATCTATCGGTGGTTACTTCGATTACTGGGG |
| | | CCAAGGCACCCTGGTGACTGTTAGCTCAGCCTCCACCAAG |
| | | GGTCCATCGGTCTTCCCCCTGGCACCCTCCTCCAAGAGCA |
| | | CCTCTGGGGGCACAGCGGCCCTGGGCTGCCTGGTCAAGG |
| | | ACTACTTCCCCGAACCGGTGACGGTGTCGTGGAACTCAGG |
| | | CGCCCTGACCAGCGGCGTGCACACCTTCCCGGCTGTCCTA |
| | | CAGTCCTCAGGACTCTACTCCCTCAGCAGCGTGGTGACCGT |
| | | GCCCTCCAGCAGCTTGGGCACCCAGACCTACATCTGCAAC |
| | | GTGAATCACAAGCCCAGCAACACCAAGGTGGACAAGAGAG |
| | | TTGAGCCCAAATCTTGTGACAAAACTCACACATGCCCACCG |
| | | TGCCCAGCACCTGAACTCCTGGGGGGACCGTCAGTCTTCC |
| | | TCTTCCCCCCAAAACCCAAGGACACCCTCATGATCTCCCGG |
| | | ACCCCTGAGGTCACATGCGTGGTGGTGGACGTGAGCCACG |
| | | AAGACCCTGAGGTCAAGTTCAACTGGTACGTGGACGGCGT |
| | | GGAGGTGCATAATGCCAAGACAAAGCCGCGGGAGGAGCAG |
| | | TACGCCAGCACGTACCGGGTGGTCAGCGTCCTCACCGTCC |
| | | TGCACCAGGACTGGCTGAATGGCAAGGAGTACAAGTGCAA |
| | | GGTCTCCAACAAAGCCCTCCCAGCCCCCATCGAGAAAACCA |
| | | TCTCCAAAGCCAAAGGGCAGCCCCGAGAACCACAGGTGTA |
| | | CACCCTGCCCCCATCCCGGGAGGAGATGACCAAGAACCAG |
| | | GTCAGCCTGACCTGCCTGGTCAAAGGCTTCTATCCCAGCGA |
| | | CATCGCCGTGGAGTGGGAGAGCAATGGGCAGCCGGAGAA |
| | | CAACTACAAGACCACGCCTCCCGTGCTGGACTCCGACGGC |
| | | TCCTTCTTCCTCTACAGCAAGCTCACCGTGGACAAGAGCAG |
| | | GTGGCAGCAGGGGAACGTCTTCTCATGCTCCGTGATGCAT |
| | | GAGGCTCTGCACAACCACTACACGCAGAAGAGCCTCTCCCT |
| | | GTCTCCGGGTAAA |
| 300 | LCDR1 | SGDALGKNTVS |
| | (Combined) | |
| 301 | LCDR2 | DDTDRPS |
| | (Combined) | |
| 302 | LCDR3 | SSTDLSTVV |
| | (Combined) | |
| 303 | LCDR1 | SGDALGKNTVS |
| | (Kabat) | |
| 304 | LCDR2 | DDTDRPS |
| | (Kabat) | |
| 305 | LCDR3 | SSTDLSTVV |
| | (Kabat) | |
| 306 | LCDR1 | DALGKNT |
| | (Chothia) | |
| 307 | LCDR2 | DDT |
| | (Chothia) | |
| 308 | LCDR3 | TDLSTV |
| | (Chothia) | |
| 309 | VL | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTVVFGGGTKLTVL |
| 310 | DNA VL | AGCTACGAACTGACCCAGCCGCTGAGCGTGAGCGTGGCCC |
| | | TGGGCCAGACCGCGAGGATTACCTGTAGCGGCGATGCTCT |
| | | GGGTAAAAACACTGTTTCTTGGTACCAGCAGAAACCGGGCC |
| | | AGGCGCCGGTGCTGGTGATCTACGACGACACTGACCGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTACTGACCTGTCTACTG |
| | | TTGTGTTTGGCGGCGGCACGAAGTTAACCGTCCTA |
| 311 | Light Chain | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTVVFGGGTKLTVLGQPKAAPSVTLFPPSSEELQANK |
| | | ATLVCLISDFYPGAVTVAWKADSSPVKAGVETTTPSKQSNNKY |
| | | AASSYLSLTPEQWKSHRSYSCQVTHEGSTVEKTVAPTECS |
| 312 | DNA Light | AGCTACGAACTGACCCAGCCGCTGAGCGTGAGCGTGGCCC |
| | Chain | TGGGCCAGACCGCGAGGATTACCTGTAGCGGCGATGCTCT |
| | | GGGTAAAAACACTGTTTCTTGGTACCAGCAGAAACCGGGCC |
| | | AGGCGCCGGTGCTGGTGATCTACGACGACACTGACCGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTACTGACCTGTCTACTG |
| | | TTGTGTTTGGCGGCGGCACGAAGTTAACCGTCCTAGGTCAG |
| | | CCCAAGGCTGCCCCCTCGGTCACTCTGTTCCCGCCCTCCT |
| | | CTGAGGAGCTTCAAGCCAACAAGGCCACACTGGTGTGTCTC |
| | | ATAAGTGACTTCTACCCGGGAGCCGTGACAGTGGCCTGGA |
| | | AGGCAGATAGCAGCCCCGTCAAGGCGGGAGTGGAGACCAC |
| | | CACACCCTCCAAACAAAGCAACAACAAGTACGCGGCCAGCA |
| | | GCTATCTGAGCCTGACGCCTGAGCAGTGGAAGTCCCACAG |
| | | AAGCTACAGCTGCCAGGTCACGCATGAAGGGAGCACCGTG |
| | | GAGAAGACAGTGGCCCCTACAGAATGTTCA |

NOV2106

| | | |
|---|---|---|
| 313 | HCDR1 | GGTFRDYAIS |
| | (Combined) | |
| 314 | HCDR2 | GIIPAFGTANYAQKFQG |
| | (Combined) | |
| 315 | HCDR3 | EQDPEFGYGGYPYEAMDV |
| | (Combined) | |
| 316 | HCDR1 | DYAIS |
| | (Kabat) | |
| 317 | HCDR2 | GIIPAFGTANYAQKFQG |
| | (Kabat) | |
| 318 | HCDR3 | EQDPEFGYGGYPYEAMDV |
| | (Kabat) | |
| 319 | HCDR1 | GGTFRDY |
| | (Chothia) | |
| 320 | HCDR2 | IPAFGT |
| | (Chothia) | |
| 321 | HCDR3 | EQDPEFGYGGYPYEAMDV |
| | (Chothia) | |
| 322 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEFGYGGYPYEAMPVWGQGTLVTVSS |
| 323 | DNA VH | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTTCGGCTACGGCGGCTACCCCTACGAG |
| | | GCTATGGACGTGTGGGGTCAGGGCACCCTGGTCACCGTGTCT |
| | | AGC |
| 324 | Heavy Chain | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEFGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 325 | DNA Heavy | CAGGTGCAGCTGGTGCAGTCAGGCGCCGAAGTGAAGAAACC |
| | Chain | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTTCGGCTACGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 326 | LCDR1 | SGDNIPQHSVH |
| | (Combined) | |
| 327 | LCDR2 | DDTERPS |
| | (Combined) | |
| 328 | LCDR3 | SSWDSSMDSVV |
| | (Combined) | |
| 329 | LCDR1 | SGDNIPQHSVH |
| | (Kabat) | |
| 330 | LCDR2 | DDTERPS |
| | (Kabat) | |
| 331 | LCDR3 | SSWDSSMDSVV |
| | (Kabat) | |
| 332 | LCDR1 | DNIPQHS |
| | (Chothia) | |
| 333 | LCDR2 | DDT |
| | (Chothia) | |
| 334 | LCDR3 | WDSSMDSV |
| | (Chothia) | |
| 335 | VL | SYELTQPLSVSVALGQTARITCSGDNIPQHSVHWVQQKPGQAPV |
| | | LVIYDDTERPSGIPERFSGSNSGNTATLTISRAQAGDEADYYCSS |
| | | WDSSMDSVVFGGGTKLTVL |
| 336 | DNA VL | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 337 | Light Chain | SYELTQPLSVSVALGQTARITCSGDNIPQHSVHWVQQKPGQAPV |
| | | LVIYDDTERPSGIPERFSGSNSGNTATLTISRAQAGDEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 338 | DNA Light | AGCTACGAGCTGACTCAGCCCCTGAGCGTCAGCGTGGCCCTG |
| | Chain | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

NOV2106_N297A

| | | |
|---|---|---|
| 339 | HCDR1 | GGTFRDYAIS |
| | (Combined) | |
| 340 | HCDR2 | GIIPAFGTANYAQKFQG |
| | (Combined) | |
| 341 | HCDR3 | EQDPEFGYGGYPYEAMDV |
| | (Combined) | |
| 342 | HCDR1 | DYAIS |
| | (Kabat) | |
| 343 | HCDR2 | GIIPAFGTANYAQKFQG |
| | (Kabat) | |
| 344 | HCDR3 | EQDPEFGYGGYPYEAMDV |
| | (Kabat) | |
| 345 | HCDR1 | GGTFRDY |
| | (Chothia) | |
| 346 | HCDR2 | IPAFGT |
| | (Chothia) | |
| 347 | HCDR3 | EQDPEFGYGGYPYEAMDV |
| | (Chothia) | |
| 348 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEFGYGGYPYEAMDVWGQGTLVTVSS |
| 349 | DNA VH | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATTCGGTTACGGTGGTTACCCGTATGA |
| | | AGCTATGGATGTTTGGGGCCAAGGCACCCTGGTGACTGTTAG |
| | | CTCA |
| 350 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEFGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 351 | DNA Heavy | CAGGTGCAATTGGTGCAGAGCGGTGCCGAAGTGAAAAAACCG |
| | Chain | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATTCGGTTACGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 352 | LCDR1 | SGDNIPQHSVH |
| | (Combined) | |
| 353 | LCDR2 | DDTERPS |
| | (Combined) | |
| 354 | LCDR3 | SSWDSSMDSVV |
| | (Combined) | |
| 355 | LCDR1 | SGDNIPQHSVH |
| | (Kabat) | |
| 356 | LCDR2 | DDTERPS |
| | (Kabat) | |
| 357 | LCDR3 | SSWDSSMDSVV |
| | (Kabat) | |
| 358 | LCDR1 | DNIPQHS |
| | (Chothia) | |
| 359 | LCDR2 | DDT |
| | (Chothia) | |
| 360 | LCDR3 | WDSSMDSV |
| | (Chothia) | |
| 361 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 362 | DNA VL | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 363 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 364 | DNA Light | AGCTACGAACTGACCCAGCCGCTGAGCGTGAGCGTGGCCCT |
| | Chain | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

NOV2107

| | | |
|---|---|---|
| 365 | HCDR1 | GGTFRDYAIS |
| | (Combined) | |
| 366 | HCDR2 | GIIPAFGTANYAQKFQG |
| | (Combined) | |
| 367 | HCDR3 | EQDPEAGYGGYPYEAMDV |
| | (Combined) | |
| 368 | HCDR1 | DYAIS |
| | (Kabat) | |
| 369 | HCDR2 | GIIPAFGTANYAQKFQG |
| | (Kabat) | |
| 370 | HCDR3 | EQDPEAGYGGYPYEAMDV |
| | (Kabat) | |
| 371 | HCDR1 | GGTFRDY |
| | (Chothia) | |
| 372 | HCDR2 | IPAFGT |
| | (Chothia) | |
| 373 | HCDR3 | EQDPEAGYGGYPYEAMDV |
| | (Chothia) | |
| 374 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEAGYGGYPYEAMDVWGQGTLVTVSS |
| 375 | DNA VH | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGGCCGGCTACGGCGGCTACCCCTACGAG |
| | | GCTATGGACGTGTGGGGTCAGGGCACCCTGGTCACCGTGTCT |
| | | AGC |
| 376 | Heavy Chain | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEAGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 377 | DNA Heavy | CAGGTGCAGCTGGTGCAGTCAGGCGCCGAAGTGAAGAAACC |
| | Chain | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGGCCGGCTACGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 378 | LCDR1 | SGDNIPQHSVH |
| | (Combined) | |
| 379 | LCDR2 | DDTERPS |
| | (Combined) | |
| 380 | LCDR3 | SSWDSSMDSVV |
| | (Combined) | |
| 381 | LCDR1 | SGDNIPQHSVH |
| | (Kabat) | |
| 382 | LCDR2 | DDTERPS |
| | (Kabat) | |
| 383 | LCDR3 | SSWDSSMDSVV |
| | (Kabat) | |
| 384 | LCDR1 | DNIPQHS |
| | (Chothia) | |
| 385 | LCDR2 | DDT |
| | (Chothia) | |
| 386 | LCDR3 | WDSSMDSV |
| | (Chothia) | |
| 387 | VL | SYELTQPLSVSVALGQTARITCSGDNIPQHSVHWVQQKPGQAPV |
| | | LVIYDDTERPSGIPERFSGSNSGNTATLTISRAQAGDEADYYCSS |
| | | WDSSMDSVVFGGGTKLTVL |
| 388 | DNA VL | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 389 | Light Chain | SYELTQPLSVSVALGQTARITCSGDNIPQHSVHWVQQKPGQAPV |
| | | LVIYDDTERPSGIPERFSGSNSGNTATLTISRAQAGDEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 390 | DNA Light | AGCTACGAGCTGACTCAGCCCCTGAGCGTCAGCGTGGCCCTG |
| | Chain | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

NOV2107_N297A

| | | |
|---|---|---|
| 391 | HCDR1 | GGTFRDYAIS |
| | (Combined) | |
| 392 | HCDR2 | GIIPAFGTANYAQKFQG |
| | (Combined) | |
| 393 | HCDR3 | EQDPEAGYGGYPYEAMDV |
| | (Combined) | |
| 394 | HCDR1 | DYAIS |
| | (Kabat) | |
| 395 | HCDR2 | GIIPAFGTANYAQKFQG |
| | (Kabat) | |
| 396 | HCDR3 | EQDPEAGYGGYPYEAMDV |
| | (Kabat) | |
| 397 | HCDR1 | GGTFRDY |
| | (Chothia) | |
| 398 | HCDR2 | IPAFGT |
| | (Chothia) | |
| 399 | HCDR3 | EQDPEAGYGGYPYEAMDV |
| | (Chothia) | |
| 400 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEAGYGGYPYEAMDVWGQGTLVTVSS |
| 401 | DNA VH | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAAGCCGGTTACGGTGGTTACCCGTATGA |
| | | AGCTATGGATGTTTGGGGCCAAGGCACCCTGGTGACTGTTAG |
| | | CTCA |
| 402 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEAGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 403 | DNA Heavy | CAGGTGCAATTGGTGCAGAGCGGTGCCGAAGTGAAAAAACCG |
| | Chain | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAAGCCGGTTACGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 404 | LCDR1 | SGDNIPQHSVH |
| | (Combined) | |
| 405 | LCDR2 | DDTERPS |
| | (Combined) | |
| 406 | LCDR3 | SSWDSSMDSVV |
| | (Combined) | |
| 407 | LCDR1 | SGDNIPQHSVH |
| | (Kabat) | |
| 408 | LCDR2 | DDTERPS |
| | (Kabat) | |
| 409 | LCDR3 | SSWDSSMDSVV |
| | (Kabat) | |
| 410 | LCDR1 | DNIPQHS |
| | (Chothia) | |
| 411 | LCDR2 | DDT |
| | (Chothia) | |
| 412 | LCDR3 | WDSSMDSV |
| | (Chothia) | |
| 413 | VL | SYELTQPLSVSVALGQTARITCSGDNIPQHSVHVWQQKPGQAPV |
| | | LVIYDDTERPSGIPERFSGSNSGNTATLTISRAQAGDEADYYCSS |
| | | WDSSMDSWFGGGTKLTVL |
| 414 | DNA VL | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 415 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 416 | DNA Light | AGCTACGAACTGACCCAGCCGCTGAGCGTGAGCGTGGCCCT |
| | Chain | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

NOV2108

| | | |
|---|---|---|
| 417 | HCDR1 | GGTFRDYAIS |
| | (Combined) | |
| 418 | HCDR2 | GIIPAFGTANYAQKFQG |
| | (Combined) | |
| 419 | HCDR3 | EQDPESGYGGYPYEAMDV |
| | (Combined) | |
| 420 | HCDR1 | DYAIS |
| | (Kabat) | |
| 421 | HCDR2 | GIIPAFGTANYAQKFQG |
| | (Kabat) | |
| 422 | HCDR3 | EQDPESGYGGYPYEAMDV |
| | (Kabat) | |
| 423 | HCDR1 | GGTFRDY |
| | (Chothia) | |
| 424 | HCDR2 | IPAFGT |
| | (Chothia) | |
| 425 | HCDR3 | EQDPESGYGGYPYEAMDV |
| | (Chothia) | |
| 426 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPESGYGGYPYEAMDVWGQGTLVTVSS |
| 427 | DNA VH | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTCCGGCTACGGCGGCTACCCCTACGAG |
| | | GCTATGGACGTGTGGGGTCAGGGCACCCTGGTCACCGTGTCT |
| | | AGC |
| 428 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPESGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 429 | DNA Heavy | CAGGTGCAGCTGGTGCAGTCAGGCGCCGAAGTGAAGAAACC |
| | Chain | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTCCGGCTACGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 430 | LCDR1 | SGDNIPQHSVH |
| | (Combined) | |
| 431 | LCDR2 | DDTERPS |
| | (Combined) | |
| 432 | LCDR3 | SSWDSSMDSVV |
| | (Combined) | |
| 433 | LCDR1 | SGDNIPQHSVH |
| | (Kabat) | |
| 434 | LCDR2 | DDTERPS |
| | (Kabat) | |
| 435 | LCDR3 | SSWDSSMDSVV |
| | (Kabat) | |
| 436 | LCDR1 | DNIPQHS |
| | (Chothia) | |
| 437 | LCDR2 | DDT |
| | (Chothia) | |
| 438 | LCDR3 | WDSSMDSV |
| | (Chothia) | |
| 439 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 440 | DNA VL | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 441 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 442 | DNA Light | AGCTACGAGCTGACTCAGCCCCTGAGCGTCAGCGTGGCCCTG |
| | Chain | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

NOV2108_N297A

| | | |
|---|---|---|
| 443 | HCDR1 | GGTFRDYAIS |
| | (Combined) | |
| 444 | HCDR2 | GIIPAFGTANYAQKFQG |
| | (Combined) | |
| 445 | HCDR3 | EQDPESGYGGYPYEAMDV |
| | (Combined) | |
| 446 | HCDR1 | DYAIS |
| | (Kabat) | |
| 447 | HCDR2 | GIIPAFGTANYAQKFQG |
| | (Kabat) | |
| 448 | HCDR3 | EQDPESGYGGYPYEAMDV |
| | (Kabat) | |
| 449 | HCDR1 | GGTFRDY |
| | (Chothia) | |
| 450 | HCDR2 | IPAFGT |
| | (Chothia) | |
| 451 | HCDR3 | EQDPESGYGGYPYEAMDV |
| | (Chothia) | |
| 452 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPESGYGGYPYEAMDVWGQGTLVTVSS |
| 453 | DNA VH | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAAAGCGGTTACGGTGGTTACCCGTATGA |
| | | AGCTATGGATGTTTGGGGCCAAGGCACCCTGGTGACTGTTAG |
| | | CTCA |
| 454 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPESGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 455 | DNA Heavy | CAGGTGCAATTGGTGCAGAGCGGTGCCGAAGTGAAAAAACCG |
| | Chain | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAAAGCGGTTACGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 456 | LCDR1 | SGDNIPQHSVH |
| | (Combined) | |
| 457 | LCDR2 | DDTERPS |
| | (Combined) | |
| 458 | LCDR3 | SSWDSSMDSVV |
| | (Combined) | |
| 459 | LCDR1 | SGDNIPQHSVH |
| | (Kabat) | |
| 460 | LCDR2 | DDTERPS |
| | (Kabat) | |
| 461 | LCDR3 | SSWDSSMDSVV |
| | (Kabat) | |
| 462 | LCDR1 | DNIPQHS |
| | (Chothia) | |
| 463 | LCDR2 | DDT |
| | (Chothia) | |
| 464 | LCDR3 | WDSSMDSV |
| | (Chothia) | |
| 465 | VL | SYELTQPLSVSVALGQTARITCSGDNIPQHSVHVWQQKPGQAPV |
| | | LVIYDDTERPSGIPERFSGSNSGNTATLTISRAQAGDEADYYCSS |
| | | WDSSMDSVVFGGGTKLTVL |
| 466 | DNA VL | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 467 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 468 | DNA Light | AGCTACGAACTGACCCAGCCGCTGAGCGTGAGCGTGGCCCT |
| | Chain | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

NOV2109

| | | |
|---|---|---|
| 469 | HCDR1 | GGTFRDYAIS |
| | (Combined) | |
| 470 | HCDR2 | GIIPAFGTANYAQKFQG |
| | (Combined) | |
| 471 | HCDR3 | EQDPEYGFGGYPYEAMDV |
| | (Combined) | |
| 472 | HCDR1 | DYAIS |
| | (Kabat) | |
| 473 | HCDR2 | GIIPAFGTANYAQKFQG |
| | (Kabat) | |
| 474 | HCDR3 | EQDPEYGFGGYPYEAMDV |
| | (Kabat) | |
| 475 | HCDR1 | GGTFRDY |
| | (Chothia) | |
| 476 | HCDR2 | IPAFGT |
| | (Chothia) | |
| 477 | HCDR3 | EQDPEYGFGGYPYEAMDV |
| | (Chothia) | |
| 478 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGFGGYPYEAMDVWGQGTLVTVSS |
| 479 | DNA VH | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTACGGCTTCGGCGGCTACCCCTACGAG |
| | | GCTATGGACGTGTGGGGTCAGGGCACCCTGGTCACCGTGTCT |
| | | AGC |
| 480 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGFGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 481 | DNA Heavy | CAGGTGCAGCTGGTGCAGTCAGGCGCCGAAGTGAAGAAACC |
| | Chain | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTACGGCTTCGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 482 | LCDR1 | SGDNIPQHSVH |
| | (Combined) | |
| 483 | LCDR2 | DDTERPS |
| | (Combined) | |
| 484 | LCDR3 | SSWDSSMDSVV |
| | (Combined) | |
| 485 | LCDR1 | SGDNIPQHSVH |
| | (Kabat) | |
| 486 | LCDR2 | DDTERPS |
| | (Kabat) | |
| 487 | LCDR3 | SSWDSSMDSVV |
| | (Kabat) | |
| 488 | LCDR1 | DNIPQHS |
| | (Chothia) | |
| 489 | LCDR2 | DDT |
| | (Chothia) | |
| 490 | LCDR3 | WDSSMDSV |
| | (Chothia) | |
| 491 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 492 | DNA VL | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 493 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 494 | DNA Light | AGCTACGAGCTGACTCAGCCCCTGAGCGTCAGCGTGGCCCTG |
| | Chain | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

NOV2109_N297A

| | | |
|---|---|---|
| 495 | HCDR1 | GGTFRDYAIS |
| | (Combined) | |
| 496 | HCDR2 | GIIPAFGTANYAQKFQG |
| | (Combined) | |
| 497 | HCDR3 | EQDPEYGFGGYPYEAMDV |
| | (Combined) | |
| 498 | HCDR1 | DYAIS |
| | (Kabat) | |
| 499 | HCDR2 | GIIPAFGTANYAQKFQG |
| | (Kabat) | |
| 500 | HCDR3 | EQDPEYGFGGYPYEAMDV |
| | (Kabat) | |
| 501 | HCDR1 | GGTFRDY |
| | (Chothia) | |
| 502 | HCDR2 | IPAFGT |
| | (Chothia) | |
| 503 | HCDR3 | EQDPEYGFGGYPYEAMDV |
| | (Chothia) | |
| 504 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEYGFGGYPYEAMDVWGQGTLVTVSS |
| 505 | DNA VH | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTTCGGTGGTTACCCGTATGA |
| | | AGCTATGGATGTTTGGGGCCAAGGCACCCTGGTGACTGTTAG |
| | | CTCA |
| 506 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGFGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 507 | DNA Heavy | CAGGTGCAATTGGTGCAGAGCGGTGCCGAAGTGAAAAAACCG |
| | Chain | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTTCGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 508 | LCDR1 | SGDNIPQHSVH |
| | (Combined) | |
| 509 | LCDR2 | DDTERPS |
| | (Combined) | |
| 510 | LCDR3 | SSWDSSMDSVV |
| | (Combined) | |
| 511 | LCDR1 | SGDNIPQHSVH |
| | (Kabat) | |
| 512 | LCDR2 | DDTERPS |
| | (Kabat) | |
| 513 | LCDR3 | SSWDSSMDSVV |
| | (Kabat) | |
| 514 | LCDR1 | DNIPQHS |
| | (Chothia) | |
| 515 | LCDR2 | DDT |
| | (Chothia) | |
| 516 | LCDR3 | WDSSMDSV |
| | (Chothia) | |
| 517 | VL | SYELTQPLSVSVALGQTARITCSGDNIPQHSVHWYQQKPGQAPV |
| | | LVIYQDTERPSGIPERFSGSNSGNTATLTISRAQAGDEADYYCSS |
| | | WDSSMDSVVFGGGTKLTVL |
| 518 | DNA VL | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 519 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEGWKSHRSYSCQVTHEGSTVEKTVAPTECS |
| 520 | DNA Light | AGCTACGAACTGACCCAGCCGCTGAGCGTGAGCGTGGCCCT |
| | Chain | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

NOV2110_N297A

| | | |
|---|---|---|
| 521 | HCDR1 | GGTFRDYAIS |
| | (Combined) | |
| 522 | HCDR2 | GIIPAFGTANYAQKFQG |
| | (Combined) | |
| 523 | HCDR3 | EQDPEYGYGGFPVEAMDV |
| | (Combined) | |
| 524 | HCDR1 | DYAIS |
| | (Kabat) | |
| 525 | HCDR2 | GIIPAFGTANYAQKFQG |
| | (Kabat) | |
| 526 | HCDR3 | EQDPEYGYGGFPYEAMDV |
| | (Kabat) | |
| 527 | HCDR1 | GGTFRDY |
| | (Chothia) | |
| 528 | HCDR2 | IPAFGT |
| | (Chothia) | |
| 529 | HCDR3 | EQDPEYGYGGFPYEAMDV |
| | (Chothia) | |
| 530 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGYGGFPYEAMDVWGQGTLVTVSS |
| 531 | DNA VH | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTACGGTGGTTTCCCGTATGA |
| | | AGCTATGGATGTTTGGGGCCAAGGCACCCTGGTGACTGTTAG |
| | | CTCA |
| 532 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGYGGFPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 533 | DNA Heavy | CAGGTGCAATTGGTGCAGAGCGGTGCCGAAGTGAAAAAACCG |
| | Chain | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTACGGTGGTTTCCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 534 | LCDR1 | SGDNIPQHSVH |
| | (Combined) | |
| 535 | LCDR2 | DDTERPS |
| | (Combined) | |
| 536 | LCDR3 | SSWDSSMDSVV |
| | (Combined) | |
| 537 | LCDR1 | SGDNIPQHSVH |
| | (Kabat) | |
| 538 | LCDR2 | DDTERPS |
| | (Kabat) | |
| 539 | LCDR3 | SSWDSSMDSVV |
| | (Kabat) | |
| 540 | LCDR1 | DNIPQHS |
| | (Chothia) | |
| 541 | LCDR2 | DDT |
| | (Chothia) | |
| 542 | LCDR3 | WDSSMDSV |
| | (Chothia) | |
| 543 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 544 | DNA VL | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 545 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 546 | DNA Light | AGCTACGAACTGACCCAGCCGCTGAGCGTGAGCGTGGCCCT |
| | Chain | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

N0V2111_N297A

| | | |
|---|---|---|
| 547 | HCDR1 | GGTFRDYAIS |
| | (Combined) | |
| 548 | HCDR2 | GIIPAFGTANYAQKFQG |
| | (Combined) | |
| 549 | HCDR3 | EQDPEYGYGGYPFEAMDV |
| | (Combined) | |
| 550 | HCDR1 | DYAIS |
| | (Kabat) | |
| 551 | HCDR2 | GIIPAFGTANYAQKFQG |
| | (Kabat) | |
| 552 | HCDR3 | EQDPEYGYGGYPFEAMDV |
| | (Kabat) | |
| 553 | HCDR1 | GGTFRDY |
| | (Chothia) | |
| 554 | HCDR2 | IPAFGT |
| | (Chothia) | |
| 555 | HCDR3 | EQDPEYGYGGYPFEAMDV |
| | (Chothia) | |
| 556 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEYGYGGYPFEAMDVWGQGTLVTVSS |
| 557 | DNA VH | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTACGGTGGTTACCCGTTCGA |
| | | AGCTATGGATGTTTGGGGCCAAGGCACCCTGGTGACTGTTAG |
| | | CTCA |
| 558 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGYGGYPFEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 559 | DNA Heavy | CAGGTGCAATTGGTGCAGAGCGGTGCCGAAGTGAAAAAACCG |
| | Chain | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTACGGTGGTTACCCGTTCGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 560 | LCDR1 | SGDNIPQHSVH |
| | (Combined) | |
| 561 | LCDR2 | DDTERPS |
| | (Combined) | |
| 562 | LCDR3 | SSWDSSMDSVV |
| | (Combined) | |
| 563 | LCDR1 | SGDNIPQHSVH |
| | (Kabat) | |
| 564 | LCDR2 | DDTERPS |
| | (Kabat) | |
| 565 | LCDR3 | SSWDSSMDSVV |
| | (Kabat) | |
| 566 | LCDR1 | DNIPQHS |
| | (Chothia) | |
| 567 | LCDR2 | DDT |
| | (Chothia) | |
| 568 | LCDR3 | WDSSMDSV |
| | (Chothia) | |
| 569 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 570 | DNA VL | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 571 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 572 | DNA Light | AGCTACGAACTGACCCAGCCGCTGAGCGTGAGCGTGGCCCT |
| | Chain | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

NOV2112

| | | |
|---|---|---|
| 573 | HCDR1 | GGTFRDYAIS |
| | (Combined) | |
| 574 | HCDR2 | GIIPAFGTANYAQKFQG |
| | (Combined) | |
| 575 | HCDR3 | EQDPSYGYGGYPYEAMDV |
| | (Combined) | |
| 576 | HCDR1 | DYAIS |
| | (Kabat) | |
| 577 | HCDR2 | GIIPAFGTANYAQKFQG |
| | (Kabat) | |
| 578 | HCDR3 | EQDPSYGYGGYPYEAMDV |
| | (Kabat) | |
| 579 | HCDR1 | GGTFRDY |
| | (Chothia) | |
| 580 | HCDR2 | IPAFGT |
| | (Chothia) | |
| 581 | HCDR3 | EQDPSYGYGGYPYEAMDV |
| | (Chothia) | |
| 582 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPSYGYGGYPYEAMDVWGQGTLVTVSS |
| 583 | DNA VH | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCTCCTACGGCTACGGCGGCTACCCCTACGAG |
| | | GCTATGGACGTGTGGGGTCAGGGCACCCTGGTCACCGTGTCT |
| | | AGC |
| 584 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPSYGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 585 | DNA Heavy | CAGGTGCAGCTGGTGCAGTCAGGCGCCGAAGTGAAGAAACC |
| | Chain | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCTCCTACGGCTACGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 586 | LCDR1 | SGDNIPQHSVH |
| | (Combined) | |
| 587 | LCDR2 | DDTERPS |
| | (Combined) | |
| 588 | LCDR3 | SSWDSSMDSVV |
| | (Combined) | |
| 589 | LCDR1 | SGDNIPQHSVH |
| | (Kabat) | |
| 590 | LCDR2 | DDTERPS |
| | (Kabat) | |
| 591 | LCDR3 | SSWDSSMDSVV |
| | (Kabat) | |
| 592 | LCDR1 | DNIPQHS |
| | (Chothia) | |
| 593 | LCDR2 | DDT |
| | (Chothia) | |
| 594 | LCDR3 | WDSSMDSV |
| | (Chothia) | |
| 595 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSWFGGGTKLTVL |
| 596 | DNA VL | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 597 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 598 | DNA Light | AGCTACGAGCTGACTCAGCCCCTGAGCGTCAGCGTGGCCCTG |
| | Chain | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

NOV2112_N297A

| | | |
|---|---|---|
| 599 | HCDR1 | GGTFRDYAIS |
| | (Combined) | |
| 600 | HCDR2 | GIIPAFGTANYAQKFQG |
| | (Combined) | |
| 601 | HCDR3 | EQDPSYGYGGYPYEAMDV |
| | (Combined) | |
| 602 | HCDR1 | DYAIS |
| | (Kabat) | |
| 603 | HCDR2 | GIIPAFGTANYAQKFQG |
| | (Kabat) | |
| 504 | HCDR3 | EQDPSYGYGGYPYEAMDV |
| | (Kabat) | |
| 605 | HCDR1 | GGTFRDY |
| | (Chothia) | |
| 606 | HCDR2 | IPAFGT |
| | (Chothia) | |
| 607 | HCDR3 | EQDPSYGYGGYPYEAMDV |
| | (Chothia) | |
| 608 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPSYGYGGYPYEAMDVWGQGTLVTVSS |
| 609 | DNA VH | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGAGCTACGGTTACGGTGGTTACCCGTATGA |
| | | AGCTATGGATGTTTGGGGCCAAGGCACCCTGGTGACTGTTAG |
| | | CTCA |
| 610 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPSYGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 611 | DNA Heavy | CAGGTGCAATTGGTGCAGAGCGGTGCCGAAGTGAAAAAACCG |
| | Chain | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGAGCTACGGTTACGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 612 | LCDR1 | SGDNIPGHGVH |
| | (Combined) | |
| 613 | LCDR2 | DDTERPS |
| | (Combined) | |
| 614 | LCDR3 | SSWDSSMDSVV |
| | (Combined) | |
| 615 | LCDR1 | SGDNIPQHSVH |
| | (Kabat) | |
| 616 | LCDR2 | DDTERPS |
| | (Kabat) | |
| 617 | LCDR3 | SSWDSSMDSVV |
| | (Kabat) | |
| 618 | LCDR1 | DNIPQHS |
| | (Chothia) | |
| 619 | LCDR2 | DDT |
| | (Chothia) | |
| 620 | LCDR3 | WDSSMDSV |
| | (Chothia) | |
| 621 | VL | SYELTQPLSVSVALGQTARITCSGDNIPQHSVHVWQQKPGQAPV |
| | | LVIYDDTERPSGIPERFSGSNSGNTATLTISRAQAGDEADYYCSS |
| | | WDSSMDSWFGGGTKLTVL |
| 622 | DNA VL | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 623 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEGWKSHRSYSCQVTHEGSTVEKTVAPTECS |
| 624 | DNA Light | AGCTACGAACTGACCCAGCCGCTGAGCGTGAGCGTGGCCCT |
| | Chain | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

NOV2113

| | | |
|---|---|---|
| 625 | HCDR1 | GGTFRDYAIS |
| | (Combined) | |
| 626 | HCDR2 | GIIPAFGTANYAQKFQG |
| | (Combined) | |
| 627 | HCDR3 | EQSPEYGYGGYPYEAMDV |
| | (Combined) | |
| 628 | HCDR1 | DYAIS |
| | (Kabat) | |
| 629 | HCDR2 | GIIPAFGTANYAQKFQG |
| | (Kabat) | |
| 630 | HCDR3 | EQSPEYGYGGYPYEAMDV |
| | (Kabat) | |
| 631 | HCDR1 | GGTFRDY |
| | (Chothia) | |
| 632 | HCDR2 | IPAFGT |
| | (Chothia) | |
| 633 | HCDR3 | EQSPEYGYGGYPYEAMDV |
| | (Chothia) | |
| 634 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREGSPEYGYGGYPYEAMDVWGQGTLVTVSS |
| 635 | DNA VH | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGTCCCCCGAGTACGGCTACGGCGGCTACCCCTACGAG |
| | | GCTATGGACGTGTGGGGTCAGGGCACCCTGGTCACCGTGTCT |
| | | AGC |
| 636 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQSPEYGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 637 | DNA Heavy | CAGGTGCAGCTGGTGCAGTCAGGCGCCGAAGTGAAGAAACC |
| | Chain | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGTCCCCCGAGTACGGCTACGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 638 | LCDR1 | SGDNIPQHSVH |
| | (Combined) | |
| 639 | LCDR2 | DDTERPS |
| | (Combined) | |
| 640 | LCDR3 | SSWDSSMDSVV |
| | (Combined) | |
| 641 | LCDR1 | SGDNIPQHSVH |
| | (Kabat) | |
| 642 | LCDR2 | DDTERPS |
| | (Kabat) | |
| 643 | LCDR3 | SSWDSSMDSVV |
| | (Kabat) | |
| 644 | LCDR1 | DNIPQHS |
| | (Chothia) | |
| 645 | LCDR2 | DDT |
| | (Chothia) | |
| 646 | LCDR3 | WDSSMDSV |
| | (Chothia) | |
| 647 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 648 | DNA VL | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 649 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 650 | DNA Light | AGCTACGAGCTGACTCAGCCCCTGAGCGTCAGCGTGGCCCTG |
| | Chain | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

NOV2113_N297A

| | | |
|---|---|---|
| 651 | HCDR1 | GGTFRDYAIS |
| | (Combined) | |
| 652 | HCDR2 | GIIPAFGTANYAQKFQG |
| | (Combined) | |
| 653 | HCDR3 | EQSPEYGYGGYPYEAMDV |
| | (Combined) | |
| 654 | HCDR1 | DYAIS |
| | (Kabat) | |
| 655 | HCDR2 | GIIPAFGTANYAQKFQG |
| | (Kabat) | |
| 556 | HCDR3 | EQSPEYGYGGYPYEAMDV |
| | (Kabat) | |
| 657 | HCDR1 | GGTFRDY |
| | (Chothia) | |
| 658 | HCDR2 | IPAFGT |
| | (Chothia) | |
| 659 | HCDR3 | EQSPEYGYGGYPYEAMDV |
| | (Chothia) | |
| 660 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQSPEYGYGGYPYEAMDVWGQGTLVTVSS |
| 661 | DNA VH | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGAGCCCGGAATACGGTTACGGTGGTTACCCGTATGA |
| | | AGCTATGGATGTTTGGGGCCAAGGCACCCTGGTGACTGTTAG |
| | | CTCA |
| 662 | Heavy Chain | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQSPEYGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 663 | DNA Heavy | CAGGTGCAATTGGTGCAGAGCGGTGCCGAAGTGAAAAAACCG |
| | Chain | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGAGCCCGGAATACGGTTACGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 664 | LCDR1 | SGDNIPQHSVH |
| | (Combined) | |
| 665 | LCDR2 | DDTERPS |
| | (Combined) | |
| 666 | LCDR3 | SSWDSSMDSVV |
| | (Combined) | |
| 667 | LCDR1 | SGDNIPQHSVH |
| | (Kabat) | |
| 668 | LCDR2 | DDTERPS |
| | (Kabat) | |
| 669 | LCDR3 | SSWDSSMDSVV |
| | (Kabat) | |
| 670 | LCDR1 | DNIPQHS |
| | (Chothia) | |
| 671 | LCDR2 | DDT |
| | (Chothia) | |
| 672 | LCDR3 | WDSSMDSV |
| | (Chothia) | |
| 673 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGQQTKLTVL |
| 674 | DNA VL | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 675 | Light Chain | SYELTQPLSVSVALGQTARITCSGDNIPQHSVHWYQQKPGQAPV |
| | | LVIYDDTERPSGIPERFSGSNSGNTATLTISRAQAGQEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 676 | DNA Light | AGCTACGAACTGACCCAGCCGCTGAGCGTGAGCGTGGCCCT |
| | Chain | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

Other antibodies and antigen-binding fragments thereof of the invention include those wherein the amino acids or nucleic acids encoding the amino acids have been mutated, yet have at least 60, 70, 80, 90 or 95 percent identity to the sequences described in Table 1. In one embodiment, it includes mutant amino acid sequences wherein no more than 1, 2, 3, 4 or 5 amino acids have been mutated in the variable regions when compared with the variable regions depicted in the sequence described in Table 1, while retaining substantially the same therapeutic activity.

In another specific embodiment, the present invention provides an isolated antibody or antigen-binding fragment thereof, which binds human CD32b and comprises the HCDR1, HCDR2, and HCDR3 sequences of SEQ ID NOs: 1, 2, and 3, respectively, and the LCDR1, LCDR2, and LCDR3 sequences of SEQ ID NOs: 14, 15, and 16, respectively.

In another specific embodiment, the present invention provides an isolated antibody or antigen-binding fragment thereof, which binds human CD32b and comprises the HCDR1, HCDR2, and HCDR3 sequences of SEQ ID NOs: 4, 5, and 6, respectively, and the LCDR1, LCDR2, and LCDR3 sequences of SEQ ID NOs: 17, 18, and 19, respectively.

In another specific embodiment, the present invention provides an isolated antibody or antigen-binding fragment thereof, which binds human CD32b and comprises the HCDR1, HCDR2, and HCDR3 sequences of SEQ ID NOs: 7, 8, and 9, respectively, and the LCDR1, LCDR2, and LCDR3 sequences of SEQ ID NOs: 20, 21, and 22, respectively.

In another specific embodiment, the present invention provides an isolated antibody or antigen-binding fragment thereof, which binds human CD32b and comprises the HCDR1, HCDR2, and HCDR3 sequences of SEQ ID NOs: 53, 54, and 55, respectively, and the LCDR1, LCDR2, and LCDR3 sequences of SEQ ID NOs: 66, 67, and 68 respectively.

In another specific embodiment, the present invention provides an isolated antibody or antigen-binding fragment thereof, which binds human CD32b and comprises the HCDR1, HCDR2, and HCDR3 sequences of SEQ ID NOs: 56, 57, and 58, respectively, and the LCDR1, LCDR2, and LCDR3 sequences of SEQ ID NOs: 69, 70, and 71 respectively.

In another specific embodiment, the present invention provides an isolated antibody or antigen-binding fragment thereof, which binds human CD32b and comprises the HCDR1, HCDR2, and HCDR3 sequences of SEQ ID NOs: 59, 60, and 61, respectively, and the LCDR1, LCDR2, and LCDR3 sequences of SEQ ID NOs: 72, 73, and 74 respectively.

In another specific embodiment, the present invention provides an isolated antibody or antigen-binding fragment thereof, which binds human CD32b and comprises the HCDR1, HCDR2, and HCDR3 sequences of SEQ ID NOs: 105, 106, and 107 respectively, and the LCDR1, LCDR2, and LCDR3 sequences of SEQ ID NOs: 118, 119, 120, respectively.

In another specific embodiment, the present invention provides an isolated antibody or antigen-binding fragment thereof, which binds human CD32b and comprises the HCDR1, HCDR2, and HCDR3 sequences of SEQ ID NOs: 108, 109, and 110 respectively, and the LCDR1, LCDR2, and LCDR3 sequences of SEQ ID NOs: 121, 122, 123, respectively.

In another specific embodiment, the present invention provides an isolated antibody or antigen-binding fragment thereof, which binds human CD32b and comprises the HCDR1, HCDR2, and HCDR3 sequences of SEQ ID NOs: 111, 112, and 113 respectively, and the LCDR1, LCDR2, and LCDR3 sequences of SEQ ID NOs: 124, 125, 126, respectively.

In another specific embodiment, the present invention provides an isolated antibody or antigen-binding fragment thereof, which binds human CD32b and comprises the HCDR1, HCDR2, and HCDR3 sequences of SEQ ID NOs: 157, 158, and 159, respectively, and the LCDR1, LCDR2, and LCDR3 sequences of SEQ ID NOs: 170, 171, 172, respectively.

In another specific embodiment, the present invention provides an isolated antibody or antigen-binding fragment thereof, which binds human CD32b and comprises the HCDR1, HCDR2, and HCDR3 sequences of SEQ ID NOs: 160, 161, and 162, respectively, and the LCDR1, LCDR2, and LCDR3 sequences of SEQ ID NOs: 173, 174, 175, respectively.

In another specific embodiment, the present invention provides an isolated antibody or antigen-binding fragment thereof, which binds human CD32b and comprises the HCDR1, HCDR2, and HCDR3 sequences of SEQ ID NOs: 163, 164, and 165, respectively, and the LCDR1, LCDR2, and LCDR3 sequences of SEQ ID NOs: 176, 177, 178, respectively.

In another specific embodiment, the present invention provides an isolated antibody or antigen-binding fragment thereof, which binds human CD32b and comprises the HCDR1, HCDR2, and HCDR3 sequences of SEQ ID NOs: 209, 210, and 211, respectively, and the LCDR1, LCDR2, and LCDR3 sequences of SEQ ID NOs: 222, 223, and 224, respectively.

In another specific embodiment, the present invention provides an isolated antibody or antigen-binding fragment thereof, which binds human CD32b and comprises the HCDR1, HCDR2, and HCDR3 sequences of SEQ ID NOs: 212, 213, and 214, respectively, and the LCDR1, LCDR2, and LCDR3 sequences of SEQ ID NOs: 225, 226, and 227, respectively.

In another specific embodiment, the present invention provides an isolated antibody or antigen-binding fragment thereof, which binds human CD32b and comprises the HCDR1, HCDR2, and HCDR3 sequences of SEQ ID NOs: 215, 216, and 217 respectively, and the LCDR1, LCDR2, and LCDR3 sequences of SEQ ID NOs: 228, 229, and 230, respectively.

In another specific embodiment, the present invention provides an isolated antibody or antigen-binding fragment thereof, which binds human CD32b and comprises the HCDR1, HCDR2, and HCDR3 sequences of SEQ ID NOs: 261, 262, and 263, respectively, and the LCDR1, LCDR2, and LCDR3 sequences of SEQ ID NOs: 274, 275, and 276, respectively.

In another specific embodiment, the present invention provides an isolated antibody or antigen-binding fragment thereof, which binds human CD32b and comprises the HCDR1, HCDR2, and HCDR3 sequences of SEQ ID NOs: 264, 265, and 266, respectively, and the LCDR1, LCDR2, and LCDR3 sequences of SEQ ID NOs: 277, 278, and 279, respectively.

In another specific embodiment, the present invention provides an isolated antibody or antigen-binding fragment thereof, which binds human CD32b and comprises the HCDR1, HCDR2, and HCDR3 sequences of SEQ ID NOs: 267, 268, and 269, respectively, and the LCDR1, LCDR2, and LCDR3 sequences of SEQ ID NOs: 280, 281, and 282, respectively.

In another specific embodiment, the present invention provides an isolated antibody or antigen-binding fragment thereof, which binds human CD32b and comprises the HCDR1, HCDR2, and HCDR3 sequences of SEQ ID NOs: 313, 314, and 315, respectively, and the LCDR1, LCDR2, and LCDR3 sequences of SEQ ID NOs: 326, 327, and 328, respectively.

In another specific embodiment, the present invention provides an isolated antibody or antigen-binding fragment thereof, which binds human CD32b and comprises the HCDR1, HCDR2, and HCDR3 sequences of SEQ ID NOs: 316, 317, and 318, respectively, and the LCDR1, LCDR2, and LCDR3 sequences of SEQ ID NOs: 329, 330, and 331, respectively.

In another specific embodiment, the present invention provides an isolated antibody or antigen-binding fragment thereof, which binds human CD32b and comprises the HCDR1, HCDR2, and HCDR3 sequences of SEQ ID NOs: 319, 320, and 321, respectively, and the LCDR1, LCDR2, and LCDR3 sequences of SEQ ID NOs: 332, 333, and 334, respectively.

In another specific embodiment, the present invention provides an isolated antibody or antigen-binding fragment thereof, which binds human CD32b and comprises the HCDR1, HCDR2, and HCDR3 sequences of SEQ ID NOs: 365, 366, and 367, respectively, and the LCDR1, LCDR2, and LCDR3 sequences of SEQ ID NOs: 378, 379, and 380, respectively.

In another specific embodiment, the present invention provides an isolated antibody or antigen-binding fragment thereof, which binds human CD32b and comprises the HCDR1, HCDR2, and HCDR3 sequences of SEQ ID NOs: 368, 369, and 370, respectively, and the LCDR1, LCDR2, and LCDR3 sequences of SEQ ID NOs: 381, 382, and 383, respectively.

In another specific embodiment, the present invention provides an isolated antibody or antigen-binding fragment thereof, which binds human CD32b and comprises the HCDR1, HCDR2, and HCDR3 sequences of SEQ ID NOs: 371, 372, and 373, respectively, and the LCDR1, LCDR2, and LCDR3 sequences of SEQ ID NOs: 384, 385, and 386, respectively.

In another specific embodiment, the present invention provides an isolated antibody or antigen-binding fragment thereof, which binds human CD32b and comprises the HCDR1, HCDR2, and HCDR3 sequences of SEQ ID NOs: 417, 418, and 419, respectively, and the LCDR1, LCDR2, and LCDR3 sequences of SEQ ID NOs: 430, 431, and 432, respectively.

In another specific embodiment, the present invention provides an isolated antibody or antigen-binding fragment thereof, which binds human CD32b and comprises the HCDR1, HCDR2, and HCDR3 sequences of SEQ ID NOs: 420, 421, and 422, respectively, and the LCDR1, LCDR2, and LCDR3 sequences of SEQ ID NOs: 433, 434, and 435, respectively.

In another specific embodiment, the present invention provides an isolated antibody or antigen-binding fragment thereof, which binds human CD32b and comprises the HCDR1, HCDR2, and HCDR3 sequences of SEQ ID NOs: 423, 424, and 425, respectively, and the LCDR1, LCDR2, and LCDR3 sequences of SEQ ID NOs: 436, 437, and 438, respectively.

In another specific embodiment, the present invention provides an isolated antibody or antigen-binding fragment thereof, which binds human CD32b and comprises the HCDR1, HCDR2, and HCDR3 sequences of SEQ ID NOs: 469, 470, and 471, respectively, and the LCDR1, LCDR2, and LCDR3 sequences of SEQ ID NOs: 482, 483, and 484, respectively.

In another specific embodiment, the present invention provides an isolated antibody or antigen-binding fragment thereof, which binds human CD32b and comprises the HCDR1, HCDR2, and HCDR3 sequences of SEQ ID NOs: 472, 473, and 474, respectively, and the LCDR1, LCDR2, and LCDR3 sequences of SEQ ID NOs: 485, 486, and 487, respectively.

In another specific embodiment, the present invention provides an isolated antibody or antigen-binding fragment thereof, which binds human CD32b and comprises the HCDR1, HCDR2, and HCDR3 sequences of SEQ ID NOs: 475, 476, and 477, respectively, and the LCDR1, LCDR2, and LCDR3 sequences of SEQ ID NOs: 488, 489, and 490, respectively.

In another specific embodiment, the present invention provides an isolated antibody or antigen-binding fragment thereof, which binds human CD32b and comprises the HCDR1, HCDR2, and HCDR3 sequences of SEQ ID NOs: 521, 522, and 523, respectively, and the LCDR1, LCDR2, and LCDR3 sequences of SEQ ID NOs: 534, 535, and 536, respectively.

In another specific embodiment, the present invention provides an isolated antibody or antigen-binding fragment thereof, which binds human CD32b and comprises the HCDR1, HCDR2, and HCDR3 sequences of SEQ ID NOs: 524, 525, and 526, respectively, and the LCDR1, LCDR2, and LCDR3 sequences of SEQ ID NOs: 537, 538, and 539, respectively.

In another specific embodiment, the present invention provides an isolated antibody or antigen-binding fragment thereof, which binds human CD32b and comprises the HCDR1, HCDR2, and HCDR3 sequences of SEQ ID NOs: 527, 528, and 529, respectively, and the LCDR1, LCDR2, and LCDR3 sequences of SEQ ID NOs: 540, 541, and 542, respectively.

In another specific embodiment, the present invention provides an isolated antibody or antigen-binding fragment thereof, which binds human CD32b and comprises the HCDR1, HCDR2, and HCDR3 sequences of SEQ ID NOs: 547, 548, and 549, respectively, and the LCDR1, LCDR2, and LCDR3 sequences of SEQ ID NOs: 560, 561, and 562, respectively.

In another specific embodiment, the present invention provides an isolated antibody or antigen-binding fragment thereof, which binds human CD32b and comprises the HCDR1, HCDR2, and HCDR3 sequences of SEQ ID NOs: 550, 551, and 552, respectively, and the LCDR1, LCDR2, and LCDR3 sequences of SEQ ID NOs: 563, 564, and 565, respectively.

In another specific embodiment, the present invention provides an isolated antibody or antigen-binding fragment thereof, which binds human CD32b and comprises the HCDR1, HCDR2, and HCDR3 sequences of SEQ ID NOs: 553, 554, and 555, respectively, and the LCDR1, LCDR2, and LCDR3 sequences of SEQ ID NOs: 566, 567, and 568, respectively.

In another specific embodiment, the present invention provides an isolated antibody or antigen-binding fragment thereof, which binds human CD32b and comprises the HCDR1, HCDR2, and HCDR3 sequences of SEQ ID NOs: 573, 574, and 575, respectively, and the LCDR1, LCDR2, and LCDR3 sequences of SEQ ID NOs: 586, 587, and 588, respectively.

In another specific embodiment, the present invention provides an isolated antibody or antigen-binding fragment thereof, which binds human CD32b and comprises the HCDR1, HCDR2, and HCDR3 sequences of SEQ ID NOs: 576, 577, and 578, respectively, and the LCDR1, LCDR2, and LCDR3 sequences of SEQ ID NOs: 589, 590, and 591, respectively.

In another specific embodiment, the present invention provides an isolated antibody or antigen-binding fragment thereof, which binds human CD32b and comprises the HCDR1, HCDR2, and HCDR3 sequences of SEQ ID NOs: 579, 580, and 581, respectively, and the LCDR1, LCDR2, and LCDR3 sequences of SEQ ID NOs: 592, 593, and 594, respectively.

In another specific embodiment, the present invention provides an isolated antibody or antigen-binding fragment thereof, which binds human CD32b and comprises the HCDR1, HCDR2, and HCDR3 sequences of SEQ ID NOs: 625, 626, and 627, respectively, and the LCDR1, LCDR2, and LCDR3 sequences of SEQ ID NOs: 638, 639, and 640, respectively.

In another specific embodiment, the present invention provides an isolated antibody or antigen-binding fragment thereof, which binds human CD32b and comprises the HCDR1, HCDR2, and HCDR3 sequences of SEQ ID NOs: 628, 629, and 630, respectively, and the LCDR1, LCDR2, and LCDR3 sequences of SEQ ID NOs: 641, 642, and 643, respectively.

In another specific embodiment, the present invention provides an isolated antibody or antigen-binding fragment thereof, which binds human CD32b and comprises the HCDR1, HCDR2, and HCDR3 sequences of SEQ ID NOs: 631, 632, and 633, respectively, and the LCDR1, LCDR2, and LCDR3 sequences of SEQ ID NOs: 644, 645, and 646, respectively.

In another specific embodiment, the present invention provides an isolated antibody or antigen-binding fragment thereof, which binds human CD32b and comprises the VH amino acid sequence of SEQ ID NO: 10 and the VL amino acid sequence of SEQ ID NO:

23.

In another specific embodiment, the present invention provides an isolated antibody or antigen-binding fragment thereof, which binds human CD32b and comprises the VH amino acid sequence of SEQ ID NO: 62 and the VL amino acid sequence of SEQ ID NO: 75.

In another specific embodiment, the present invention provides an isolated antibody or antigen-binding fragment thereof, which binds human CD32b and comprises the VH amino acid sequence of SEQ ID NO: 114 and the VL amino acid sequence of SEQ ID NO: 127.

In another specific embodiment, the present invention provides an isolated antibody or antigen-binding fragment thereof, which binds human CD32b and comprises the VH amino acid sequence of SEQ ID NO: 166 and the VL amino acid sequence of SEQ ID NO: 179.

In another specific embodiment, the present invention provides an isolated antibody or antigen-binding fragment thereof, which binds human CD32b and comprises the VH amino acid sequence of SEQ ID NO: 218 and the VL amino acid sequence of SEQ ID NO: 231.

In another specific embodiment, the present invention provides an isolated antibody or antigen-binding fragment thereof, which binds human CD32b and comprises the VH amino acid sequence of SEQ ID NO: 270 and the VL amino acid sequence of SEQ ID NO: 283.

In another specific embodiment, the present invention provides an isolated antibody or antigen-binding fragment thereof, which binds human CD32b and comprises the VH amino acid sequence of SEQ ID NO: 322 and the VL amino acid sequence of SEQ ID NO: 335.

In another specific embodiment, the present invention provides an isolated antibody or antigen-binding fragment thereof, which binds human CD32b and comprises the VH amino acid sequence of SEQ ID NO: 374 and the VL amino acid sequence of SEQ ID NO: 387.

In another specific embodiment, the present invention provides an isolated antibody or antigen-binding fragment thereof, which binds human CD32b and comprises the VH amino acid sequence of SEQ ID NO: 426 and the VL amino acid sequence of SEQ ID NO: 439.

In another specific embodiment, the present invention provides an isolated antibody or antigen-binding fragment thereof, which binds human CD32b and comprises the VH amino acid sequence of SEQ ID NO: 478 and the VL amino acid sequence of SEQ ID NO: 491.

In another specific embodiment, the present invention provides an isolated antibody or antigen-binding fragment thereof, which binds human CD32b and comprises the VH amino acid sequence of SEQ ID NO: 530 and the VL amino acid sequence of SEQ ID NO: 543.

In another specific embodiment, the present invention provides an isolated antibody or antigen-binding fragment thereof, which binds human CD32b and comprises the VH amino acid sequence of SEQ ID NO: 556 and the VL amino acid sequence of SEQ ID NO: 569.

In another specific embodiment, the present invention provides an isolated antibody or antigen-binding fragment thereof, which binds human CD32b and comprises the VH amino acid sequence of SEQ ID NO: 582 and the VL amino acid sequence of SEQ ID NO: 595.

In another specific embodiment, the present invention provides an isolated antibody or antigen-binding fragment thereof, which binds human CD32b and comprises the VH amino acid sequence of SEQ ID NO: 634 and the VL amino acid sequence of SEQ ID NO: 647.

In another specific embodiment, the present invention provides an isolated antibody or antigen-binding fragment thereof, which binds human CD32b and comprises the heavy chain amino acid sequence of SEQ ID NO: 12 and the light chain amino acid sequence of SEQ ID NO: 25.

In another specific embodiment, the present invention provides an isolated antibody or antigen-binding fragment thereof, which binds human CD32b and comprises the heavy chain amino acid sequence of SEQ ID NO: 38 and the light chain amino acid sequence of SEQ ID NO: 51.

In another specific embodiment, the present invention provides an isolated antibody or antigen-binding fragment thereof, which binds human CD32b and comprises the heavy chain amino acid sequence of SEQ ID NO: 64 and the light chain amino acid sequence of SEQ ID NO: 77.

In another specific embodiment, the present invention provides an isolated antibody or antigen-binding fragment thereof, which binds human CD32b and comprises the heavy chain amino acid sequence of SEQ ID NO: 90 and the light chain amino acid sequence of SEQ ID NO: 103.

In another specific embodiment, the present invention provides an isolated antibody or antigen-binding fragment thereof, which binds human CD32b and comprises the heavy chain amino acid sequence of SEQ ID NO: 116 and the light chain amino acid sequence of SEQ ID NO: 129.

In another specific embodiment, the present invention provides an isolated antibody or antigen-binding fragment thereof, which binds human CD32b and comprises the heavy chain amino acid sequence of SEQ ID NO: 142 and the light chain amino acid sequence of SEQ ID NO: 155.

In another specific embodiment, the present invention provides an isolated antibody or antigen-binding fragment thereof, which binds human CD32b and comprises the heavy chain amino acid sequence of SEQ ID NO: 168 and the light chain amino acid sequence of SEQ ID NO: 181.

In another specific embodiment, the present invention provides an isolated antibody or antigen-binding fragment thereof, which binds human CD32b and comprises the heavy chain amino acid sequence of SEQ ID NO: 194 and the light chain amino acid sequence of SEQ ID NO: 207.

In another specific embodiment, the present invention provides an isolated antibody or antigen-binding fragment thereof, which binds human CD32b and comprises the heavy chain amino acid sequence of SEQ ID NO: 220 and the light chain amino acid sequence of SEQ ID NO: 233.

In another specific embodiment, the present invention provides an isolated antibody or antigen-binding fragment thereof, which binds human CD32b and comprises the heavy chain amino acid sequence of SEQ ID NO: 246 and the light chain amino acid sequence of SEQ ID NO: 259.

In another specific embodiment, the present invention provides an isolated antibody or antigen-binding fragment thereof, which binds human CD32b and comprises the heavy chain amino acid sequence of SEQ ID NO: 272 and the light chain amino acid sequence of SEQ ID NO: 285.

In another specific embodiment, the present invention provides an isolated antibody or antigen-binding fragment thereof, which binds human CD32b and comprises the heavy chain amino acid sequence of SEQ ID NO: 298 and the light chain amino acid sequence of SEQ ID NO: 311.

In another specific embodiment, the present invention provides an isolated antibody or antigen-binding fragment thereof, which binds human CD32b and comprises the heavy chain amino acid sequence of SEQ ID NO: 324 and the light chain amino acid sequence of SEQ ID NO: 337.

In another specific embodiment, the present invention provides an isolated antibody or antigen-binding fragment thereof, which binds human CD32b and comprises the heavy chain amino acid sequence of SEQ ID NO: 350 and the light chain amino acid sequence of SEQ ID NO: 363.

In another specific embodiment, the present invention provides an isolated antibody or antigen-binding fragment thereof, which binds human CD32b and comprises the heavy chain amino acid sequence of SEQ ID NO: 376 and the light chain amino acid sequence of SEQ ID NO: 389.

In another specific embodiment, the present invention provides an isolated antibody or antigen-binding fragment thereof, which binds human CD32b and comprises the heavy chain amino acid sequence of SEQ ID NO: 402 and the light chain amino acid sequence of SEQ ID NO: 415.

In another specific embodiment, the present invention provides an isolated antibody or antigen-binding fragment thereof, which binds human CD32b and comprises the heavy chain amino acid sequence of SEQ ID NO: 428 and the light chain amino acid sequence of SEQ ID NO: 441.

In another specific embodiment, the present invention provides an isolated antibody or antigen-binding fragment thereof, which binds human CD32b and comprises the heavy chain amino acid sequence of SEQ ID NO: 454 and the light chain amino acid sequence of SEQ ID NO: 467.

In another specific embodiment, the present invention provides an isolated antibody or antigen-binding fragment thereof, which binds human CD32b and comprises the heavy chain amino acid sequence of SEQ ID NO: 480 and the light chain amino acid sequence of SEQ ID NO: 493.

In another specific embodiment, the present invention provides an isolated antibody or antigen-binding fragment thereof, which binds human CD32b and comprises the heavy chain amino acid sequence of SEQ ID NO: 506 and the light chain amino acid sequence of SEQ ID NO: 519.

In another specific embodiment, the present invention provides an isolated antibody or antigen-binding fragment thereof, which binds human CD32b and comprises the heavy chain amino acid sequence of SEQ ID NO: 532 and the light chain amino acid sequence of SEQ ID NO: 545.

In another specific embodiment, the present invention provides an isolated antibody or antigen-binding fragment thereof, which binds human CD32b and comprises the heavy chain amino acid sequence of SEQ ID NO: 558 and the light chain amino acid sequence of SEQ ID NO: 571.

In another specific embodiment, the present invention provides an isolated antibody or antigen-binding fragment thereof, which binds human CD32b and comprises the heavy chain amino acid sequence of SEQ ID NO: 584 and the light chain amino acid sequence of SEQ ID NO: 597.

In another specific embodiment, the present invention provides an isolated antibody or antigen-binding fragment thereof, which binds human CD32b and comprises the heavy chain amino acid sequence of SEQ ID NO: 610 and the light chain amino acid sequence of SEQ ID NO: 623.

In another specific embodiment, the present invention provides an isolated antibody or antigen-binding fragment thereof, which binds human CD32b and comprises the heavy chain amino acid sequence of SEQ ID NO: 636 and the light chain amino acid sequence of SEQ ID NO: 649.

In another specific embodiment, the present invention provides an isolated antibody or antigen-binding fragment thereof, which binds human CD32b and comprises the heavy chain amino acid sequence of SEQ ID NO: 662 and the light chain amino acid sequence of SEQ ID NO: 675.

Since each of these antibodies can bind to CD32b, the VH, VL, full length light chain, and full length heavy chain sequences (amino acid sequences and the nucleotide sequences encoding the amino acid sequences) can be “mixed and matched” to create other CD32b-binding antibodies and antigen-binding fragments thereof of the invention. Such “mixed and matched” CD32b-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise a full length light chain sequence from a particular full length heavy chain/full length light chain pairing should be replaced with a structurally similar full length light chain sequence.

In another aspect, the present invention provides CD32b-binding antibodies that comprise the heavy chain and light chain CDR1s, CDR2s and CDR3s as described in Table 1, or combinations thereof. The CDR regions are delineated using the Kabat system (Kabat et al. 1991 Sequences of Proteins of Immunological Interest, Fifth Edition, U.S. Department of Health and Human Services, NIH Publication No. 91-3242), or using the Chothia system (Chothia et al. 1987 J. Mol. Biol. 196: 901-917; and Al-Lazikani et al. 1997 J. Mol. Biol. 273: 927-948). Other methods for delineating the CDR regions may alternatively be used. For example, the CDR definitions of both Kabat and Chothia may be combined such that, the CDRs may comprise some or all of the amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Given that each of these antibodies can bind to CD32b and that antigen-binding specificity is provided primarily by the CDR1, 2 and 3 regions, the VH CDR1, 2 and 3 sequences and VL CDR1, 2 and 3 sequences can be “mixed and matched” (i.e., CDRs from different antibodies can be mixed and match, although each antibody must contain a VH CDR1, 2 and 3 and a VL CDR1, 2 and 3 to create other CD32b-binding binding molecules of the invention. Such “mixed and matched” CD32b-binding antibodies can be tested using the binding assays known in the art and those described in the Examples (e.g., ELISAs). When VH CDR sequences are mixed and matched, the CDR1, CDR2 and/or CDR3 sequence from a particular VH sequence should be replaced with a structurally similar CDR sequence (s). Likewise, when VL CDR sequences are mixed and matched, the CDR1, CDR2 and/or CDR3 sequence from a particular VL sequence should be replaced with a structurally similar CDR sequence (s). It will be readily apparent to the ordinarily skilled artisan that novel VH and VL sequences can be created by mutating one or more VH and/or VL CDR region sequences with structurally similar sequences from the CDR sequences shown herein for monoclonal antibodies of the present invention.

Accordingly, the present invention provides an isolated monoclonal antibody or antigen binding region thereof comprising a heavy chain variable region CDR1 comprising an amino acid sequence selected from any of SEQ ID NOs: 1, 4, 7, 53, 56, 59, 105, 108, 111, 157, 160, 163, 209, 212, 215, 261, 264, 267, 313, 316, 319, 365, 368, 371, 417, 420, 423, 469, 472, 475, 521, 524, 527, 547, 550, 553, 573, 576, 579, 625, 628, and 631; a heavy chain variable region CDR2 comprising an amino acid sequence selected from any of SEQ ID NOs: 2, 5, 8, 54, 57, 60, 106, 109, 112, 158, 161, 164, 210, 213, 216, 262, 265, 268, 314, 317, 320, 366, 369, 372, 418, 421; 424, 470, 473, 476, 522, 525, 528, 548, 551, 554, 574, 577, 580, 626, 629, and 632; a heavy chain variable region CDR3 comprising an amino acid sequence selected from any of SEQ ID NOs: 3, 6, 9, 55, 58, 61, 107, 110, 113, 159, 162, 165, 211, 214, 217, 263, 266, 269, 315, 318, 321, 367, 370, 373, 419, 422, 425, 471, 474, 477, 523, 526, 529, 549, 552, 555, 575, 578, 581, 627, 630, and 633; a light chain variable region CDR1 comprising an amino acid sequence selected from any of SEQ ID NOs: 14, 17, 20, 66, 69, 72, 118, 121, 124, 170, 173, 176, 222, 225, 228, 274, 277, 280, 326, 329, 332, 378, 381, 384, 430, 433, 436, 482, 485, 488, 534, 537, 540, 560, 563, 566, 586, 589, 592, 638, 641, 644; a light chain variable region CDR2 comprising an amino acid sequence selected from any of SEQ ID NOs: 15, 18, 21, 67, 70, 73, 119, 122, 125, 171, 174, 177, 223, 226, 229, 275, 278, 281, 327, 330, 333, 379, 382, 385, 431, 434, 437, 483, 486, 489, 535, 538, 541, 561, 564, 567, 587, 590, 593, 639, 642, and 645; and a light chain variable region CDR3 comprising an amino acid sequence selected from any of SEQ ID NOs: 16, 19, 22, 68, 71, 74, 120, 123, 126, 172, 175, 178, 224, 227, 230, 276, 279, 282, 328, 331, 334, 380, 383, 386, 432, 435, 438, 484, 487, 490, 536, 539, 542, 562, 565, 568, 588, 591, 594, 640, 643, and 646; wherein the antibody specifically binds CD32b.

The present invention also provides an isolated monoclonal antibody or antigen binding region thereof comprising a heavy chain variable region comprising an amino acid sequence selected from any of SEQ ID NOs: 10, 62, 114, 166, 218, 270, 322, 374, 426, 478, 530, 556, 582, and 634; and a light chain variable region comprising an amino acid sequence selected from any of SEQ ID NOs: 23, 75, 127, 179, 231, 283, 335, 387, 439, 491, 543, 569, 595, and 647.

The present invention also provides an isolated monoclonal antibody or antigen binding region thereof comprising a heavy chain comprising an amino acid sequence selected from any of SEQ ID NOs: 12, 38, 64, 90, 116, 142, 168, 194, 220, 246, 272, 298, 324, 350, 376, 402, 428, 454, 480, 506, 532, 558, 584, 610, 636, and 662; and a light chain comprising an amino acid sequence selected from any of SEQ ID NOs: 25, 51, 77, 103, 129, 155, 181, 207, 233, 259, 285, 311, 337, 363, 389, 415, 441, 467, 493, 519, 545, 571, 597, 623, 649, and 675.

In one embodiment, an antibody that specifically binds to CD32b is an antibody that is described in Table 1. In one embodiment, an antibody that specifically binds to CD32b is NOV0281. In one embodiment, an antibody that specifically binds to CD32b is NOV0281_N297A. In one embodiment, an antibody that specifically binds to CD32b is NOV0308. In one embodiment, an antibody that specifically binds to CD32b is NOV0308_N297A. In one embodiment, an antibody that specifically binds to CD32b is NOV0563. In one embodiment, an antibody that specifically binds to CD32b is NOV0563_N297A. In one embodiment, an antibody that specifically binds to CD32b is NOV1216. In one embodiment, an antibody that specifically binds to CD32b is NOV1216_N297A. In one embodiment, an antibody that specifically binds to CD32b is NOV1218. In one embodiment, an antibody that specifically binds to CD32b is NOV1218_N297A. In one embodiment, an antibody that specifically binds to CD32b is NOV1219. In one embodiment, an antibody that specifically binds to CD32b is NOV1219_N297A. In one embodiment, an antibody that specifically binds to CD32b is NOV2106. In one embodiment, an antibody that specifically binds to CD32b is NOV02106_N297A. In one embodiment, an antibody that specifically binds to CD32b is NOV2107. In one embodiment, an antibody that specifically binds to CD32b is NOV2107_N297A. In one embodiment, an antibody that specifically binds to CD32b is NOV2108. In one embodiment, an antibody that specifically binds to CD32b is NOV2108_N297A. In one embodiment, an antibody that specifically binds to CD32b is NOV2109 In one embodiment, an antibody that specifically binds to CD32b is NOV2109_N297A. In one embodiment, an antibody that specifically binds to CD32b is NOV2110_N297A. In one embodiment, an antibody that specifically binds to CD32b is NOV2111_N297A. In one embodiment, an antibody that specifically binds to CD32b is NOV2112. In one embodiment, an antibody that specifically binds to CD32b is NOV2112_N297A. In one embodiment, an antibody that specifically binds to CD32b is NOV2113. In one embodiment, an antibody that specifically binds to CD32b is NOV2113_N297A.

In some embodiments of the CD32b-binding antibodies, or antigen binding fragments thereof disclosed herein, the antibodies comprise a wild type (WT) Fc sequence. In some embodiments, the antibodies are afucosylated. In other embodiments, the antibodies comprise a modified Fc region comprising mutations which enhance ADCC (eADCC) activity of the antibodies. In yet other embodiments, the antibodies comprise a modified Fc region comprising mutations which silence the ADCC activity of the Fc region (Fc silent mutants).

In one embodiment, the CD32b-binding antibody is afucosylated NOV2108, comprising a WT Fc. In a specific embodiment, the CD32b-binding antibody comprises an HCDR1, HCDR2, and HCDR3 comprising the amino acid sequences of SEQ ID NOs: 417, 418, and 419, respectively, and a LCDR1, LCDR2, and LCDR3 comprising the amino acid sequences of SEQ ID NOs: 430, 431, and 432 respectively, and wherein the antibody is afucosylated. In another specific embodiment, the CD32b-binding antibody comprises a VH comprising the amino acid sequence of SEQ ID NO:426 and a VL comprising the amino acid sequence of SEQ ID NO:439, and wherein the antibody is afucosylated. In yet another embodiment, the CD32b-binding antibody comprises a heavy chain comprising the amino acid sequence of SEQ ID NO:428 and a light chain comprising the amino acid sequence of SEQ ID NO: 441, wherein the antibody is afucosylated.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally occurring somatic mutations or intentional introduction of site-directed mutations. However, in the VH or VL framework regions, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene. Typically, a recombinant human antibody will display no more than 10 amino acid differences from the amino acid sequence encoded by the human germline immunoglobulin gene in the VH or VL framework regions. In certain cases, the human antibody may display no more than 5, or even no more than 4, 3, 2, or 1 amino acid difference from the amino acid sequence encoded by the germline immunoglobulin gene.

### Homologous Antibodies

In yet another embodiment, the present invention provides an antibody or an antigen-binding fragment thereof comprising amino acid sequences that are homologous to the sequences described in Table 1, and said antibody binds to CD32b, and retains the desired functional properties of those antibodies described in Table 1.

For example, the invention provides an isolated monoclonal antibody (or a functional antigen-binding fragment thereof) comprising a heavy chain variable region and a light chain variable region, wherein the heavy chain variable region comprises an amino acid sequence that is at least 80%, at least 90%, or at least 95% identical to an amino acid sequence selected from the group consisting of SEQ ID NOs: 10, 62, 114, 166, 218, 270, 322, 374, 426, 478, 530, 556, 582, and 634; the light chain variable region comprises an amino acid sequence that is at least 80%, at least 90%, or at least 95% identical to an amino acid sequence selected from the group consisting of SEQ ID NOs: 23, 75, 127, 179, 231, 283, 335, 387, 439, 491, 543, 569, 595, and 647; wherein the antibody specifically binds to human CD32b protein.

In one embodiment, the VH and/or VL amino acid sequences may be 50%, 60%, 70%, 80%, 90%, 95%, 96%, 97%, 98% or 99% identical to the sequences set forth in Table 1. In one embodiment, the VH and/or VL amino acid sequences may be identical except an amino acid substitution in no more than 1, 2, 3, 4 or 5 amino acid positions. An antibody having VH and VL regions having high (i.e., 80% or greater) identity to the VH and VL regions of those described in Table 1 can be obtained by mutagenesis (e.g., site-directed or PCR-mediated mutagenesis) of nucleic acid molecules encoding SEQ ID NOs: 10, 62, 114, 166, 218, 270, 322, 374, 426, 478, 530, 556, 582, or 634; and 23, 75, 127, 179, 231, 283, 335, 387, 439, 491, 543, 569, 595, or 647 respectively, followed by testing of the encoded altered antibody for retained function using the functional assays described herein.

In one embodiment, the full length heavy chain and/or full length light chain amino acid sequences may be 50% 60%, 70%, 80%, 90%, 95%, 96%, 97%, 98% or 99% identical to the sequences set forth in Table 1. An antibody having a full length heavy chain and full length light chain having high (i.e., 80% or greater) identity to the full length heavy chains of any of SEQ ID NOs: 12, 38, 64, 90, 116, 142, 168, 194, 220, 246, 272, 298, 324, 350, 376, 402, 428, 454, 480, 506, 532, 558, 584, 610, 636, and 662; and full length light chains of any of SEQ ID NOs: 25, 51, 77, 103, 129, 155, 181, 207, 233, 259, 285, 311, 337, 363, 389, 415, 441, 467, 493, 519, 545, 571, 597, 623, 649, and 675, respectively, can be obtained by mutagenesis (e.g., site-directed or PCR-mediated mutagenesis) of nucleic acid molecules encoding such polypeptides respectively, followed by testing of the encoded altered antibody for retained function using the functional assays described herein.

In one embodiment, the full length heavy chain and/or full length light chain nucleotide sequences may be 60%, 70%, 80%, 90%, 95%, 96%, 97%, 98% or 99% identical to the sequences set forth in Table 1.

In one embodiment, the variable regions of heavy chain and/or light chain nucleotide sequences may be 60%, 70%, 80%, 90%, 95%, 96%, 97%, 98% or 99% identical to the sequences set forth in Table 1.

As used herein, the percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

Additionally or alternatively, the protein sequences of the present invention can further be used as a “query sequence” to perform a search against public databases to, for example, identify related sequences. For example, such searches can be performed using the BLAST program (version 2.0) of Altschul, et al., 1990 J. Mol. Biol. 215:403-10.

Antibodies with Conservative Modifications

In one embodiment, an antibody of the invention has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the CD32b-binding antibodies and antigen-binding fragments thereof of the invention. Accordingly, the invention provides an isolated monoclonal antibody, or a functional antigen-binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 comprises an amino acid sequence selected from any of SEQ ID NOs: 1, 4, 7, 53, 56, 59, 105, 108, 111, 157, 160, 163, 209, 212, 215, 261, 264, 267, 313, 316, 319, 365, 368, 371, 417, 420, 423, 469, 472, 475, 521, 524, 527, 547, 550, 553, 573, 576, 579, 625, 628, and 631, or conservative variants thereof; the heavy chain variable region CDR2 comprises an amino acid sequence selected from any of SEQ ID NOs: 2, 5, 8, 54, 57, 60, 106, 109, 112, 158, 161, 164, 210, 213, 216, 262, 265, 268, 314, 317, 320, 366, 369, 372, 418, 421; 424, 470, 473, 476, 522, 525, 528, 548, 551, 554, 574, 577, 580, 626, 629, and 632, or conservative variants thereof; the heavy chain variable region CDR3 comprises an amino acid sequence selected from any of SEQ ID NOs: 3, 6, 9, 55, 58, 61, 107, 110, 113, 159, 162, 165, 211, 214, 217, 263, 266, 269, 315, 318, 321, 367, 370, 373, 419, 422, 425, 471, 474, 477, 523, 526, 529, 549, 552, 555, 575, 578, 581, 627, 630, and 633, or conservative variants thereof; the light chain variable region CDR1 comprises an amino acid sequence selected from any of SEQ ID NOs: 14, 17, 20, 66, 69, 72, 118, 121, 124, 170, 173, 176, 222, 225, 228, 274, 277, 280, 326, 329, 332, 378, 381, 384, 430, 433, 436, 482, 485, 488, 534, 537, 540, 560, 563, 566, 586, 589, 592, 638, 641, 644, or conservative variants thereof; the light chain variable region CDR2 comprises an amino acid sequence selected from any of SEQ ID NOs: 15, 18, 21, 67, 70, 73, 119, 122, 125, 171, 174, 177, 223, 226, 229, 275, 278, 281, 327, 330, 333, 379, 382, 385, 431, 434, 437, 483, 486, 489, 535, 538, 541, 561, 564, 567, 587, 590, 593, 639, 642, and 645, or conservative variants thereof; and the light chain variable region CDR3 comprises an amino acid sequence selected from any of SEQ ID NOs: 16, 19, 22, 68, 71, 74, 120, 123, 126, 172, 175, 178, 224, 227, 230, 276, 279, 282, 328, 331, 334, 380, 383, 386, 432, 435, 438, 484, 487, 490, 536, 539, 542, 562, 565, 568, 588, 591, 594, 640, 643, and 646, or conservative variants thereof; wherein the antibody or the antigen-binding fragment thereof specifically binds to CD32b and mediates both macrophage and NK cell killing of antibody bound, CD32b positive target cells.

In one embodiment, an antibody of the invention optimized for expression in a mammalian cell has a heavy chain variable region and a light chain variable region, wherein one or more of these sequences have specified amino acid sequences based on the antibodies described herein or conservative modifications thereof, and wherein the antibodies retain the desired functional properties of the CD32b-binding antibodies and antigen-binding fragments thereof of the invention. Accordingly, the invention provides an isolated monoclonal antibody optimized for expression in a mammalian cell comprising a heavy chain variable region and a light chain variable region wherein: the heavy chain variable region comprises an amino acid sequence selected from any of SEQ ID NOs: 10, 62, 114, 166, 218, 270, 322, 374, 426, 478, 530, 556, 582, and 6342, and conservative modifications thereof; and the light chain variable region comprises an amino acid sequence selected from any of SEQ ID NOs: 23, 75, 127, 179, 231, 283, 335, 387, 439, 491, 543, 569, 595, and 647, and conservative modifications thereof; wherein the antibody specifically binds to CD32b and mediates both macrophage and NK cell killing of antibody bound, CD32b positive target cells.

In one embodiment, an antibody of the invention optimized for expression in a mammalian cell has a full length heavy chain sequence and a full length light chain sequence, wherein one or more of these sequences have specified amino acid sequences based on the antibodies described herein or conservative modifications thereof, and wherein the antibodies retain the desired functional properties of the CD32b-binding antibodies and antigen-binding fragments thereof of the invention. Accordingly, the invention provides an isolated monoclonal antibody optimized for expression in a mammalian cell comprising a full length heavy chain and a full length light chain wherein: the full length heavy chain comprises an amino acid sequence selected from any of SEQ ID NOs: 12, 38, 64, 90, 116, 142, 168, 194, 220, 246, 272, 298, 324, 350, 376, 402, 428, 454, 480, 506, 532, 558, 584, 610, 636, and 662, and conservative modifications thereof; and the full length light chain comprises an amino acid sequence selected from any of SEQ ID NOs: 25, 51, 77, 103, 129, 155, 181, 207, 233, 259, 285, 311, 337, 363, 389, 415, 441, 467, 493, 519, 545, 571, 597, 623, 649, and 675, and conservative modifications thereof; wherein the antibody specifically binds to CD32b and mediates both macrophage and NK cell killing of antibody bound, CD32b positive target cells.

Antibodies that Bind to the Same Epitope

The present invention provides antibodies that bind to the same epitope as do the CD32b-binding antibodies listed in Table 1. Additional antibodies can therefore be identified based on their ability to cross-compete (e.g., to competitively inhibit the binding of, in a statistically significant manner) with other antibodies and antigen-binding fragments thereof of the invention inCD32b binding assays. The ability of a test antibody to inhibit the binding of antibodies and antigen-binding fragments thereof of the present invention to CD32b protein demonstrates that the test antibody can compete with that antibody for binding to CD32b; such an antibody may, according to non-limiting theory, bind to the same or a related (e.g., a structurally similar or spatially proximal) epitope on CD32B as the antibody with which it competes. In a certain embodiment, the antibody that binds to the same epitope on CD32B as the antibodies and antigen-binding fragments thereof of the present invention is a human monoclonal antibody. Such human monoclonal antibodies can be prepared and isolated as described herein.

Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen. A high throughput process for “binning” antibodies based upon their cross-competition is described in International Patent Application No. WO 2003/48731. As will be appreciated by one of skill in the art, practically anything to which an antibody can specifically bind could be an epitope. An epitope can comprises those residues to which the antibody binds.

Generally, antibodies specific for a particular target antigen will preferentially recognize an epitope on the target antigen in a complex mixture of proteins and/or macromolecules.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715 Similarly, conformational epitopes are readily identified by determining spatial conformation of amino acids CD32b such as by, e.g., hydrogen/deuterium exchange, x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., (1981) Proc. Natl. Acad. Sci USA 78:3824-3828; for determining antigenicity profiles, and the Kyte-Doolittle technique, Kyte et al., (1982) J. Mol. Biol. 157:105-132; for hydropathy plots.

### Engineered and Modified Antibodies

An antibody of the invention further can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region (s), for example to alter the effector function (s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L et al., 1998 Nature 332:323-327; Jones, P. et al., 1986 Nature 321:522-525; Queen, C. et al., 1989 Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.)

Such framework sequences can be obtained from public DNA databases or published references that include germine antibody gene sequences or rearranged antibody sequences. For example, germine DNA sequences for human heavy and light chain variable region genes can be found in the “VBase” human germline sequence database (available on the Internet at www.mrc-cpe.cam.ac.uk/vbase), as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. fol. Biol. 227:776-798; and Cox, J. P. L. et al., 1994 Eur. J Immunol. 24:827-836; the contents of each of which are expressly incorporated herein by reference. For example, germline DNA sequences for human heavy and light chain variable region genes and rearranged antibody sequences can be found in “IMGT” database (available on the Internet at www.imgt.org; see Lefranc, M. P. et al., 1999 Nucleic Acids Res. 27:209-212; the contents of each of which are expressly incorporated herein by reference.)

An example of framework sequences for use in the antibodies and antigen-binding fragments thereof of the invention are those that are structurally similar to the framework sequences used by selected antibodies and antigen-binding fragments thereof of the invention, e.g., consensus sequences and/or framework sequences used by monoclonal antibodies of the invention. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation (s) and the effect on antibody binding, or other functional property of interest, can be evaluated in in vitro or in vivo assays as described herein and provided in the Examples. Conservative modifications (as discussed above) can be introduced. The mutations may be amino acid substitutions, additions or deletions. Moreover, typically no more than one, two, three, four or five residues within a CDR region are altered.

Grafting Antigen-Binding Domains into Alternative Frameworks or Scaffolds

A wide variety of antibody/immunoglobulin frameworks or scaffolds can be employed so long as the resulting polypeptide includes at least one binding region which specifically binds to CD32b. Such frameworks or scaffolds include the 5 main idiotypes of human immunoglobulins, antigen-binding fragments thereof, and include immunoglobulins of other animal species, preferably having humanized aspects. Single heavy-chain antibodies such as those identified in camelids are of particular interest in this regard. Novel frameworks, scaffolds and fragments continue to be discovered and developed by those skilled in the art.

In one aspect, the invention pertains to a method of generating non-immunoglobulin based antibodies using non-immunoglobulin scaffolds onto which CDRs of the invention can be grafted. Known or future non-immunoglobulin frameworks and scaffolds may be employed, as long as they comprise a binding region specific for the target CD32b protein. Known non-immunoglobulin frameworks or scaffolds include, but are not limited to, fibronectin (Compound Therapeutics, Inc., Waltham, Mass.), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (SciI Proteins GmbH, Halle, Germany).

The fibronectin scaffolds are based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). These fibronectin-based scaffolds are not an immunoglobulin, although the overall fold is closely related to that of the smallest functional antibody fragment, the variable region of the heavy chain, which comprises the entire antigen recognition unit in camel and llama IgG. Because of this structure, the non-immunoglobulin antibody mimics antigen binding properties that are similar in nature and affinity for those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module is a 33 amino acid polypeptide consisting of two anti-parallel alpha-helices and a beta-turn. Binding of the variable regions is mostly optimized by using ribosome display.

Avimers are derived from natural A-domain containing protein such as LRP-1. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

The human CD32B-binding antibodies can be generated using methods that are known in the art. For example, the humaneering technology used for converting non-human antibodies into engineered human antibodies. U.S. Patent Publication No. 20050008625 describes an in vivo method for replacing a nonhuman antibody variable region with a human variable region in an antibody while maintaining the same or providing better binding characteristics relative to that of the nonhuman antibody. The method relies on epitope guided replacement of variable regions of a non-human reference antibody with a fully human antibody. The resulting human antibody is generally unrelated structurally to the reference nonhuman antibody, but binds to the same epitope on the same antigen as the reference antibody. Briefly, the serial epitope-guided complementarity replacement approach is enabled by setting up a competition in cells between a “competitor” and a library of diverse hybrids of the reference antibody (“test antibodies”) for binding to limiting amounts of antigen in the presence of a reporter system which responds to the binding of test antibody to antigen. The competitor can be the reference antibody or derivative thereof such as a single-chain Fv fragment. The competitor can also be a natural or artificial ligand of the antigen which binds to the same epitope as the reference antibody. The only requirements of the competitor are that it binds to the same epitope as the reference antibody, and that it competes with the reference antibody for antigen binding. The test antibodies have one antigen-binding V-region in common from the nonhuman reference antibody, and the other V-region selected at random from a diverse source such as a repertoire library of human antibodies. The common V-region from the reference antibody serves as a guide, positioning the test antibodies on the same epitope on the antigen, and in the same orientation, so that selection is biased toward the highest antigen-binding fidelity to the reference antibody.

Many types of reporter system can be used to detect desired interactions between test antibodies and antigen. For example, complementing reporter fragments may be linked to antigen and test antibody, respectively, so that reporter activation by fragment complementation only occurs when the test antibody binds to the antigen. When the test antibody- and antigen-reporter fragment fusions are co-expressed with a competitor, reporter activation becomes dependent on the ability of the test antibody to compete with the competitor, which is proportional to the affinity of the test antibody for the antigen. Other reporter systems that can be used include the reactivator of an auto-inhibited reporter reactivation system (RAIR) as disclosed in U.S. patent application Ser. No. 10/208,730 (Publication No. 20030198971), or competitive activation system disclosed in U.S. patent application Ser. No. 10/076,845 (Publication No. 20030157579).

With the serial epitope-guided complementarity replacement system, selection is made to identify cells expresses a single test antibody along with the competitor, antigen, and reporter components. In these cells, each test antibody competes one-on-one with the competitor for binding to a limiting amount of antigen. Activity of the reporter is proportional to the amount of antigen bound to the test antibody, which in turn is proportional to the affinity of the test antibody for the antigen and the stability of the test antibody. Test antibodies are initially selected on the basis of their activity relative to that of the reference antibody when expressed as the test antibody. The result of the first round of selection is a set of “hybrid” antibodies, each of which is comprised of the same non-human V-region from the reference antibody and a human V-region from the library, and each of which binds to the same epitope on the antigen as the reference antibody. One of more of the hybrid antibodies selected in the first round will have an affinity for the antigen comparable to or higher than that of the reference antibody.

In the second V-region replacement step, the human V-regions selected in the first step are used as guide for the selection of human replacements for the remaining non-human reference antibody V-region with a diverse library of cognate human V-regions. The hybrid antibodies selected in the first round may also be used as competitors for the second round of selection. The result of the second round of selection is a set of fully human antibodies which differ structurally from the reference antibody, but which compete with the reference antibody for binding to the same antigen. Some of the selected human antibodies bind to the same epitope on the same antigen as the reference antibody. Among these selected human antibodies, one or more binds to the same epitope with an affinity which is comparable to or higher than that of the reference antibody.

In addition, human CD32b-binding antibodies can also be commercially obtained from companies which customarily produce human antibodies, e.g., KaloBios, Inc. (Mountain View, Calif.).

### Camelid Antibodies

Antibody proteins obtained from members of the camel and dromedary (Camelus bactrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals See PCT/EP93/02214 (WO 94/04678 published 3 Mar. 1994).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J 17: 3512-3520. Engineered libraries of camelid antibodies and antibody fragments are commercially available, for example, from Ablynx, Ghent, Belgium. As with other antibodies and antigen-binding fragments thereof of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. Further, these molecules can be fully expressed in prokaryotic cells such as E. coli and are expressed as fusion proteins with bacteriophage and are functional.

Accordingly, a feature of the present invention is a camelid antibody or nanobody having high affinity for CD32b. In one embodiment herein, the camelid antibody or nanobody is naturally produced in the camelid animal, i.e., is produced by the camelid following immunization with CD32b or a peptide fragment thereof, using techniques described herein for other antibodies. Alternatively, the CD32b-binding camelid nanobody is engineered, i.e., produced by selection for example from a library of phage displaying appropriately mutagenized camelid nanobody proteins using panning procedures with CD32b as a target as described in the examples herein. Engineered nanobodies can further be customized by genetic engineering to have a half life in a recipient subject of from 45 minutes to two weeks. In a specific embodiment, the camelid antibody or nanobody is obtained by grafting the CDRs sequences of the heavy or light chain of the human antibodies of the invention into nanobody or single domain antibody framework sequences, as described for example in PCT/EP93/02214.

### Bispecific Molecules and Multivalent Antibodies

In another aspect, the present invention features bispecific or multispecific molecules comprising a CD32b-binding antibody, or a fragment thereof, of the invention. An antibody of the invention, or antigen-binding regions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for CD32b and a second binding specificity for a second target epitope. For example, the second target epitope is another epitope of CD32b different from the first target epitope.

Additionally, for the invention in which the bispecific molecule is multi-specific, the molecule can further include a third binding specificity, in addition to the first and second target epitope.

In one embodiment, the bispecific molecules of the invention comprise as a binding specificity at least one antibody, or an antibody fragment thereof, including, e.g., an Fab, Fab′, F (ab′)2, Fv, or a single chain Fv. The antibody may also be a light chain or heavy chain dimer, or any minimal fragment thereof such as a Fv or a single chain construct as described in Ladner et al. U.S. Pat. No. 4,946,778.

Diabodies are bivalent, bispecific molecules in which VH and VL domains are expressed on a single polypeptide chain, connected by a linker that is too short to allow for pairing between the two domains on the same chain. The VH and VL domains pair with complementary domains of another chain, thereby creating two antigen binding sites (see e.g., Holliger et al., 1993 Proc. Natl. Acad. Sci. USA 90:6444-6448; Poijak et al., 1994 Structure 2:1121-1123). Diabodies can be produced by expressing two polypeptide chains with either the structure VHA-VLB and VHB-VLA (VH-VL configuration), or VLA-VHB and VLB-VHA (VL-VH configuration) within the same cell. Most of them can be expressed in soluble form in bacteria. Single chain diabodies (scDb) are produced by connecting the two diabody-forming polypeptide chains with linker of approximately 15 amino acid residues (see Holliger and Winter, 1997 Cancer Immunol. Immunother., 45 (3-4):128-30; Wu et al., 1996 Immunotechnology, 2 (1):21-36). scDb can be expressed in bacteria in soluble, active monomeric form (see Holliger and Winter, 1997 Cancer Immunol. Immunother., 45 (34): 128-30; Wu et al., 1996 Immunotechnology, 2 (1):21-36; Pluckthun and Pack, 1997 Immunotechnology, 3 (2): 83-105; Ridgway et al., 1996 Protein Eng., 9 (7):617-21). A diabody can be fused to Fc to generate a “di-diabody” (see Lu et al., 2004 J. Biol. Chem., 279 (4):2856-65).

Other antibodies which can be employed in the bispecific molecules of the invention are murine, chimeric and humanized monoclonal antibodies.

The bispecific molecules of the present invention can be prepared by conjugating the constituent binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis (2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

When the binding specificities are antibodies, they can be conjugated by sulfhydryl bonding of the C-terminus hinge regions of the two heavy chains. In a particularly embodiment, the hinge region is modified to contain an odd number of sulfhydryl residues, for example one, prior to conjugation.

Alternatively, both binding specificities can be encoded in the same vector and expressed and assembled in the same host cell. This method is particularly useful where the bispecific molecule is a mAb×mAb, mAb×Fab, Fab×F (ab′)2 or ligand×Fab fusion protein. A bispecific molecule of the invention can be a single chain molecule comprising one single chain antibody and a binding determinant, or a single chain bispecific molecule comprising two binding determinants Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

In another aspect, the present invention provides multivalent compounds comprising at least two identical or different antigen-binding portions of the antibodies and antigen-binding fragments thereof of the invention binding to CD32b. The antigen-binding portions can be linked together via protein fusion or covalent or non-covalent linkage. Alternatively, methods of linkage has been described for the bispecific molecules. Tetravalent compounds can be obtained for example by cross-linking antibodies and antigen-binding fragments thereof of the invention with an antibody or antigen-binding fragment that binds to the constant regions of the antibodies and antigen-binding fragments thereof of the invention, for example the Fc or hinge region.

Trimerizing domain are described for example in Borean patent EP 1 012 280B1. Pentamerizing modules are described for example in PCT/EP97/05897.

Antibodies with Extended Half Life

The present invention provides for antibodies that specifically bind to CD32b which have an extended half-life in vivo.

Many factors may affect a protein's half life in vivo. For examples, kidney filtration, metabolism in the liver, degradation by proteolytic enzymes (proteases), and immunogenic responses (e.g., protein neutralization by antibodies and uptake by macrophages and dentritic cells). A variety of strategies can be used to extend the half life of the antibodies and antigen-binding fragments thereof of the present invention. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nancarriers, slow release formulations, or medical devices.

To prolong the serum circulation of antibodies in vivo, inert polymer molecules such as high molecular weight PEG can be attached to the antibodies or a fragment thereof with or without a multifunctional linker either through site-specific conjugation of the PEG to the N- or C-terminus of the antibodies or via epsilon-amino groups present on lysine residues. To pegylate an antibody, the antibody, antigen-binding fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10)alkoxy- or aryloxy-polyethylene glycol or polyethylene glycol-maleimide. In one embodiment, the antibody to be pegylated is an aglycosylated antibody. Linear or branched polymer derivatization that results in minimal loss of biological activity will be used. The degree of conjugation can be closely monitored by SD S-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known in the art and can be applied to the antibodies and antigen-binding fragments thereof of the invention. See for example, EP 0 154 316 by Nishimura et al. and EP 0 401 384 by Ishikawa et al.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a normative amino acid any place an amber codon is positioned, converting the amber from a stop codon to one that signals incorporation of the chemically specified amino acid.

Recombinant pegylation technology (rPEG) can also be used for serum halflife extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum halflife of the protein is greatly increased. In contrast to traditional PEGylation, which requires chemical conjugation and repurification, the manufacturing process is greatly simplified and the product is homogeneous.

Polysialylation is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology include the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375.

Further, antibodies can be conjugated to albumin in order to make the antibody or antibody fragment more stable in vivo or have a longer half life in vivo. The techniques are well-known in the art, see, e.g., International Publication Nos. WO 93/15199, WO 93/15200, and WO 01/77137; and European Patent No. EP 413,622.

The strategies for increasing half life is especially useful in nanobodies, fibronectin-based binders, and other antibodies or proteins for which increased in vivo half life is desired.

### Antibody Conjugates

The present invention provides antibodies or antigen-binding fragments thereof that specifically bind to CD32b recombinantly fused or chemically conjugated (including both covalent and non-covalent conjugations) to a heterologous protein or polypeptide (or antigen-binding fragment thereof, preferably to a polypeptide of at least 10, at least 20, at least 30, at least 40, at least 50, at least 60, at least 70, at least 80, at least 90 or at least 100 amino acids) to generate fusion proteins. In particular, the invention provides fusion proteins comprising an antigen-binding fragment of an antibody described herein (e.g., a Fab fragment, Fd fragment, Fv fragment, F (ab)₂ fragment, a VH domain, a VH CDR, a VL domain or a VL CDR) and a heterologous protein, polypeptide, or peptide. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies and antigen-binding fragments thereof of the invention (e.g., antibodies and antigen-binding fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16 (2):76-82; Hansson, et al., 1999, J. Mol. Biol. 287:265-76; and Lorenzo and Blasco, 1998, Biotechniques 24 (2):308-313 (each of these patents and publications are hereby incorporated by reference in its entirety). Antibodies and antigen-binding fragments thereof, or the encoded antibodies and antigen-binding fragments thereof, may be altered by being subjected to random mutagenesis by error-prone PCR, random nucleotide insertion or other methods prior to recombination. A polynucleotide encoding an antibody antigen-binding fragment thereof that specifically binds to CD32b may be recombined with one or more components, motifs, sections, parts, domains, fragments, etc. of one or more heterologous molecules.

Moreover, the antibodies and antigen-binding fragments thereof can be fused to marker sequences, such as a peptide to facilitate purification. In one embodiment, the marker amino acid sequence is a hexa-histidine peptide (SEQ ID NO: 684), such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine (SEQ ID NO: 684) provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In one embodiment, CD32b binding antibodies and antigen-binding fragments thereof of the present invention may be conjugated to a diagnostic or detectable agent. Such antibodies can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I), carbon (14C), sulfur (35S), tritium (3H), indium (115In, 113In, 112In, and 111In), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149 Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, and 117Tin; and positron emitting metals using various positron emission tomographies, and nonradioactive paramagnetic metal ions.

The present invention further encompasses uses of antibodies and antigen-binding fragments thereof conjugated to a therapeutic moiety. An antibody antigen-binding fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, an antibody antigen-binding fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, alpha-interferon, beta-interferon, nerve growth factor, platelet derived growth factor, tissue plasminogen activator, an apoptotic agent, an anti-angiogenic agent; or, a biological response modifier such as, for example, a lymphokine.

Moreover, an antibody can be conjugated to therapeutic moieties such as a radioactive metal ion, such as alpha-emitters such as 213Bi or macrocyclic chelators useful for conjugating radiometal ions, including but not limited to, 131In, 131LU, 131Y, 131Ho, 131Sm, to polypeptides. In one embodiment, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4 (10):2483-90; Peterson et al., 1999, Bioconjug. Chem. 10 (4):553-7; and Zimmerman et al., 1999, Nucl. Med. Biol. 26 (8):943-50, each incorporated by reference in their entireties.

Techniques for conjugating therapeutic moieties to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Methods of Producing Antibodies of the Invention

### Nucleic Acids Encoding the Antibodies

The invention provides substantially purified nucleic acid molecules which encode polypeptides comprising segments or domains of the CD32b-binding antibody chains described above. Some of the nucleic acids of the invention comprise the nucleotide sequence encoding the heavy chain variable region shown in any of SEQ ID NOs: 10, 62, 114, 166, 218, 270, 322, 374, 426, 478, 530, 556, 582, or 634, and/or the nucleotide sequence encoding the light chain variable region shown in any of SEQ ID NOs: 23, 75, 127, 179, 231, 283, 335, 387, 439, 491, 543, 569, 595, or 647. In a specific embodiment, the nucleic acid molecules are those identified in Table 1. Some other nucleic acid molecules of the invention comprise nucleotide sequences that are substantially identical (e.g., at least 65, 80%, 95%, or 99%) to the nucleotide sequences of those identified in Table 1. When expressed from appropriate expression vectors, polypeptides encoded by these polynucleotides are capable of exhibiting CD32b antigen binding capacity.

Also provided in the invention are polynucleotides which encode at least one CDR region and usually all three CDR regions from the heavy or light chain of the CD32b-binding antibody set forth in Table 1. Some other polynucleotides encode all or substantially all of the variable region sequence of the heavy chain and/or the light chain of the CD32b-binding antibody set forth in Table 1. Because of the degeneracy of the code, a variety of nucleic acid sequences will encode each of the immunoglobulin amino acid sequences.

The nucleic acid molecules of the invention can encode both a variable region and a constant region of the antibody. Some of the nucleic acid sequences of the invention comprise nucleotides encoding a mature heavy chain variable region sequence that is identical or substantially identical (e.g., at least 80%, 90%, or 99%) to the mature heavy chain variable region sequence set forth in any of SEQ ID NOs: 12, 38, 64, 90, 116, 142, 168, 194, 220, 246, 272, 298, 324, 350, 376, 402, 428, 454, 480, 506, 532, 558, 584, 610, 636, or 662. Some of the nucleic acid sequences of the invention comprise nucleotide encoding a mature light chain variable region sequence that is identical or substantially identical (e.g., at least 80%, 90%, or 99%) to the mature light chain variable region sequence set forth in any of SEQ ID NOs: 25, 51, 77, 103, 129, 155, 181, 207, 233, 259, 285, 311, 337, 363, 389, 415, 441, 467, 493, 519, 545, 571, 597, 623, 649, and 675.

The polynucleotide sequences can be produced by de novo solid-phase DNA synthesis or by PCR mutagenesis of an existing sequence (e.g., sequences as described in the Examples below) encoding a CD32b-binding antibody or its binding fragment. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the invention are expression vectors and host cells for producing the CD32b-binding antibodies described above. Various expression vectors can be employed to express the polynucleotides encoding the CD32b-binding antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet. 15:345, 1997). For example, nonviral vectors useful for expression of the CD32b-binding polynucleotides and polypeptides in mammalian (e.g., human) cells include pThioHis A, B & C, pcDNA3.1/His, pEBVHis A, B & C, (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding a CD32b-binding antibody chain antigen-binding fragment. In one embodiment, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of a CD32b-binding antibody chain antigen-binding fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted CD32b-binding antibody sequences. More often, the inserted CD32b-binding antibody sequences are linked to a signal sequences before inclusion in the vector. Vectors to be used to receive sequences encoding CD32b-binding antibody light and heavy chain variable domains sometimes also encode constant regions or parts thereof. Such vectors allow expression of the variable regions as fusion proteins with the constant regions thereby leading to production of intact antibodies and antigen-binding fragments thereof. Typically, such constant regions are human.

The host cells for harboring and expressing the CD32b-binding antibody chains can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express CD32b-binding polypeptides of the invention. Insect cells in combination with baculovirus vectors can also be used.

In one embodiment, mammalian host cells are used to express and produce the CD32b-binding polypeptides of the present invention. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed including the CHO cell lines, various Cos cell lines, HeLa cells, myeloma cell lines, transformed B-cells and hybridomas. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen, et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP poIIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express CD32b-binding antibody chains or binding fragments can be prepared using expression vectors of the invention which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### Generation of Monoclonal Antibodies of the Invention

Monoclonal antibodies (mAbs) can be produced by a variety of techniques, including conventional monoclonal antibody methodology e.g., the standard somatic cell hybridization technique of Kohler and Milstein, 1975 Nature 256: 495. Many techniques for producing monoclonal antibody can be employed e.g., viral or oncogenic transformation of B lymphocytes.

An animal system for preparing hybridomas is the murine system. Hybridoma production in the mouse is a well established procedure Immunization protocols and techniques for isolation of immunized splenocytes for fusion are known in the art. Fusion partners (e.g., murine myeloma cells) and fusion procedures are also known.

In a certain embodiment, the antibodies of the invention are humanized monoclonal antibodies. Chimeric or humanized antibodies and antigen-binding fragments thereof of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In a certain embodiment, the antibodies of the invention are human monoclonal antibodies. Such human monoclonal antibodies directed against CD32b can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (mu and gamma) and kappa light chain immunoglobulin sequences, together with targeted mutations that inactivate the endogenous mu and kappa chain loci (see e.g., Lonberg, et al., 1994 Nature 368 (6474): 856-859). Accordingly, the mice exhibit reduced expression of mouse IgM or K, and in response to immunization, the introduced human heavy and light chain transgenes undergo class switching and somatic mutation to generate high affinity human IgG-kappa monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13: 65-93, and Harding, F. and Lonberg, N., 1995 Ann. N.Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et al., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92103918, WO 93/12227, WO 94/25585, WO 97113852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise CD32b-binding antibodies and antigen-binding fragments thereof of the invention. For example, an alternative transgenic system referred to as the Xenomouse (Abgenix, Inc.) can be used. Such mice are described in, e.g., U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise CD32b-binding antibodies of the invention. For example, mice carrying both a human heavy chain transchromosome and a human light chain transchromosome, referred to as “TC mice” can be used; such mice are described in Tomizuka et al., 2000 Proc. Natl. Acad. Sci. USA 97:722-727. Furthermore, cows carrying human heavy and light chain transchromosomes have been described in the art (Kuroiwa et al., 2002 Nature Biotechnology 20:889-894) and can be used to raise CD32b-binding antibodies of the invention.

Human monoclonal antibodies of the invention can also be prepared using phage display methods for screening libraries of human immunoglobulin genes. Such phage display methods for isolating human antibodies are established in the art or described in the examples below. See for example: U.S. Pat. Nos. 5,223,409; 5,403,484; and U.S. Pat. No. 5,571,698 to Ladner et al; U.S. Pat. Nos. 5,427,908 and 5,580,717 to Dower et al; U.S. Pat. Nos. 5,969,108 and 6,172,197 to McCafferty et al; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Framework or Fc Engineering

Engineered antibodies and antigen-binding fragments thereof of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis. Such “backmutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In one embodiment, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered Clq binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in PCT Publication WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fc-gamma receptor by modifying one or more amino acids. This approach is described further, for example, in PCT Publication WO 00/42072 by Presta and by Lazar et al., 2006 PNAS 103(110): 4005-4010. Moreover, the binding sites on human IgG1 for Fc-gamma RI, Fc-gamma RII, Fc-gamma RIII and FcRn have been mapped and variants with improved binding have been described (see Shields, R. L. et al., 2001 J. Biol. Chen. 276:6591-6604).

In still another embodiment, the glycosylation of an antibody is modified. For example, an aglycoslated antibody can be made (i.e., the antibody lacks glycosylation). Glycosylation can be altered to, for example, increase the affinity of the antibody for “antigen’. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated or afucosylated antibody having reduced amounts of fucosyl residues, or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, LecI3 cells, with reduced ability to attach fucose to Asn (297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta (1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180). Von Horsten et al. in 2010 Glycobiology 20(12):1607-18 also describe a method of producing non-fucosylated antibodies by co-expression of antibodies with a heterologous GDP-6-deoxy-D-lyxo-4-hexulose reductase in CHO cells.

### Methods of Engineering Altered Antibodies

As discussed above, the CD32b-binding antibodies having VH and VL sequences or full length heavy and light chain sequences shown herein can be used to create new CD32b-binding antibodies by modifying full length heavy chain and/or light chain sequences, VH and/or VL sequences, or the constant region (s) attached thereto. Thus, in another aspect of the invention, the structural features of CD32b-binding antibody of the invention are used to create structurally related CD32b-binding antibodies that retain at least one functional property of the antibodies and antigen-binding fragments thereof of the invention, such as binding to human CD32b and also inhibiting one or more functional properties of CD32b.

For example, one or more CDR regions of the antibodies and antigen-binding fragments thereof of the present invention, or mutations thereof, can be combined recombinantly with known framework regions and/or other CDRs to create additional, recombinantly-engineered, CD32b-binding antibodies and antigen-binding fragments thereof of the invention, as discussed above. Other types of modifications include those described in the previous section. The starting material for the engineering method is one or more of the VH and/or VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence (s) is used as the starting material to create a “second generation” sequence (s) derived from the original sequence (s) and then the “second generation” sequence (s) is prepared and expressed as a protein.

The altered antibody sequence can also be prepared by screening antibody libraries having fixed CDR3 sequences or minimal essential binding determinants as described in US20050255552 and diversity on CDR1 and CDR2 sequences. The screening can be performed according to any screening technology appropriate for screening antibodies from antibody libraries, such as phage display technology.

Standard molecular biology techniques can be used to prepare and express the altered antibody sequence. The antibody encoded by the altered antibody sequence (s) is one that retains one, some or all of the functional properties of the CD32b-binding antibodies described herein, which functional properties include, but are not limited to, specifically binding to human CD32b protein and/or inhibiting one or more functional properties of CD32b.

The functional properties of the altered antibodies can be assessed using standard assays available in the art and/or described herein, such as those set forth in the Examples (e.g., ELISAs).

In one embodiment of the methods of engineering antibodies and antigen-binding fragments thereof of the invention, mutations can be introduced randomly or selectively along all or part of a CD32b-binding antibody coding sequence and the resulting modified CD32b-binding antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### Characterization of the Antibodies of the Invention

The antibodies and antigen-binding fragments thereof of the invention can be characterized by various functional assays. For example, they can be characterized by their ability to inhibit CD32b.

The ability of an antibody to bind to CD32b can be detected by labelling the antibody of interest directly, or the antibody may be unlabeled and binding detected indirectly using various sandwich assay formats known in the art.

In one embodiment, the CD32b-binding antibodies and antigen-binding fragments thereof of the invention block or compete with binding of a reference CD32b-binding antibody to CD32b polypeptide. These can be fully human or humanized CD32b-binding antibodies described above. They can also be other human, mouse, chimeric or humanized CD32b-binding antibodies which bind to the same epitope as the reference antibody. The capacity to block or compete with the reference antibody binding indicates that CD32b-binding antibody under test binds to the same or similar epitope as that defined by the reference antibody, or to an epitope which is sufficiently proximal to the epitope bound by the reference CD32b-binding antibody. Such antibodies are especially likely to share the advantageous properties identified for the reference antibody. The capacity to block or compete with the reference antibody may be determined by, e.g., a competition binding assay. With a competition binding assay, the antibody under test is examined for ability to inhibit specific binding of the reference antibody to a common antigen, such as CD32b polypeptide. A test antibody competes with the reference antibody for specific binding to the antigen if an excess of the test antibody substantially inhibits binding of the reference antibody. Substantial inhibition means that the test antibody reduces specific binding of the reference antibody usually by at least 10%, 25%, 50%, 75%, or 90%.

There are a number of known competition binding assays that can be used to assess competition of an antibody with a reference antibody for binding to a particular protein, in this case, CD32b. These include, e.g., solid phase direct or indirect radioimmunoassay (RIA), solid phase direct or indirect enzyme immunoassay (EIA), sandwich competition assay (see Stahli et al., Methods in Enzymology 9:242-253, 1983); solid phase direct biotin-avidin EIA (see Kirkland et al., J. Immunol. 137:3614-3619, 1986); solid phase direct labeled assay, solid phase direct labeled sandwich assay (see Harlow & Lane, supra); solid phase direct label RIA using I-125 label (see Morel et al., Molec. Immunol. 25:7-15, 1988); solid phase direct biotin-avidin EIA (Cheung et al., Virology 176:546-552, 1990); and direct labeled RIA (Moldenhauer et al., Scand. J. Immunol. 32:77-82, 1990). Typically, such an assay involves the use of purified antigen bound to a solid surface or cells bearing either of these, an unlabelled test CD32b-binding antibody and a labelled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

To determine if the selected CD32b-binding monoclonal antibodies bind to unique epitopes, each antibody can be biotinylated using commercially available reagents (e.g., reagents from Pierce, Rockford, Ill.). Competition studies using unlabeled monoclonal antibodies and biotinylated monoclonal antibodies can be performed using CD32b polypeptide coated-ELISA plates. Biotinylated MAb binding can be detected with a strep-avidin-alkaline phosphatase probe. To determine the isotype of a purified CD32b-binding antibody, isotype ELISAs can be performed. For example, wells of microtiter plates can be coated with 1 μg/ml of anti-human IgG overnight at 4 degrees C. After blocking with 1% BSA, the plates are reacted with 1 μg/ml or less of the monoclonal CD32b-binding antibody or purified isotype controls, at ambient temperature for one to two hours. The wells can then be reacted with either human IgG1 or human IgM-specific alkaline phosphatase-conjugated probes. Plates are then developed and analyzed so that the isotype of the purified antibody can be determined.

To demonstrate binding of monoclonal CD32b-binding antibodies to live cells expressing CD32b polypeptide, flow cytometry can be used. Briefly, cell lines expressing CD32b (grown under standard growth conditions) can be mixed with various concentrations of CD32b-binding antibody in PBS containing 0.1% BSA and 10% fetal calf serum, and incubated at 37 degrees C. for 1 hour. After washing, the cells are reacted with Fluorescein-labeled anti-human IgG antibody under the same conditions as the primary antibody staining. The samples can be analyzed by FACScan instrument using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

CD32b-binding antibodies and antigen-binding fragments thereof of the invention can be further tested for reactivity with CD32b polypeptide or antigenic fragment by Western blotting. Briefly, purified CD32b polypeptides or fusion proteins, or cell extracts from cells expressing CD32b can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Examples of functional assays are also described in the Example section below.

### Prophylactic and Therapeutic Uses

The present invention provides methods of treating a disease or disorder associated with increased CD32b activity or expression by administering to a subject in need thereof an effective amount of any antibody or antigen-binding fragment thereof of the invention. In a specific embodiment, the present invention provides a method of treating indications including, but not limited to, B cell malignancies including Hodgkins lymphoma, Non-Hodgkins lymphoma, multiple myeloma, diffuse large B cell lymphoma, acute lymphocytic leukemia, chronic lymphocytic leukemia, small lymphocytic lymphoma, diffuse small cleaved cell lymphoma, MALT lymphoma, mantel cell lymphoma, marginal zone lymphoma and follicular lymphoma as well as other diseases including systemic light chain amyloidosis.

In one embodiment, the present invention provides methods of treating a CD32b-related disease or disorder by administering to a subject in need thereof an effective amount of the antibodies and antigen-binding fragments thereof of the invention. Examples of known CD32b related diseases or disorders for which the disclosed CD32b binding antibodies, or antigen-binding fragments thereof, may be useful include but is not limited to: B cell malignancies including Hodgkins lymphoma, Non-Hodgkins lymphoma, multiple myeloma, diffuse large B cell lymphoma, acute lymphocytic leukemia, chronic lymphocytic leukemia, small lymphocytic lymphoma, diffuse small cleaved cell lymphoma, MALT lymphoma, mantel cell lymphoma, marginal zone lymphoma and follicular lymphoma as well as other diseases including systemic light chain amyloidosis.

In addition, the antibodies or antigen-binding fragments thereof of the invention can be used, inter alia, in combination with another antibody that binds to a cell surface antigen co-expressed with CD32b, to increase efficacy of the other antibody. In some embodiments, CD32b and the cell surface antigen are co-expressed on B cells. In some embodiments, the cell surface antigen is selected from the group consisting of CD20, CD38, CD52, CS1/SLAMF7, KiR, CD56, CD138, CD19, CD40, Thy-1, Ly-6, CD49, Fas, Cd95, APO-1, EGFR, HER2, CXCR4, HLA molecules, GM1, CD22, CD23, CD80, CD74, or DRD. In some embodiments, the other CD32b-binding antibodies, or antigen-binding fragment thereof, of the invention are used in combination with an antibody selected from the group consisting of rituximab, obinutumumab, ofatumumab, daratuximab, elotuzumab, alemtuzumab, or any other antibody that targets a cell surface antigen co-expressed with CD32b. An explanation for the observation that the anti-CD32b antibodies, or antigen-binding fragments thereof, of the invention enhance the activity of other antibodies that bind to cell surface antigens co-expressed with CD32b is that the anti-CD32b antibodies bind to CD32b and block CD32b from binding the Fc region of the cell surface antigen-binding antibody, which allows the cell surface antigen-binding antibody to engage immune effectors cells and mediate cell killing functions (e.g. via ADCC), and potentially prevents the cell surface antigen-binding antibody from being internalized into the cell and therefore not mediate cell killing (e.g. via ADCC).

Furthermore, the CD32b binding antibodies or antigen-binding fragments thereof of the invention can be used, inter alia, to treat, e.g., prevent, delay or reverse disease progression of patients who have become resistant or refractory to treatments using antibodies that bind to cell surface antigens that are co-expressed with CD32b. By blocking CD32b with the CD32b-binding antibodies, or antigen binding fragments thereof, disclosed herein, the efficacy of the cell surface antigen binding antibodies may be enhanced and therefore resistance to such antibodies reversed, in full or in part.

In one embodiment, the isolated anti-CD32b antibodies or antigen-binding fragments thereof described herein can be administered to a patient in need thereof in conjunction with a therapeutic method or procedure, such as described herein or known in the art. In addition, anti-CD32b antibodies, or antigen-binding fragments thereof, of the present disclosure, either alone or in combination with one or more antibodies that bind a cell surface antigen that is co-expressed with CD32b may be further combined with another therapeutic agent as discussed below.

For example, the combination therapy can include a composition of the present invention co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the antibody molecules are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

By “in combination with,” it is not intended to imply that the therapy or the therapeutic agents must be administered at the same time and/or formulated for delivery together, although these methods of delivery are within the scope described herein. The anti-CD32b antibody molecules can be administered concurrently with, prior to, or subsequent to, one or more other additional therapies or therapeutic agents. The anti-CD32b antibody molecule and the other agent or therapeutic protocol can be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

Exemplary combinations of anti-CD32b antibodies, or antigen-binding fragments thereof, of the present disclosure include using such antibodies in combination with compounds that are standard of care agents for treating hematologic malignancies, including multiple myeloma, non-Hodgkins lymphoma, and chronic lymphocytic lymphoma, such as ofatumumab, ibrutinib, belinostat, romidepsin, brentuximab vedotin, obinutuzumab, pralatrexate, pentostatin, dexamethasone, idelalisib, ixazomib, liposomal doxyrubicin, pomalidomide, panobinostat, elotuzumab, daratumumab, alemtuzumab, thalidomide, and lenalidomide.

In one embodiment, the anti-CD32b antibody molecule is administered in combination with a modulator, e.g., agonist, of a costimulatory molecule. In one embodiment, the modulator is IL15. In one embodiment, the agonist of the costimulatory molecule is chosen from an agonist (e.g., an agonistic antibody or antigen-binding fragment thereof, or a soluble fusion) of STING, OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

Exemplary GITR agonists include, e.g., GITR fusion proteins and anti-GITR antibodies (e.g., bivalent anti-GITR antibodies), such as, a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. No. 7,812,135, U.S. Pat. No. 8,388,967, U.S. Pat. No. 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, the anti-CD32b antibody molecule is administered in combination with an inhibitor of an inhibitory (or immune checkpoint) molecule chosen from PD-1, PD-L1, PD-L2, CTLA-4, TIM-3, LAG-3, CEACAM (e. g., CEACAM-1, CEACAM-3, and/or CEACAM-5), VISTA, BTLA, TIGIT, LAIR1, CD160, 2B4, TGFR beta, and IDO (indoleamine-2,3 dioxygenase). Inhibition of an inhibitory molecule can be performed by inhibition at the DNA, RNA or protein level.

In certain embodiments, the anti-CD32b molecules described herein are administered in combination with one or more inhibitors of PD-1, PD-L1 and/or PD-L2 known in the art. The inhibitort may be an antibody, an antigen binding fragment thereof, an immunoadhesin, a fusion protein, or oligopeptide.

In some embodiments, the anti-PD-1 antibody is chosen from any of the antibodies disclosed in WO2015/112900, MDX-1106, Merck 3475 or CT-011. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224. In some embodiments, the PD-L1 inhibitor is anti-PD-L1 antibody. In some embodiments, the anti-PD-L1 binding antagonist is chosen from YW243.55.S70, MPDL3280A, MEDI-4736, MSB-0010718C, or MDX-1105. MDX-1105, also known as BMS-936559, is an anti-PD-L1 antibody described in WO2007/005874. Antibody YW243.55.S70 (heavy and light chain variable region sequences shown in SEQ ID Nos. 20 and 21, respectively) is an anti-PD-L1 described in WO 2010/077634.

MDX-1106, also known as MDX-1106-04, ONO-4538 or BMS-936558, is an anti-PD-1 antibody described in WO2006/121168. Merck 3745, also known as MK-3475 or SCH-900475, is an anti-PD-1 antibody described in WO2009/114335. Pidilizumab (CT-011; Cure Tech) is a humanized IgG1k monoclonal antibody that binds to PD-1. Pidilizumab and other humanized anti-PD-1 monoclonal antibodies are disclosed in WO2009/101611. In other embodiments, the anti-PD-1 antibody is pembrolizumab. Pembrolizumab (Trade name Keytruda formerly lambrolizumab also known as MK-3475) disclosed, e.g., in Hamid, O. et al. (2013) New England Journal ofMedicine 369 (2): 134-44. AMP-224 (B7-DCIg; Amplimmune; e.g., disclosed in WO2010/027827 and WO2011/066342), is a PD-L2 Fc fusion soluble receptor that blocks the interaction between PD-1 and B7-H1. Other anti-PD-1 antibodies include AMP 514 (Amplimmune), among others, e.g., anti-PD-1 antibodies disclosed in U.S. Pat. No. 8,609,089, US 2010028330, and/or US 20120114649.

In some embodiments, the anti-PD-1 antibody is MDX-1106. Alternative names for MDX-1106 include MDX-1106-04, ONO-4538, BMS-936558 or Nivolumab. In some embodiments, the anti-PD-1 antibody is Nivolumab (CAS Registry Number: 946414-94-4). Nivolumab (also referred to as BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO2006/121168. Lambrolizumab (also referred to as pembrolizumab or MK03475; Merck) is a humanized IgG4 monoclonal antibody that binds to PD-1. Pembrolizumab and other humanized anti-PD-1 antibodies are disclosed in U.S. Pat. No. 8,354,509 and WO2009/114335. Other anti-PD1 antibodies include AMP 514 (Amplimmune), among others, e.g., anti-PD1 antibodies disclosed in U.S. Pat. No. 8,609,089, US 2010028330, and/or US 20120114649.

MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. Other anti-PD-L1 binding agents include YW243.55.570 (heavy and light chain variable regions are shown in SEQ ID NOs 20 and 21 in WO2010/077634) and MDX-1105 (also referred to as BMS-936559, and, e.g., anti-PD-L1 binding agents disclosed in WO2007/005874).

In some embodiments, the anti-PD-L1 antibody is MSB0010718C. MSB0010718C (also referred to as A09-246-2; Merck Serono) is a monoclonal antibody that binds to PD-L1. Pembrolizumab and other humanized anti-PD-L1 antibodies are disclosed in WO2013/079174.

AMP-224 (B7-DCIg; Amplimmune; e.g., disclosed in WO2010/027827 and WO2011/066342), is a PD-L2 Fc fusion soluble receptor that blocks the interaction between PD1 and B7-H1.

In some embodiments, the anti-LAG-3 antibody is BMS-986016. BMS-986016 (also referred to as BMS986016; Bristol-Myers Squibb) is a monoclonal antibody that binds to LAG-3. BMS-986016 and other humanized anti-LAG-3 antibodies are disclosed in US 2011/0150892, WO2010/019570, and WO2014/008218.

In one embodiment, the inhibitor is a soluble ligand (e.g., a CTLA-4-Ig), or an antibody or antibody fragment that binds to CTLA4. Exemplary anti-CTLA4 antibodies include Tremelimumab (IgG2 monoclonal antibody available from Pfizer, formerly known as ticilimumab, CP-675,206); and Ipilimumab (CTLA-4 antibody, also known as MDX-010, CAS No. 477202-00-9).

In one embodiment, the inhibitor of CEACAM (e.g., CEACAM-1, -3 and/or -5) is an anti-CEACAM antibody molecule. Carcinoembryonic antigen cell adhesion molecules (CEACAM), such as CEACAM-1 and CEACAM-5, are believed to mediate, at least in part, inhibition of an anti-tumor immune response (see e.g., Markel et al. J Immunol. 2002 Mar. 15; 168(6):2803-10; Markel et al. J Immunol. 2006 Nov. 1; 177(9):6062-71; Markel et al. Immunology. 2009 February; 126(2):186-200; Markel et al. Cancer Immunol Immunother. 2010 February; 59(2):215-30; Ortenberg et al. Mol Cancer Ther. 2012 June; 11(6):1300-10; Stern et al. J Immunol. 2005 Jun. 1; 174(11):6692-701; Zheng et al. PLoS One. 2010 Sep. 2; 5(9). pii: e12529). For example, CEACAM-1 has been described as a heterophilic ligand for TIM-3 and as playing a role in TIM-3-mediated T cell tolerance and exhaustion (see e.g., WO 2014/022332; Huang, et al. (2014) Nature doi:10.1038/nature13848). In embodiments, co-blockade of CEACAM-1 and TIM-3 has been shown to enhance an anti-tumor immune response in xenograft colorectal cancer models (see e.g., WO 2014/022332; Huang, et al. (2014), supra). In other embodiments, co-blockade of CEACAM-1 and PD-1 reduce T cell tolerance as described, e.g., in WO 2014/059251. Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146), or crossreacts with CEACAM-1 and CEACAM-5 as described in, e.g., WO 2013/054331 and US 2014/0271618.

Exemplary combinations of anti-CD32b antibody molecules (alone or in combination with other stimulatory agents) and standard of care for cancer, include at least the following.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” refers to radiation therapy delivered by a spatially confined radioactive material inserted into the body at or near a tumor or other proliferative tissue disease site. The term is intended without limitation to include exposure to radioactive isotopes (e.g., At-211, 1-131, 1-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as 1-125, 1-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of 1-125 or 1-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

The anti-CD32b antibody molecules, alone or in combination with an antibody that binds a cell surface antigen co-expressed with CD32b, and/or in combination with an immunomodulator (e.g., an anti-PD1, an anti-LAG3, anti-PD-L1 or anti-TIM-3 antibody molecule), may be used in combination with one or more of the existing modalities for treating cancers, including, but not limited to: surgery; radiation therapy (e.g., external-beam therapy which involves three dimensional, conformal radiation therapy where the field of radiation is designed, local radiation (e.g., radiation directed to a preselected target or organ), or focused radiation). Focused radiation can be selected from the group consisting of stereotactic radiosurgery, fractionated stereotactic radiosurgery, and intensity-modulated radiation therapy. The focused radiation can have a radiation source selected from the group consisting of a particle beam (proton), cobalt-60 (photon), and a linear accelerator (x-ray), e.g., as described in WO 2012/177624.

As will be appreciated by the skilled artisan, the combination therapies involving the antibodies or antigen-binding fragments thereof of the present invention, including those described in Table 1, may include combination therapies involving multiple classes of the agents described above. When the therapeutic agents of the present invention are administered together with another agent or agents, the two (or more) can be administered sequentially in any order or simultaneously. In some aspects, an antibody of the present invention is administered to a subject who is also receiving therapy with one or more other agents or methods. In other aspects, the binding molecule is administered in conjunction with surgical treatments. A combination therapy regimen may be additive, or it may produce synergistic results

### Diagnostic Uses

In one aspect, the invention encompasses diagnostic assays for determining CD32b and/or nucleic acid expression as well as CD32b function, in the context of a biological sample (e.g., blood, serum, cells, tissue) or from an individual who is afflicted with a disease or disorder.

Diagnostic assays, such as competitive assays rely on the ability of a labelled analogue (the “tracer”) to compete with the test sample analyte for a limited number of binding sites on a common binding partner. The binding partner generally is insolubilized before or after the competition and then the tracer and analyte bound to the binding partner are separated from the unbound tracer and analyte. This separation is accomplished by decanting (where the binding partner was preinsolubilized) or by centrifuging (where the binding partner was precipitated after the competitive reaction). The amount of test sample analyte is inversely proportional to the amount of bound tracer as measured by the amount of marker substance. Dose-response curves with known amounts of analyte are prepared and compared with the test results in order to quantitatively determine the amount of analyte present in the test sample. These assays are called ELISA systems when enzymes are used as the detectable markers. In an assay of this form, competitive binding between antibodies and CD32b-binding antibodies results in the bound CD32b, preferably the CD32b epitopes of the invention, being a measure of antibodies in the serum sample, including neutralising antibodies in the serum sample.

A significant advantage of the assay is that measurement is made of neutralising antibodies directly (i.e., those which interfere with binding of CD32b, specifically, epitopes). Such an assay, particularly in the form of an ELISA test has considerable applications in the clinical environment and in routine blood screening.

In the clinical diagnosis or monitoring of patients with disorders associated with CD32b, the detection of elevated levels of CD32b protein or mRNA, in comparison to the levels in a corresponding biological sample from a normal subject is indicative of a patient with disorders associated with CD32b.

In vivo diagnostic or imaging is described in US2006/0067935. Briefly, these methods generally comprise administering or introducing to a patient a diagnostically effective amount of CD32b binding molecule that is operatively attached to a marker or label that is detectable by non-invasive methods. The antibody-marker conjugate is allowed sufficient time to localize and bind to CD32b. The patient is then exposed to a detection device to identify the detectable marker, thus forming an image of the location of the CD32b binding molecules in the tissue of a patient. The presence of CD32b binding antibody or an antigen-binding fragment thereof is detected by determining whether an antibody-marker binds to a component of the tissue. Detection of an increased level in CD32b proteins or a combination of protein in comparison to a normal individual may be indicative of a predisposition for and/or on set of disorders associated with CD32b. These aspects of the invention are also for use in tissue imaging methods and combined diagnostic and treatment methods.

The invention also pertains to the field of predictive medicine in which diagnostic assays, prognostic assays, pharmacogenomics, and monitoring clinical trials are used for prognostic (predictive) purposes to thereby treat an individual prophylactically.

The invention also provides for prognostic (or predictive) assays for determining whether an individual is at risk of developing a disorder associated with dysregulation of CD32b. For example, mutations in CD32b gene can be assayed in a biological sample. Such assays can be used for prognostic or predictive purpose to thereby prophylactically treat an individual prior to the onset of a disorder characterized by or associated with CD32b, nucleic acid expression or activity.

Another aspect of the invention provides methods for determining CD32b nucleic acid expression or CD32b activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention provides a method of monitoring the influence of agents (e.g., drugs) on the expression or activity of CD32b in clinical trials.

### Pharmaceutical Compositions

The invention provides pharmaceutical compositions comprising the CD32b-binding antibody or binding fragment thereof formulated together with a pharmaceutically acceptable carrier. The compositions can additionally contain one or more other therapeutical agents that are suitable for treating or preventing a CD32b-associated disease (e.g., B cell malignancies including Hodgkins lymphoma, Non-Hodgkins lymphoma, multiple myeloma, diffuse large B cell lymphoma, acute lymphocytic leukemia, chronic lymphocytic leukemia, small lymphocytic lymphoma, diffuse small cleaved cell lymphoma, MALT lymphoma, mantel cell lymphoma, marginal zone lymphoma and follicular lymphoma as well as other diseases including systemic light chain amyloidosis). Pharmaceutically acceptable carriers enhance or stabilize the composition, or facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present invention can be administered by a variety of methods known in the art. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

The composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the CD32b-binding antibody is employed in the pharmaceutical compositions of the invention. The CD32b-binding antibodies are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the antibodies and antigen-binding fragments thereof of the invention employed in the pharmaceutical composition at levels lower than that required to achieve the desired therapeutic effect and gradually increase the dosage until the desired effect is achieved. In general, effective doses of the compositions of the present invention, for the treatment of an allergic inflammatory disorder described herein vary depending upon many different factors, including means of administration, target site, physiological state of the patient, whether the patient is human or an animal, other medications administered, and whether treatment is prophylactic or therapeutic. Treatment dosages need to be titrated to optimize safety and efficacy. For systemic administration with an antibody, the dosage ranges from about 0.0001 to 100 mg/kg, and more usually 0.01 to 15 mg/kg, of the host body weight. An exemplary treatment regime entails systemic administration once per every two weeks or once a month or once every 3 to 6 months.

Antibody is usually administered on multiple occasions. Intervals between single dosages can be weekly, monthly or yearly. Intervals can also be irregular as indicated by measuring blood levels of CD32b-binding antibody in the patient. In some methods of systemic administration, dosage is adjusted to achieve a plasma antibody concentration of 1-1000 μg/ml and in some methods 25-500 μg/ml. Alternatively, antibody can be administered as a sustained release formulation, in which case less frequent administration is required. Dosage and frequency vary depending on the half-life of the antibody in the patient. In general, humanized antibodies show longer half life than that of chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### Examples

The following examples are provided to further illustrate the invention but not to limit its scope. Other variants of the invention will be readily apparent to one of ordinary skill in the art and are encompassed by the appended claims.

### Example 1: Identification of Cd32B Antibodies

Antibodies from Morphosys HuCAL PLATINUM® Phage Library Pannings

For the selection of antibodies specifically recognizing human CD32b (human FCGR2B, UniProtKB P31994 amino acids 43-222 (SEQ ID NO:680), with APP and avi-tag) but not human CD32a-R (human FCGR2A, UniProtKB P12318 variant H167R, amino acids 34-218 (SEQ ID NO:681) with APP and avi-tag) the Morphosys HuCAL PLATINUM® library was used. The phagemid library is based on the HuCAL® concept (Knappik et al., 2000, J Mol Biol 296: 57-86) and employs the CysDisplay™ technology for displaying the Fab on the phage surface (WO01/05950). The panning strategy which ultimately resulted in the human CD32b specific antibodies were selected using a liquid phase panning strategy.

### Liquid Phase Panning on Human CD32b

The antigen selection process was performed over three rounds, using biotinylated human CD32b. Phage solution was blocked with blocking reagent before depleting the solution of possible NeutrAvidin binders on NeutrAvidin coated wells. Rescued phages were incubated with the biotinylated human CD32b for 1 hour, before phage-antigen complexes were captured on NeutrAvidin coated wells. Unbound phages were washed off using PBST (PBS supplemented with 0.05% Tween) and then with PBS. For elution of specifically bound phages, 25 mM DTT (Dithiothreitol) was added for 10 minutes (min) at RT. The DTT eluates were used for infection of E. coli (Escherichia coli) TG-F+ cells. After infection, the bacteria were centrifuged and the pellet was resuspended in 100 ml 2YT (Yeast-Trypton) Medium/Cam (chloramphenicol)/1% Glucose and incubated overnight at 37° C. and shacked at 220 rpm. The overnight culture was used for phage rescue, polyclonal amplification of selected clones, and phage production used for the next round. The second and third round of liquid phase panning was performed according to the protocol of the first round except for more stringent washing conditions.

A 4th analytical panning round was performed in order to select human CD32b specific antibodies, not binding to human CD32a-R. This round was based on the output of the 3rd round panning on human CD32b and performed on all 3 different proteins. The output of this 4th analytical round underwent a Next Generation Sequencing (NGS) analysis, rather than a classical ELISA screening.

### ELISA Screening

Using ELISA screening, single Fab clones were identified specifically binding to human CD32b and not to human CD32a-R. Fabs are tested using Fab containing crude E. coli lysates.

For identification of human CD32b antigen binding Fab fragments, Maxisorp™ (Nunc) 384 well plates were coated with 10 ug/ml NeutrAvidin before adding the biotinylated antigens: human CD32b and human CD32a-R. After blocking of plates with Superblock, Fab-containing E. coli lysates were added. Binding of Fabs was detected by goat anti-human Fab specific antibody (Fab format), AP-conjugated (Jackson Immuno Research). AttoPhos substrate was added and fluorescence emission at 535 nm was recorded with excitation at 430 nm.

### Next Generation Sequencing (NGS) Analysis

The DNA of the 4th analytical panning round was extracted and the HCDR3 region was amplified in two consecutive PCR reactions. The PCR reactions were also used to add the Illumina adaptor sequences to the 3′ and the 5′ end of the PCR fragment. Additionally, the Illumina indices were added in one adapter region in order to multiplex the samples for the sequencing reaction.

The raw data in FastQ format were used to extract amino acid sequences, align the sequences and count the occurrence of individual sequences. By comparing occurrences of individual clones deriving from different panning strategies, clones with desired binding profile (enriched on human CD32b and depleted on human CD32a-R) could be identified.

Interesting clones were isolated from the polyclonal output pool by assembly PCR. Primers flanking the light and the heavy chain, as well as HCDR3 specific primers were used to retrieve desired clones.

### Conversion to IgG and IgG Expression

In order to express full length IgG in CAP-T cells, variable domain fragments of heavy (VH) and light chains (VL) were subcloned from Display vectors (pMORPHx30) into appropriate pMorph®_hIg vectors for human IgG1. The cell culture supernatant was harvested 7 days post transfection. After sterile filtration, the solution was subjected to Protein A affinity chromatography using a liquid handling station. Samples were eluted in a 50 nM Citrate, 140 nM NaOH and pH neutralized with 1M Tris buffer and sterile filtered (0.2 μm pore size). Protein concentrations were determined by UV-spectrophotometry at 280 nm and purity of IgGs was analyzed under denaturing, reducing conditions in SDS-PAGE.

### Summary of Panning Strategies and Screening

In addition to classical phage display panning followed by ELISA screening, a novel approach using a 4th analytical panning round with a subsequent NGS analysis was performed. Using the classical approach, two antibodies were identified: NOV0281 and NOV0308. Using the novel NGS analysis approach, 3 additional antibodies were identified: NOV0563, NOV0627 and NOV0628 (discussed below).

### Example 2: Engineering of NOV0627 and NOV0628

The framework regions of NOV0628 were germlined to the closest human germlines (VH3-23 and Vlambda-3j). In addition the potential asparate isomerization site in CDR-H2 (SYDGSE) was changed from DG to DA to give antibody NOV1218 and from DG to EG to give antibody NOV1219.

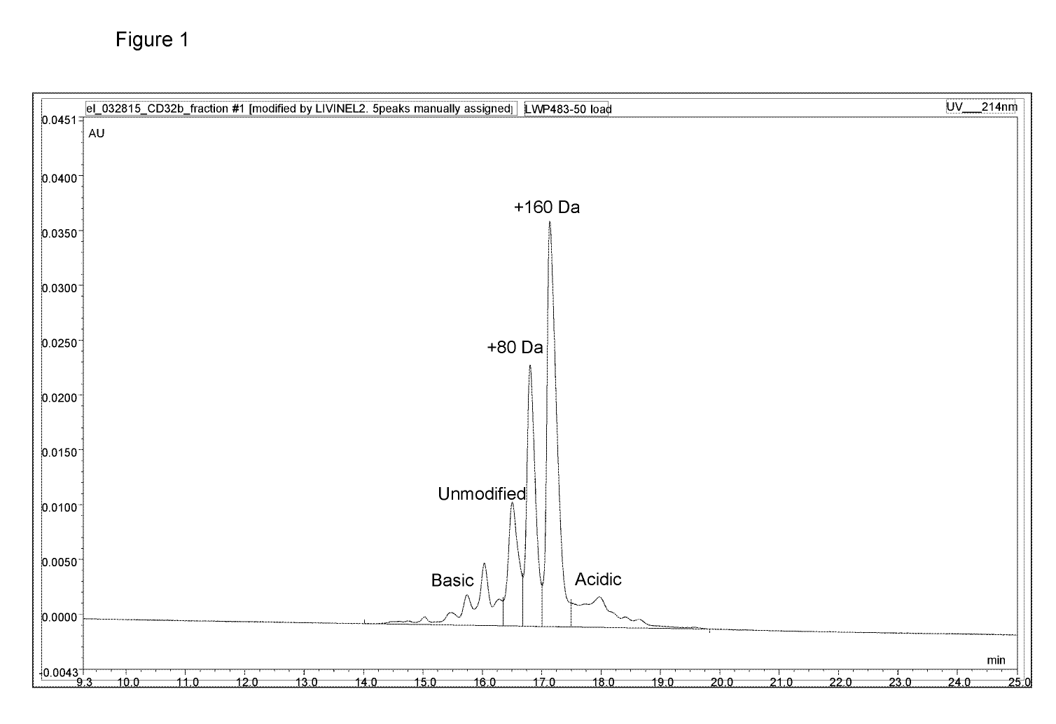

The framework regions of NOV0627 were germlined to the closest human germlines (VH1-69 and Vlambda-3j) giving antibody NOV1216. Capillary zone electrophoresis (CZE) analysis of mammalian expressed NOV1216 in IgG revealed that the antibody existed as three predominant species, unmodified, +80 daltons, and +160 daltons (FIG. 1, Table 2). CZE analysis was performed on a Beckman Coulter PA800 Enhanced instrument with uncoated fused-silica capillary. The total capillary length is 40 cm with inner diameter of 50 μm and the capillary length from inlet to detector is 30 cm. The electrophoresis running buffer consists of 400 mM 6-aminocaproic acid/acetic acid (pH 5.7) with 2 mM Triethylenetetramine and 0.03% polysorbate 20. Sample at 1 mg/mL was kept in autosampler at 15° C. and injected at 0.5 psi for 12 s. The separation was conducted for 30 min at 25° C. at a separation voltage of 20 kV. Detection was by UV absorbance at 214 nm. Between injections, the capillary was flushed with electrophoresis running buffer at 20 psi for 3 min.

TABLE 2
Summary of CZE analysis of NOV1216.

| Peak Name | Corrected Area % |
|---|---|
| basic | 13.8 |
| unmodified | 12.9 |
| +80 Da | 22.6 |
| +160 Da | 39.2 |
| acidic | 11.5 |

Mass spectrometry analysis of mammalian expressed NOV1216 in IgG format revealed that one of the four tyrosines in the CDR-H3 (EQDPEYGYGGYPYEAMDV, SeqID: 159) is susceptible for post translational modification via sulfation. This was hypothesized to be the source of the +80 and +160 dalton species. An effort to remove the P™ by mutating specific residues in CDR-H3 was initiated. Although there is no common recognition sequence for tyrosine sulfation there are reports that tyrosines flanked by acidic or small amino acids are more prone for sulfation (Nedumpully-Govindan et al., 2014, Bioinformatics 30:2302-2309). Table 3 gives an overview of the CDR-H3 mutants which were generated. In brief, the first tyrosine which is flanked by acidic and small amino acids was exchanged by phenylalanine (NOV2106), alanine (NOV2107) and serine (NOV2108), second to forth tyrosine were exchanged by phenylalanine (NOV2109, 2110, 2111). In addition, the two acidic amino acids in front of the first tyrosine were exchanged to serine (NOV2112 and 2113).

TABLE 3
Overview of NOV1216 HCDR3 mutants

| NOV identifier | SEQ ID NO: | CDR-H3 sequence |
|---|---|---|
| NOV1216 | 159 | EQDPEYGYGGYPYEAMDV |
| NOV2106 | 315 | EQDPEFGYGGYPYEAMDV |
| NOV2107 | 367 | EQDPEAGYGGYPYEAMDV |
| NOV2108 | 419 | EQDPESGYGGYPYEAMDV |
| NOV2109 | 471 | EQDPEYGFGGYPYEAMDV |
| NOV2110 | 523 | EQDPEYGYGGFPYEAMDV |
| NOV2111 | 549 | EQDPEYGYGGYPFEAMDV |
| NOV2112 | 575 | EQDPSYGYGGYPYEAMDV |
| NOV2113 | 627 | EQSPEYGYGGYPYEAMDV |

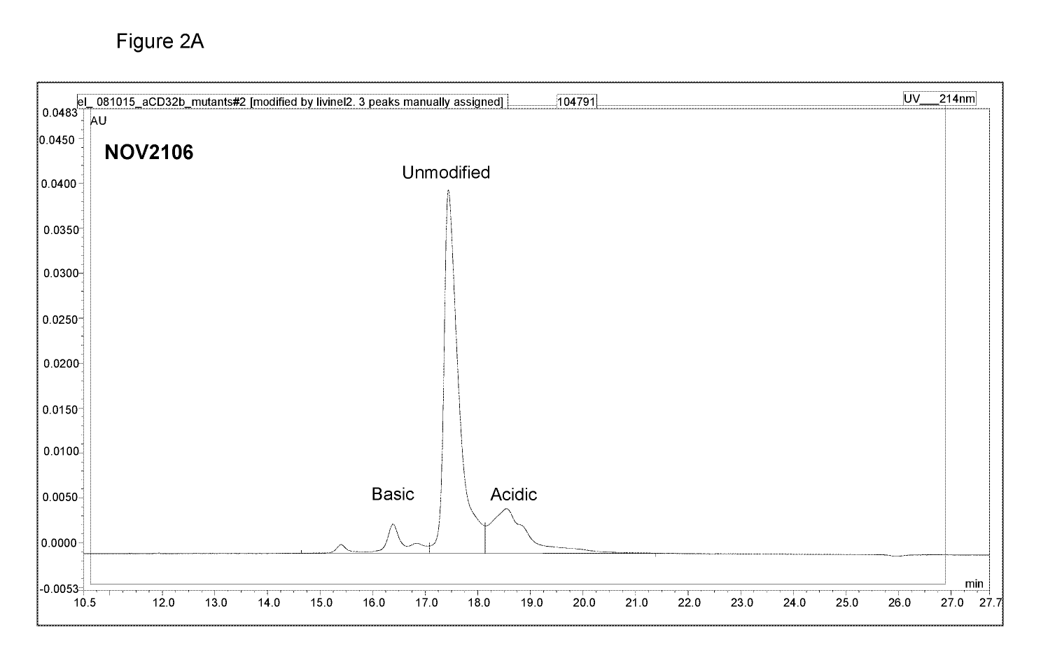

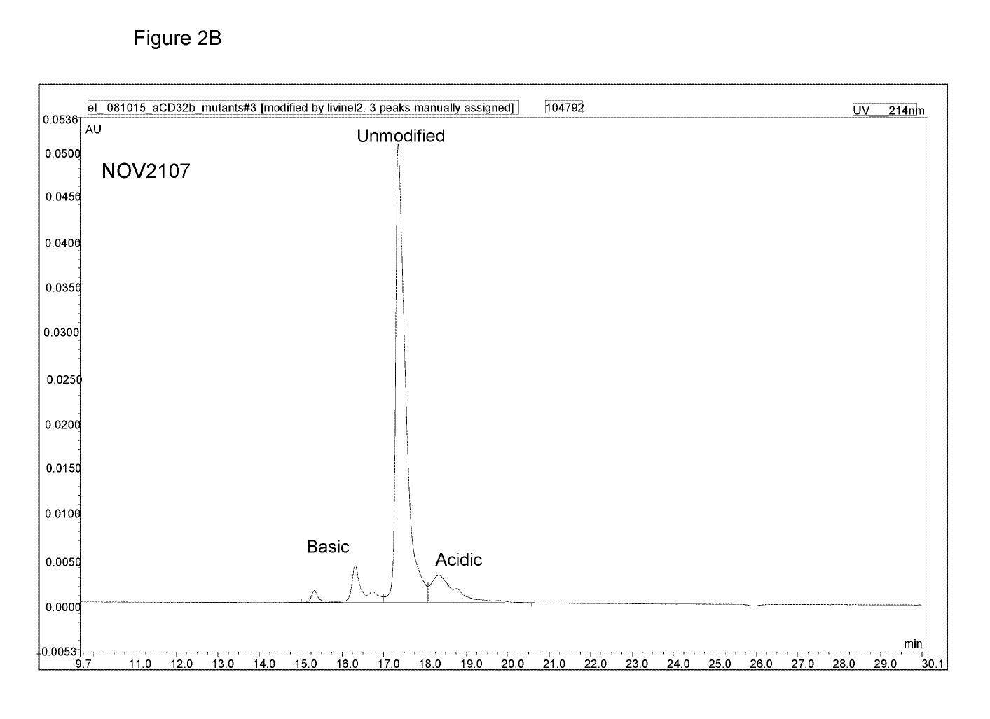

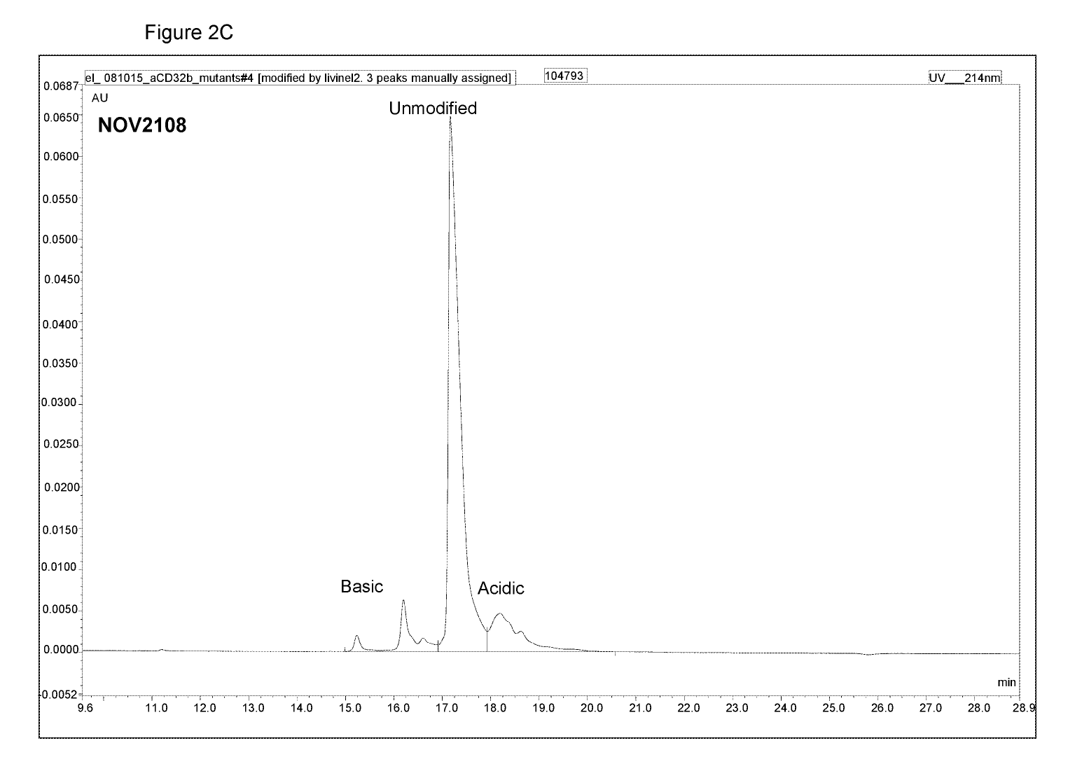

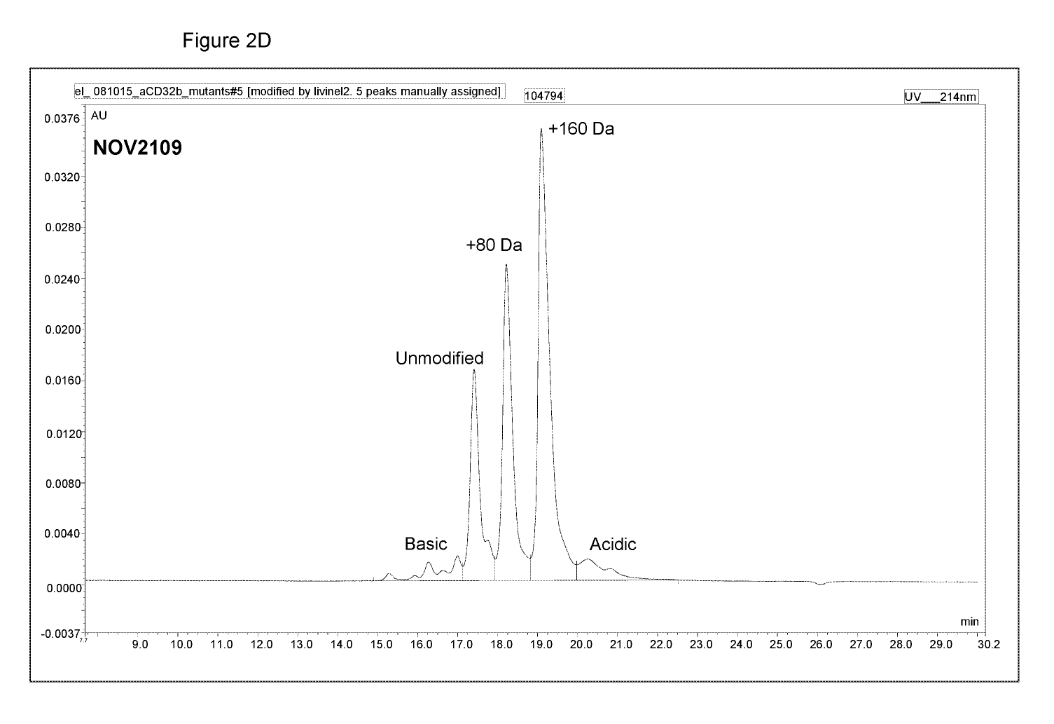

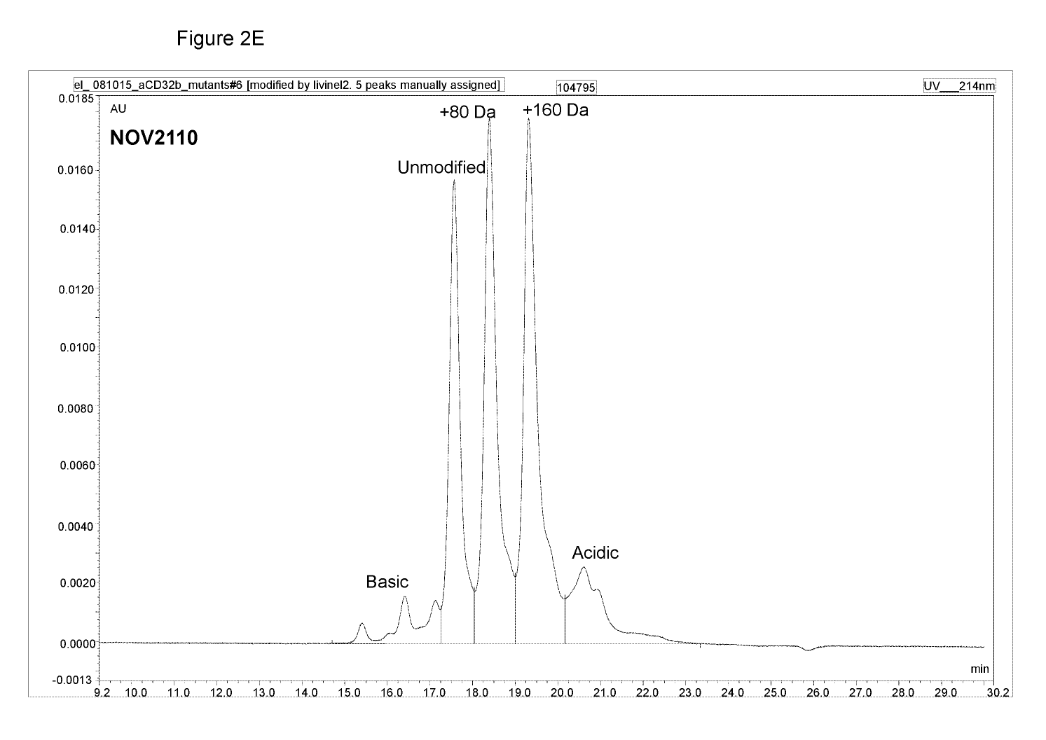

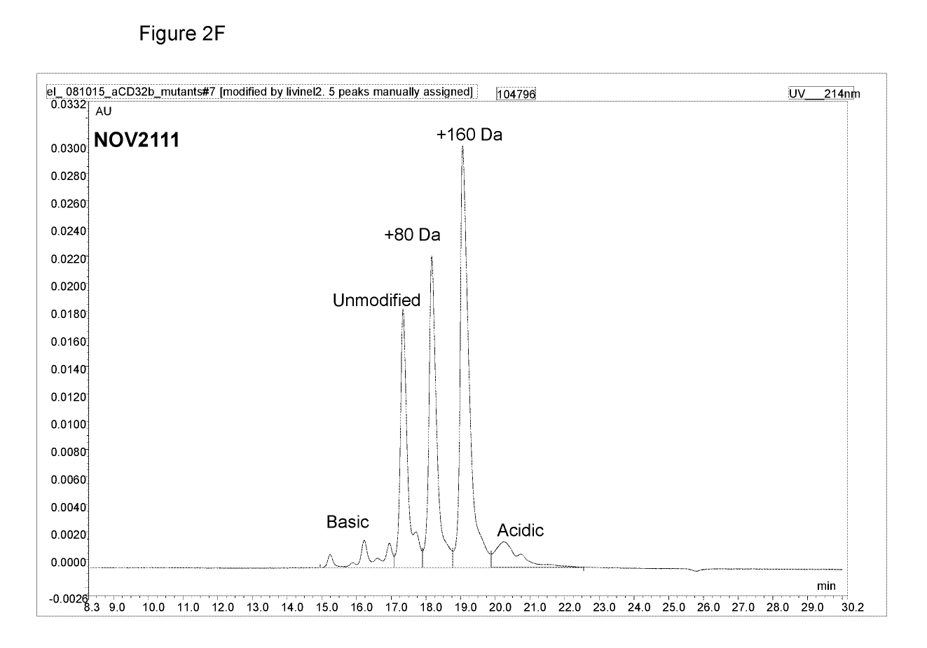

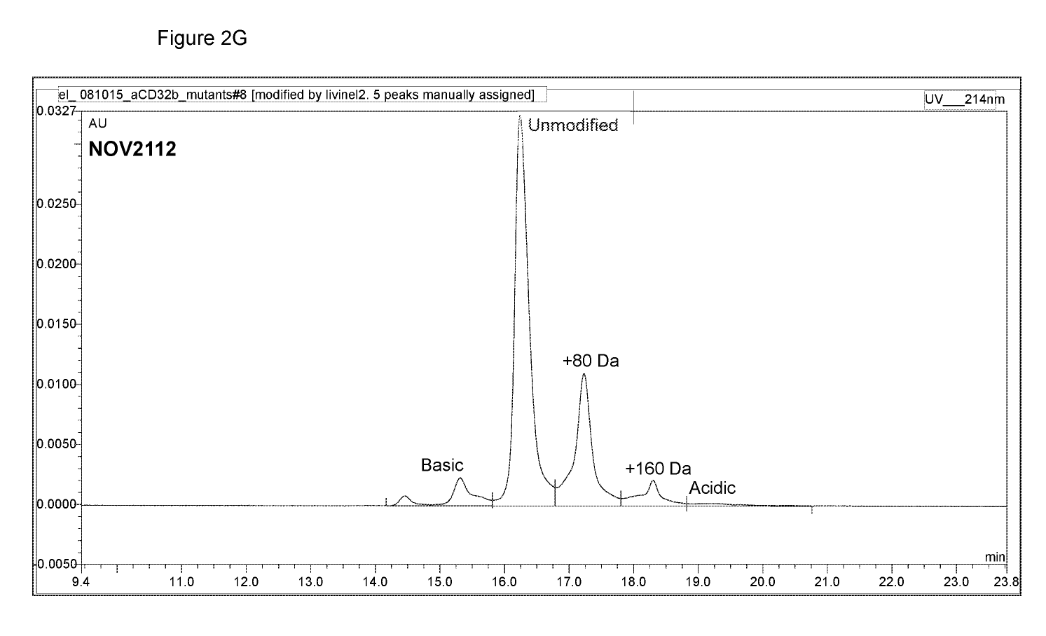

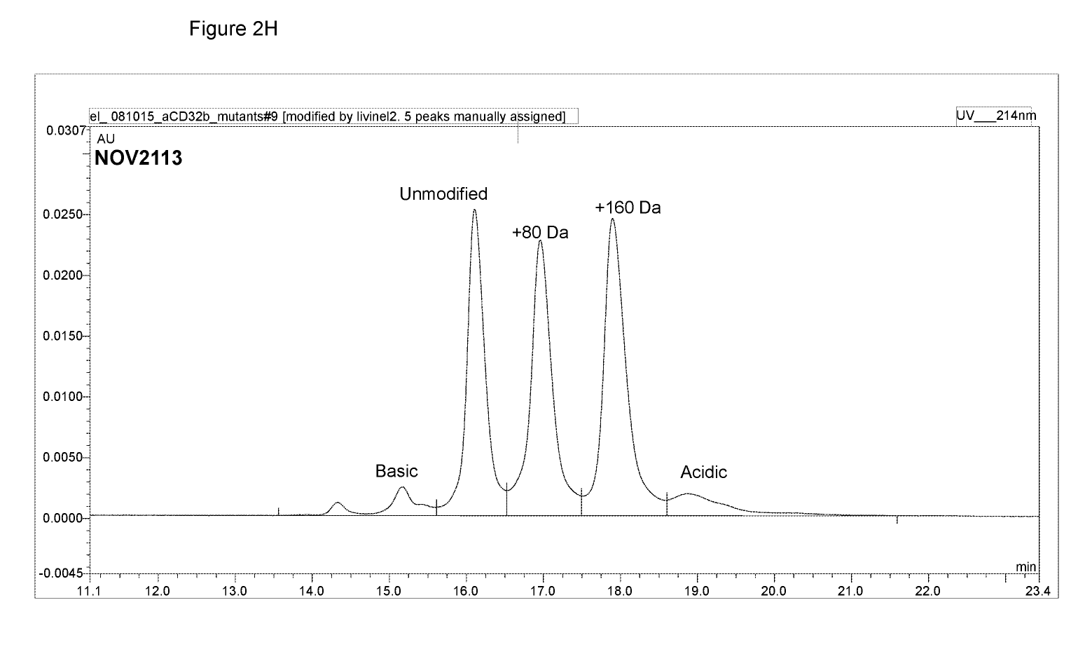

Capillary zone electrophoresis of the CDR-H3 mutants outlined in Table 3 is summarized in FIGS. 2A-2H and Table 4. Replacement of the first tyrosine with phenylalanine (NOV2106), alanine (NOV2107) or serine (NOV2108) successfully prevented the sulfation event and resulted in IgG1 antibodies that lacked the +80 and +160 dalton modifications. The remaining CDR-H3 mutants retained +80 and +160 dalton species in a manner consistent with NOV1216, supporting the hypothesis that only the first tyrosine in CDR-H3 was being modified. Likewise, mutation of the second acidic amino acid in front of the first tyrosine (NOV1213) did not resolve the +80 and +160 species. Mutation of the first acidic amino acid in front of the first tyrosine (NOV1212) did not prevent tyrosine sulfation, however, it did reduce the fraction modified by +160 Da (FIGS. 2A-2H, Table 4).

TABLE 4
Summary of capillary zone electrophoresis
analysis of huCD32b-binding antibodies.

| | Unmodified, | +80 | +160 | Basic, | Acidic, |
|---|---|---|---|---|---|
| Sample | %¹ | Da, %¹ | Da, %¹ | %¹ | %¹ |
| NOV1216 | 23.5 | 24.5 | 36.3 | 7.7 | 8.0 |
| NOV2106 | 70.3 | 0 | 0 | 9.1 | 20.6 |
| NOV2107 | 78.4 | 0 | 0 | 9.9 | 11.7 |
| NOV2108 | 77.0 | 0 | 0 | 9.3 | 13.7 |
| NOV2109 | 19.1 | 27.3 | 43.4 | 5.1 | 5.1 |
| NOV2110 | 23.4 | 29.7 | 31.6 | 6.2 | 9.1 |
| NOV2111 | 21.8 | 26.5 | 39.0 | 6.2 | 6.4 |
| NOV2112 | 62.6 | 23.8 | 5.2 | 7.5 | 0.9 |
| NOV2113 | 28.5 | 29.5 | 31.3 | 4.9 | 5.8 |

¹Percent corrected area

### Example 3: Production of Afucosylated IgG Antibodies

Afucosylated IgG antibodies were produced by applying the GlymaxX technology (Probiogen AG, Berlin.). In short, HEK293T cells were transiently transfected with expression plasmids encoding both light and heavy chain of the antibody. At the same time an expression plasmid encoding the enzyme GDP-6-deoxy-D-lyxo-4-hexulose reductase (“RMD”, “deflecting enzyme”, provided by Probiogen AG, Berlin) was co-transfected into the cells. The activity of the enzyme in the successfully transfected cells leads to inhibition of the fucose de-novo synthesis pathway. Cells expressing both the enzyme and the IgG genes produce afucosylated IgG proteins. Polyethylenimine was used as a transfection reagent. Cell culture supernatants were harvested by centrifugation and the IgG protein purified by standard chromatographic methods using Protein A and preparative size exclusion for polishing (MabSelect SURE, GE Healthcare and HiLoad 26/600 Superdex 200 pg). Purity of IgG was analyzed under denaturing, reducing and non-reducing conditions in SDS-PAGE and in native state by HP-SEC. The percentage of heavy chains carrying an N-glycan structure without core fucose was determined by mass spectrometry.

Afucosylated IgG antibodies were produced also by CHO cells. CHO cells were cultivated in shakers containing a chemical defined medium enriched in amino acids, vitamins and trace elements (Culture medium with 10 nM MTX). The batch cultivation was performed at temperature of 37° C. and shaking. After 14 days of batch cultivation process, samples of batch culture were collected to determine the viable cell density and viability using a Vi-Cell cell viability analyzer (Beckman Coulter) and to determine the protein titers in the cell culture medium. At the end of the batch (day 14), the cultivation process was stopped. The conditioned medium from the shake-flask (30 ml culture) was harvested and filtered using a 0.22 μm Steriflip filter.

The IgG protein was purified by standard chromatographic methods using Protein A and preparative size exclusion for polishing (MabSelect SURE, GE Healthcare and HiLoad 26/600 Superdex 200 pg). Purity of IgG was analyzed under denaturing, reducing and non-reducing conditions in SDS-PAGE and in native state by HP-SEC. The percentage of heavy chains carrying an N-glycan structure without core fucose was determined by mass spectrometry.

### Example 4. Binding of huCD32B-Binding Antibodies to Cho Cells Expressing huCD32A or huCD32B Variants

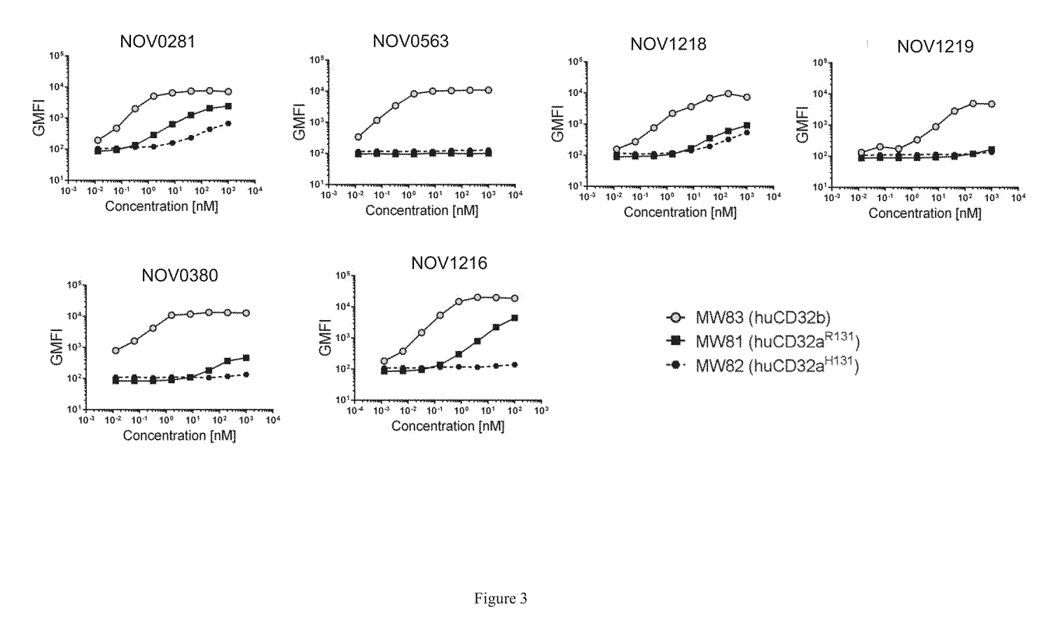

HuCD32b and huCD32a have high degree of sequence homology. To assess the specificity of huCD32b-binding antibodies, their binding was evaluated by flow cytometry using stable CHO cell lines expressing WT huCD32a variants (i.e. huCD32a^{H131} or huCD32a^{R131}) or WT human CD32b1. CHO cells were collected following detachment with PBS containing 2 mM EDTA and pelleted. Cell pellets were washed once in PBS and suspended in FACS Buffer (PBS1× containing 2% BSA, 2 mM EDTA and 0.1% NaN3), counted and suspended at 0.25×10⁶ cells per ml. 50'000 cells/well (200 μl) were then dispensed in V-bottomed 96 well plates. Plates were spun for 5 min at 1600 rpm and the supernatant discarded. Cells were then suspended in 50 μl of FACS Buffer containing the indicated concentrations of huCD32b-binding antibodies (all on a human IgG1 [N297A] scaffold) and incubated 30 min at 4° C. After 3 successive washes with FACS buffer, cells were suspended in 50 μl FACS buffer containing a 1/100 dilution of the F(ab′)2 anti-human F(ab′)2-PE (Jackson Immunoresearch#109-116-097) and further incubated 30 min at 4° C. Cells were washed twice and suspended in 200 μl FACS buffer and acquired on a FACS Canto II (acquisition of 5000 cells in the live cell gate). The Geometric Mean Fluorescence (GMFI in the PE channel) was used as a measure of the binding intensity of each antibody. FIG. 3 shows examples of huCD32b-binding antibodies displaying different degrees of discrimination between huCD32b and huCD32a variants. All huCD32b-binding antibodies have more robust binding to huCD32b than huCD32a variants.

### Example 5. Binding of huCD32B-Binding Antibodies to Cho Cells Expressing huCD16 Variants and huCD64

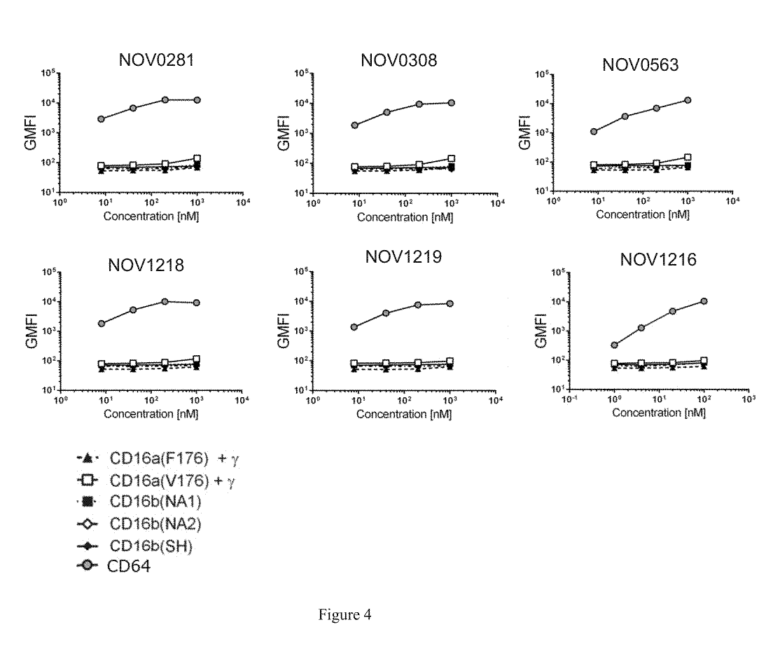

The binding of huCD32b specific antibodies was evaluated by flow cytometry using stable CHO cell lines expressing the low affinity human CD16 variants (i.e huCD16a or huCD16b variants) and the high affinity huCD64 (FcγRI). CHO cells transfected with huCD16a variants were also transfected with the common Fcγ chain in order to allow for surface expression. CHO cells were collected following detachment with PBS containing 2 mM EDTA and pelleted. Cell pellets were washed once in PBS and suspended in FACS Buffer (PBS1× containing 2% BSA, 2 mM EDTA and 0.1% NaN3), counted and suspended at 0.25×10⁶ cells per ml. 50'000 cells/well (200 μl) were then dispensed in V-bottomed 96 well plates. Plates were spun for 5 min at 1600 rpm and the supernatant discarded. Cells were then suspended in 50 μl of FACS Buffer containing the indicated concentrations of huCD32b-binding antibodies (all on a human IgG1 [N297A] scaffold) and incubated 30 min at 4° C. After 3 successive washes with FACS buffer, cells were suspended in 50 μl FACS buffer containing a 1/100 dilution of the F(ab′)2 anti-human F(ab′)2-PE (Jackson Immunoresearch#109-116-097) and further incubated 30 min at 4° C. Cells were washed twice and suspended in 200 μl FACS buffer and acquired on a FACS Canto II (acquisition of 5000 cells in the live cell gate). The Geometric Mean Fluorescence (GMFI in the PE channel) was used as a measure of the binding intensity of each antibody. All huCD32b-binding antibodies tested displayed no reactivity to CHO cells expressing huCD16 variants and partial dose dependent binding to the high affinity huCD64 receptors (FIG. 4). The dose-dependent binding to huCD64 receptor likely occurred via binding of the Fc portion of the antibodies tested to the high affinity Fc binding domain of huCD64 as this occurred independently of the epitope specificity of Abs and was blocked by pre-incubation of CHO-huCD64 cells with human IgG1 (data not shown).

### Example 6: Binding of Human Cd32B-Binding Antibodies to Human Primary B Cells

CD32b is the sole Fc receptor expressed on B cells. The binding of huCD32b specific antibodies to primary human B cells was evaluated by flow cytometry on purified B cells isolated from buffy coats by negative selection using the Human B Cell Enrichment Kit (STEMCELL Technologies #19054) according to the supplier's instructions. Purified B cells were suspended in FACS Buffer (PBS1× containing 2% BSA, 2 mM EDTA), counted and suspended at 0.5×106 cells per ml. 100′000 cells/well (200 μl) were then dispensed in V-bottomed 96 well plates. Plates were spun for 5 min at 1500 rpm and the supernatant discarded. Cells were then suspended in 50 μl of FACS Buffer containing the indicated concentrations of biotinylated huCD32b-binding antibodies (all on a human IgG1 [N297A] scaffold) and incubated 20 min at 4° C. Biotinylation of antibodies was performed using the Lightning-Link biotin conjugation kit (Type A) from Innova Biosciences (Cat. No 704-0010) according to the supplier's instructions. After 2 successive washes with FACS buffer, cells were suspended in 50 μl FACS buffer containing a 1/500 dilution of Streptavidin-PE (Invitrogen 521388) and 1 μl of an APC-conjugated anti-huCD20 Ab (clone 2H7 from Biolegend 302310) and further incubated for 20 min at 4° C. Cells were washed twice and suspended in 200 μl FACS buffer and acquired on a FACS Fortessa. The Geometric Mean Fluorescence (GMFI in the PE channel) in the CD20+ B cell gate was used as a measure of the binding intensity of each antibody. All huCD32b-binding antibodies demonstrated robust binding to human B cells, with NOV1216, NOV0281, and NOV0308 having the greatest binding affinity (1.4, 5.4, and 8.7 nM, respectively; FIG. 5).

### Example 7: Binding of huCD32B-Binding Antibodies to Human BJAB Cells

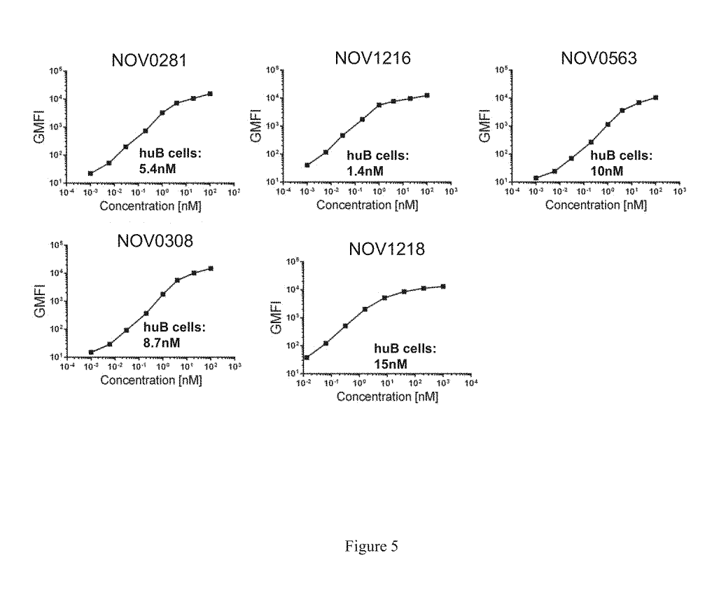

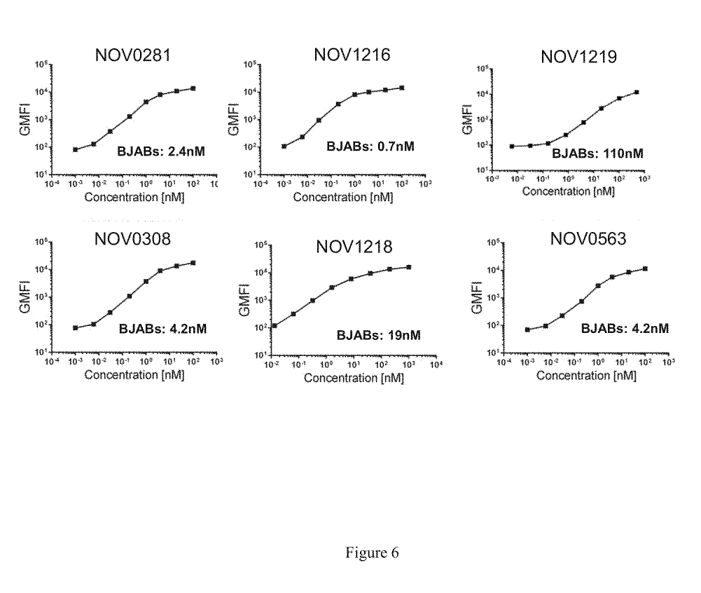

The binding of huCD32b specific antibodies to the BJAB cell line was evaluated by flow cytometry. BJAB cells were collected and suspended in FACS Buffer (PBS1× containing 2% BSA, 2 mM EDTA), counted and suspended at 0.25×10⁶ cells per ml. 50'000 cells/well (200 μl) were then dispensed in V-bottomed 96 well plates. Plates were spun for 5 min at 1500 rpm and the supernatant discarded. Cells were then suspended in 50 μl of FACS Buffer containing the indicated concentrations of biotinylated huCD32b-binding antibodies (all on a human IgG1 [N297A] scaffold) and incubated 20 min at 4° C. Biotinylation of antibodies was performed using the Lightning-Link biotin conjugation kit (Type A) from Innova Biosciences (Cat. No 704-0010) according to the supplier's instructions. After 2 successive washes with FACS buffer, cells were suspended in 50 μl FACS buffer containing a 1/500 dilution of Streptavidin-PE (Invitrogen 521388) and further incubated for 20 min at 4° C. Cells were washed twice and suspended in 200 μl FACS buffer and acquired on a FACS Fortessa. The Geometric Mean Fluorescence (GMFI in the PE channel) was used as a measure of the binding intensity of each antibody. All huCD32b-binding antibodies demonstrated robust binding to parental BJAB cells, with NOV1216, NOV0281, NOV0308, and NOV0563 having the greatest binding affinity (FIG. 6).

### Example 8: Epitope Recognition by Anti-Human Cd32B-Binding Antibodies

### a) Epitope Analysis by FACS Binding

Summary of WT and Mutant huCD32b Transfected CHO Cells Used to Characterize the Binding Epitope of Anti-CD32b Antibodies

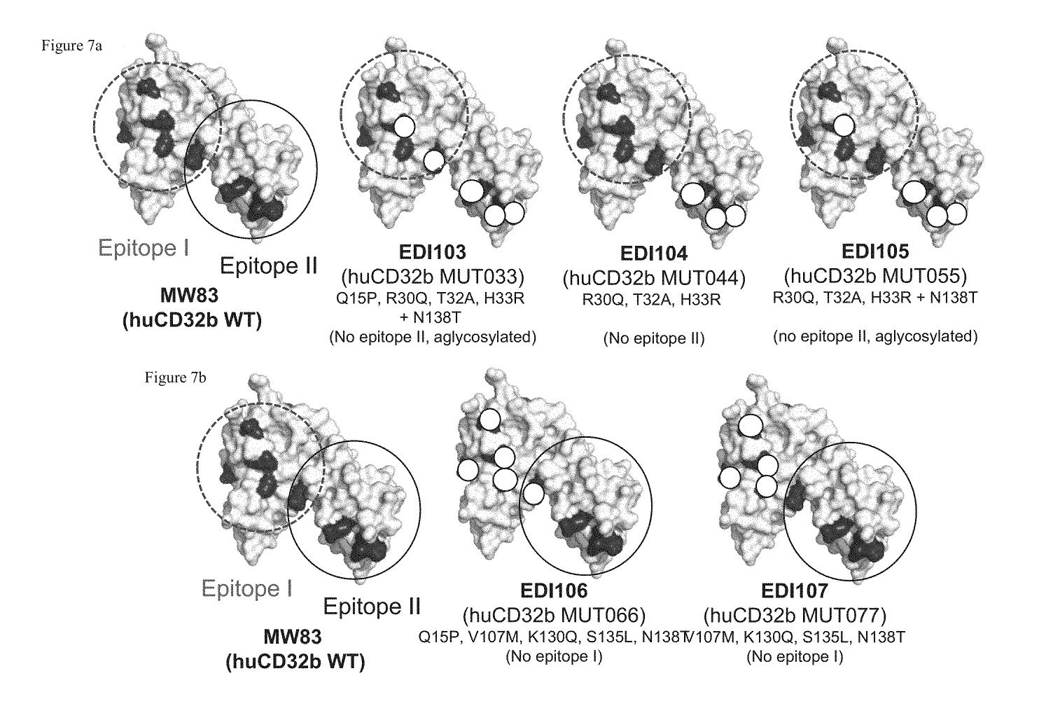

Stable CHO cell lines expressing WT human CD32b or CD32b encompassing the amino acid mutations discussed below were generated using the Flp-In™ technology. Stable cell transfectants were selected using Hygromycin B. Residues highlighted in black in the 3D model structure of human CD32b highlight amino acids differing between huCD32b and huCD32a (FIG. 7a). EDI103, EDI104, EDI105, EDI106 and EDI107 CHO cells express huCD32b with specific amino acid mutations reverting the indicated amino acid to the corresponding amino acids in human CD32a. The amino acid modified in each cell line are highlighted by the open circles on the corresponding 3D structure (Sondermann et al., The EMBO Journal (1999) 18, 1095-1103) and specified for each cell line. The assessment of the binding of huCD32b-binding antibodies to these different huCD32b variants allows the identification of the major epitope areas recognized by the antibody. The left part of the huCD32b structure was defined as epitope I and corresponds to the Fc binding domain of huCD32b. The right side was defined as epitope II and is not involved in Fc binding. In EDI103, EDI104 and EDI105 mutants, epitope II was disrupted by rendering it identical to huCD32a (FIG. 7a). In EDI106 and EDI107 mutants, epitope I of huCD32b was disrupted by rendering it identical to huCD32a (FIG. 7b).

FACS Binding Experiments Designed to Characterize Binding Epitopes Recognized by Anti-huCD32b-Binding Antibodies

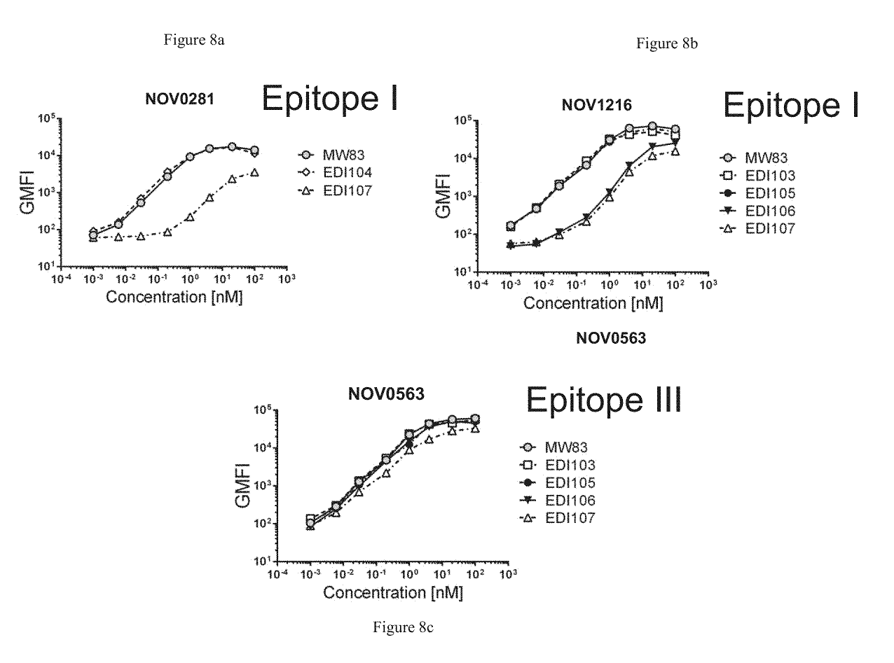

The binding epitope of huCD32b specific antibodies was evaluated by flow cytometry using stable CHO cell lines expressing WT human CD32b or mutant CD32b variants in which the amino acids differing between huCD32b and huCD32a in the Fc binding domain (epitope I) or the opposite end of the CD32b molecule (epitope II) were abrogated by reverting specific huCD32b residues into the corresponding amino acids in CD32a. EDI103, EDI104 and EDI105 CHO variants express huCD32b mutants with epitope 2 amino acids identical to huCD32a while EDI106 and EDI107 express huCD32b with epitope I amino acids identical to human CD32a (FIG. 7a). CHO cells were collected following detachment with PBS containing 2 mM EDTA and pelleted. Cell pellets were washed once and in PBS and suspended in FACS Buffer (PBS1× containing 2% BSA, 2 mM EDTA and 0.1% NaN3), counted and suspended at 0.25×10⁶ cells per ml. 50'000 cells/well (200 μl) were then dispensed in V-bottomed 96 well plates. Plates were spun for 5 min at 1600 rpm and the supernatant discarded. Cells were then suspended in 50 μl of FACS Buffer containing the indicated concentrations of huCD32b-binding antibodies (all on a human IgG1 [N297A] scaffold) and incubated 30 min at 4° C. After 3 successive washes with FACS buffer, cells were suspended in 50 μl FACS buffer containing a 1/100 dilution of the F(ab′)2 anti-human F(ab′)2-PE (Jackson Immunoresearch#109-116-097) and further incubated 30 min at 4° C. Cells were washed twice and suspended in 200 μl FACS buffer and acquired on a FACS Canto II (acquisition of 5000 cells in the live cell gate). The Geometric Mean Fluorescence (GMFI in the PE channel) was used as a measure of the binding intensity of each antibody. FIG. 8 shows examples of huCD32b-binding antibodies displaying different binding epitopes based on the reduced binding to CHO cells expressing specific huCD32b-mutants. NOV0281 and NOV1216 displayed reduced binding to epitope I deficient EDI106 and EDI107 huCD32b mutants indicating that these antibodies mainly recognize epitope I (i.e. the Fc binding domain area) (FIG. 8a, FIG. 8b). The antibody NOV0563 displayed similar binding to all huCD32b CHO variants tested suggesting that such antibody either recognizes an epitope in between areas covered by epitope I and epitope II or alternatively an additional area in the back of the 3D huCD32b structure encompassing another single amino acid difference between huCD32b and huCD32a, defined here as epitope III (FIG. 8c). A summary of the binding data in FIG. 8a, FIG. 8b, and FIG. 8c is presented in Table 5A.

TABLE 5A
Summary of huCD32b-binding antibodies binding to CHO cells
expressing huCD32b with either disrupted Epitope 1
(Fc binding domain) or Epitope II.

| | | Robust binding | | | | |
|---|---|---|---|---|---|---|
| | | to huCD32b | | | | |
| | Robust | with Epitope | | | | |
| | binding to | I (Fc binding | | Robust binding to | | |
| | WT | domain) | | huCD32b with | | |
| | CD32b | disrupted | | Epitope II disrupted | | |
| Antibody | MW83 | EDI106 | EDI107 | EDI103 | EDI104 | EDI105 |
| NOV1216 | yes | no | no | yes | nd | yes |
| NOV0281 | yes | nd | no | nd | yes | nd |
| NOV0563 | yes | yes | yes | yes | nd | yes |

nd = not determined

### NOV2108 Recognizes the CD32b Fc Binding Domain (Epitope I)

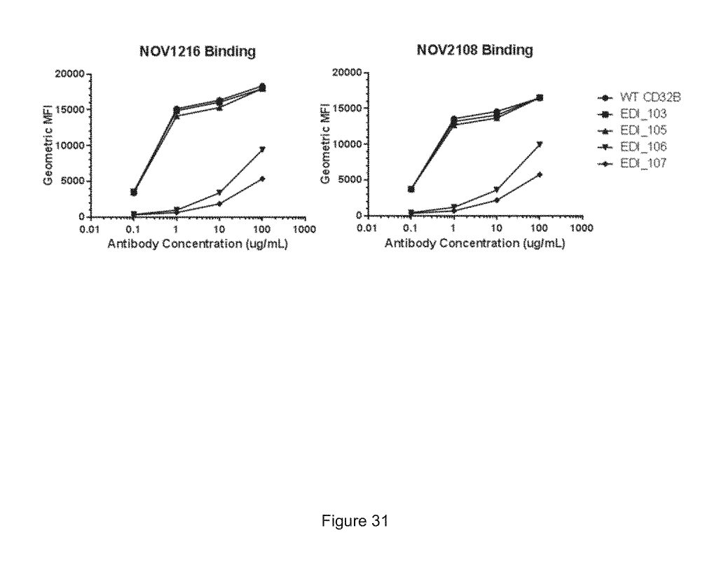

The binding epitope of huCD32b specific antibodies NOV2108 and NOV1216 was evaluated by flow cytometry using stable CHO cell lines expressing WT human CD32a, CD32b or mutant CD32b variants in which the amino acids differing between huCD32b and huCD32a in the Fc binding domain (epitope I) or the opposite end of the CD32b molecule (epitope II) were abrogated by reverting specific huCD32b residues into the corresponding amino acids in CD32a. In EDI103 and EDI105 mutants, epitope II was disrupted by rendering it identical to huCD32a (FIG. 7a). In EDI106 and EDI107 mutants, epitope I of huCD32b was disrupted by rendering it identical to huCD32a (FIG. 7b). Adherent CHO cell lines were grown in DMEM (Lonza cat. no.: 12-604F), 10% FBS (Seradigm Prod. No 1500-500, Lot #112B15), 600 μg/ml Hygromycin B (Life Tech 10687-010). Confluent cells were harvested by rinsing with PBS (Lonza Cat. No. 17-516F) and treating with 0.25% Trypsin (Gibco 25200-056) in culture. Following detachment cells were pelleted, washed once in PBS, and resuspended in FACS Buffer (PBS1× containing 2% FBS). Each cell line was resuspended 2×10⁶ cells/ml before aliquoting 100 μl/well in a 96 well u-bottom plate (Falcon 351177). Plates were spun down for 5 min at 1200 rpm and the supernatant was discarded. Cells were then resuspended in 100 μl of FACS Buffer containing the indicated concentrations of Alexa Fluor 647-labeled (Molecular Probes A20186) huCD32b-reactive antibodies (all on a human IgG1 [N297A] scaffold) and incubated 30 min at 4° C. For FIG. 31, a four point, 1:10 serial dilution starting at 100 ug/ml was prepared for AlexaFluor 647-labeled NOV1216 and AlexaFluor 647-labeled NOV2108. Each CHO cell line was incubated with one antibody as indicated in the figure separately. After 3 successive washes with FACS buffer, cells were suspended in 100 μl FACS buffer. After the final wash, cells were resuspended in 100 μl FACS buffer and acquired on a FACS Canto II (acquisition of 5000 cells in the live cell gate). The Geometric Mean Fluorescence Intensity (GMFI in the AF647 channel) was used as a measure of the binding intensity of each antibody.

NOV2108 and NOV1216 displayed reduced binding to epitope I deficient EDI106 and EDI107 huCD32b mutants (FIG. 31) indicating that these antibodies recognize epitope I (i.e. the Fc binding domain) Both antibodies showed similar binding to WT CD32b and epitope II deficient EDI103 and 105 huCD32b mutants indicating that epitope II is not required for the binding of the two antibodies. A summary of the binding data is summarized in Table 5B.

TABLE 5B
Summary of huCD32b reactive antibodies binding to CHO cells expressing
huCD32b with either disrupted Epitope 1 (Fc binding domain) or Epitope II.

| | | | Robust binding | | | | |
|---|---|---|---|---|---|---|---|
| | Robust | Robust | to huCD32b | | | | |
| | binding | binding to | with Epitope I | | Robust binding to | | |
| | to WT | WT | (Fc binding domain) | | huCD32b with | | |
| | CD32a | CD32b | disrupted | | Epitope II disrupted | | |
| Antibody | MW83 | MW83 | EDI106 | EDI107 | EDI103 | EDI104 | EDI105 |
| NOV1216 | nd | yes | no | no | yes | nd | yes |
| NOV2108 | no | yes | no | no | yes | nd | yes |

nd = not determined

b) Epitope Mapping of NOV2108 on huCD32b by Hydrogen-Deuterium Exchange

Hydrogen-deuterium exchange (HDx) in combination with mass spectrometry (MS) (Woods V L, Hamuro Y (2001) High Resolution, High-Throughput Amide Deuterium Exchange-Mass Spectrometry (DXMS) Determination of Protein Binding Site Structure and Dynamics: Utility in Pharmaceutical Design. J. Cell. Biochem. Supp.; 84(37): 89-98) was used to map the putative binding site of Fab antibody NOV2108 on human CD32b (aa1-175) (SEQ ID NO:682). In HDx, exchangeable amide hydrogens of proteins are replaced by deuterium. This process is sensitive to protein structure/dynamics and solvent accessibility and, therefore, able to report on locations that undergo a decrease in deuterium uptake upon ligand binding. Changes in deuterium uptake are sensitive to both direct binding and allosteric events.

HDx-MS experiments were performed using methods similar to those described in the literature (Chalmers M J, Busby S A, Pascal B D, He Y, Hendrickson C L, Marshall A G, Griffin P R (2006), Probing protein Ligand Interactions by Automated Hydrogen/deuterium Exchange Mass Spectrometry. Anal. Chem.; 78(4): 1005-1014). In these experiments, the deuterium uptake of human CD32b (aa1-175) was measured in the absence and presence of antibody NOV2108 in Fab format. Regions in human CD32b (aa1-175) that show a decrease in deuterium uptake upon binding of the antibody are likely to be involved in the epitope; however, due to the nature of the measurement it is also possible to detect changes remote from the direct binding site (allosteric effects). Usually, the regions that have the greatest amount of protection are involved in direct binding although this may not always be the case. In order to delineate direct binding events from allosteric effects, orthogonal measurements (e. g. X-ray crystallography, alanine mutagenesis, etc.) are required.

The human CD32b (aa1-175) epitope mapping experiments were performed on a Waters HDx-MS platform, which includes a LEAP autosampler, nanoACQUITY UPLC System, and Synapt G2 mass spectrometer. The deuterium buffer used to label the protein backbone of human CD32b (aa1-175) with deuterium was 125 mM PBS, 150 mM NaCl, pH 7.2; the overall percentage of deuterium in the solution was 95%. For human CD32b (aa1-175) deuterium labeling experiments in the absence of antibody, 175 μmol of human CD32b

(aa1-175), volume of 9 μl, was diluted using 100 μl of the deuterium buffer for 25 minutes at 4° C. The labeling reaction was then quenched with 100 μl of chilled quench buffer at 2° C. for five minutes followed by injection onto the LC-MS system for automated pepsin digestion and peptide analysis. For human CD32b (aa1-175) deuterium labeling experiments in the presence of NOV2108, 175 μmol of human CD32b (aa1-175) is mixed with 210 μmol NOV2108 antibody in Fab format, total volume of 9 μl. The solution is then diluted using 100 μl of the deuterium buffer for 25 minutes at 4° C. The labeling reaction was then quenched with 100 μl of chilled quench buffer at 2° C. for five minutes followed by injected onto the LC-MS system for automated pepsin digestion and peptide analysis.

All experiments are carried out using a minimum three analytical triplicates. All deuterium exchange experiments were quenched using 0.5M TCEP and 3M Urea (pH=2.5). After quenching, the antigen was injected into the UPLC system where it is subjected to on-line pepsin digestion at 12° C. followed by a rapid 8 minute 2 to 35% acetonitrile gradient over a Waters CSH C18 1×100 mm column (maintained at 1° C.) at a flow rate of 40 uL/min.

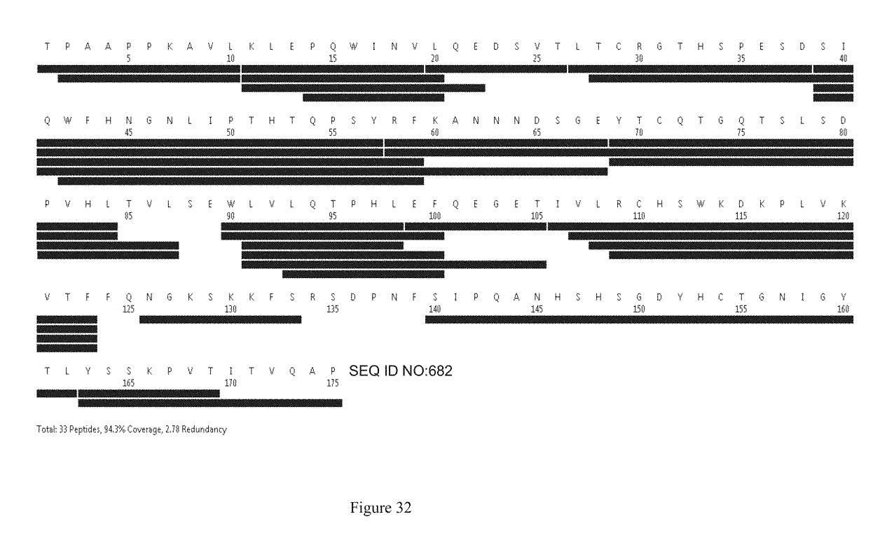

For human CD32b (aa1-175) 94% of the sequence was monitored by the deuterium exchange experiments as indicated in FIG. 32. In this figure each bar represents a peptide that is monitored in all deuterium exchange experiments.

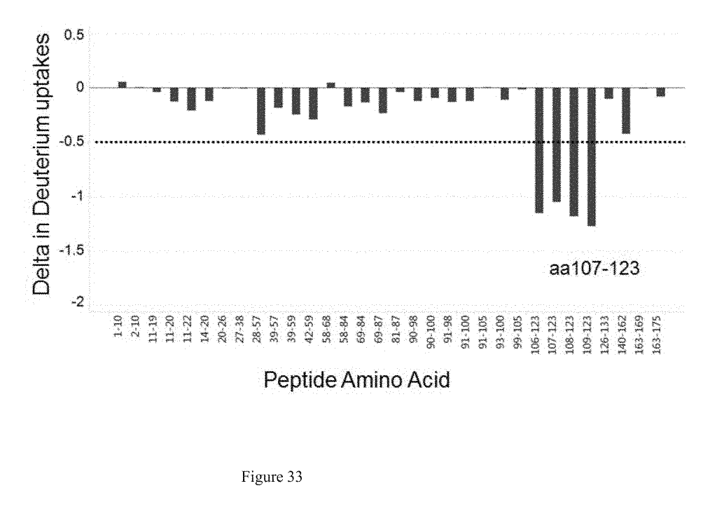

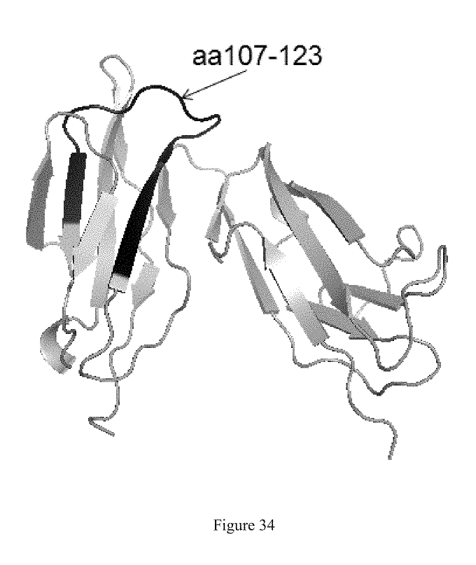

For differential experiments between antibody NOV2108 Fab bound and unbound states it is informative to examine the difference in deuterium uptake between the two states. In FIG. 33 a negative value indicates that the human CD32b-antibody complex undergoes less deuterium uptake relative to human CD32b. A decrease in deuterium uptake can be due to protection of the region from exchangeable deuterium or stabilization of the hydrogen bonding network. In contrast, a positive value indicates that the complex undergoes more deuterium uptake relative to human CD32b. An increase in deuterium uptake can be due to destabilization of hydrogen bonding networks (i.e. localized unfolding of the protein). In these experiments we did not observe any significant destabilization due to the binding of NOV2108 Fab to CD32b.

When examining the differential change in deuterium exchange between two different states, such as unbound human CD32b and human CD32b complexed with antibody NOV2108, methods are utilized to determine if the changes are significant. In one method (Houde et al., J Pharm Sci 100(6):2071-2086 (2011)), as long as the difference is greater than 0.5 Da (denoted by the dashed line in FIG. 33), the difference is considered significant. Using the previously mentioned method, upon the binding of Ab NOV2108 Fab, a single region, aa107-123 (VLRCHSWKDKPLVKVTF (SEQ ID NO: 685)), becomes significantly protected. Previously published data suggest that several residues are critical for Fc binding: aa112-119(SWKDKPLV (SEQ ID NO: 686)) and aa133-138(SRSDPNF (SEQ ID NO: 687)) (Hulett M D, Witort E, Brinkworth R I, McKenzie I F, and Hogarth P M. (1995), Multiple Regions of Human FcgRII (CD32) Contribute to the Binding of IgG. The J. Bio Chem.; 36 (270): 21188-21194). The region aa112-119(SWKDKPLV (SEQ ID NO: 686)) is protected by NOV2108 binding in our HDx-MS experiments. The region corresponding to 133-138(SRSDPNF (SEQ ID NO: 687)) is not able to be monitored in our HDx-MS experiment; this region corresponds to C′/E loop. In FIG. 34, the region (in black color) protected by Ab NOV2108 is mapped onto a published human CD32b crystal structure (Sondermann P., Huber R. and Jacob U. (1999), Crystal structure of the soluble form of the human fcgamma-receptor IIb: a new member of the immunoglobulin superfamily at 1.7 Å resolution. The EMBO J.; 5(18):1095-1103). This region includes the B/C loop structure as well as B+C β-sheets. These data support observations from functional assays indicating that NOV2108 binds the CD32b Fc binding domain.

### Example 9: Determination of Human Cd32B-Binding Antibodies Binding to Cells Featuring a Range of Human Cd32B Expression

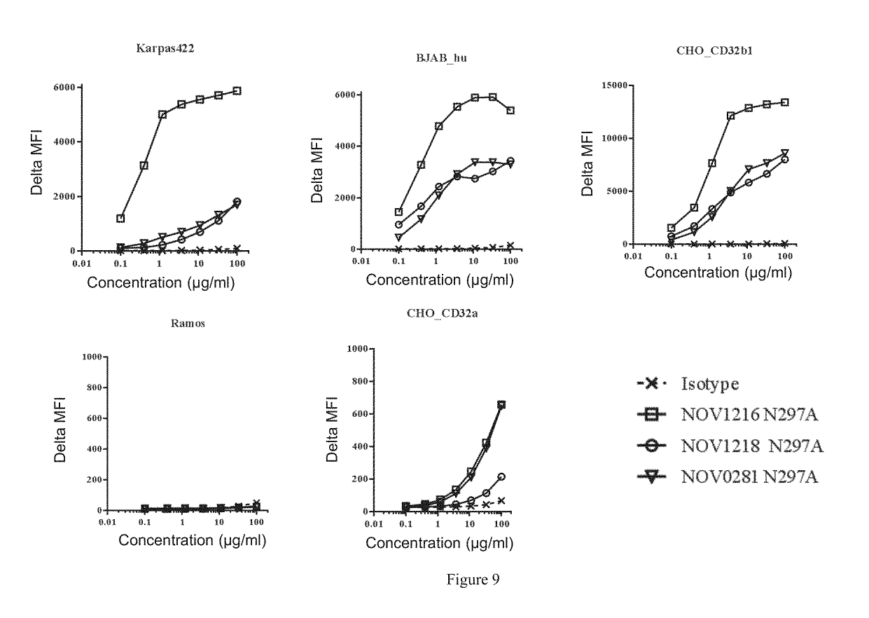

To determine anti-CD32b antibody binding to cells featuring various levels of CD32b expression, FACS analysis was performed on the KARPAS422 (Sigma Aldrich 06101702) human cancer cell line which endogenously expresses huCD32b; BJAB (DSMZ; ACC 757). Stable CHO cell line expressing CD32b and CD23a were also evaluated as were RAMOS cells which lack both CD32b and CD32a. For adherent CHO cell lines, cells were suspended by treating cells in culture with 0.25% Trypsin (Gibco 25200-056). Once cells lifted, they were washed and resuspended with MACs buffer (Miltenyi biotec 130-091-222 with BSA stock (Miltenyi biotec 130-091-376)). For suspension lines (Karpas422, BJAB, Ramos) cells, 11×10⁶ cells were spun down, washed and resuspended with MACs buffer. All cell lines were resuspended to 4×10⁶ cells/ml before aliquoting 50 μl/well in a 96 well round bottom plate (Costar 29442-066). A sevenpoint, 1:3 serial dilution of Alexa-647 labeled (Molecular Probes A20186) N297A antibodies was prepared with 25 μl being added to each well. A non-targeting IgG1 [N297A scaffold] antibody was used as a negative control. Cells were incubated with antibody (all on a human IgG1 [N297A] scaffold) for 30 minutes on ice. Cells were washed, then resuspended in 100 μl MACs buffer with 7AAD (eBiocience 00-6993-50) at 10 μl/ml, and analyzed on a BD FACs Canto (BD Biosciences). For all CD32b positive cell lines, binding of CD32b-binding antibodies was dose dependent (FIG. 9). The antibodies demonstrated limited binding to the CD32b negative Ramos or the CHO_CD32a cell lines. As anticipated, the non-targeting isotype control antibody did not bind to cells.

### Example 10: Determination of Cdr-H3 Mutant Human Cd32B-Binding Antibodies Binding Cells Featuring a Range of Human Cd32B Expression, Cd32a Expression, or Neither Fcgamma Receptors

To determine the binding of CDR-H3 mutant anti-CD32b antibodies to cells, FACS analysis was performed on KARPAS422 (Sigma Aldrich 06101702), DAUDI (ATCC; CCL-213), and parental BJAB (DSMZ; ACC 757) human cancer cell lines which endogenously express huCD32b, as well as stable BJAB and CHO cell lines expressing CD32b. Stable CHO cell line expressing CD32a was also evaluated as was parental CHO cells which lack both CD32b and CD32a.

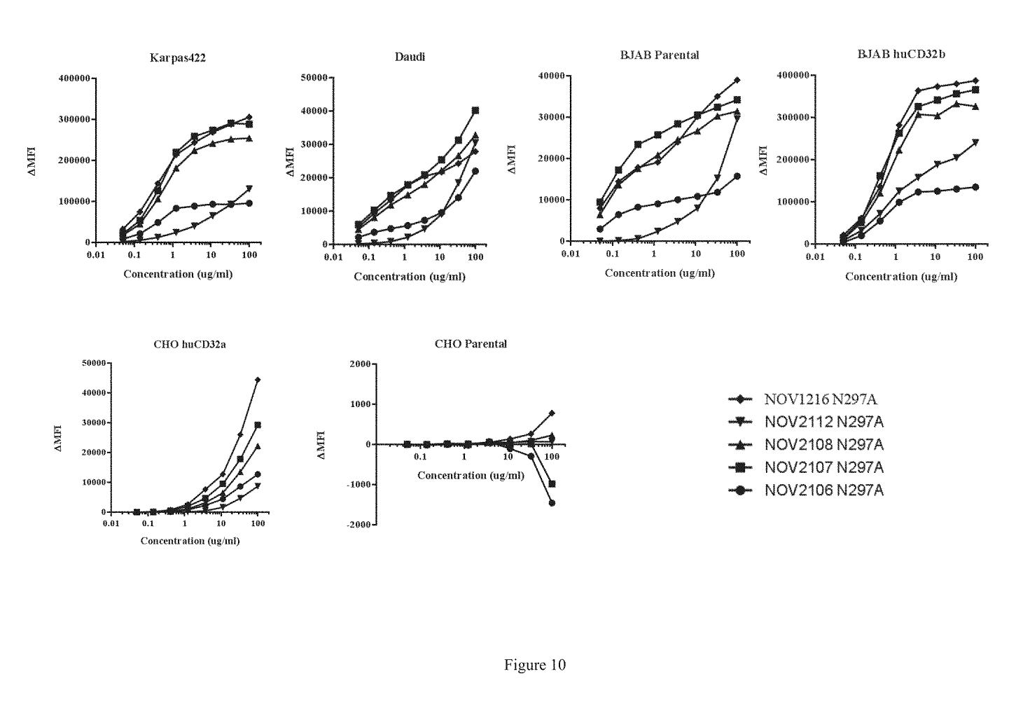

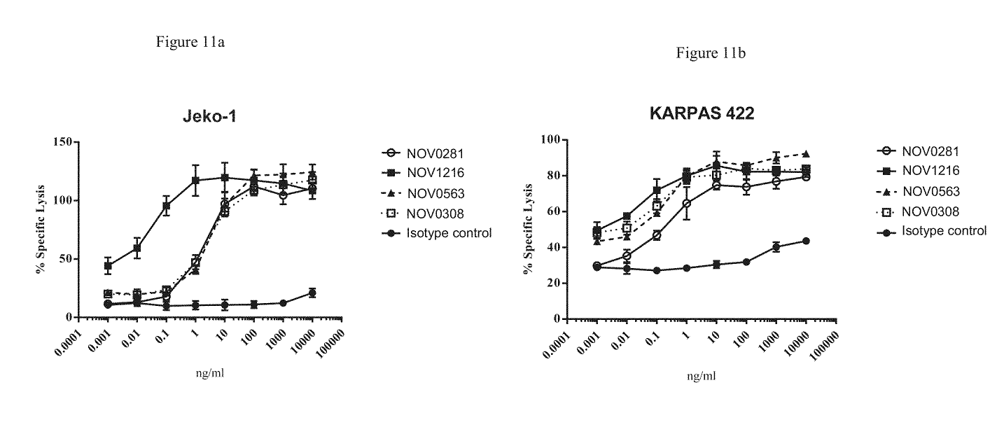

For KARPAS422, DAUDI and BJAB cell lines, 11×10⁶ cells were spun down, washed and resuspended with MACs buffer (Miltenyi biotec 130-091-222 with BSA stock (Miltenyi biotec 130-091-376)). For adherent CHO cell lines, cells were suspended by treating cells in culture with 0.25% Trypsin (Gibco 25200-056). Once cells lifted, they were washed and then resuspended with MACs buffer (Miltenyi biotec 130-091-222 with BSA stock (Miltenyi biotec 130-091-376)). All cell lines were resuspended to 4×10⁶ cells/ml before aliquoting 50 μl/well in a 96 well round bottom plate (Costar 29442-066). An eight point, 1:3 serial dilution of Alexa-647 labeled (Molecular Probes A20186) antibodies (all on a human IgG1 [N297A] scaffold) were prepared with 25 μl being added to each well. A non-targeting antibody (human IgG1 [N297A] scaffold) was used as a negative control. Cells were incubated with antibody for 30 minutes on ice, then washed, resuspended MACs buffer with 7AAD (eBiocience 00-6993-50) at 10 μl/ml, and analyzed on a Novoctye 3000 (ACEA Biosciences 2010011). Geometric mean of signal per sample was determined using Weasel software. For all human CD32b positive cell lines, HCD-R3 mutants NOV2107 and NOV2108 showed the most robust binding which was similar to the parental antibody NOV1216 (FIG. 10). For all antibodies tested, only minimal binding to human CD32a transfected CHO cells, relative to cells expressing human CD32b, was observed and no/very minimal binding to CD32a/CD32b null CHO parental cells. These data demonstrate the specificity of the antibodies to human CD32b.

### Example 11: Assessment of Primary NK Cell Driven, Specific ADCC Activity Against Jeko-1 and Karpas422 Cancer Cell Lines Mediated by Fc Wt Anti-Cd32B Antibodies

Fc wildtype anti-CD32b antibodies (human IgG1) were evaluated for their activity in a primary NK cell based antibody-dependent cell-mediated cytotoxicity (ADCC) assay. In brief, PBMCs were isolated from a donor's blood via a ficoll gradient. NK cells were then negatively selected using Miltenyi beads (catalog#130-092-657). These effector cells were stimulated overnight with 10 ng/ml Il-2 (Peprotech catalog#200-02). The following day, Jeko-1 and Karpas422 cells were stained with Calcein acetoxy-methyl ester (Calcein-AM; Molecular Probes catalog# C3100MP), washed twice, and transferred to a 96-well U-bottom microtiter plate at a concentration of 10,000 cells per well. The cells were then pre-incubated for 20 min with a serial dilution of the above mentioned antibodies before adding the effector cells at an effector to target ratio of 3:1. Following the co-incubation, the microtiter plate was centrifuged and an aliquot of the supernatant fluid was transferred to another microtiter plate (Corning Costar, catalog #3904) and the concentration of free Calcein in solution was determined with a fluorescence counter (Envision, Perkin Elmer).

In order to calculate the antibody specific lysis of the target cells, a parallel incubation of target cells without antibody or effector cells served as a baseline control (spontaneous release), whereas the positive control or maximal release was determined by lysis of target cells only with a 1% Triton-X 100 solution. Percent specific lysis was calculated using this equation: (sample−spontaneous)/(maximum release−spontaneous)*100%.

All Fc wildtype anti-CD32b antibodies demonstrated concentration dependent specific cell lysis of both cancer cell lines evaluated as illustrated in FIG. 11a and FIG. 11b. Ab NOV1216 demonstrated markedly increased activity against Jeko1 relative to the other antibodies profiled (FIG. 11a). Against the KARPAS422 cell line, NOV1216, NOV0281, NOV0308 and NOV0563 showed roughly similar activity with NOV1216 being slightly more active (FIG. 11b). As anticipated, the non-targeted IgG1 Fc wildtype negative control antibody was not active in these assays.

### Example 12: In Vivo Antitumor Activity of Fc Wt Human Cd32B-Binding Antibodies in Established, Disseminated Jeko1 Xenografts

The antitumor activity of five Fc WT human IgG1 CD32b-binding antibodies were evaluated in SCID.Beige mice harboring established mantle cell lymphoma Jeko1 disseminated xenografts. Female SCID.Beige mice were injected intravenously (i.v.) via the tail vain with 1×10⁶ Jeko1 cells stably transfected with a constitutively active promoter driving luciferase expression. Cells were suspended in PBS and mice were i.v. inoculated with a final volume of 0.2 ml cell suspension. Whole body tumor burden, restricted largely to bone marrow space (e.g., hind femurs, vertebra, mandible; data not shown) and expressed as relative light units (RLU), was assessed by injecting mice intraperitoneally (i.p.) with 10 ml/kg luciferin (15 mg/ml) and imaged with a Xenogen IVIS-200 optical in vivo imaging system (Perkin Elmer) starting 10 minutes after luciferin administration. Background RLU was assessed by imaging a mouse that was not administered luciferin.

Mice were imaged and enrolled in the study 10 days post cell inoculation with an average tumor burden of 1.2×10⁶ RLU. After being randomly assigned to one of five groups (n=5/group), mice were administered a single 5 mg/kg i.v. injection of PBS, NOV0281, NOV1216, NOV0308, or NOV0563. Mice were weighed and imaged twice weekly to assess change in body weight and whole body tumor burden.

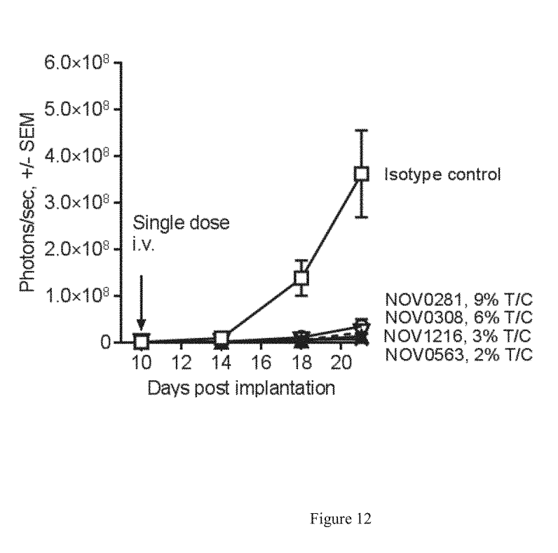

Tumor burden was assessed 22d post cell implantation (10d post treatment administration), expressed as percent T/C (delta RLU of non-targeted IgG1 treated mice divided by delta RLU of treated mice). As anticipated, tumor burden increased rapidly following administration of the non-targeted negative control antibody. All CD32b-binding antibodies were effective at controlling tumor growth following a single intravenous injection, with NOV1216 and NOV0563 being the most active (3 and 2% T/C, respectively) (FIG. 12).

### Example 13: Dose Response In Vivo Efficacy Study of Fc Wt NOV1216 in Mice Bearing Established Daudi Xenografts

To further assess in vivo activity of Fc WT NOV1216, a dose response efficacy study was conducted in mice harboring established Burkett's lymphoma Daudi xenografts. Female nude mice were implanted subcutaneously with 5×10⁶ Daudi cells (100 μl injection volume) suspended in 50% phenol red-free matrigel (BD Biosciences) diluted with PBS. Mice were enrolled in the study 18 days post implantation with average tumor volume of 140 mm³. After being randomly assigned to one of five experimental groups (n=6/group), mice were administered weekly intravenous injections of one of the following: PBS, Fc silent NOV1216 N297A (20 mg/kg qw*12) or Fc WT NOV1216 (5, 10, or 20 mg/kg qw*12). Tumor burden was assessed 35d post cell implantation and 18d post treatment administration, expressed as percent T/C (delta tumor volume of PBS treated mice divided by delta tumor volume of treated mice). Time to endpoint, defined as tumors reaching 800 mm³, was also evaluated.

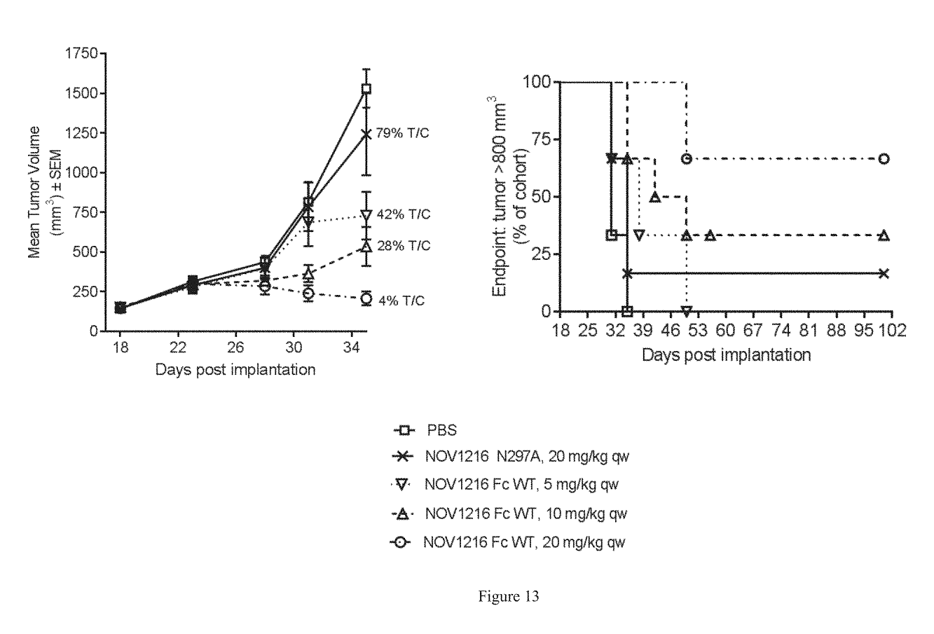

Dose dependent anti-tumor activity and time to endpoint was observed with Fc WT NOV1216. The highest dose demonstrated the most robust anti-tumor activity (4% T/C at 35d post implantation) and longest time to endpoint (FIG. 13). The Fc silent NOV1216 N297A antibody had very limited effect on tumor volume and time to endpoint. These data demonstrate that NOV1216 has robust and durable Fc dependent antitumor activity against established Burkett's lymphoma Daudi xenografts in nude mice.

### Example 14: Assessment of Fc Modification on Cd16a Activation in a Reporter Assay or Primary NK Cell Driven Cell Lysis

The ability to enhance NOV1216 ADCC function by either afucosylation (antibody was produced with N-glycan structure lacking core fucose as described in Example 3 above) or Fc engineering (eADCC Fc mutations S239D/A330L/I332E) was investigated in vitro. Fc activity was evaluated in the Jurkat-NFAT reporter assay and a primary NK cell ADCC assay.

Ability of Fc WT, Afucosylated, and Fc Modified (eADCC or N297A) CD32b-Binding NOV1216 to Activate Human CD16a in the Jurkat-NFAT Reporter System

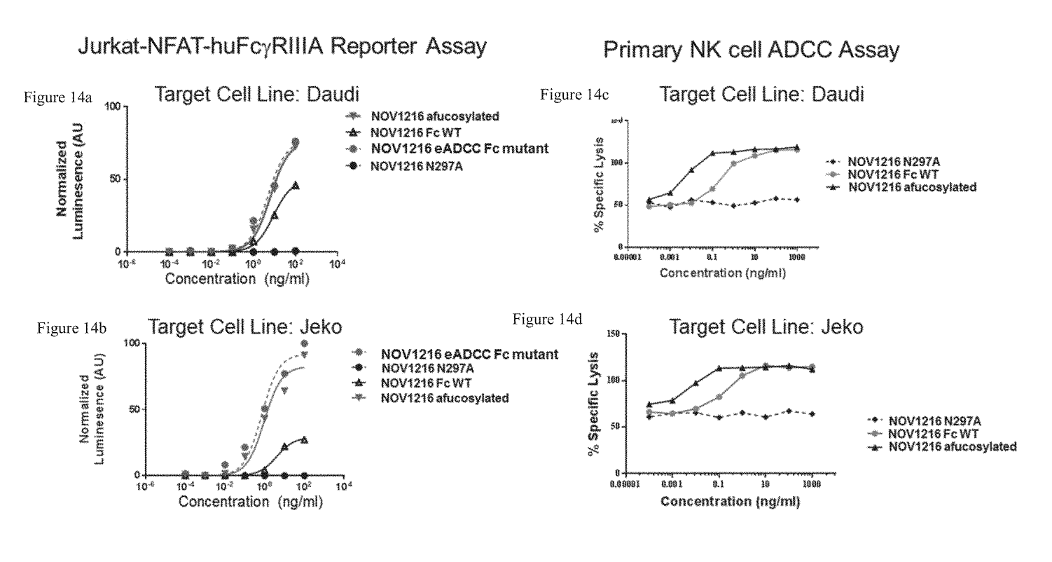

The Jurkat-NFAT reporter assay was used to assess the ability of CD32b-binding antibodies to bind CD32b positive target cells and subsequently activate CD16a on Jurkat-NFAT v158 reporter cells. Target cell lines with variable amounts of CD32b expression (DAUDI; ATCC CCL-213 and Jeko-1; DSMZ ACC533) were used. NOV1216 Fc WT and versions with multiple Fc engineering strategies were profiled in this assay. These included Fc enhanced (afucosylated and eADCC Fc mutations) and Fc silent (N297A) versions of NOV1216. Cell lines were collected, washed in PBS (Gibco 14190-144), resuspended in assay media (RPMI Glutamax (61870-036)+10% FBS (Gibco 26140-079)) to 0.5×10⁶ cells/ml, and 30 μl/well aliquoted into a 96 well white plate (Costar #3917). The Jurkat NFAT v158 reporter cell line was collected, washed in PBS, resuspended in assay media to 3×10⁶ cells/ml, and aliquoted at 30 μl/well resulting in a final effector to target ratio of 6:1. A seven point 1:10 serial dilution of each antibody (Fc wild type, N297A, or eADCC Fc mutant) was prepared in triplicate. Control wells included Jurkat NFAT v158 reporter cell alone, Jurkat NFAT v158 reporter cell line and antibody, or Jurkat NFAT v158 reporter cell line and CD32b positive target cell line. Bright Glo (Promega #E2620) was added to each well (60 μl/well) except the appropriate negative control wells and the plates were subsequently read on an Envision (Perkin Elmer). The resulting luminescence signal is normalized to the highest signal for each antibody within a cell line. This highest signal was designated “100” and all other antibody signals within a cell line were normalized to it. With both Daudi and Jeko-1 target cell lines, afucosylated and eADCC Fc mutant NOV1216 yielded similar CD16a activation which was greater than that observed with Fc WT NOV1216 (FIG. 14a, FIG. 14b). As anticipated, the Fc silent NOV1216 N297A did not activate CD16a in this reporter assay.

### Ability of Fc WT and Fc Modified (Afucosylated or N297A) CD32b-Binding NOV1216 to Elicit Primary NK Cell Driven ADCC Activity Against CD32b Positive Target Cells

The Fc dependent, ADCC activity of the CD32b antibodies was measured by the ability of isolated human natural killer cells to kill CD32b positive target cells. The CD32b target cells used in this assay were DAUDI (ATCC CCL-213) and Jeko-1 (DSMZ ACC533). In brief, PBMCs were isolated from a Leukopak (HemaCare catalog# PB001F-3) via a ficoll gradient (GE Healthcare 17-1440-02). NK cells were then negatively selected using Miltenyi beads (catalog#130-092-657) and then incubated in basic media overnight (RPMI/10% FBS/1% antimitotic/antibiotic). The following day, CD32b positive target cells were stained with Calcein acetoxy-methyl ester (Calcein-AM; Molecular Probes catalog# C3100MP), washed twice, and transferred to a 96-well U-bottom microtiter plate at a concentration of 10,000 cells per well. The cells were then pre-incubated for 20 min with a serial dilution of the antibodies before adding the effector cells at an effector to target ratio of 20:1. Following the 4.0 hour co-incubation, the microtiter plate was centrifuged and an aliquot of the supernatant fluid was transferred to another microtiter plate (Corning Costar, catalog #3904) and the concentration of free Calcein in solution was determined with an EnVision plate reader (Perkin Elmer).

In order to calculate the antibody specific lysis of the target cells, a parallel incubation of target cells without antibody or effector cells served as a baseline control (spontaneous release), whereas the positive control or maximal release was determined by lysis of target cells only with a 1% Triton-X 100 solution. Percent specific lysis was calculated using this equation: (sample−spontaneous)/(maximum release−spontaneous)*100%. In both cell lines evaluated, the afucosylated version of NOV1216 was more active than the Fc WT version (FIG. 14c, FIG. 14d). As anticipated, the Fc silent N297A version of NOV1216 was not active.

Ability of Fc WT and Fc Modified (eADCC Fc Mutant or N297A) CD32b-Binding Antibodies to Elicit Primary NK Cell Driven ADCC Activity Against CD32b Positive Jeko-1 Cells

In a third experiment, the Fc dependent, ADCC activity of a panel of CD32b antibodies was measured by the ability of isolated human natural killer cells to kill CD32b positive Jeko-1 cells (DSMZ ACC533). In brief, PBMCs were isolated from a Leukopak (HemaCare catalog# PB001F-3) via a ficoll gradient (GE Healthcare 17-1440-02). NK cells were then negatively selected using Miltenyi beads (catalog#130-092-657). These effector cells were stimulated overnight with 10 ng/ml I1-2 (Peprotech#200-02). The following day, Jeko-1 cells were stained with Calcein acetoxy-methyl ester (Calcein-AM; Molecular Probes catalog# C3100MP), washed twice, and transferred to a 96-well U-bottom microtiter plate at a concentration of 10,000 cells per well. The cells were then pre-incubated for 20 min with a serial dilution of the antibodies before adding the effector cells at an effector to target ratio of 3:1. Following co-incubation, the microtiter plate was centrifuged and an aliquot of the supernatant fluid was transferred to another microtiter plate (Corning Costar, catalog #3904) and the concentration of free Calcein in solution was determined with EnVision plate reader (Perkin Elmer).

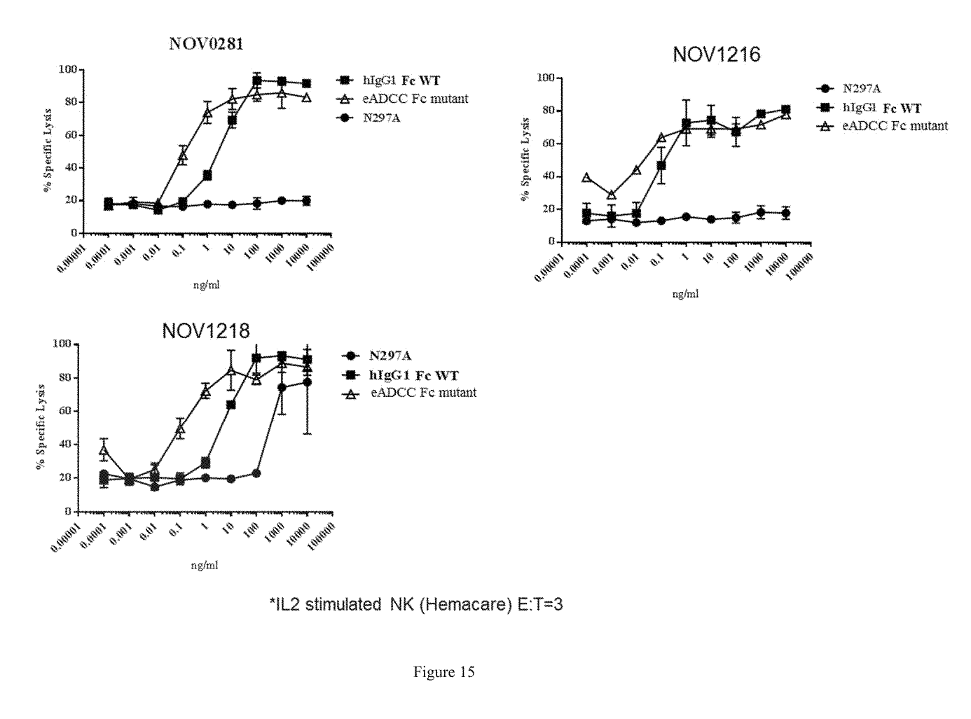

In order to calculate the antibody specific lysis of the target cells, a parallel incubation of target cells without antibody or effector cells served as a baseline control (spontaneous release), whereas the positive control or maximal release was determined by lysis of target cells only with a 1% Triton-X 100 solution. Percent specific lysis was calculated using this equation: (sample−spontaneous)/(maximum release−spontaneous)*100%. In the case of each antibody profiled (NOV0281, NOV1216, and NOV1218), the eADCC Fc modification increased ADCC activity over that of the Fc WT IgG1 (FIG. 15). As anticipated, the Fc silent N297A versions of the antibodies were minimally active in this assay.

### Example 15: Assessment of Fc Wt, eADCC Fc Mutant, and N297A Versions of NOV1216 to Activate Cd16A in a Reporter Assay with Target Cells Featuring a Range of Human Cd32B Expression

The Jurkat-NFAT reporter assay was used to assess the ability of CD32b-binding antibodies to activate CD16a on Jurkat-NFAT v158 reporter cells with a panel of target cell lines featuring a range of human CD32b expression. The CD32b positive target cell lines were as follows: Lama-84 (DSMZ ACC168), Jeko-1 (DSMZ ACC 553), Karpas-620 (DSMZ ACC 514), MOLP-2 (DSMZ ACC 607), and Raji (ATCC CCL-86). The CD32b negative Ramos cell line (ATCC CRL-1596), served as a negative control. Fc WT, eADCC Fc mutant (S239D/A330L/I332E), and N297A versions of NOV1216 were profiled in this experiment.

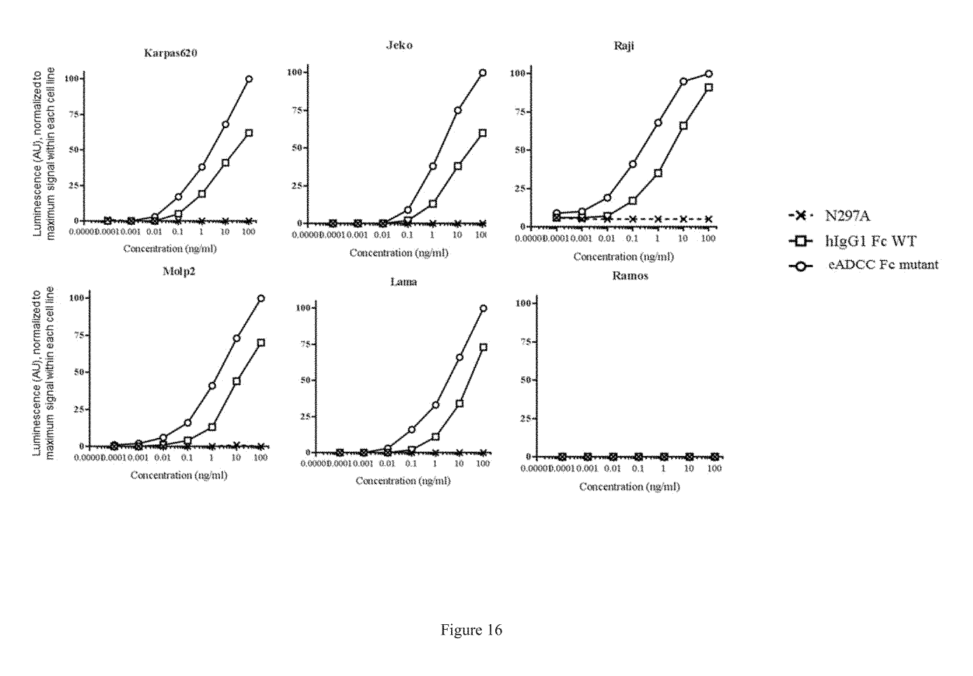

In brief, cell lines were collected, washed in PBS (Gibco 14190-144), resuspended in assay media (RPMI Glutamax (61870-036)+10% FBS (Gibco 26140-079)) to 0.5×10⁶ cells/ml, and 30 μl/well aliquoted into a 96 well white plate (Costar #3917). The Jurkat NFAT v158 reporter cell line was collected, washed in PBS, resuspended in assay media to 3×10⁶ cells/ml, and aliquoted at 30 μl/well resulting in a final effector to target ratio of 6:1. A seven point 1:10 serial dilution of each antibody (Fc wild type, N297A, or eADCC Fc mutant) was prepared in triplicate. Control wells included Jurkat NFAT v158 reporter cell alone, Jurkat NFAT v158 reporter cell line and antibody, or Jurkat NFAT v158 reporter cell line and CD32b positive target cell line. Bright Glo (Promega #E2620) was added at 60 μl/well to each well except the appropriate negative control wells and the plates were subsequently read on an Envision (Perkin Elmer). The resulting luminescence signal is normalized to the highest signal for each antibody with in a cell line. This highest signal was designated “100” and all other antibody signals within a cell line were normalized to it. Both the Fc WT and eADCC Fc mutant versions of NOV1216 elicited activation of CD16a in this assay, with the latter Fc enhanced version yielding a more robust signal (FIG. 16). This was observed across all cell lines profiled with the exception of the CD32b negative Ramos cell line. As anticipated, the Fc silent NOV1216 N297A did not activate CD16a in this reporter assay.

### Example 16: Assessment of Afucosylated CDR-H3 Mutant Cd32B-Binding Antibody Activation of Cd16A in a Reporter Assay with Target Cells Featuring a Range of Human Cd32B Expression

The Jurkat-NFAT reporter assay was used to assess the ability of afucosylated (afuc) CD32b-binding CDR-H3 antibodies to activate CD16a on Jurkat-NFAT v158 reporter cells with a panel of target cell lines featuring a range of human CD32b expression. The CD32b positive target cell lines were as follows: Daudi (ATCC CCL-213), parental BJAB (DSMZ, ACC 757), and KARPAS422 (Sigma Aldrich 06101702) and stable BJAB cells expression human CD32b.

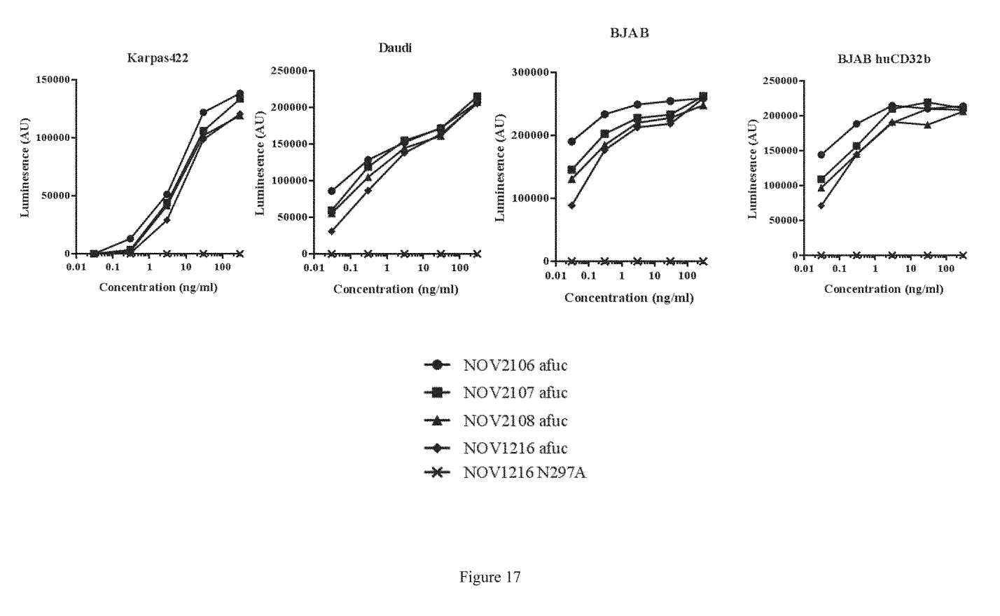

In brief, cell lines were collected, washed in PBS (Gibco 14190-144), resuspended in assay media (RPMI Glutamax (61870-036)+10% FBS (Gibco 26140-079)) to 0.5×10⁶ cells/ml, and 30 μl/well aliquoted into a 96 well white plate (Costar #3917). The Jurkat NFAT v158 reporter cell line was collected, washed in PBS, resuspended in assay media to 3×10⁶ cells/ml, and aliquoted at 30 μl/well resulting in a final effector to target ratio of 6:1. A five point 1:10 serial dilution of each afucosylated antibody (NOV1216, NOV2106, NOV2107, NOV2108) was prepared in triplicate. Control wells included Jurkat NFAT v158 reporter cell alone, Jurkat NFAT v158 reporter cell line and antibody, or Jurkat NFAT v158 reporter cell line and CD32b positive target cell line. Bright Glo (Promega #E2620; 60 μl) was added to all wells, with the exception of the appropriate negative control wells, and the plates were subsequently read on an Envision (Perkin Elmer). All three of the afucosylated CD32b-binding CDR-H3 mutants (NOV2106, NOV2107, NOV2108) and afucosylated NOV1216 potently activated CD16a (FIG. 17). Robust CD16a activation was observed across each of the three CD32b positive cell lines. As anticipated, the N297A Fc silent version of NOV1216 did not activate CD16a in this reporter assay.

### Example 17: Assessment of Afucosylated CDR-H3 Mutant Antibody ADCC Activity in a Primary NK Cell Assay

### Activity of Afucosylated NOV1216 and CDR-H3 Mutants NOV2106, NOV2107, and NOV2108 in a Primary NK Cell ADCC Assay

A primary NK cell ADCC assay was utilized to assess the Fc dependent activity of afucosylated CDR-H3 mutants and afucosylated NOV1216. CD32b positive Daudi (ATCC CCL-213) and KARPAS422 (Sigma Aldrich 06101702) cells served as target cells.

In brief, PBMCs were isolated from a Leukopak (HemaCare catalog# PB001F-3) via a ficoll gradient. NK cells were then negatively selected using Miltenyi beads (catalog#130-092-657) and then incubated in basic media overnight (RPMI/10% FBS/1% antimitotic/antibiotic). The following day, Daudi and Karpas 422 cells were stained with Calcein acetoxy-methyl ester (Calcein-AM; Molecular Probes catalog# C3100MP), washed twice, and transferred to a 96-well U-bottom microtiter plate at a concentration of 10,000 cells per well. The cells were then pre-incubated for 20 min with a serial dilution of the antibodies before adding the effector cells at an effector to target ratio of 20:1. Following the co-incubation, the microtiter plate was centrifuged and an aliquot of the supernatant fluid was transferred to another microtiter plate (Corning Costar, catalog #3904) and the concentration of free Calcein in solution was determined with a fluorescence counter (Envision, Perkin Elmer).

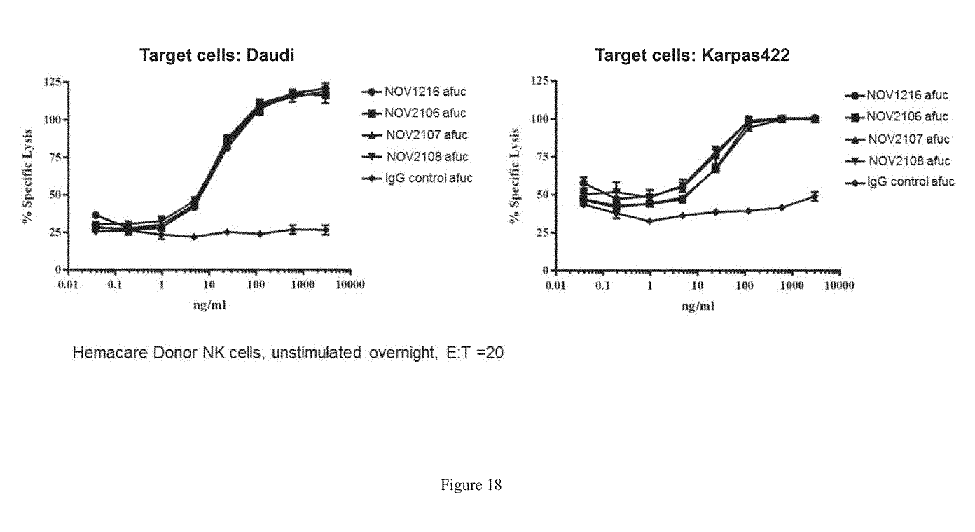

In order to calculate the antibody specific lysis of the target cells, a parallel incubation of target cells without antibody or effector cells served as a baseline control (spontaneous release), whereas the positive control or maximal release was determined by lysis of target cells only with a 1% Triton-X 100 solution. Percent specific lysis was calculated using this equation: (sample−spontaneous)/(maximum release−spontaneous)*100%. All three afucosylated CDR-H3 mutant antibodies (NOV2106, NOV2107, and NOV2108) and afucosylated NOV1216 demonstrated robust specific cell lysis of both Daudi and Karpas422 target cell lines (FIG. 18). As anticipated, the non-targeted afucosylated antibody was not active in this assay.

### Activity of Afucosylated NOV1216 and CDR-H3 Mutants NOV2107 and NOV2108 in a Primary NK Cell ADCC Assay

In an additional experiment, a primary NK cell ADCC assay was utilized to assess the Fc dependent activity of afucosylated CDR-H3 mutant antibodies and afucosylated NOV1216. CD32b positive Daudi (ATCC CCL-213) cells served as target cells.

In brief, PBMCs were isolated from an outsourced Leukopak (HemaCare catalog# PB 001F-3) via a ficoll gradient. NK cells were then negatively selected using Miltenyi beads (catalog#130-092-657) and stimulated overnight with 100 pg/ml IL-2 (Peprotech #200-02). The following day, Daudi and Karpas 422 cells were stained with Calcein acetoxy-methyl ester (Calcein-AM; Molecular Probes catalog# C3100MP), washed twice, and transferred to a 96-well U-bottom microtiter plate at a concentration of 10,000 cells per well. The cells were then pre-incubated for 20 min with a serial dilution of the antibodies before adding the effector cells at an effector to target ratio of 3:1. Following the co-incubation, the microtiter plate was centrifuged and an aliquot of the supernatant fluid was transferred to another microtiter plate (Corning Costar, catalog #3904) and the concentration of free Calcein in solution was determined with a fluorescence counter (Envision, Perkin Elmer).

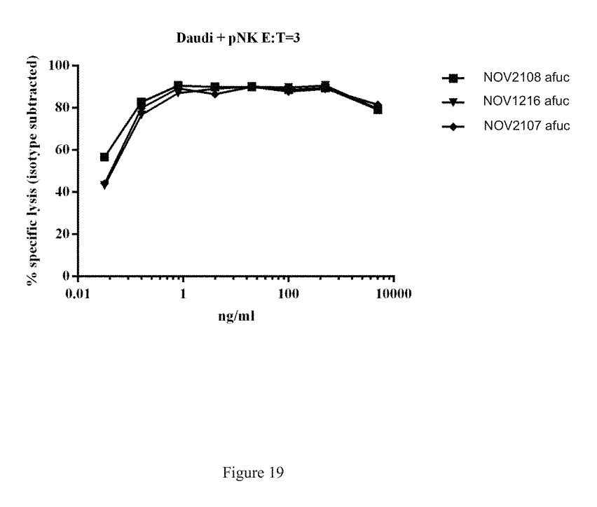

In order to calculate the antibody specific lysis of the target cells, a parallel incubation of target cells without antibody or effector cells served as a baseline control (spontaneous release), whereas the positive control or maximal release was determined by lysis of target cells only with a 1% Triton-X 100 solution. Percent specific lysis was calculated using this equation: (sample−spontaneous)/(maximum release−spontaneous)*100%. Both afucosylated CDR-H3 mutant antibodies, NOV2107 and NOV2108, as well as afucosylated NOV1216 demonstrated robust specific cell lysis of Daudi target cells (FIG. 19).

### Example 18: In Vivo Activity of Fc Wt, eADCC Fc Mutant, and N297A Versions of NOV1216 Against the Daudi Xenograft Model

To explore the effect of the eADCC Fc mutations (S239D/A330L/I332E) on NOV1216 activity in vivo, an efficacy study was conducted in mice harboring established Burkett's lymphoma Daudi xenografts. Female nude mice were implanted subcutaneously with 5×10⁶ Daudi cells (100 μl injection volume) suspended in 50% phenol red-free matrigel (BD Biosciences) diluted with PBS. Mice were enrolled in the study 18 days post implantation with average tumor volume of 140 mm³. After being randomly assigned to one of 4 experimental groups (n=6/group), mice were administered weekly intravenous injections of one of the following: PBS, Fc silent NOV1216 N297A (20 mg/kg qw*12), Fc WT NOV1216 (10 mg/kg qw*12), or NOV1216 eADCC Fc mutant (10 mg/kg qw*3). Tumor burden was assessed 35d post cell implantation and 18d post treatment administration, expressed as percent T/C (delta tumor volume of PBS treated mice divided by delta tumor volume of treated mice). Time to endpoint, defined as tumors reaching 800 mm³, was also evaluated.

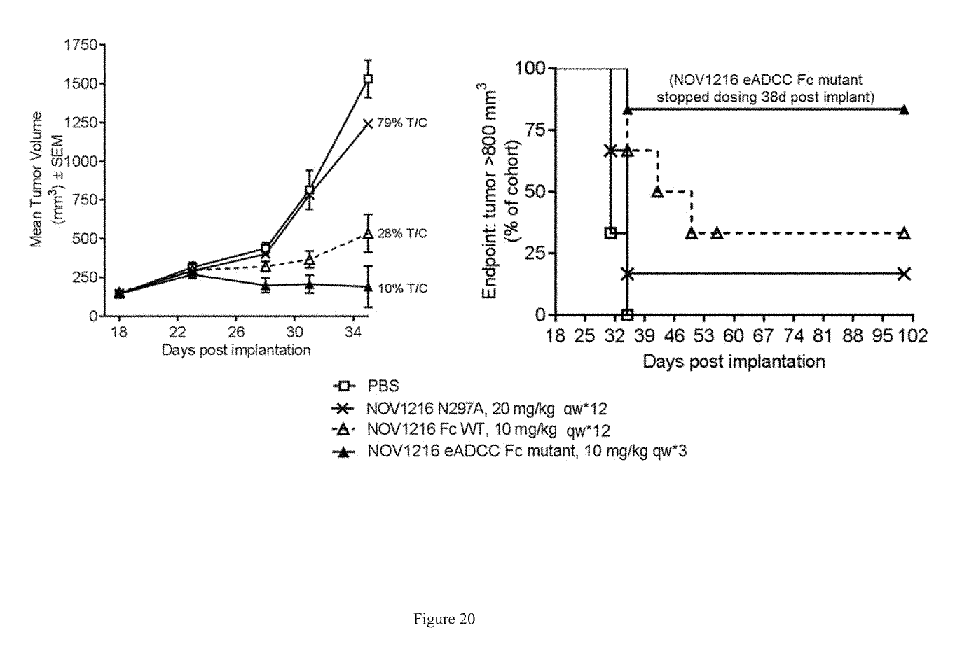

Consistent with in vitro observations, NOV1216 harboring the eADCC Fc mutations was more active than Fc WT NOV1216 in vivo as illustrated by a smaller tumor volume at 34d post cell implantation and time to endpoint (FIG. 20). The Fc silent NOV1216 N297A antibody had very limited effect on tumor volume and time to endpoint. These data demonstrate that Fc enhanced NOV1216 eADCC Fc mutant was more active than Fc wt NOV1216 in an established in vivo xenograft model. Importantly, the anti-tumor response of NOV1216 eADCC Fc mutant was quite durable as evidenced by the fact that time to endpoint was extended despite receiving only three i.v. doses, i.e. qw*3, relative to the other experimental groups which were dosed qw*12.

### Example 19: In Vivo Anti-Tumor Activity of Afucosylated NOV1216 and Afucosylated CDR-H3 Mutants Against Daudi Xenografts

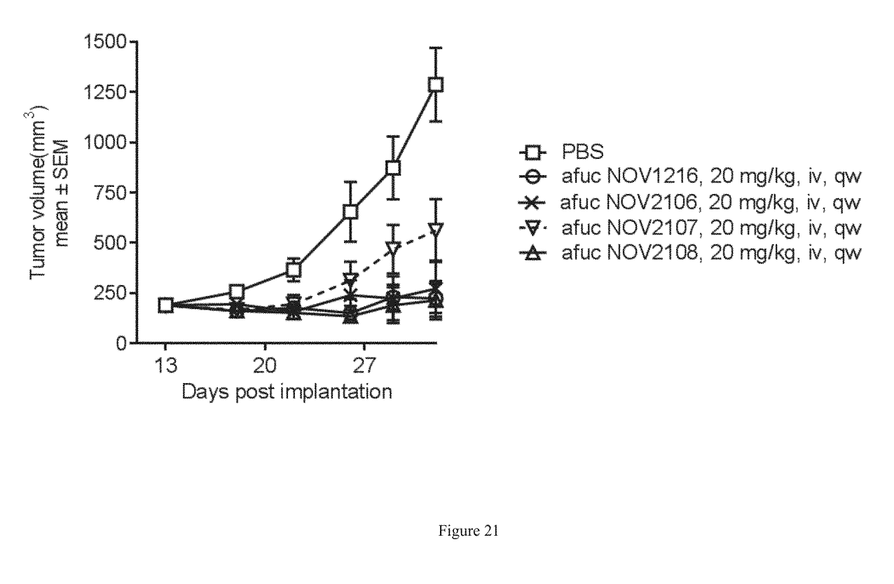

A multi-dose efficacy study in established Burkett's lymphoma Daudi xenografts was conducted to assess the in vivo activity of the CD32b-binding, afucosylated NOV1216 antibody and the afucosylated CDR-H3 mutant antibodies, NOV2106, NOV2107 and NOV2108. Female nude mice were implanted subcutaneously with 5×10⁶ Daudi cells in a suspension containing 50% phenol red-free matrigel (BD Biosciences) in PBS (100 μl total injection volume). Mice were enrolled in the study 13 days post implantation with average tumor volume of 197 mm³. After being randomly assigned to one of four groups (n=6/group), mice were administered weekly intravenous injections of PBS (10 ml/kg qw*3) or 20 mg/kg qw*3 of one of the following afucosylated antibodies: NOV1216, NOV2106, NOV2107, or NOV2108. All four CD32b-binding antibodies were active yielding robust tumor growth control (FIG. 21).

### Example 20: Blocking Cd32B with Fc Silent NOV1216 N297A Enhances the Ability of Rituximab and Obinutuzumab to Activate Cd16A in a Reporter Assay

Studies were conducted to evaluate the impact of human CD32b expression by CD20 positive cells on the ability of rituximab and obinutuzumab to activate CD16a in the Jurkat-NFAT reporter assay. The consequence of combining Fc silent NOV1216 with rituximab or obinutuzumab on their ability to activate CD16a was also evaluated.

CD32b negative parental Ramos cells were obtained from ATCC (CRL-1596) and Ramos cells stably expressing human CD32b were generated. In brief, for the generation of stable Ramos cell lines exogenously expressing human CD32b, Gateway Technology was used to insert the full length human CD32b1 sequence (UniProtKB P31994-1) into the lentiviral expression vector OPS_v19_pLenti6.3-EF1a-gw with Gateway LR Clonase II Enzyme mix (Invitrogen 11791-020). To generate virus, the huCD32b₁/V19 plasmid was then mixed with the packaging vectors PCG and VSV-G in TransIT-193 transfection reagent (Minis MIR2700) and Optimem Serum Free Medium (Invitrogen #11058021). The mixture was incubated at room temperature for 20 minutes and then added to HEK-293T cells on Biocoat Collagen coated 10 cm plates (BD #356450). The next day the medium was changed to DMEM (Gibco 11965-092)+10% FBS (Gibco 26140-079)+1×NEAA (Gibco 11965-092) and returned to 37° C. for 72 hours. At viral harvesting, supernatant was collected, pooled and filtered through 0.45 uM cellulose acetate filters (Corning #430314).

For the transduction of stable Ramos cell lines with the virus, 1×10⁶ cells were plated in a flat bottom 24 well plate (Costar 3526). To the cells, 1 ml of warmed to 37° C. CD32b1/V19 virus was added with 8 ug/ml of polybrene (Sigma H9268). Cells were spun at room temperature for 1.5 hours at 2250 rpm. Viral supernatant was then removed and 3 ml of fresh media was added to the cells which were then transferred to a 6 well plate (Costar 3516). The cells were incubated at 37° C. for two days before being transferred to a T25 flask. Once cells were fully recovered, selective media containing Blasticidin was applied. The final stable line was a pooled population uniformly expressing high levels of human CD32b1 relative to non-transduced parental lines as determined by flow cytometry.

Once the cell lines were developed, they were collected, washed in PBS (Gibco 14190-144), resuspended in assay media (RPMI Glutamax (61870-036)+10% FBS (Gibco 26140-079)) to 0.5×10⁶ cells/ml, and 30 μl/well aliquoted into a 96 well white plate (Costar #3917). The Jurkat NFAT v158 reporter cell line was collected, washed in PBS, resuspended in assay media to 3×10⁶ cells/ml, and aliquoted at 30 μl/well resulting in a final effector to target ratio of 6:1. A seven point 1:10 serial dilution of rituximab or obinutuzumab was prepared in triplicate. Fc silent NOV1216 N297A was excluded from control wells containing only rituximab or obinutuzumab to serve as a baseline controls or combined with rituximab or obinutuzumab at 30 μg/ml. All serial dilutions were plated in triplicate. Control wells included Jurkat NFAT v158 reporter cell alone, Jurkat NFAT v158 reporter cell line and antibody, or Jurkat NFAT v158 reporter cell line and target positive target cell line. Bright Glo (Promega #E2620) was added at 60 μl/well to each well, with the exception of the appropriate negative control wells, and the plates were subsequently read on an Envision (Perkin Elmer).

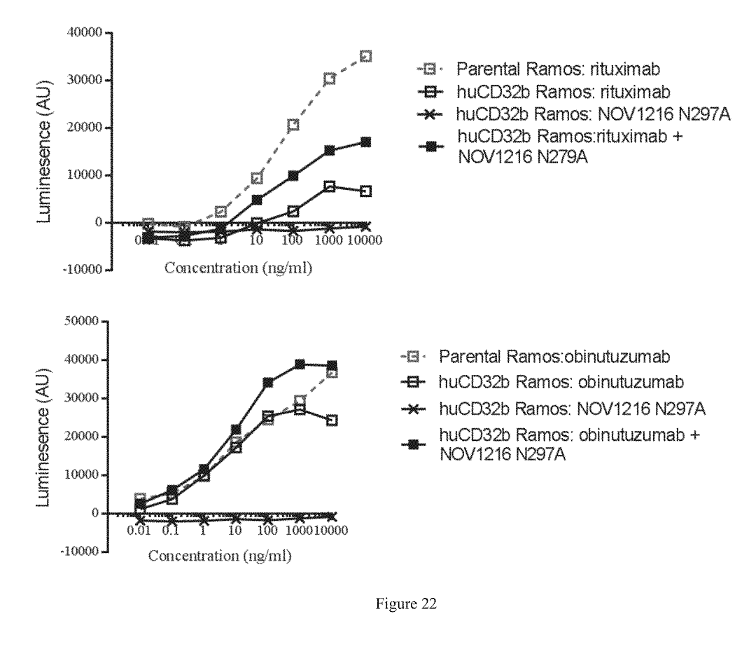

Both rituximab and obinutuzumab bound to Ramos cells efficiently and activated CD16a on the reporter cells, whereas this activation was weaker when human CD32b was overexpressed on the Ramos cells, suggesting that CD32b was interfering with CD16a activation by the CD20 targeted rituximab (FIG. 22, top panel) and obinutuzumab (FIG. 22, bottom panel). When incubated with the Ramos huCD32b cells alone, NOV1216 N297A (Fc silent) was unable to activate CD16a on the reporter cells. However, in combination with rituximab or obinutuzumab NOV1216 N297A increased the activation of CD16a by Ramos huCD32b over cells incubated with rituximab or obinutuzumab alone. Taken together, these data demonstrated that NOV1216 N297A enhanced CD16a activation by rituximab and obinutuzumab when CD32b and CD20 are co-expressed on the same target cells. The enhancement is believed to be due to blocking of CD32b binding to the Fc portion of rituximab and obinutuzumab.

### Example 21: Blocking Cd32B with Fc Silent NOV1216 N297A or Fc Silent N297A CDR-H3 Mutant Antibodies Enhances the Ability of Rituximab to Activate Cd16A

Studies were conducted to evaluate the impact of combining Fc silent NOV1216 and Fc silent CDR-H3 mutant antibodies, NOV2106, NOV2107, and NOV2018, with rituximab on the ability of rituximab to activate CD16a with CD20 and CD32b positive BJAB cells as the target cell line.

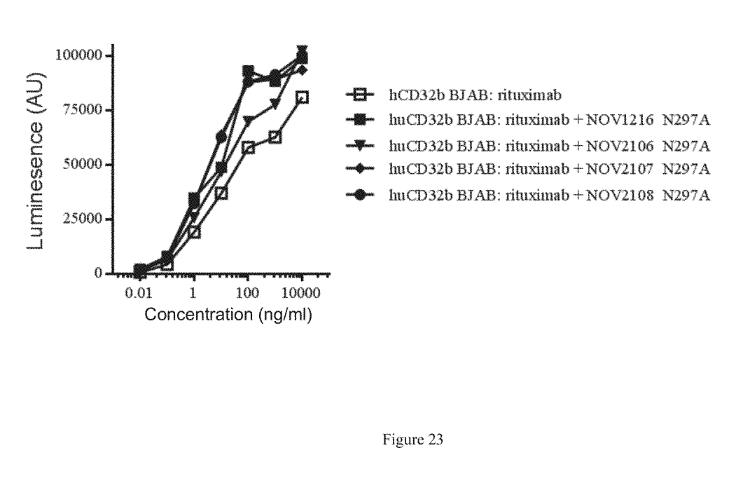

BJAB cells were obtained from (DSMZ; ACC 757) and engineered to stably express human CD32b1 (produced using the same methods outlined in Example 20). In brief, cell lines were collected, washed in PBS (Gibco 14190-144), resuspended in assay media (RPMI Glutamax (61870-036)+10% FBS (Gibco 26140-079)) to 0.5×10⁶ cells/ml, and 30 μl/well aliquoted into a 96 well white plate (Costar #3917). The Jurkat NFAT v158 reporter cell line was collected, washed in PBS, resuspended in assay media to 3×10⁶ cells/ml, and aliquoted at 30 μl/well resulting in a final effector to target ratio of 6:1. A seven point 1:10 serial dilution of rituximab was prepared in triplicate. Fc silent N297A variants of NOV1216, NOV2106, NOV2107, or NOV2108 was excluded from control wells containing only rituximab to serve as a baseline controls or combined with rituximab at 30 μg/ml. Control wells included Jurkat NFAT v158 reporter cell alone, Jurkat NFAT v158 reporter cell line and antibody, or Jurkat NFAT v158 reporter cell line and target positive target cell line. Bright Glo (Promega #E2620) was added at 60 μl/well to each well except the appropriate negative control wells and the plates were subsequently read on an Envision (Perkin Elmer). Rituximab bound to hCD32b BJAB cells efficiently activated CD16a on the reporter cells In combination with rituximab the Fc silent N297A variants of NOV1216, NOV2106, NOV2107, or NOV2108 increased the activation of CD16a by BJAB huCD32b over cells incubated with rituximab alone (FIG. 23). Taken together, these data demonstrated that the Fc silent, CD32b targeting antibodies enhanced CD16a activation by rituximab when CD32b and CD20 are co-expressed on the same target cells. One explanation is that the enhancement is due to blocking of CD32b binding to the Fc portion of rituximab.

### Example 22: In Vivo Anti-Tumor Activity of NOV1216 eADCC Fc Mutant as a Single Agent or in Combination with Rituximab or Obinutuzumab in the Daudi Xenograft Model

In vitro findings described above demonstrate that expression of CD32b reduces Fc dependent activity of both rituximab (type I) and obinutuzumab (type II) CD20 targeted therapeutics and CD32b targeted Ab combined robustly with each of these CD20 targeted therapeutics. To explore these observations in vivo, a combination efficacy study was conducted in mice harboring established Burkett's lymphoma Daudi xenografs. Female nude mice were implanted subcutaneously with 5×10⁶ Daudi cells. Cells were suspended in a suspension containing 50% phenol red-free matrigel (BD Biosciences) in PBS. The total injection volume containing cells in suspension was 100 μl. Mice were enrolled in the study 18 days post implantation with average tumor volume of 201 mm³ After being randomly assigned to one of six groups (n=7/group), mice were administered weekly intravenous injections (10 mg/kg qw) of rituximab, obinutuzumab, NOV1216 eADCC Fc mutant (S239D/A330L/I332E), rituximab+NOV1216 eADCC Fc mutant (10 mg/kg qw each), or obinutuzumab+NOV1216 eADCC Fc mutant (10 mg/kg qw each). Tumor burden was assessed 31d post cell implantation and 18d post treatment administration and expressed as percent T/C (delta tumor volume of PBS treated mice divided by delta tumor volume of treated mice). Time to endpoint, defined as tumors reaching 800 mm³, was also evaluated.

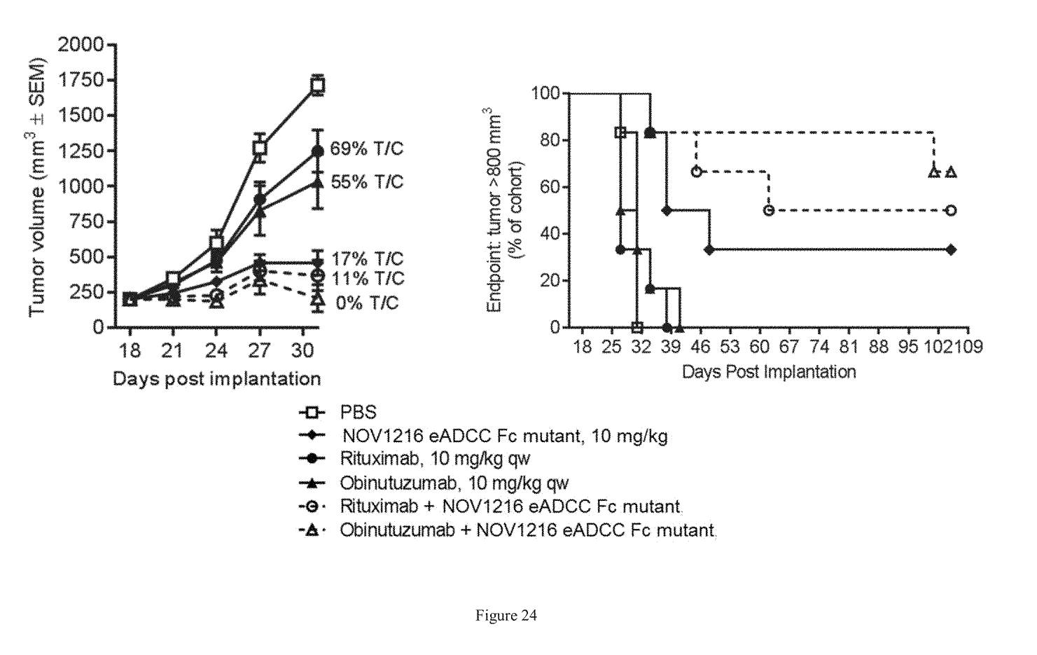

At 31d post treatment initiation, limited anti-tumor activity was observed with single agent rituximab or obinutuzumab (69 and 55% T/C, respectively), while NOV1216 eADCC Fc mutant demonstrated robust anti-tumor activity (17% T/C) (FIG. 24). This translated into differences in time to endpoint. The combination of NOV1216 eADCC Fc mutant and either rituximab or obinutuzumab resulted in increased time to endpoint relative to each single agent (FIG. 24).

### Example 23: Blocking Cd32B with Fc Silent N297A CDR-H3 Mutant Antibody NOV2108 Enhances the Ability of Daratumumab to Activate Cd16A

CD38 is expressed on multiple myeloma cells and an anti-CD38 antibody daratumumab has recently been approved by the FDA for treatment of multiple myeloma. When CD32b and CD38 are co-expressed on the same cell, it is possible that CD32b could bind to the Fc of daratumumab and lead to internalization of the therapeutic antibody or sequestration of the daratumumab Fc from activating FcγRs expressed on effector cells. This example evaluates whether NOV2108 can block the binding of CD32b to the Fc of daratumumab and thereby allow more robust activation of CD16a (FcγRIIIa) by daratumumab.

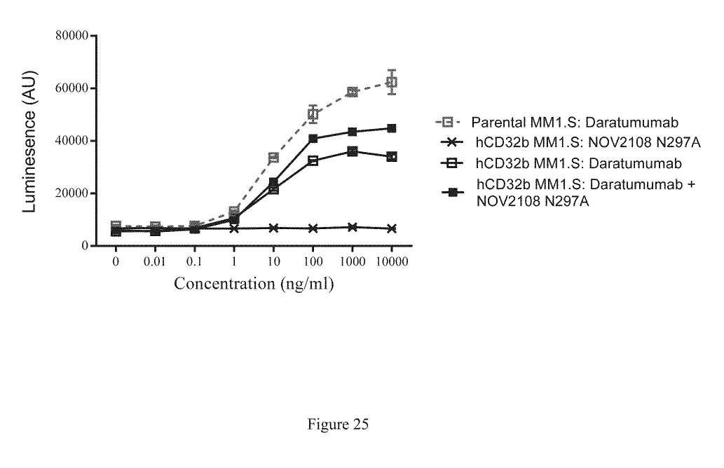

MM1.S cells were obtained from ATCC (CRL-2974). The parental MM1.S cells and MM1.S cells stable expressing human CD32b1 (produced using the same methods outlined in Example 20) were collected, washed in PBS (Gibco 14190-144), resuspended in assay media (RPMI Glutamax (Gibco 61870-036)+10% FBS (Gibco 26140-079)) and aliquoted into a 96 well white plate (costar #3917) at 15,000 cells/well. The Jurkat NFAT v158 reporter cell line was added to each well at 90,000 cells/well. An eight point 1:10 serial dilution of daratumumab was prepared with the starting concentration at 10 ug/ml. To each well containing daratumumab and NOV2108 combination, a saturating amount of NOV2108-N297A antibody is added at 10 ug/ml. All conditions are plated in triplicate. Control wells include reporter cells alone, reporter cells and antibody, or reporter cells and MM1.S or MM1.S huCD32b cells. Plates were incubated at a 37° C. incubator with 5% CO₂ for 4 hours. Following the co-incubation, Britelite plus (Perkin Elmer, catalog#6066769; 70 μl) was added to all wells, with the exception of the background control wells. Resulting luminescence was subsequently read on an Envision (Perkin Elmer) and then plotted using Prism software. Daratumumab bound to MM1.S cells efficiently activated CD16a on the reporter cells, whereas this activation was weaker when human CD32b was overexpressed on the MM1.S cells, suggesting that CD32b was interfering with CD16a activation by daratumumab (FIG. 25). When incubated with the MM1.S huCD32b cells alone, NOV2108-N297A (Fc silent) was unable to activate CD16a on the reporter cells. However, in combination with daratumumab, NOV2108-N297A increased the activation of CD16a by MM1.S huCD32b over cells incubated with daratumumab alone. Taken together, these data demonstrated that NOV2108-N297A enhanced CD16a activation by daratumumab when CD32b and CD38 are co-expressed on the same target cells. One explanation for the observed enhancement is that the anti-CD32b antibody blocks CD32b binding to the Fc portion of daratumumab, making the Fc portion available for interacting with activatory Fc gamma receptors (e.g. CD16a).

### Example 24: Wildtype and Fc Enhanced NOV1216 and NOV2108 Efficiently Mediate Daudi Target Cell Killing by Human Macrophages

Macrophages have been shown as potent effector cells for antibody-mediated tumor cell clearance (see Uchida et al., J Exp Med. 199(12):1659-69 (2004); Pallasch et al., Cell 156(3):590-602 (2014); Overdijk et al., MAbs 7(2):311-21 (2015); Dilillo et al., Cell 161(5):1035-45 (2015)). This example evaluated the efficiency of the Fc WT, Fc silent N297A mutant, and afucosylated versions of antibody NOV1216; the Fc WT, Fc silent N297A mutant, and afucosylated versions of antibody NOV2108; and Fc WT and Fc silent N297A versions of anti-CD32b antibody Clone 10 from WO 2012/022985 to mediate target cell killing by macrophages. The CDR, VH and VL sequences of antibody Clone 10 appear to be identical to antibody 6G11 from WO2015/173384.

The macrophage-mediated cell killing assay was conducted to measure the ability of human monocyte-derived macrophages (hMDM) to kill opsonized CD32b luciferized Daudi cells. In brief, PBMCs were isolated from a Leukopak (HemaCare, catalog# PB001F-3) using Ficoll gradient centrifugation. Monocytes were then negatively selected using Miltenyi human monocyte isolation kit II (catalog#130-091-153). Isolated monocytes were further seeded on a 96-well flat-bottom microtiter plate (Corning, catalog#3596) at a concentration of 300,000 cells per well and cultured for 7 days in complete macrophage medium [(X-VIVO15 (Lonza, catalog#04-744Q)+10% FBS)] supplemented with 10 ng/ml M-CSF (PeproTech, catalog#300-25). Luciferized Daudi cells were harvested and pre-incubated for 10 min with a serial dilution of the antibodies. These target cells with corresponding antibodies were transferred to hMDM plates at 10,000 cells/well. Target cells with or without antibodies (no macrophages) were included as controls. Plates were incubated at a 37° C. incubator with 5% CO₂ for 4 hours. Following the co-incubation, Britelite plus (Perkin Elmer, catalog#6066769; 70 μl) was added to all wells, with the exception of the background control wells (Daudi cells only). Target cells with Britelite served as maximal signal controls whereas target cells without Britelite served as background controls. An aliquot of the supernatant fluid was transferred to another microtiter plate (Corning Costar, catalog #3917) and the luminescence signal was subsequently measured on an Envision (Perkin Elmer). The percent killing of target cells was calculated using the following formula: [1−(sample−background)/maximal)]×100%.

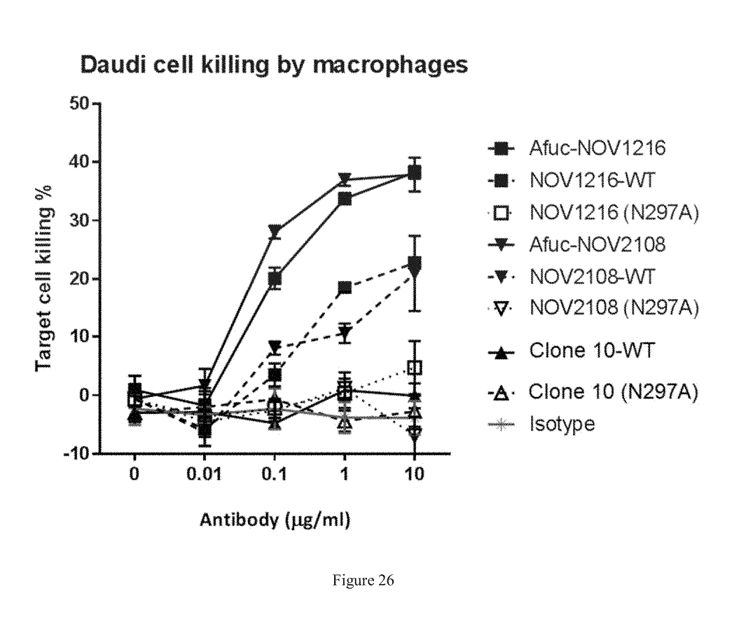

Fc wildtype (WT) antibodies NOV1216 or NOV2108 mediated robust killing of Daudi cells whereas WT Clone 10 antibody showed minimum effect (FIG. 26). Afucosylation further enhanced the macrophage-mediated target cell killing by NOV1216 or NOV2108. No macrophage-mediated killing was observed on Daudi cells incubated with isotype (anti-chicken lysozyme antibody) control, indicating that cell killing requires specific binding of the antibodies to CD32b expressed on Daudi cells. In addition, the Fc-silenced (N297A) mutant antibodies (NOV1216, NOV2108 or Clone 10) did not mediate target cell killing by macrophages, suggesting that activation of macrophage Fcγ receptors is required for cell killing in this assay.

### Example 25: Impact of Cd32B-Binding Antibodies 2B6 and NOV1216 (Fc Wt and Fc Modified) on Basal and Crosslinked Anti-Igm-Stimulated pCD32B Levels in Primary Human B Cells

Cross-linked anti-IgM is known to activate B cells and subsequently yield phosphorylation of the CD32b ITIM. A series of experiments were conducted to assess the impact of various CD32b-binding antibodies on basal pCD32b levels (tyrosine 292) as well as anti-IgM stimulated pCD32 levels.

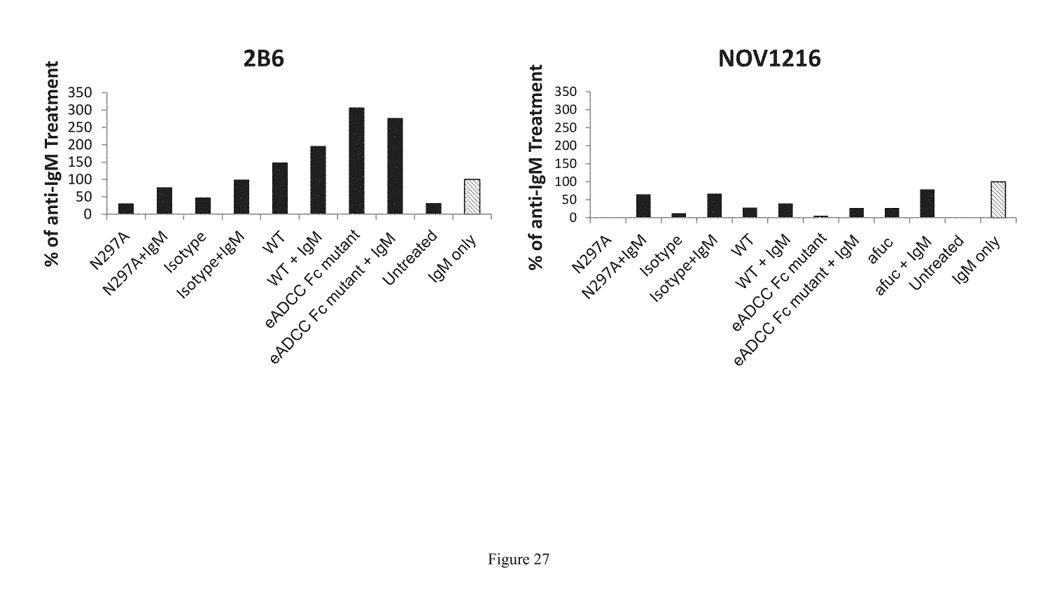

In brief, PBMCs were isolated from donated human whole blood by Ficoll gradient. B cells were then isolated using the Miltenyi B cell isolation kit II (Miltenyi Biotech 130-091-151) and protocol. B cells were plated in a 24 well plate (costar 3526) at 1×10⁶ cells/well in RPMI. In experimental wells set up to assess the impact of CD32b-binding antibodies, 2B6 (see Rankin et al., 2006 Blood 108(7):2384-2391 and U.S. Pat. No. 7,521,542) or NOV1216 antibodies at a final concentration of 5 nM (Fc WT, eADCC Fc mutant (S239D/A330L/I332E), afucosylated, and N297A versions) on pCD32b levels in the presence or absence of crosslinked anti-IgM. Control wells had no treatment, crosslinked anti-IgM only, CD32b-binding antibody only, or afucosylated non-targeted antibody only (isotype control). Following 10 minutes of incubation at 37° C., B cells were harvested and lysed with Ripa buffer (Boston Bioproducts BP-115) containing Halt protease inhibitor (Thermo Scientific 78430) and Phosphostop (Roche 04-906-837-001). Protein lysate was reduced, ran on a PVDF gel (BioRad 170-4157), transferred to a PVDF membrane (BioRad 567-1084), and blocked with Odyssey blocking buffer (Licor 927-40000). The membrane was probed with pCD32b (Abam ab68423) and beta actin (Abcam ab8226) primary antibodies overnight, both at 1:25000 dilution. Following four washes (Tris Buffered Saline with Tween (TBST); Boston BioProducts 1BB-181X), secondary antibodies (IR800 anti-mouse, Licor 925-32210 and IR680 anti-rabbit, Licor 925-68071) were added at 1:10000 dilution in Odyssey blocking buffer. The membrane was subsequently washed (four times in TBST, once in Tris Buffered Saline (Boston BioProducts BM-30IX)) and then read on an Odyssey CLx. The pCD32b signal was normalized to Beta-Actin and expressed as a ratio of anti-IgM treatment only, which was set to 100. As anticipated, crosslinked anti-IgM resulted in an increase in CD32b ITIM phosphorylation (FIG. 27). Antibody 2B6 (Fc wt, N297A, and eADCC Fc mutant, versions) was a potent agonist of CD32b ITIM as indicated by a marked increase in pCD32b levels (FIG. 27, left panel). This is in contrast to NOV1216 (Fc wt, N297A, eADCC Fc mutant, and afucosylated versions), which lacked a robust pCD32b agonistic activity (FIG. 27, right panel). The agonistic activity of 2B6 was found to be dependent on engaging Fc, i.e. the Fc silent N297A version did not yield CD32b ITIM phosphorylation. All versions of NOV1216 had the ability to subtly reduce crosslinked anti-IgM activation of CD32b (FIG. 27). This was not observed with 2B6.

### Example 26: Ability of Afucosylated Cd32B-Binding Antibody NOV1216 to Modulate Rituximab Stimulated Cd32B ITIM in Primary B Cells, Daudi Cells and Karpas422 Cells

Rituximab is known to cause CD32b ITIM phosphorylation on human B cells and CD20 positive cancer cell lines. Several experiments were conducted to explore the ability of afucosylated CD32b-binding antibody NOV1216 to modulate this rituximab-driven increase in pCD32b in primary B cells and CD20 positive Daudi (ATCC; CCL-213) and Karpas422 (Sigma Aldrich 06101702) cancer cell lines. The effect of afucosylated NOV1216 on basal levels of CD32b ITIM phosphorylation in these cells was also investigated. In brief, PBMCs were isolated from whole blood by ficoll separation. B cells were then isolated from PBMCs using the Miltenyi B cell isolation kit II (Miltenyi Biotech 130-091-151) and protocol. B cells, Daudi cells, and Karpas422 cells were plated in a 24 well plate (costar 3526) at 1×10⁶ cells/well in RPMI. Half of the experimental wells were stimulated with rituximab (50 nM). Afucosylated NOV1216 was added to both untreated or rituximab stimulated wells at a final concentration of 50 nM. Control wells consisted of untreated, rituximab only, or afucosylated NOV1216 only.

Following 30 minutes of incubation at 37° C., cells were harvested and lysed with Ripa buffer (Boston Bioproducts BP-115) containing Halt protease inhibitor (Thermo Scientific 78430) and Phosphostop (Roche 04-906-837-001). Protein lysate was reduced, ran on a PVDF gel (BioRad 170-4157), transferred to a PVDF membrane (BioRad 567-1084), and blocked with Odyssey blocking buffer (Licor 927-40000). The membrane was probed with pCD32b (Abam ab68423) and beta actin (Abcam ab8226) primary antibodies overnight, both at 1:25000 dilution. Following four washes (Tris Buffered Saline with Tween (TBST); Boston BioProducts 1BB-181X), secondary antibodies (IR800 anti-mouse, Licor 925-32210 and IR680 anti-rabbit, Licor 925-68071) were added at 1:10000 dilution in Odyssey blocking buffer. The membrane was subsequently washed (four times in TBST, once in Tris Buffered Saline (Boston BioProducts BM-30IX)) and then read on an Odyssey CLx.

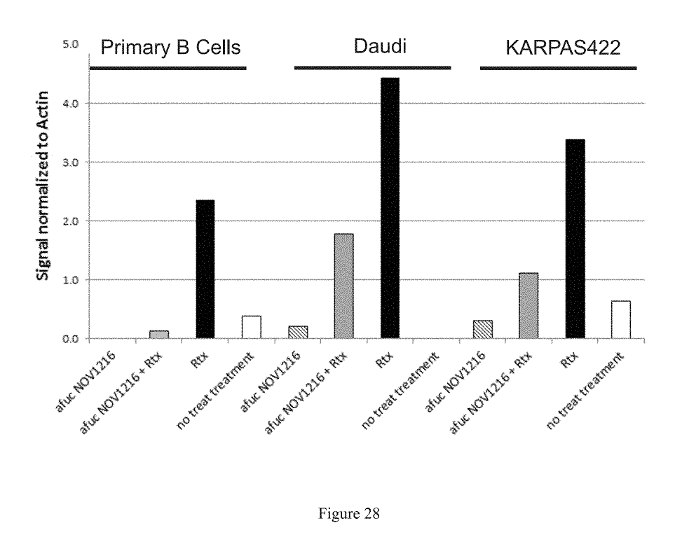

As seen in FIG. 28, afucosylated NOV1216 had little to no impact on CD32b ITIM phosphorylation relative to untreated controls. As anticipated, addition of rituximab to these cell populations resulted in a robust agonism of CD32b as evidenced by the increase in pCD32b levels. Co-incubation of afucosylated CD32b-binding NOV1216 with rituximab markedly reduced the rituximab-driven increase in pCD32b levels (FIG. 28). This was seen in primary B cells as well as CD20 and CD32b positive Daudi and Karpas422 cancer cell lines.

### Example 27: Expression of Cd32B Protein on Primary Patient Multiple Myeloma Samples and Two Established Cell Lines

The CD32b Fc receptor is expressed on both normal and malignant plasma cells. The binding of huCD32b specific antibody to normal human plasma cells from fresh unprocessed bone marrow (Lonza) and multiple myeloma bone marrow mononuclear cell patient samples (Conversant) was evaluated by flow cytometry. Unprocessed bone marrow were washed with PBS and then treated with RBC Lysis Buffer (eBioscience) to remove any contaminating red blood cells. Normal plasma cells were isolated from bone marrow mononuclear cells using Plasma Cell Isolation Kit II (Miltenyi Biotec 130-093-628) according to manufacturer's instructions. Multiple myeloma patient samples were rapidly thawed in a 37° C. water bath and diluted dropwise with pre-warmed RPMI medium. Samples were washed with RPMI medium and then treated with RBC Lysis Buffer (eBioscience) to remove any contaminating red blood cells. Tumor B cell lines JeKo-1 (mantle cell lymphoma) and MOLP-2 (multiple myeloma) were used as controls to assess huCD32b staining.

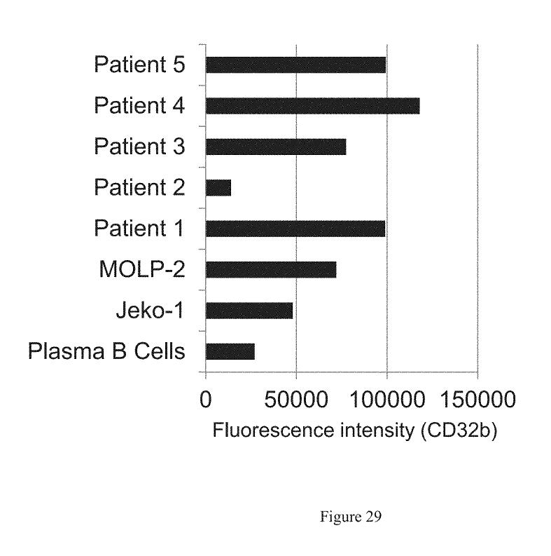

Normal and malignant plasma cell samples were resuspended in 0.5 ml FACS Buffer (PBS containing 2% BSA, 2 mM EDTA) supplemented with 20% FBS and distributed into a 96-well round bottom plate (100 ul per well). Control tumor samples were counted and 2×10⁵ cells per well were distributed into a 96-well round bottom plate. The samples were then stained in an equal volume of 2× antibody cocktail containing FITC-CD38, PE-CD138, PE-Cy7-CD45, and AlexaFluor 647-CD32b clone 2B6 [N297A] or AlexaFluor 647-hIgG1 isotype control [N297A]. Samples were incubated 30 min on ice. After 2 successive washes with FACS buffer, cells were resuspended in 7-AAD staining solution diluted in FACS buffer and acquired on the BD LSR II flow cytometer. The Median Fluorescence Intensity (MFI in the AlexaFluor 647 channel) in the CD45+CD38+CD138+ gate was used as a measure of the binding intensity for the CD32b antibody. Normal plasma cells had less intense CD32b staining than the control tumor B cell lines while 4 out of 5 multiple myeloma patient samples had more intense CD32b staining than both control tumor B cell lines and normal plasma cells (FIG. 29). These data indicate that CD32b may be a desirable target for treating B cell malignancies including multiple myeloma.

### Example 28: Wildtype and Fc Enhanced NOV2108 Efficiently Mediate Daudi Target Cell Killing by Human NK Cells

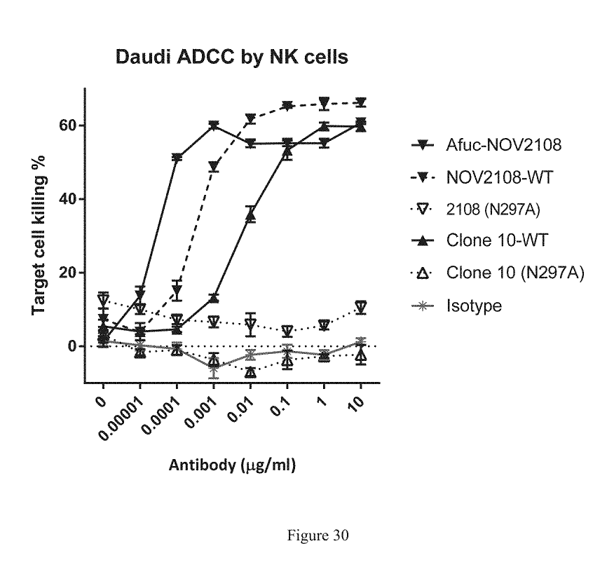

In this example, the anti-CD32b antibody clone 10 discussed above in Example 24 and NOV2108 were tested for their ability to mediated ADCC by NK cells. NOV2108 in afucosylated (Afuc), wildtype (WT) and N297A (silenced) formats as well as clone 10 (WT and N297A) were tested in the ADCC assay with isolated human natural killer cells to kill DAUDI cells. In brief, PBMCs were isolated from a Leukopak (HemaCare catalog# PB001F-3) via a ficoll gradient (GE Healthcare 17-1440-02). NK cells were then negatively selected using Miltenyi beads (catalog#130-092-657) and then incubated in IL2-containing medium overnight (RPMI/10% FBS with 0.1 ng/ml IL-2). Luciferised Daudi cells were pre-incubated for 20 min with a serial dilution of the antibodies in a 96-well microtiter plate (Corning Costar, catalog #3917) at a concentration of 10,000 cells per well. NK cells were then added at an effector to target ratio of 3:1. Following a 2 hour co-incubation, Britelite plus (Perkin Elmer, catalog#6066769; 70 μl) was added to all wells, with the exception of the background control wells (Daudi cells only). Target cells (no Ab or NK) with Britelite served as maximal signal controls whereas target cells without Britelite served as background controls. The luminescence signal was subsequently measured on an Envision (Perkin Elmer). The percent killing of target cells was calculated using the following formula: [1−(sample−background)/maximal)]×100%. NOV2108-WT mediated more potent ADCC than the clone 10-WT Ab, whereas afucosylated NOV2108 showed further enhanced killing of Daudi cells (FIG. 30).

In both the NK- and macrophage-mediated killing assays (Example 24), NOV2108 and clone 10 with identical Fc format (WT) were compared, and NOV2108-WT mediated more robust target cell killing than clone 10-WT by both effector cell types. Therefore, NOV2108 is an improved anti-CD32b ADCC antibody when compared with clone 10.

### Example 29: Assessing the Role of Anti-Cd32B Antibodies with Different Fc Function Mutations in Modulating Alemtuzumab or Rituximab Resistance in the Bone Marrow of the GMB Leukemia Model

Leskov et al. in “Rapid generation of human B-cell lymphomas via combined expression of Myc and Bcl2 and their use as a preclinical model for biological therapies,” Oncogene 32(8):1066-72” (Leskov et al., 2013) report an aggressive human B cell leukemia model, GMB, by co-expressing both human proto-oncogenes myc and bcl-2 in developing B cells in humanized mice. GMB leukemia cells are susceptible to alemtuzumab, a humanized monoclonal antibody specific for human CD52, leading to their elimination from the spleen, liver and blood, but not bone marrow of NSG mice. Using this model, macrophages were shown to be a key determinant of antibody-mediated cytotoxicity in the refractory bone marrow microenvironment. Interestingly, one mechanism of resistance to alemtuzumab therapy was shown to be the upregulation of CD32b (FcγRIIb) on leukemic cells in the bone marrow, but not spleen, indicating specific microenvironmental factors regulating ADCC activity (Pallasch et al (2014) “Sensitizing protective tumor microenvironments to antibody mediated therapy.” Cell 156: 590-162). Moreover, knockdown of CD32b via shRNA in the alemtuzumab resistant GBM cells re-sensitized the cells to alemtuzumab-mediated ADCC killing. These data suggest that increased CD32b expression is a mechanism of resistance to alemtuzumab. It is postulated that targeting CD32b with a mAb that blocks the CD32b Fc binding domain may yield similar results as depleting CD32b via shRNA. Additionally, co-administration of alemtuzumab (or other mAb with Fc-dependent mode of action) and an anti-CD32b mAb may delay the onset of resistance.

GMB leukemia cells are susceptible to alemtuzumab-mediated killing in a macrophage-dependent manner (Pallasch et al. 2014). In the published study, GMB leukemia cells were transferred into non-humanized NSG mice that lack human immune cells. Alemtuzumab successfully eliminated GMB leukemia cells from the spleen, liver and blood, but not bone marrow of NSG mice.

The role of anti-CD32b antibodies (NOV1206 WT, Fc silent, ADCC enhanced (S239D/A330L/I332E Fc enhanced mutant)) in modulating alemtuzumab or rituximab resistance will be monitored in the GMB leukemia model by dosing anti-CD32b targeting mAb and measuring the delay or prevention of alemtuzumab or rituximab resistance in the GMB in vivo leukemia model by targeting CD32b to restore sensitivity of the leukemia cells to alemtuzumab in vivo. If alemtuzumab is not available, rituximab will be used instead pending confirmation that rituximab resistant GBM cells in BM demonstrate upregulated CD32b expression.

In this example, NSG mice will be inoculated with GMB leukemia cells and randomly assigned to one of the following experimental arms:

### Group 1: PBS

Group 2: Alemtuzumab (or rituximab) dosed as in Pallasch et al paper
Group 3: anti-CD32b mAb (with Fc silencing mutation N297A) [20 mg/kg i.v. qw]
Group 4: anti-CD32b mAb (Fc enhanced or WT Fc) [20 mg/kg i.v. qw]
Group 5: anti-CD32b mAb (Fc enhanced or WT Fc) [20 mg/kg i.v. qw]+alemtuzumab or rituximab
Group 6: anti-CD32b mAb (with Fc silencing mutation N297A) [20 mg/kg i.v. qw]+alemtuzumab or rituximab
Group 7: Alemtuzumab (or rituximab) with cyclophosphamide dosed as in Pallasch et al paper.

GMB cells will be collected from the bone marrow of mice in Group 2 upon resistance to alemtuzumab and assessed for CD32b expression by FACS (a time-matched cohort of untreated mice will serve as controls). Group 3 will be a control to assess the Fc independent, single agent activity of the anti-CD32b mAb. Groups 5 and 6 should reveal the therapeutic impact of targeting CD32b with an Fc WT (or FC enhanced) or Fc silent (N297A) mAb on GMB disease burden and on the durability of response, particularly in the bone marrow space. Group 6 should reveal the specific impact of blocking CD32b with the CD32b targeted antibody (CDR specific activity) on the depth and durability of response to alemtuzumab or rituximab, particularly in the bone marrow, in the absence of Fc function of the CD32b antibody. This will help delineate the therapeutic benefit derived from the Fc dependent and CDR dependent (Fc independent) activity of the anti-CD32b mAb.

NSG mice will be inoculated with GMB leukemia cells and treated with alemtuzumab or rituximab until the onset of resistance in the bone marrow as described by Pallasch et al. (2014). If alemtuzumab is not available, rituximab will be used instead pending confirmation that rituximab resistant cells in BM demonstrate upregulated CD32b expression. At the onset of alemtuzumab or rituximab resistance in the bone marrow, the mice will be randomly assigned to one of the following experimental treatment groups. Additionally, at this time a cohort of mice will be euthanized and GMB leukemia cells in the bone marrow space will be collected for assessment of CD32b expression via FACS and compared to that of untreated mice. Based on findings from the Pallasch paper, Alemtuzumab resistant GMB cells in the bone marrow are anticipated to have increased CD32b expression.

### Group 1: PBS

Group 2: Alemtuzumab or rituximab
Group 3: anti-CD32b mAb (N297A)
Group 4: anti-CD32b mAb (Fc enhanced or WT Fc)
Group 5: anti-CD32b mAb (Fc enhanced or WT Fc)+alemtuzumab
Group 6: anti-CD32b mAb (N297A)+alemtuzumab or rituximab
Group 7: Alemtuzumab or rituximab+cyclophosphamide

Groups 1, 2, and 3 are control groups and are not anticipated to impact the course of disease. Group 4 should reveal the therapeutic benefit of treating alemtuzumab or rituximab resistant GMB with an aFc enhanced anti-CD32b mAb. Groups 5 and 6 should reveal the potential of an Fc WT (or Fc enhanced) and Fc silent (respectively) anti-CD32b mAb to reverse alemtuzumab or rituximab resistance in the bone marrow niche. The latter group, with an Fc silencing mutation, should specifically reveal the potential effect of CD32b Fc-binding domain blockade (CDR specific activity of the anti-CD32b mAb) on the response of GMB cells to alemtuzumab or rituximab.

### Example 30: Assessment of Complement Dependent Cytotoxicity (CDC) Activity of Anti-Cd32B Ab

A series of in vitro studies were conducted to assess the ability of afucosylated NOV2108 to kill CD32b positive cells by complement dependent cytotoxicity (CDC). In the CDC assay KARPAS-422 cells are incubated with different antibody concentrations and a fixed concentration of rabbit complement. Concentration-dependent killing of the KARPAS-422 cells is analyzed after 2 h, by measuring the viability of the cells via the intracellular ATP concentration, i.e. the luminescence produced by the ATP-consuming luciferin-luciferase system.

KARPAS-422 cells were harvested and adjusted to a concentration of 1.7×10⁵ cells/mL and 50 μl of the suspension were added into all wells of a white flat-bottomed 96 well microtiter plate. Then, eight serial dilutions of afucosylated NOV2108 (62.8 mg/mL) and MabThera (lot#H0165B09, 10 mg/mL) in assay buffer were prepared in triplicate in a U-bottom microtiter plate to result in final assay concentrations of 30,000 ng/mL, 6000 ng/mL, 1200 ng/mL, 240 ng/mL, 48 ng/mL, 10 ng/mL, 2 ng/mL, and 0.4 ng/mL and 50 μl of the dilutions were transferred to the assay plate containing the KARPAS-422 cells. Finally 50 μl of rabbit complement, diluted 1:8 in assay buffer, were added to the assay plate and the plate was gently rocked on a plate shaker for 60 s.

As controls, assay buffer was mock-diluted analogously to the samples. Additionally, a blank control containing cells without sample and complement, a negative control lacking the antibody and a positive control lacking the antibody but containing 1% Triton X-100 for complete lysis of the cells were included in octuplicate.

After 2-h incubation to 37° C., 5% CO2, 100 μL of reconstituted CellTiterGlo solution was added to all wells and the plate was incubated for 30 min at room temperature with gently shaking during the first 15 min. Finally, luminescence was measured.

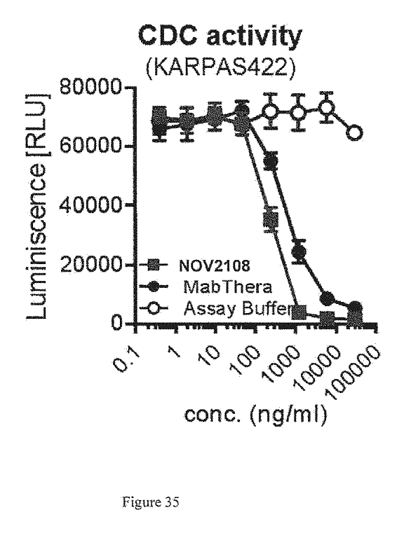

NOV2108 and the positive control MabThera demonstrated dose dependent killing of KARPAS-422 cells in this CDC assay (FIG. 35). These data demonstrate that afucosylated NOV2108 is able to engage complement and kill CD32b positive cells by CDC. As expected the buffer control did not reduce the number of viable cells in this experiment.

### Example 31: Macrophages are Cd32B-Positive but are More Resistant to Anti-Cd32B Ab-Mediated Lysis (by NK Cells) or Phagocytosis (by Other Macrophages)

Macrophages are known to express CD32b as well as other members of the FcγR family. It is possible that macrophages can be targeted by an anti-CD32b antibody and killed via ADCC or ADCP mechanism.

### Macrophages Express CD32b.

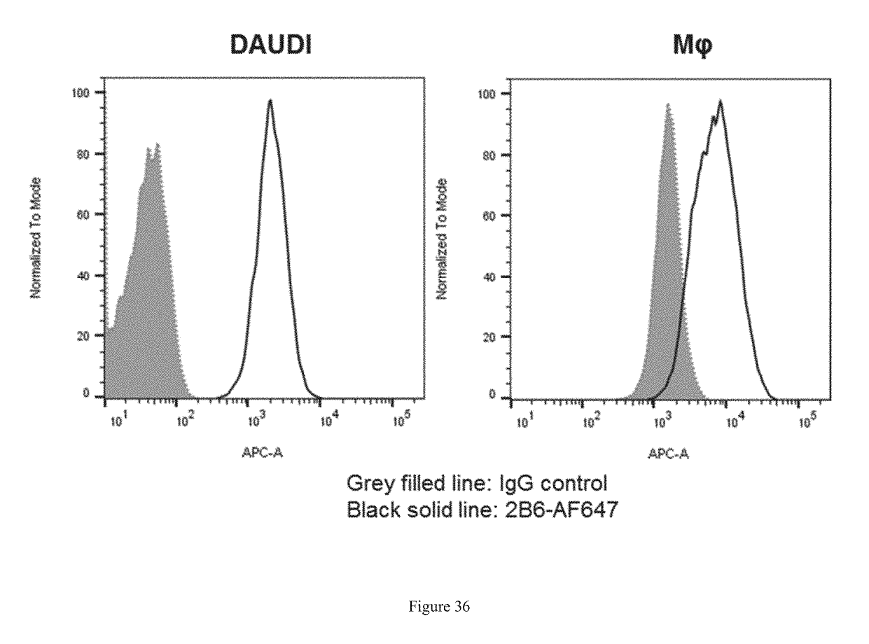

It was first determined whether the anti-CD32b antibody binds to macrophages. Human monocytes-derived macrophages were differentiated as described in Example 24. Macrophages attached to a 96-well flat bottom plate were incubated with Alexaflour 647-labeled anti-CD32b Ab 2B6 (N297A Fc-silenced mutant) at 0.5 ug/ml staining solution PBS+2% IFS for 30 min on ice. After two successive washes with FACS buffer, cells were suspended in 120 μl FACS buffer and acquired on a FACS Fortessa. Daudi cells were used as a positive control and stained as suspension cells with the same staining condition. An Alexaflour647-labeled anti-chicken lysozyme Ab (N297A mutant) was used as IgG control. FACS histogram shows relative level of staining as MFI(x-axis) versus the number of events recorded(y-axis) Staining by anti-CD23b Ab 2B6 (solid line) is overlaid with that of the IgG control (filled dotted line). Macrophages showed background binding to the IgG control, as expected by the multiple FcγRs, especially FcγRI, a high affinity Fc receptor (FIG. 36a). 2B6 binding to macrophages are higher than the IgG control, indicating that macrophages are CD32b positive. However, the shift between 2B6 and IgG control for macrophages are smaller than Daudi cells (FIG. 36a, FIG. 36b).

Macrophages are Less Sensitive than Daudi to Anti-CD32b Ab-Mediated ADCC by NK Cells.

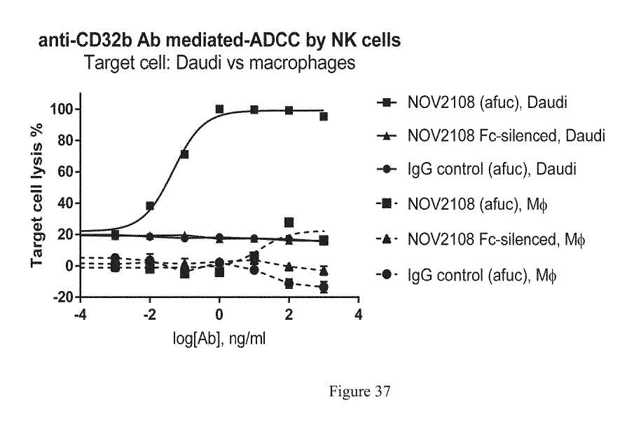

Next we compared whether macrophages to Daudi in an in vitro ADCC assay with anti-CD32b Ab NOV2108 (afucosylated). NK cells were isolated from a different donor as described in example 17. Adherent macrophages were labeled with Calcein AM in the 96-well flat-bottom plate (4 ug/ml in RPMI with 10% FBS, 60 ul/well) for 1 hr. The number of target cells for macrophages or Daudi were 60,000/well and 120,000 NK cells/well were used for an effector: target ratio of 2:1. ADCC assay were performed as described in Example 17. Target cell lysis was measured after 2 hr. Daudi cells were efficiently lysed by NK cells whereas macrophages were more resistant to ADCC (FIG. 37), with low level of lysis observed only at higher concentration of afucosylated NOV2108.

### Macrophages are Resistant to Anti-CD32b Ab-Mediated ADCP

NOV2108 can mediate efficient killing of CD32bpos cell lines Daudi (example 24) via ADCP mechanism. Because macrophages are CD32bpos we sought to determine whether macrophages can phagocytose each other in the presence of anti-CD32b Ab. We used time-lapse confocal imaging to visualize phagocytosis of cells labeled with Cell Tracker dyes (Molecular Probes). For macrophage differentiation petri dishes were used to reduce cell attaching to the surface. Effector cell macrophages were labeled with 0.2 μM Cell tracker green (Cat# C7025) for 10 min in serum free RPMI medium. Target cells daudi or macrophage were labeled with 0.5 uM Cell tracker red (Cat# C34552) for 10 min. Effector macrophages (green) were labeled and plated on an 8-well μ-Slide (Ibidi, cat#80826) one day before imaging whereas the target cells were labeled immediately before imaging.

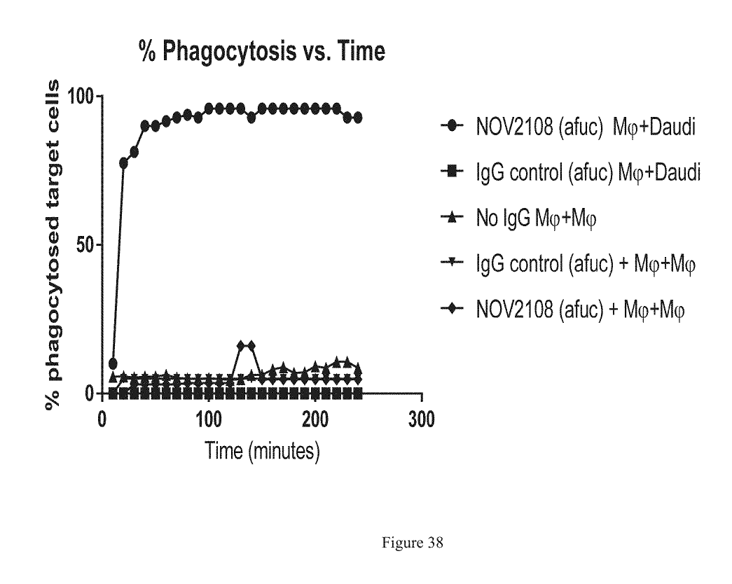

Imaging was performed on a Zeiss spinning disk confocal microscope (Axio Observer.Z1) with a 40×/1.30 Oil Ph3 objective. Z-stack images were taken to image the entire cell (lateral resolution ˜0.5 um, axial resolution ˜2 um). Laser power was set to 3.00%, 3.50%, 5.80% and 4.00% for 405 nm (SYTOX® Blue), 488 nm (CellTracker™ Green CMFDA Dye), 561 nm (CellTracker™ Red CMTPX Dye) and 633 nm (Antibody labeled Alexa-647) lasers, respectively. Camera exposure was set to 30 ms, 40 ms, 60 ms, and 35 ms exposure for 405 nm, 488 nm, 561 nm, and 633 nm channels, respectively. A microscope incubator was used to keep the cells at 37 degrees Celsius with 5% CO2 for entire imaging time. Images were acquired for four positions per well, in 10 minute intervals over four hours. All image acquisition and image processing was performed with Zen Blue software. To quantify the number of cells phagocytosed CellTracker™ Red CMTPX labeled Daudi cells or macrophages were counted manually frame by frame for up to 240 minutes (24 timepoints). The percentage of cells phagocytosed per frame was then calculated. Finally, the percentage per timepoint of 3-4 positions per well were averaged to get the mean percentage of phagocytosis per treatment well. All data shown in FIG. 38 represent replicates of 4 positions per well for each treatment condition.

Red-labeled Daudi cells were efficiently phagocytosed by green macrophages (reaching 80% within 30 min and 95% by 60 min). We detected minimum numbers of macrophage phagocytosed by each other during the 4 hr experiment, and there was no difference between wells where afucosylated NOV2108 was added and where IgG control was added.

### Example 32: Binding Affinities of Anti-Cd32B Abs for Cd32B

Three independent direct binding assays with IgGs covalently immobilized on the biosensor and huCD32b receptor serving as analyte were performed to determine the binding affinities of the IgGs for huCD32b. Kinetic data were acquired by subsequent injections of analyte dilution series on all flow cells. Flow cell 1 (chip 1) served as a reference.

550 RU of afucosylated NOV2108 and Fc silent NOV2108 [N297A] were immobilized on a CMS sensor chip using standard amine coupling chemistry. Additionally, a silent anti-chicken-lysozyme-hIgG1 [N297A], used as negative control, to exclude binding via the Fc part to CD32b was immobilized on the chip. A dilution series of huCD32b-deglyco, 0.61-5000 nM (1:2 dilution series) in running buffer was injected over the surface (flow rate: 30 μl/min, association time: 60 sec, dissociation time: 120 sec). The chip surface was regenerated with one basic wash step before each analyte injection (30 μl/min; contact time: 30 sec, stabilization period: 250 sec). Data were evaluated using the Biacore T200 evaluation software version 1.0. The raw data were double referenced, i.e. the response of the measuring flow cell was corrected for the response of the reference flow cell, and in a second step the response of a blank injection was subtracted. Outlier sensorgrams were removed if necessary. The sensorgrams were fitted by applying a 1:1 binding model to calculate kinetic rate constants and dissociation equilibrium constants. Rmax was set at global whereas RI was fitted locally. Data were processed individually for each run. The generated values were used to calculate average values and standard deviations of the respective kinetic constants.

The Fc silent version of NOV2108 (N297A) binds CD32b with a KD of 18±3 nM (see Table 6). NOV2108 (afucosylated format) showed a similar affinity as Fc silent NOV2108 in a single experiment with a KD of 16 nM. No binding was observed for the interaction of the silenced anti-chicken-lysozyme IgG to human CD32b. Therefore, binding via the Fc part to CD32b can be excluded.

TABLE 6
Association rate constants, dissociation rate constants and dissociation
equilibrium constants of the antibody-CD32b interactions.

| Antibody immobilized | kₐ (1/Ms) | k_{d} (1/s) | K_{D} (nM) |
|---|---|---|---|
| anti-hCD32b_NOV2108- | 1.5 ± 1E+7 | 2.9 ± 2.3E−1 | 18 ± 3 |
| hIgG1 (N297A) | | | |
| NOV2108, | 6.8E+6 | 1.1E−1 | 16 |
| afucosylated antibody | | | |
| Control hIgG1 (N297A) | | no binding | |

### Example 33: Afucosylation of NOV2108 Promotes Enhanced B Cell Killing and Retains Viability of Monocytes and Granulocytes

### Assessment of Killing Selectivity Induced by the Fc Wt and Afucosylated Anti-Hu CD32b Reactive mAb NOV2108 in Human Whole Blood

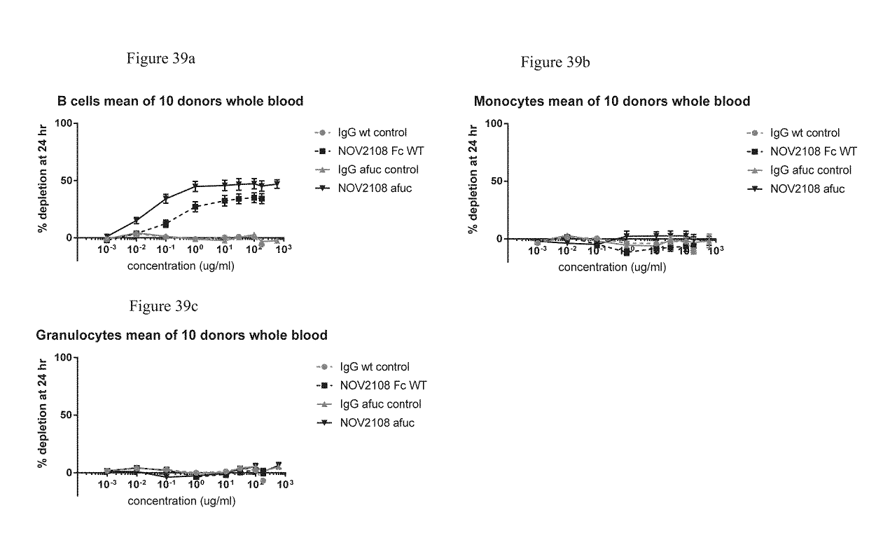

The potential of the Fc wt and afucosylated anti-hu CD32b mAb NOV2108 to induce killing of CD32a/b-positive immune cell subsets was evaluated in human whole blood. Varying concentrations of the test and control antibodies (Fc WT and Afucosylated (afuc) of matched isotypes) were incubated with heparinized whole blood from 10 different healthy donors for 24 h. Absolute counts of B cells, monocytes and granulocytes were measured on a flow cytometer after immunophenotyping of stimulated whole blood with marker antibodies against CD19, CD14 and CD45 after exclusion of dead cells using a viability dye. The percentage of depletion was calculated based on the change of absolute counts induced by the test antibody in comparison to the absolute counts measured with the buffer control: 100−(absolute counts (test condition*100/absolute counts (buffer)). The afucosylated Fc variant of NOV2108 overall induced stronger B cell killing compared to the the Fc WT variant (FIG. 39a) and did not affect the viability of monocytes (FIG. 39b) and granulocytes (FIG. 39c).

### Example 34: Assessment of Primary NK Cell Driven, Specific ADCC Activity Against Karpas620 Cancer Cell Lines by Fc Wt and Fc Modified Anti-Cd32B Antibodies

A primary NK cell ADCC assay was utilized to assess the Fc dependent activity of CD32b reactive antibodies against CD32b positive, Karpas620 cells. In brief, PBMCs were isolated from a Leukopak (HemaCare catalog# PB001F-3) via a ficoll gradient. NK cells were then negatively selected using Miltenyi beads (catalog#130-092-657) and then incubated in basic media overnight (RPMI/10% FBS/15 mM HEPES/1% L-glutamine/1% Penicillin Streptomycin) in the presence of 100 pg/ml of rhIL-2 (PeproTech, catalog#200-02). The following day, Karpas620 cells were stained with Calcein acetoxy-methyl ester (Calcein-AM; Molecular Probes catalog# C3100MP), washed twice, and transferred to a 96-well U-bottom microtiter plate at a concentration of 10,000 cells per well. The cells were then pre-incubated for 20 min with a serial dilution of the antibodies before adding the effector cells at an effector to target ratio of 5:1. Following the co-incubation, the microtiter plate was centrifuged and an aliquot of the supernatant fluid was transferred to another microtiter plate (Corning Costar, catalog #3904) and the concentration of free Calcein in solution was determined with a fluorescence counter (Envision, Perkin Elmer). Target cells only and target cells with 1% Triton (Sigma, 93443) were included as controls. Target cells only served as spontaneous release whereas target cells with 1% triton served as maximal release. The percent specific target cell lysis was calculated using the following formula: [(sample−spontaneous release)/(maximal release−spontaneous release)]×100%.

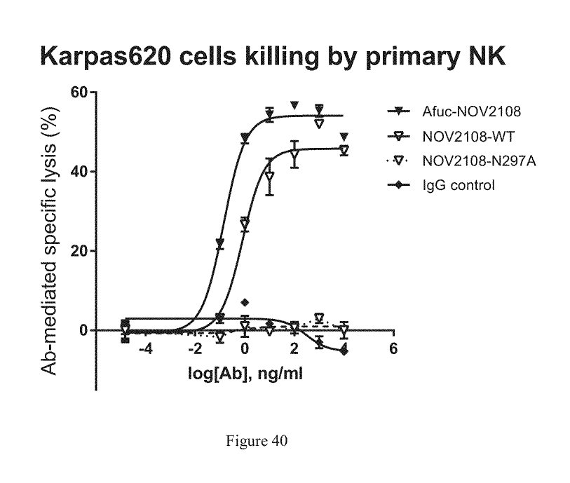

Three versions of the anti-CD32b antibody NOV2108 were tested: Fc WT, afucosylated (Fc-enhanced) and N297A (Fc-silenced). Fc WT NOV2108 mediated efficient ADCC on Karpas620 cells, and the activity was enhanced by the afucosylated NOV2108 (FIG. 40). As expected, the Fc silent N297A version of NOV2108 was as inactive as the IgG isotype, confirming that the NK cell activation and MM cell lysis requires a functional Fc.

### Example 35: Pre-Treated PBMC with Lenalidomide Potentiates ADCC Activity of NOV1216-AFUC

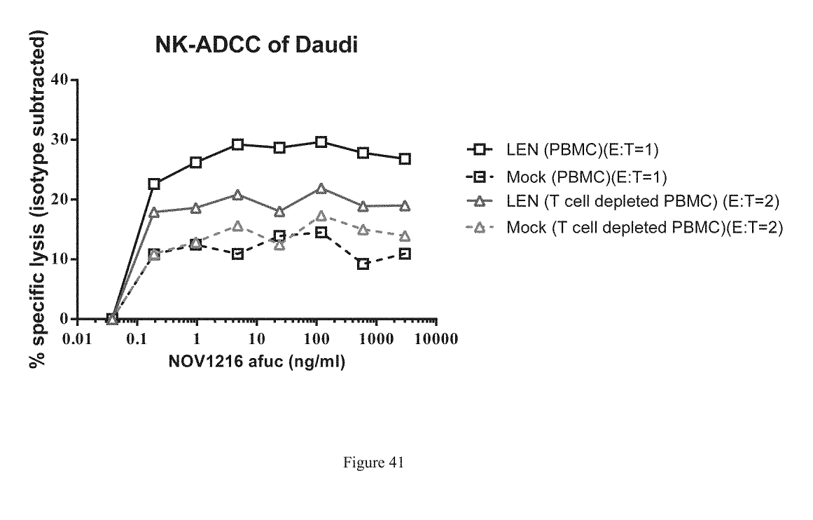

Lenalidomide (LEN), an immune-modulating drug can modulate anti-tumor effect of lymphocyte function, which in turn activate NK cells and increased cytotoxicity. In order to determine if LEN could potentiate the ADCC activity, PBMC or T cell depleted PBMC were used as effector cells and Daudi was used as a target. In brief, PBMCs were isolated from a Leukopak (HemaCare catalog# PB001F-3) via a ficoll gradient. T cells were positively depleted out from PBMC by using CD3 beads (Miltenyi, catalog#130-050-101). PBMC or T cell depleted PBMC were incubated in basic medium without recombinant IL-2 (RPMI/10% FBS/15 mM HEPES/1% L-glutamine/1% Penicillin Streptomycin), which was supplemented with 3 μM LEN or equal volume of DMSO (mock) for 72 hours prior to NK cell isolation and ADCC assay (as described in example 17). NK cells isolated from PBMCs pre-treated with LEN showed higher ADCC activity than NK cells from mock treated PBMC on Daudi cells in the presence of anti-CD32b Ab afucosylated NOV1216 (FIG. 41). This data provides support to the combination of anti-CD32b antibodies with Lenalidomide in the treatment of CD32b+ lymphoma and myeloma.

It has been suggested that LEN can activate T cells and increase IL-2 secretion by T cells, which in turn activates NK cells. Therefore we depleted T cells from the PBMCs upon isolation and repeated the 72 hr pre-treatment with LEN. T-cell depletion alone had minimal effect on NOV1216-mediated ADCC activity by NK cells isolated from mock-treated PBMC. Only NK cells were used in the ADCC assay as effector cells, therefore the direct effect of LEN on NK cell activity is not significant. However, the enhanced ADCC activity by LEN pretreatment of PBMC was largely abrogated when T cells were depleted prior to LEN treatment, supporting the important role of T cells in the response to LEN and activation of NK cells.

### Example 36: In Vivo Activity Associated with Combining Anti-Cd32B eADCC Fc Mutant Antibody and HDAC Inhibitor Panobinostat in Mice Bearing Cd32B Low KMS-12-BM Subcutaneous Xenografts

This example explores the therapeutic benefit of combining an eADCC Fc mutant CD32b targeted antibody with the marketed HDAC inhibitor panobinostat in mice bearing the CD32b low MM xenograft KMS-12-BM.

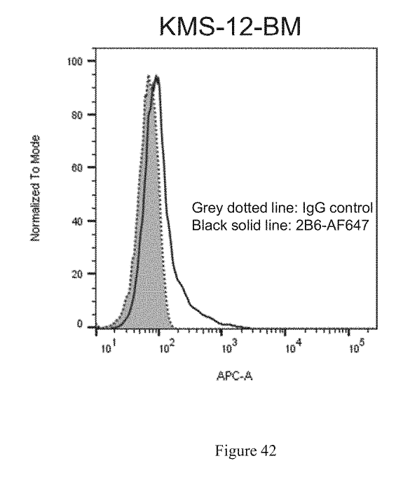

The level of CD32b expression on the KMS-12-BM cell line was determined via flow cytometry using the 2B6 antibody. KMS-12-BM cells were counted and suspended at 1×10⁶ cells per ml in FACS Buffer (PBS1× containing 2% FBS). 200'000 cells/well (200 μl) were then dispensed in U-bottomed 96 well plates. Plates were spun for 5 min at 1200 rpm and the supernatant discarded. Cells were then suspended in 100 μl of FACS Buffer containing 1 ug/ml of 2B6 antibody or IgG control and incubated 30 min at 4° C. After two successive washes with FACS buffer, cells were suspended in 120 μl FACS buffer and acquired on a FACS Fortessa. FACS histogram shows relative level of staining as MFI (x-axis) versus the number of events recorded (y-axis) Staining by the anti-CD32b mAb (solid line) is overlaid with that of the IgG control (filled dotted line) (FIG. 42). These data demonstrate that KMS-12-BM express very little CD32b.

Female nude mice were implanted subcutaneously with 10×10⁶KMS-12-BM cells (100 μl injection volume) suspended in 50% phenol red-free matrigel (BD Biosciences) diluted with PBS. Mice were enrolled in the study 7 days post implantation with average tumor volume of 210 mm³. After being randomly assigned to one of 4 experimental groups (n=7/group), mice were intravenously administered the following treatments: (1) PBS, (2) NOV2108 (eADCC mouse IgG2a (S239D/I332E), 10 mg/kg q2w), (3) panobinostat (12 mg/kg q2d*5 followed by 4d break in 14 day cycles), and the combination of (1)+(3) at the aforementioned doses and schedules. Tumor burden and body weight was assessed twice per week. Time to endpoint, defined as tumors reaching 800 mm³, was also evaluated. The eADCC mouse IgG2a version of NOV2108 was utilized to reflect the therapeutic potential associtated with optimal interaction between therapeutic Ab Fc and FcγR on mouse immune effector cells.

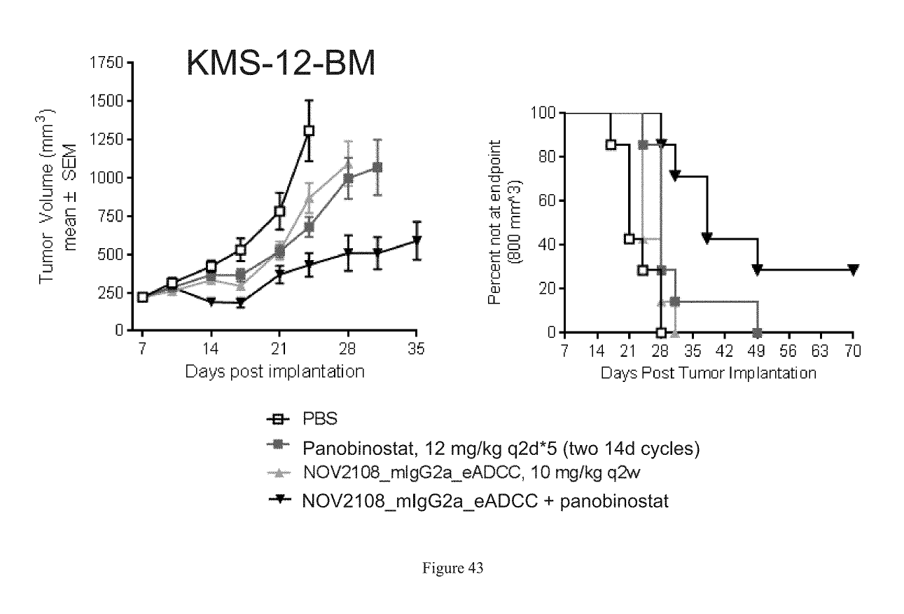

The single agent treatments of NOV2108 (eADCC Fc mutant mouse IgG2a) and panobinostat had limited impact on mean tumor volume (FIG. 43). The combination of these two treatments resulted in increased anti-tumor activity. Specifically, the combination treatment yielded more significant (P<0.05) antitumor activity (percent tumor volume change) than the single agent groups (day 28 represents the final point when all three experimental groups remained on treatment). The combination also increased time to endpoint (800 mm³). These data indicate that the HDAC inhibitor panobinostat sensitizes CD32b low MM xenograft to the CD32b targeted NOV2108 (eADCC Fc mutant mouse IgG2a). The data provide rational for testing the combination of an anti-CD32b targeted antibody and an HDAC inhibitor, e.g. panobinostat, in patients with MM.

### Example 37: Dose Response In Vivo Activity of Afucosylated Anti-Cd32B Antibody NOV2108 in Nude Mice Bearing Daudi Xenografts

An in vivo efficacy experiment was conducted in nude mice bearing subcutaneous Daudi xenografts to explore the dose depend antitumor activity of the afucosylated anti-CD32b NOV2108 human IgG1. NOV1216, was also included in this experiment as an eADCC Fc mutant (S239D/I332E) mouse IgG2a framework. Female nude mice were implanted subcutaneously with 5×10⁶ Daudi cells (100 μl injection volume) suspended in 50% phenol red-free matrigel (BD Biosciences) diluted with PBS. Mice were enrolled in the study 10 days post implantation with average tumor volume of roughly 220 mm³. After being randomly assigned to one of 6 experimental groups (n=7/group), mice were intravenously administered the following treatments: (1) PBS, (2) non-targeted afucosylated isotype control (30 mg/kg qw), (3) afucosylated NOV2108 (3 mg/kg qw), (4) afucosylated NOV2108 (10 mg/kg qw), (5) afucosylated NOV2108 (30 mg/kg qw), and (6) eADCC Fc mutant mouse IgG2a NOV1216 (10 mg/kg q3w). Tumor volume and body weight was assessed twice per week. The eADCC mouse IgG2a version of NOV1216 was utilized to reflect the therapeutic potential associated with optimal interaction between therapeutic Ab Fc and FcγR on mouse immune effector cells.

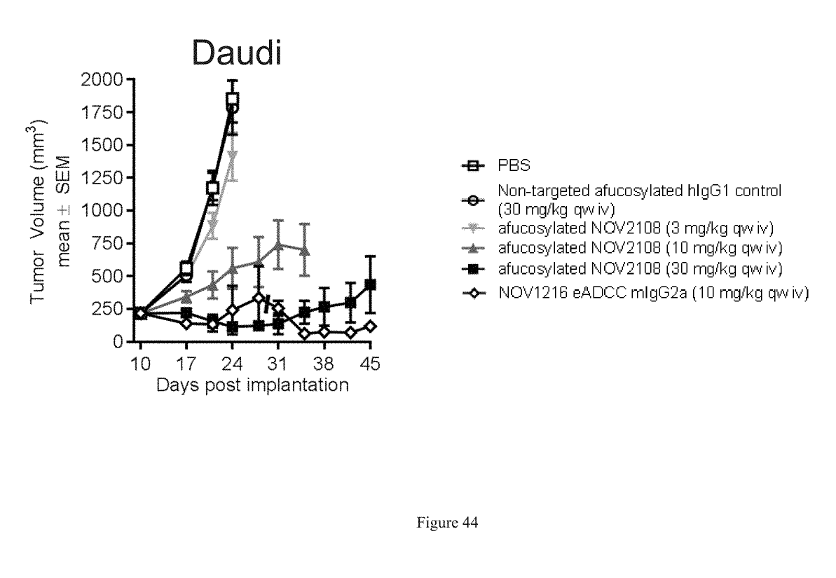

Afucosylated NOV2108 demonstrated dose dependent antitumor activity in mice bearing subcutaneously engrafted Daudi xenografts (FIG. 44). One mouse from NOV1216 eADCC mIgG2a group failed to respond to treatment and was removed from study due to excessive tumor volume at day 28. Tumor growth of mice administered a 3 mg/kg qw dose was not distinguishable from that of mice administered PBS or non-targeted control antibody (30 mg/kg qw). However, afucosylated NOV2108 administered 10 or 30 mg/kg qw yielded marked tumor growth inhibition. NOV1216, which has a highly similar variable region to that of NOV2108, administered as an eADCC Fc mutant mouse IgG2a yielded marked anti-tumor activity roughly similar to that observed with afucosylated NOV2108 administered at a much higher dose (30 mg/kg qw). These data highlight the therapeutic benefit associated with an optimal interaction between host immune effector cell FcγRs and therapeutic mAb Fc region.

### Example 38: Antitumor Activity of Afucosyalted NOV2108 in Nude Mice Bearing Karpas620 Mm Subcutaneous Xenografts

An in vivo efficacy experiment was conducted in nude mice bearing subcutaneous KARPAS620 MM xenografts to explore the dose depend antitumor activity of the afucosylated anti-CD32b NOV2108 human IgG1. Female nude mice were implanted subcutaneously with 1×10⁷ KARPAS620 cells (100 μl injection volume) suspended in 50% phenol red-free matrigel (BD Biosciences) diluted with PBS. Mice were enrolled in the study 10 days post implantation with average tumor volume of roughly 220 mm³ After being randomly assigned to one of three experimental groups (n=8/group), mice were intravenously administered the following treatments: (1) PBS, (2) afucosylated NOV2108 (10 mg/kg qw), and (3) afucosylated NOV2108 (30 mg/kg qw). Tumor volume and body weight was assessed twice per week.

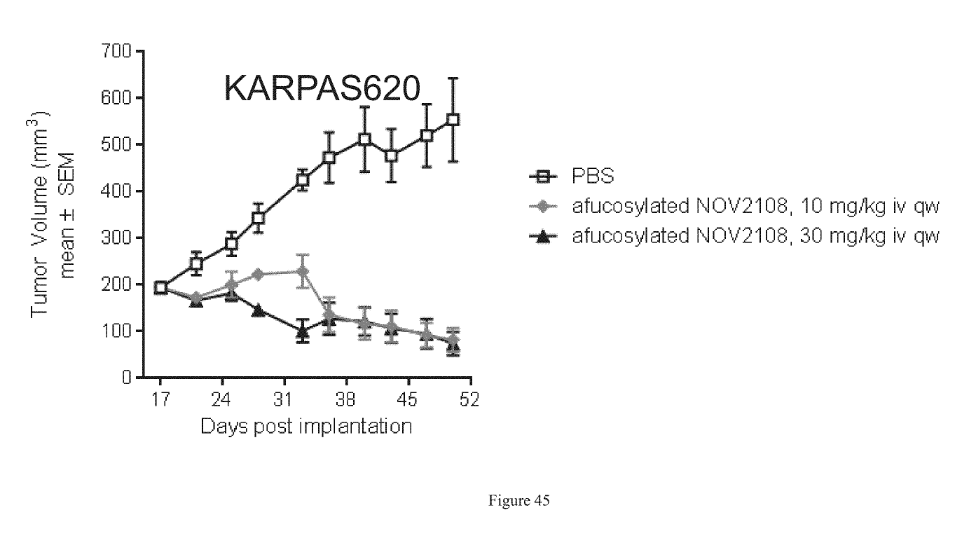

Afucosylated NOV2108 demonstrated marked antitumor activity in mice bearing subcutaneously engrafted KARPAS620 xenografts (FIG. 45) Similar antitumor activity was observed at both dose levels suggesting that this may be the maximal antitumor activity achievable. These data provide evidence for the therapeutic benefit afucosylated NOV2108 may have in patients with MM.

### Example 39: Impact of Intravenous Administration of eADCC Fc Mutant NOV2108 on Intratumor Macrophage Content in Nude Mice Bearing Daudi Xenografts

An in vivo experiment was conducted in nude mice bearing subcutaneous Daudi xenografts to explore the impact intravenous administration of eADCC Fc mutant NOV2108 (S239D/A330L/I332E) has on intratumor macrophage content as determined by F4/80 IHC positivity. Female nude mice were implanted subcutaneously with 5×10⁶ Daudi cells (100 μl injection volume) suspended in 50% phenol red-free matrigel (BD Biosciences) diluted with PBS. Mice were enrolled in the study 10 days post implantation with average tumor volume of roughly 200 mm³ The experiment consisted of two parts. The first cohort (n=3/group) received (1) PBS, (2) eADCC Fc mutant non-targeted isotype control 10 mg/kg qw*2, or (3) eADCC Fc mutant NOV2108 10 mg/kg qw*2. Tumors were collected and evaluated for F4/80 immunoreactivity via IHC at 3d post second dose (10d post first dose). The second cohort received a single intravenous dose of eADCC Fc mutant NOV2108. Tumors were subsequently collected and evaluated for F4/80 immunoreactivity via IHC at day 7, 10, 14, and 21 post dose (n=3 per time point).

At each predetermined time point, tumors were immediately excised, fixed in 10% buffered formalin for 24 hours and transferred into 70% EtOH until processing (embedding in paraffin using routine histological procedures; tissue sections were cut at 3.5 um). The rabbit monoclonal anti-mouse F4/80 IgG (Clone SP115; Spring Bioscience) was used. Normal mouse lymphoid tissues served as a positive control.

An optimized IHC protocol (Ventana Biotin-free DAB Detection Systems; Ventana DISCOVERY XT Biomarker Platform) included standard exposure to Ventana Cell Conditioning #1 antigen retrieval reagent. The primary antibody was diluted to a concentration of 1:200 in DAKO Cytomation Antibody Diluent, applied in 100 ul volume and incubated for 60 minutes at room temperature. Subsequent incubation with Ventana OmniMap prediluted HRP-conjugated anti-rabbit secondary antibody (Cat #760-4311) was performed for 4 minutes. The secondary antibody was then detected using the ChromoMap DAB kit and slides were counterstained for 4 minutes with Ventana Hematoxylin, followed by Ventana Bluing Reagent for 4 minutes. Slides were dehydrated in increasing concentrations of ethanol (95-100%), then in xylenes, followed by coverslipping. Coverslipped slides were evaluated by light microscopy and scanned by Leica/Aperio ScanScope slide scanner (Vista, Calif.). Digital images were then viewed and analyzed by Indica Labs HALO (Corrales, N. Mex.) launching images from Leica eSlide Manager/Aperio Spectrum. Representative histologic images were captured using the figure maker module within in Indica Labs HALO (Corrales, N. Mex.). Scanned images of the stained slides were launched in Indica Labs HALO (Corrales, N. Mex.) opening from integrated Leica eSlide Manager/Aperio Spectrum (Vista, Calif.). Data are presented as percent positive tissue.

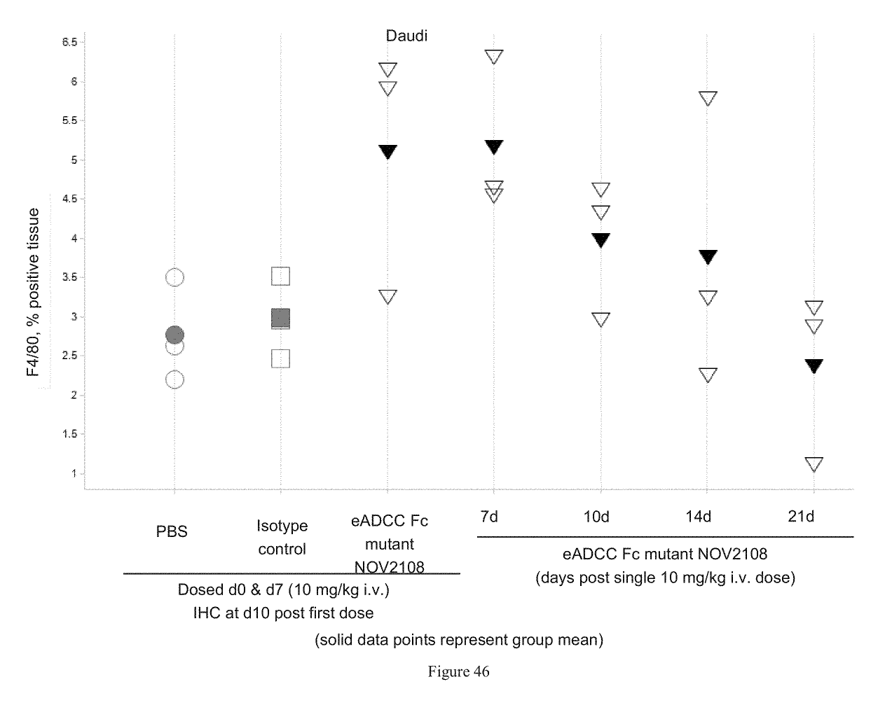

Relative to PBS treated controls, eADCC Fc mutant NOV2108 resulted in an increase in F4/80 immunoreactivity in DAUDI xenografts at 3d following a 10 mg/kg qw*2 dosing regimen (FIG. 46). In this figure, open shapes represent data from one animal whereas the filled shape represents the treatment. These data indicate that i.v. administration of, eADCC Fc mutant NOV2108 results in an increase in intratumor macrophage numbers. This was not observed in mice administered a non-targeted eADCC Fc mutant negative control antibody confirming that CDR mediated binding to CD32b on Daudi cells was required to recruit macrophages to the tumor. Additionally, when administered as a single 10 mg/kg intravenous dose, eADCC Fc mutant NOV2108 yielded an increase in intratumor macrophage numbers at 7d post dose. The intratumor macrophage content dropped at subsequent time points, approximating pre-dose levels at later time points post dose. These data support a role of mouse macrophages in mediating the Fc and CDR dependent activity of eADCC Fc mutant NOV2108 in vivo. The data also provide rationale for using intratumor immune cell infiltrate as a biomarker to guide dose scheduling.

Unless defined otherwise, the technical and scientific terms used herein have the same meaning as that usually understood by a specialist familiar with the field to which the disclosure belongs.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below.

Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of nucleic acid starting material, clone of interest, or library type is believed to be a matter of routine for a person of ordinary skill in the art with knowledge of the aspects described herein. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

1. An isolated antibody or antigen-binding fragment thereof, which comprises:
(a) A heavy chain variable region CDR1 comprising an amino acid sequence selected from any one of SEQ ID NOs: 1, 4, 7, 53, 56, 59, 105, 108, 111, 157, 160, 163, 209, 212, 215, 261, 264, 267, 313, 316, 319, 365, 368, 371, 417, 420, 423, 469, 472, 475, 521, 524, 527, 547, 550, 553, 573, 576, 579, 625, 628, and 631;
(b) a heavy chain variable region CDR2 comprising an amino acid sequence selected from any of SEQ ID NOs: 2, 5, 8, 54, 57, 60, 106, 109, 112, 158, 161, 164, 210, 213, 216, 262, 265, 268, 314, 317, 320, 366, 369, 372, 418, 421; 424, 470, 473, 476, 522, 525, 528, 548, 551, 554, 574, 577, 580, 626, 629, and 632;
(c) a heavy chain variable region CDR3 comprising an amino acid sequence selected from any of SEQ ID NOs: 3, 6, 9, 55, 58, 61, 107, 110, 113, 159, 162, 165, 211, 214, 217, 263, 266, 269, 315, 318, 321, 367, 370, 373, 419, 422, 425, 471, 474, 477, 523, 526, 529, 549, 552, 555, 575, 578, 581, 627, 630, and 633;
(d) a light chain variable region CDR1 comprising an amino acid sequence selected from any of SEQ ID NOs: 14, 17, 20, 66, 69, 72, 118, 121, 124, 170, 173, 176, 222, 225, 228, 274, 277, 280, 326, 329, 332, 378, 381, 384, 430, 433, 436, 482, 485, 488, 534, 537, 540, 560, 563, 566, 586, 589, 592, 638, 641, 644;
(e) a light chain variable region CDR2 comprising an amino acid sequence selected from any of SEQ ID NOs: 15, 18, 21, 67, 70, 73, 119, 122, 125, 171, 174, 177, 223, 226, 229, 275, 278, 281, 327, 330, 333, 379, 382, 385, 431, 434, 437, 483, 486, 489, 535, 538, 541, 561, 564, 567, 587, 590, 593, 639, 642, and 645; and
(f) a light chain variable region CDR3 comprising an amino acid sequence selected from any of SEQ ID NOs: 16, 19, 22, 68, 71, 74, 120, 123, 126, 172, 175, 178, 224, 227, 230, 276, 279, 282, 328, 331, 334, 380, 383, 386, 432, 435, 438, 484, 487, 490, 536, 539, 542, 562, 565, 568, 588, 591, 594, 640, 643, and 646;
wherein the antibody selectively binds human CD32b.

2. The antibody or antigen-binding fragment thereof of claim 1, wherein the antibody comprises: a heavy chain variable region comprising an amino acid sequence selected from any of SEQ ID NOs: 10, 62, 114, 166, 218, 270, 322, 374, 426, 478, 530, 556, 582, and 634; and a light chain variable region comprising an amino acid sequence selected from any of SEQ ID NOs: 23, 75, 127, 179, 231, 283, 335, 387, 439, 491, 543, 569, 595, and 647.

3. The antibody or antigen-binding fragment thereof of claim 1, wherein the antibody comprises: a heavy chain comprising an amino acid sequence selected from any of SEQ ID NOs: 12, 64, 116, 168, 220, 272, 324, 376, 428, 480, 584, and 636; and a light chain comprising an amino acid sequence selected from any of SEQ ID NOs: 25, 77, 129, 181, 233, 285, 337, 389, 441, 493, 597, and 649.

4. The antibody or antigen-binding fragment thereof of claim 1, wherein the antibody comprises: a heavy chain comprising an amino acid sequence selected from any of SEQ ID NOs: 38, 90, 142, 194, 246, 298, 350, 402, 454, 506, 532, 558, 610, and 662; and a light chain comprising an amino acid sequence selected from any of SEQ ID NOs: 51, 103, 155, 207, 259, 311, 363, 415, 467, 519, 545, 571, 623, and 675.

5. The antibody or antigen-binding fragment thereof of claim 1, wherein the antibody comprises:
(a) HCDR1, HCDR2, and HCDR3 sequences of SEQ ID NOs: 1, 2, and 3, respectively, and LCDR1, LCDR2, and LCDR3 sequences of SEQ ID NOs: 14, 15, and 16, respectively;
(b) HCDR1, HCDR2, and HCDR3 sequences of SEQ ID NOs: 4, 5, and 6, respectively, and LCDR1, LCDR2, and LCDR3 sequences of SEQ ID NOs: 17, 18, and 19, respectively;
(c) HCDR1, HCDR2, and HCDR3 sequences of SEQ ID NOs: 7, 8, and 9, respectively, and LCDR1, LCDR2, and LCDR3 sequences of SEQ ID NOs: 20, 21, and 22, respectively;
(d) HCDR1, HCDR2, and HCDR3 sequences of SEQ ID NOs: 53, 54, and 55, respectively, and LCDR1, LCDR2, and LCDR3 sequences of SEQ ID NOs: 66, 67, and 68 respectively;
(e) HCDR1, HCDR2, and HCDR3 sequences of SEQ ID NOs: 56, 57, and 58, respectively, and LCDR1, LCDR2, and LCDR3 sequences of SEQ ID NOs: 69, 70, and 71 respectively;
(f) HCDR1, HCDR2, and HCDR3 sequences of SEQ ID NOs: 59, 60, and 61, respectively, and LCDR1, LCDR2, and LCDR3 sequences of SEQ ID NOs: 72, 73, and 74 respectively;
(g) HCDR1, HCDR2, and HCDR3 sequences of SEQ ID NOs: 105, 106, and 107 respectively, and LCDR1, LCDR2, and LCDR3 sequences of SEQ ID NOs: 118, 119, 120, respectively;
(h) HCDR1, HCDR2, and HCDR3 sequences of SEQ ID NOs: 108, 109, and 110 respectively, and LCDR1, LCDR2, and LCDR3 sequences of SEQ ID NOs: 121, 122, 123, respectively;
(i) HCDR1, HCDR2, and HCDR3 sequences of SEQ ID NOs: 111, 112, and 113 respectively, and LCDR1, LCDR2, and LCDR3 sequences of SEQ ID NOs: 124, 125, 126, respectively;
(j) HCDR1, HCDR2, and HCDR3 sequences of SEQ ID NOs: 157, 158, and 159, respectively, and LCDR1, LCDR2, and LCDR3 sequences of SEQ ID NOs: 170, 171, 172, respectively;
(k) HCDR1, HCDR2, and HCDR3 sequences of SEQ ID NOs: 160, 161, and 162, respectively, and LCDR1, LCDR2, and LCDR3 sequences of SEQ ID NOs: 173, 174, 175, respectively;
(l) HCDR1, HCDR2, and HCDR3 sequences of SEQ ID NOs: 163, 164, and 165, respectively, and LCDR1, LCDR2, and LCDR3 sequences of SEQ ID NOs: 176, 177, 178, respectively;
(m) HCDR1, HCDR2, and HCDR3 sequences of SEQ ID NOs: 209, 210, and 211, respectively, and LCDR1, LCDR2, and LCDR3 sequences of SEQ ID NOs: 222, 223, and 224, respectively;
(n) HCDR1, HCDR2, and HCDR3 sequences of SEQ ID NOs: 212, 213, and 214, respectively, and LCDR1, LCDR2, and LCDR3 sequences of SEQ ID NOs: 225, 226, and 227, respectively;
(o) HCDR1, HCDR2, and HCDR3 sequences of SEQ ID NOs: 215, 216, and 217 respectively, and LCDR1, LCDR2, and LCDR3 sequences of SEQ ID NOs: 228, 229, and 230, respectively;
(p) HCDR1, HCDR2, and HCDR3 sequences of SEQ ID NOs: 261, 262, and 263, respectively, and LCDR1, LCDR2, and LCDR3 sequences of SEQ ID NOs: 274, 275, and 276, respectively;
(q) HCDR1, HCDR2, and HCDR3 sequences of SEQ ID NOs: 264, 265, and 266, respectively, and LCDR1, LCDR2, and LCDR3 sequences of SEQ ID NOs: 277, 278, and 279, respectively;
(r) HCDR1, HCDR2, and HCDR3 sequences of SEQ ID NOs: 267, 268, and 269, respectively, and LCDR1, LCDR2, and LCDR3 sequences of SEQ ID NOs: 280, 281, and 282, respectively;
(s) HCDR1, HCDR2, and HCDR3 sequences of SEQ ID NOs: 313, 314, and 315, respectively, and LCDR1, LCDR2, and LCDR3 sequences of SEQ ID NOs: 326, 327, and 328, respectively;
(t) HCDR1, HCDR2, and HCDR3 sequences of SEQ ID NOs: 316, 317, and 318, respectively, and LCDR1, LCDR2, and LCDR3 sequences of SEQ ID NOs: 329, 330, and 331, respectively;
(u) HCDR1, HCDR2, and HCDR3 sequences of SEQ ID NOs: 319, 320, and 321, respectively, and LCDR1, LCDR2, and LCDR3 sequences of SEQ ID NOs: 332, 333, and 334, respectively;
(v) HCDR1, HCDR2, and HCDR3 sequences of SEQ ID NOs: 365, 366, and 367, respectively, and LCDR1, LCDR2, and LCDR3 sequences of SEQ ID NOs: 378, 379, and 380, respectively;
(w) HCDR1, HCDR2, and HCDR3 sequences of SEQ ID NOs: 368, 369, and 370, respectively, and LCDR1, LCDR2, and LCDR3 sequences of SEQ ID NOs: 381, 382, and 383, respectively;
(x) HCDR1, HCDR2, and HCDR3 sequences of SEQ ID NOs: 371, 372, and 373, respectively, and LCDR1, LCDR2, and LCDR3 sequences of SEQ ID NOs: 384, 385, and 386, respectively;
(y) HCDR1, HCDR2, and HCDR3 sequences of SEQ ID NOs: 417, 418, and 419, respectively, and LCDR1, LCDR2, and LCDR3 sequences of SEQ ID NOs: 430, 431, and 432, respectively;
(z) HCDR1, HCDR2, and HCDR3 sequences of SEQ ID NOs: 420, 421, and 422, respectively, and LCDR1, LCDR2, and LCDR3 sequences of SEQ ID NOs: 433, 434, and 435, respectively;
(aa) HCDR1, HCDR2, and HCDR3 sequences of SEQ ID NOs: 423, 424, and 425, respectively, and LCDR1, LCDR2, and LCDR3 sequences of SEQ ID NOs: 436, 437, and 438, respectively;
(bb) HCDR1, HCDR2, and HCDR3 sequences of SEQ ID NOs: 469, 470, and 471, respectively, and LCDR1, LCDR2, and LCDR3 sequences of SEQ ID NOs: 482, 483, and 484, respectively;
(cc) HCDR1, HCDR2, and HCDR3 sequences of SEQ ID NOs: 472, 473, and 474, respectively, and LCDR1, LCDR2, and LCDR3 sequences of SEQ ID NOs: 485, 486, and 487, respectively;
(dd) HCDR1, HCDR2, and HCDR3 sequences of SEQ ID NOs: 475, 476, and 477, respectively, and LCDR1, LCDR2, and LCDR3 sequences of SEQ ID NOs: 488, 489, and 490, respectively;
(ee) HCDR1, HCDR2, and HCDR3 sequences of SEQ ID NOs: 521, 522, and 523, respectively, and LCDR1, LCDR2, and LCDR3 sequences of SEQ ID NOs: 534, 535, and 536, respectively;
(ff) HCDR1, HCDR2, and HCDR3 sequences of SEQ ID NOs: 524, 525, and 526, respectively, and LCDR1, LCDR2, and LCDR3 sequences of SEQ ID NOs: 537, 538, and 539, respectively;
(gg) HCDR1, HCDR2, and HCDR3 sequences of SEQ ID NOs: 527, 528, and 529, respectively, and LCDR1, LCDR2, and LCDR3 sequences of SEQ ID NOs: 540, 541, and 542, respectively;
(hh) HCDR1, HCDR2, and HCDR3 sequences of SEQ ID NOs: 547, 548, and 549, respectively, and LCDR1, LCDR2, and LCDR3 sequences of SEQ ID NOs: 560, 561, and 562, respectively;
(ii) HCDR1, HCDR2, and HCDR3 sequences of SEQ ID NOs: 550, 551, and 552, respectively, and LCDR1, LCDR2, and LCDR3 sequences of SEQ ID NOs: 563, 564, and 565, respectively;
(jj) HCDR1, HCDR2, and HCDR3 sequences of SEQ ID NOs: 553, 554, and 555, respectively, and LCDR1, LCDR2, and LCDR3 sequences of SEQ ID NOs: 566, 567, and 568, respectively;
(kk) HCDR1, HCDR2, and HCDR3 sequences of SEQ ID NOs: 573, 574, and 575, respectively, and LCDR1, LCDR2, and LCDR3 sequences of SEQ ID NOs: 586, 587, and 588, respectively;
(ll) HCDR1, HCDR2, and HCDR3 sequences of SEQ ID NOs: 576, 577, and 578, respectively, and LCDR1, LCDR2, and LCDR3 sequences of SEQ ID NOs: 589, 590, and 591, respectively;
(mm) HCDR1, HCDR2, and HCDR3 sequences of SEQ ID NOs: 579, 580, and 581, respectively, and LCDR1, LCDR2, and LCDR3 sequences of SEQ ID NOs: 592, 593, and 594, respectively;
(nn) HCDR1, HCDR2, and HCDR3 sequences of SEQ ID NOs: 625, 626, and 627, respectively, and LCDR1, LCDR2, and LCDR3 sequences of SEQ ID NOs: 638, 639, and 640, respectively;
(oo) HCDR1, HCDR2, and HCDR3 sequences of SEQ ID NOs: 628, 629, and 630, respectively, and LCDR1, LCDR2, and LCDR3 sequences of SEQ ID NOs: 641, 642, and 643, respectively; or
(pp) HCDR1, HCDR2, and HCDR3 sequences of SEQ ID NOs: 631, 632, and 633, respectively, and LCDR1, LCDR2, and LCDR3 sequences of SEQ ID NOs: 644, 645, and 646, respectively.

6. The isolated antibody or antigen-binding fragment thereof of claim 1, comprising:
(a) A VH sequence of SEQ ID NO: 10 and a VL sequence of SEQ ID NO: 23;
(b) A VH sequence of SEQ ID NO: 62 and a VL sequence of SEQ ID NO: 75;
(c) A VH sequence of SEQ ID NO: 114 and VL sequence of SEQ ID NO: 127;
(d) A VH sequence of SEQ ID NO: 166 and a VL sequence of SEQ ID NO: 179;
(e) A VH sequence of SEQ ID NO: 218 and a VL sequence of SEQ ID NO: 231;
(f) A VH sequence of SEQ ID NO: 270 and a VL sequence of SEQ ID NO: 283;
(g) A VH sequence of SEQ ID NO: 322 and a VL sequence of SEQ ID NO: 335;
(h) A VH sequence of SEQ ID NO: 374 and VL sequence of SEQ ID NO: 387;
(i) A VH sequence of SEQ ID NO: 426 and a VL sequence of SEQ ID NO: 439;
(j) A VH sequence of SEQ ID NO: 478 and a VL sequence of SEQ ID NO: 491;
(k) A VH sequence of SEQ ID NO: 530 and a VL sequence of SEQ ID NO: 543;
(l) A VH sequence of SEQ ID NO: 556 and a VL sequence of SEQ ID NO: 569;
(m) A VH sequence of SEQ ID NO: 582 and a VL sequence of SEQ ID NO: 595; or
(n) A VH sequence of SEQ ID NO: 634 and a VL sequence of SEQ ID NO: 647.

7. The isolated antibody or antigen-binding fragment thereof of claim 1, comprising:
(a) A heavy chain sequence of SEQ ID NO: 12; and a light chain sequence of SEQ ID NO: 25;
(b) A heavy chain sequence of SEQ ID NO: 64; and a light chain sequence of SEQ ID NO: 77;
(c) A heavy chain sequence of SEQ ID NO: 116; and a light chain sequence of SEQ ID NO: 129;
(d) A heavy chain sequence of SEQ ID NO: 168; and a light chain sequence of SEQ ID NO: 181;
(e) A heavy chain sequence of SEQ ID NO: 220; and a light chain sequence of SEQ ID NO: 233;
(f) A heavy chain sequence of SEQ ID NO: 272; and a light chain sequence of SEQ ID NO: 285;
(g) A heavy chain sequence of SEQ ID NO: 324; and a light chain sequence of SEQ ID NO: 337;
(h) A heavy chain sequence of SEQ ID NO: 376; and a light chain sequence of SEQ ID NO: 389;
(i) A heavy chain sequence of SEQ ID NO: 428; and a light chain sequence of SEQ ID NO: 441;
(j) A heavy chain sequence of SEQ ID NO: 480; and a light chain sequence of SEQ ID NO: 493;
(k) A heavy chain sequence of SEQ ID NO: 584; and a light chain sequence of SEQ ID NO: 597; or
(l) A heavy chain sequence of SEQ ID NO: 636; and a light chain sequence of SEQ ID NO: 649.

8. The isolated antibody or antigen-binding fragment thereof of claim 1, comprising:
(a) A heavy chain sequence of SEQ ID NO: 38; and a light chain sequence of SEQ ID NO: 51;
(b) A heavy chain sequence of SEQ ID NO: 90; and a light chain sequence of SEQ ID NO: 103;
(c) A heavy chain sequence of SEQ ID NO: 142; and a light chain sequence of SEQ ID NO: 155;
(d) A heavy chain sequence of SEQ ID NO: 194; and a light chain sequence of SEQ ID NO: 207;
(e) A heavy chain sequence of SEQ ID NO: 246; and a light chain sequence of SEQ ID NO: 259;
(f) A heavy chain sequence of SEQ ID NO: 298; and a light chain sequence of SEQ ID NO: 311;
(g) A heavy chain sequence of SEQ ID NO: 350; and a light chain sequence of SEQ ID NO: 363;
(h) A heavy chain sequence of SEQ ID NO: 402; and a light chain sequence of SEQ ID NO: 415;
(i) A heavy chain sequence of SEQ ID NO: 454; and a light chain sequence of SEQ ID NO: 467;
(j) A heavy chain sequence of SEQ ID NO: 506; and a light chain sequence of SEQ ID NO: 519;
(k) A heavy chain sequence of SEQ ID NO: 532; and a light chain sequence of SEQ ID NO: 545;
(l) A heavy chain sequence of SEQ ID NO: 558; and a light chain sequence of SEQ ID NO: 571;
(m) A heavy chain sequence of SEQ ID NO: 610; and a light chain sequence of SEQ ID NO: 623; or
(n) A heavy chain sequence of SEQ ID NO: 662; and a light chain sequence of SEQ ID NO: 675.

9. The isolated antibody or antigen binding fragment thereof of claim 1, comprising:
(a) a HCDR1 comprising the amino acid sequence selected from SEQ ID NOs: 157, 160, or 163;
(b) a HCDR2 comprising the amino acid sequence selected from SEQ ID NOs: 158, 161, or 164;
(c) a HCDR3 comprising the amino acid sequence selected from SEQ ID NOs: 159, 315, 367, 419, 471, 523, 549, 575, or 627;
(d) a LCDR1 comprising the amino acid sequence selected from SEQ ID NOs: 170, 173, or 176;
(e) a LCDR2 comprising the amino acid sequence selected from SEQ ID NOs: 171, 174, or 177; and
(f) a LCDR3 comprising the amino acid sequence of SEQ ID NO: 172.

10. The isolated antibody or antigen binding fragment thereof of claim 1, comprising:
(a) a HCDR1 comprising the amino acid sequence selected from SEQ ID NOs: 157, 160, or 163;
(b) a HCDR2 comprising the amino acid sequence selected from SEQ ID NOs: 158, 161, or 164;
(c) a HCDR3 comprising the amino acid sequence EQX₁PX₂X₃GX₄GGX₅PX₆EAMDV (SEQ ID NO: 683), wherein X₁ is D or S, X₂ is E or S, X₃ is Y, F, A, or S; X₄ is Y or F; X₅ is F or Y, and X₆ is Y or F;
(d) a LCDR1 comprising the amino acid sequence selected from SEQ ID NOs: 170, 173, or 176;
(e) a LCDR2 comprising the amino acid sequence selected from SEQ ID NOs: 171, 174, or 177; and
(f) a LCDR3 comprising the amino acid sequence of SEQ ID NO: 172.

11. The isolated antibody or antigen-binding fragment thereof of claim 10, comprising:
(a) a HCDR1 comprising the amino acid sequence selected from SEQ ID NO: 157, 160, or 163;
(b) a HCDR2 comprising the amino acid sequence selected from SEQ ID NO: 158, 161, or 164;
(c) a HCDR3 comprising the amino acid sequence of SEQ ID NO: 159, 315, 367, or 419;
(d) a LCDR1 comprising the amino acid sequence selected from SEQ ID NOs: 170, 173, or 176;
(e) a LCDR2 comprising the amino acid sequence selected from SEQ ID NOs: 171, 174, or 177; and
(f) a LCDR3 comprising the amino acid sequence of SEQ ID NO: 172.

12. The isolated antibody or antigen-binding fragment thereof of claim 10, comprising:
(a) a HCDR1 comprising the amino acid sequence selected from SEQ ID NO: 417;
(b) a HCDR2 comprising the amino acid sequence selected from SEQ ID NO: 418;
(c) a HCDR3 comprising the amino acid sequence of SEQ ID NO: 419;
(d) a LCDR1 comprising the amino acid sequence selected from SEQ ID NOs: 430;
(e) a LCDR2 comprising the amino acid sequence selected from SEQ ID NOs: 431; and
(f) a LCDR3 comprising the amino acid sequence of SEQ ID NO: 432.

13. The antibody or antigen-binding fragment thereof of claim 1 comprising:
(a) a HCDR1 comprising the amino acid sequence selected from SEQ ID NO: 417;
(b) a HCDR2 comprising the amino acid sequence selected from SEQ ID NO: 418;
(c) a HCDR3 comprising the amino acid sequence of SEQ ID NO: 419;
(d) a LCDR1 comprising the amino acid sequence selected from SEQ ID NOs: 430;
(e) a LCDR2 comprising the amino acid sequence selected from SEQ ID NOs: 431; and
(f) a LCDR3 comprising the amino acid sequence of SEQ ID NO: 432;
wherein the antibody or antigen-binding fragment thereof is afucosylated.

14. The afucosylated antibody or antigen-binding fragment thereof of claim 13, comprising a variable heavy chain region comprising the amino acid sequence of SEQ ID NO: 426 and a light chain variable region comprising the amino acid sequence of SEQ ID NO: 441.

15. The afucosylated antibody or antigen-binding fragment thereof of claim 13, comprising a heavy chain comprising the amino acid sequence of SEQ ID NO: 428 and a light chain comprising the amino acid sequence of SEQ ID NO: 441.

16. An isolated antibody or antigen-binding fragment thereof, wherein the antibody or antigen-binding fragment thereof comprises a heavy chain variable region comprising an amino acid sequence that is at least 90% identical to the amino acid sequence selected from the group consisting of SEQ ID NOs: 10, 62, 114, 166, 218, 270, 322, 374, 426, 478, 530, 556, 582, and 634; and a light chain variable region comprising an amino acid sequence that is at least 90% identical to the amino acid sequence selected from the group consisting of SEQ ID NOs: 23, 75, 127, 179, 231, 283, 335, 387, 439, 491, 543, 569, 595, and 647; wherein the antibody specifically binds to human CD32b protein.

17. The isolated antibody or antigen-binding fragment thereof of claim 16, wherein the antibody or antigen-binding fragment thereof comprises a heavy chain comprising an amino acid sequence that is at least 90% identical to the amino acid sequence selected from the group consisting of SEQ ID NOs: 12, 38, 64, 90, 116, 142, 168, 194, 220, 246, 272, 298, 324, 350, 376, 402, 428, 454, 480, 506, 532, 558, 584, 610, 636, and 662; and a light chain comprising an amino acid sequence that is at least 90% identical to the amino acid sequence selected from the group consisting of SEQ ID NOs: 25, 51, 77, 103, 129, 155, 181, 207, 233, 259, 285, 311, 337, 363, 389, 415, 441, 467, 493, 519, 545, 571, 597, 623, 649, and 675; wherein the antibody specifically binds to human CD32b protein.

18. The isolated antibody or antigen-binding fragment thereof of claim 1, wherein the antibody is afucosylated.

19. The isolated antibody or antigen-binding fragment thereof of claim 1, wherein the Fc portion of the antibody is modified to enhance ADCC activity.

20. The isolated antibody or antigen-binding fragment thereof of claim 1, wherein the antibody or antigen-binding fragment thereof selectively binds human CD32b over human CD32a.

21. The isolated antibody or antigen-binding fragment thereof of claim 1, wherein the antibody or antigen-binding fragment thereof is an IgG selected from the group consisting of an IgG1, an IgG2, an IgG3 and an IgG4.

22. The isolated antibody or antigen-binding fragment thereof of claim 1, wherein the isolated antibody or antigen-binding fragment is selected from the group consisting of: a monoclonal antibody, a chimeric antibody, a single chain antibody, a Fab and a scFv.

23. The isolated antibody or antigen-binding fragment thereof of claim 1, wherein the antibody or antigen-binding fragment thereof is chimeric, humanized or fully human.

24. The isolated antibody or antigen-binding fragment thereof of claim 1, wherein the isolated antibody or antigen-binding fragment inhibits binding of human CD32b to immunoglobulin Fc domains.

25. The isolated antibody or antigen-binding fragment thereof of claim 1, wherein the isolated antibody or antigen-binding fragment thereof is a component of an immunoconjugate.

26. A multivalent antibody, wherein one arm of the antibody comprises the isolated antibody or antigen-binding fragments of claim 1.

27. The multivalent antibody of claim 26, wherein the antibody is a bispecific antibody.

28. A composition comprising the isolated antibody or antigen-binding fragment thereof of claim 1, in combination with one or more additional antibodies that bind a cell surface antigen that is co-expressed with CD32b on a cell.

29. The composition of claim 28, wherein the cell surface antigen and CD32b are co-expressed on B cells.

30. The composition of claim 28, wherein the cell surface antigen is selected from the group consisting of CD20, CD38, CD52, CS1/SLAMF7, CD56, CD138, KiR, CD19, CD40, Thy-1, Ly-6, CD49, Fas, Cd95, APO-1, EGFR, HER2, CXCR4, HLA molecules, GM1, CD22, CD23, CD80, CD74, or DRD.

31. The composition of claim 28, wherein the cell surface antigen is selected from the group consisting of CD20, CD38, CS1/SLAMF7 and CD52.

32. The composition of claim 28, wherein the additional antibody is selected from the group consisting of rituximab, elotuzumab, ofatumumab, obinutumumab, daratumumab, and alemtuzumab.

33. The composition of claim 28 further comprising an additional therapeutic compound.

34. A composition comprising the isolated antibody or antigen-binding fragment thereof of claim 1 in combination with an additional therapeutic compound.

35. The composition of claim 34, wherein the additional therapeutic compound is an immunomodulator.

36. The composition of claim 35, wherein the immunomodulator is IL15 or the immunomodulator is an agonist of a costimulatory molecule selected from OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3, CD83 ligand, and STING.

37. The composition of claim 35, wherein the immunomodulator is an inhibitor molecule of a target selected from PD-1, PD-L1, PD-L2, CTLA-4, TIM-3, LAG-3, CEACAM-1, CEACAM-3, CEACAM-5, VISTA, BTLA, TIGIT, LAIR1, CD160, 2B4, TGFR beta, and IDO.

38. The composition of claim 34, wherein the additional therapeutic compound is selected from ofatumumab, ibrutinib, belinostat, romidepsin, brentuximab vedotin, obinutuzumab, pralatrexate, pentostatin, dexamethasone, idelalisib, ixazomib, liposomal doxyrubicin, pomalidomide, panobinostat, elotuzumab, daratumumab, alemtuzumab, thalidomide, and lenalidomide.

39. The composition of claim 33, wherein the additional therapeutic compound is selected from ibrutinib, belinostat, romidepsin, brentuximab vedotin, pralatrexate, pentostatin, dexamethasone, idelalisib, ixazomib, liposomal doxyrubicin, pomalidomide, panobinostat, thalidomide, and lenalidomide

40. A pharmaceutical composition comprising the isolated antibody or antigen-binding fragment thereof of claim 1, or a multivalent antibody comprising the antibody or antigen-binding fragment thereof of claim 1, and a pharmaceutically acceptable carrier.

41. (canceled)

42. A method of treating a CD32b-related condition in a subject in need thereof comprising administering to the subject a therapeutically effective amount of the antibody or antigen-binding fragment thereof of claim 1, or a multivalent antibody comprising the antibody or antigen-binding fragment thereof of claim 1.

43. (canceled)

44. (canceled)

45. (canceled)

46. The method of claim 42 wherein the CD32b-related condition is selected from B cell malignancies, Hodgkins lymphoma, Non-Hodgkins lymphoma, multiple myeloma, diffuse large B cell lymphoma, acute lymphocytic leukemia, chronic lymphocytic leukemia, small lymphocytic lymphoma, diffuse small cleaved cell lymphoma, MALT lymphoma, mantel cell lymphoma, marginal zone lymphoma, follicular lymphoma, or systemic light chain amyloidosis.

47. A nucleic acid encoding the antibody or antigen-binding fragment thereof of claim 1.

48. A vector comprising the nucleic acid of claim 47.

49. A host cell comprising the nucleic acid of claim 47, or comprising a vector comprising the nucleic acid of claim 47.

50. A method of producing the antibody or antigen-binding fragment thereof of claim 1, the method comprising: culturing a host cell expressing a nucleic acid encoding the antibody; and collecting the antibody from the culture.

51. (canceled)

52. A method of treating a patient who is resistant or refractory to treatment using an antibody that binds to a cell surface antigen that is co-expressed with CD32b on a cell, comprising co-administering the antibody with any one of the isolated anti-CD32b antibodies or an antigen-binding fragment thereof of claim 1, or a multivalent antibody comprising the antibody or antigen-binding fragment thereof of claim 1.

53. (canceled)

54. (canceled)

55. An isolated antibody or antigen binding fragment thereof that specifically binds to CD32b within the Fc binding domain of CD32b.

56. The isolated antibody or antigen binding fragment of claim 55, wherein the antibody binds within amino acid residues 107-123 (VLRCHSWKDKPLVKVTF (SEQ ID NO: 685)) of CD32b.

57. The isolated antibody or antigen binding fragment of claim 55, wherein the antibody prevents or reduces CD32b binding to the immunoglobulin Fc domain of a second antibody that binds to a tumor antigen co-expressed with CD32b on a B-cell.

58. The isolated antibody or antigen binding fragment of claim 57, wherein the second antibody binds to a tumor antigen selected from the group consisting of CD20, CD38, CD52, CS1/SLAMF7, CD56, CD138, KiR, CD19, CD40, Thy-1, Ly-6, CD49, Fas, Cd95, APO-1, EGFR, HER2, CXCR4, HLA molecules, GM1, CD22, CD23, CD80, CD74, or DRD.

59. The isolated antibody or antigen binding fragment of claim 57, wherein the second antibody binds to a tumor antigen selected from the group consisting of CD20, CD38, CS1/SLAMF7 and CD52.

60. The isolated antibody or antigen binding fragment of claim 57, wherein the second antibody is selected from the group consisting of rituximab, elotuzumab, ofatumumab, obinutumumab, daratumumab, and alemtuzumab.

61. The isolated antibody or antigen binding fragment of claim 55 comprising the antibody of claim 1.

62. An isolated antibody or antigen binding fragment thereof that specifically binds to CD32b and inhibits or reduces CD32b immunoreceptor tyrosine-based inhibition motif (ITIM) signaling mediated by a second antibody that binds to a tumor antigen co-expressed with CD32b on a B-cell.

63. A method of inhibiting or reducing CD32b ITIM signaling that is induced by administration of a therapeutic antibody that binds to a tumor antigen co-expressed with CD32b on a B-cell comprising administering an isolated antibody or antigen binding fragment thereof that specifically binds to the Fc binding domain of CD32b.

64. The method of claim 63, wherein the isolated antibody or antigen binding fragment thereof does not stimulate ITIM signaling.

65. The method of claim 63, wherein the therapeutic antibody binds to a tumor antigen selected from the group consisting of CD20, CD38, CD52, CS1/SLAMF7, CD56, CD138, KiR, CD19, CD40, Thy-1, Ly-6, CD49, Fas, Cd95, APO-1, EGFR, HER2, CXCR4, HLA molecules, GM1, CD22, CD23, CD80, CD74, or DRD.

66. The method of claim 63, wherein the therapeutic antibody binds to a tumor antigen selected from the group consisting of CD20, CD38, CS1/SLAMF7 and CD52.

67. The method of claim 63, wherein the therapeutic antibody is selected from the group consisting of rituximab, elotuzumab, ofatumumab, obinutumumab, daratumumab, and alemtuzumab.

68. (canceled)


SEQUENCE LISTING (687 sequences)

SEQ ID NO: 1 - protein, 10 aa, Artificial Sequence
  source: /note=“Description of Artificial Sequence Synthetic peptide”
GGTFSDYAIS

SEQ ID NO: 2 - protein, 17 aa, Artificial Sequence
  source: /note=“Description of Artificial Sequence Synthetic peptide”
GIIPISGTANYAQKFQG

SEQ ID NO: 3 - protein, 15 aa, Artificial Sequence
  source: /note=“Description of Artificial Sequence Synthetic peptide”
DHSSSSYDYQYGLAV

SEQ ID NO: 4 - protein, 5 aa, Artificial Sequence
  source: /note=“Description of Artificial Sequence Synthetic peptide”
DYAIS

SEQ ID NO: 5 - protein, 17 aa, Artificial Sequence
  source: /note=“Description of Artificial Sequence Synthetic peptide”
GIIPISGTANYAQKFQG

SEQ ID NO: 6 - protein, 15 aa, Artificial Sequence
  source: /note=“Description of Artificial Sequence Synthetic peptide”
DHSSSSYDYQYGLAV

SEQ ID NO: 7 - protein, 7 aa, Artificial Sequence
  source: /note=“Description of Artificial Sequence Synthetic peptide”
GGTFSDY

SEQ ID NO: 8 - protein, 6 aa, Artificial Sequence
  source: /note=“Description of Artificial Sequence Synthetic peptide”
IPISGT

SEQ ID NO: 9 - protein, 15 aa, Artificial Sequence
  source: /note=“Description of Artificial Sequence Synthetic peptide”
DHSSSSYDYQYGLAV

SEQ ID NO: 10 - protein, 124 aa, Artificial Sequence
  source: /note=“Description of Artificial Sequence Synthetic polypeptide”
QVQLVQSGAEVKKPGSSVKVSCKASGGTFSDYAISWVRQAPGQGLEWMGGIIPISGTANYAQKFQGRVTITADESTSTAYMELSSLRSEDTAVYYCARDHSSSSYDYQYGLAVWGQGTLVTVSS

SEQ ID NO: 11 - DNA, 372 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagcgactacgctattagctgggtcagacaggccccaggtcagggcctggagtggatgggcggaattatccctattagcggcaccgctaactacgctcagaaatttcagggtagagtgactatcaccgccgacgagtctactagcaccgcctatatggaactgtctagcctgagatcagaggacaccgccgtctactactgcgctagagatcactctagctctagctacgactatcagtacggcctggccgtgtggggtcagggcaccctggtcaccgtgtctagc

SEQ ID NO: 12 - protein, 454 aa, Artificial Sequence
  source: /note=“Description of Artificial Sequence Synthetic polypeptide”
QVQLVQSGAEVKKPGSSVKVSCKASGGTFSDYAISWVRQAPGQGLEWMGGIIPISGTANYAQKFQGRVTITADESTSTAYMELSSLRSEDTAVYYCARDHSSSSYDYQYGLAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1362 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagcgactacgctattagctgggtcagacaggccccaggtcagggcctggagtggatgggcggaattatccctattagcggcaccgctaactacgctcagaaatttcagggtagagtgactatcaccgccgacgagtctactagcaccgcctatatggaactgtctagcctgagatcagaggacaccgccgtctactactgcgctagagatcactctagctctagctacgactatcagtacggcctggccgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 14 - protein, 11 aa, Artificial Sequence
  source: /note=“Description of Artificial Sequence Synthetic peptide”
SGDKLGDYYVH

SEQ ID NO: 15 - protein, 7 aa, Artificial Sequence
  source: /note=“Description of Artificial Sequence Synthetic peptide”
QDSKRPS

SEQ ID NO: 16 - protein, 11 aa, Artificial Sequence
  source: /note=“Description of Artificial Sequence Synthetic peptide”
GATDLSPWSIV

SEQ ID NO: 17 - protein, 11 aa, Artificial Sequence
  source: /note=“Description of Artificial Sequence Synthetic peptide”
SGDKLGDYYVH

SEQ ID NO: 18 - protein, 7 aa, Artificial Sequence
  source: /note=“Description of Artificial Sequence Synthetic peptide”
QDSKRPS

SEQ ID NO: 19 - protein, 11 aa, Artificial Sequence
  source: /note=“Description of Artificial Sequence Synthetic peptide”
GATDLSPWSIV

SEQ ID NO: 20 - protein, 7 aa, Artificial Sequence
  source: /note=“Description of Artificial Sequence Synthetic peptide”
DKLGDYY

SEQ ID NO: 21 - protein, 3 aa, Artificial Sequence
  source: /note=“Description of Artificial Sequence Synthetic peptide”
QDS

SEQ ID NO: 22 - protein, 8 aa, Artificial Sequence
  source: /note=“Description of Artificial Sequence Synthetic peptide”
TDLSPWSI

SEQ ID NO: 23 - protein, 108 aa, Artificial Sequence
  source: /note=“Description of Artificial Sequence Synthetic polypeptide”
DIELTQPPSVSVSPGETASITCSGDKLGDYYVHWYQQKPGQAPVLVIYQDSKRPSGIPERFSGSNSGNTATLTISGTQAEDEADYYCGATDLSPWSIVFGGGTKLTVL

SEQ ID NO: 24 - DNA, 324 bp, Artificial Sequence
  source: /note=“Description of Artificial Sequence Synthetic polynucleotide”
gatatcgagctgactcagccccctagcgtcagcgtcagccctggcgagacagcctctatcacctgtagcggcgataagctgggcgactactacgtgcactggtatcagcagaagcccggtcaggcccccgtgctggtgatctatcaggactctaagcggcctagcggaatccccgagcggtttagcggctctaatagcggtaacaccgctaccctgactattagcggcactcaggccgaggacgaggccgactactactgcggcgctaccgacctgagcccctggtctatcgtgttcggcggaggcactaagctgaccgtgctg

SEQ ID NO: 25 - protein, 214 aa, Artificial Sequence
  source: /note=“Description of Artificial Sequence Synthetic polypeptide”
DIELTQPPSVSVSPGETASITCSGDKLGDYYVHWYQQKPGQAPVLVIYQDSKRPSGIPERFSGSNSGNTATLTISGTQAEDEADYYCGATDLSPWSIVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - DNA, 642 bp, Artificial Sequence
  source: /note=“Description of Artificial Sequence Synthetic polynucleotide”
gatatcgagctgactcagccccctagcgtcagcgtcagccctggcgagacagcctctatcacctgtagcggcgataagctgggcgactactacgtgcactggtatcagcagaagcccggtcaggcccccgtgctggtgatctatcaggactctaagcggcctagcggaatccccgagcggtttagcggctctaatagcggtaacaccgctaccctgactattagcggcactcaggccgaggacgaggccgactactactgcggcgctaccgacctgagcccctggtctatcgtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 27 - protein, 10 aa, Artificial Sequence
  source: /note=“Description of Artificial Sequence Synthetic peptide”
GGTFSDYAIS

SEQ ID NO: 28 - protein, 17 aa, Artificial Sequence
  source: /note=“Description of Artificial Sequence Synthetic peptide”
GIIPISGTANYAQKFQG

SEQ ID NO: 29 - protein, 15 aa, Artificial Sequence
  source: /note=“Description of Artificial Sequence Synthetic peptide”
DHSSSSYDYQYGLAV

SEQ ID NO: 30 - protein, 5 aa, Artificial Sequence
  source: /note=“Description of Artificial Sequence Synthetic peptide”
DYAIS

SEQ ID NO: 31 - protein, 17 aa, Artificial Sequence
  source: /note=“Description of Artificial Sequence Synthetic peptide”
GIIPISGTANYAQKFQG

SEQ ID NO: 32 - protein, 15 aa, Artificial Sequence
  source: /note=“Description of Artificial Sequence Synthetic peptide”
DHSSSSYDYQYGLAV

SEQ ID NO: 33 - protein, 7 aa, Artificial Sequence
  source: /note=“Description of Artificial Sequence Synthetic peptide”
GGTFSDY

SEQ ID NO: 34 - protein, 6 aa, Artificial Sequence
  source: /note=“Description of Artificial Sequence Synthetic peptide”
IPISGT

SEQ ID NO: 35 - protein, 15 aa, Artificial Sequence
  source: /note=“Description of Artificial Sequence Synthetic peptide”
DHSSSSYDYQYGLAV

SEQ ID NO: 36 - protein, 124 aa, Artificial Sequence; identical to SEQ ID NO: 10
  source: /note=“Description of Artificial Sequence Synthetic polypeptide”

SEQ ID NO: 37 - DNA, 372 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgttttctgactacgctatctcttgggtgcgccaggccccgggccagggcctcgagtggatgggcggtatcatcccgatctctggcactgcgaactacgcccagaaatttcagggccgggtgaccattaccgccgatgaaagcaccagcaccgcctatatggaactgagcagcctgcgcagcgaagatacggccgtgtattattgcgcgcgtgaccattcttcttcttcttacgactaccagtacggtctggctgtttggggccaaggcaccctggtgactgttagctca

SEQ ID NO: 38 - protein, 454 aa, Artificial Sequence
  source: /note=“Description of Artificial Sequence Synthetic polypeptide”
QVQLVQSGAEVKKPGSSVKVSCKASGGTFSDYAISWVRQAPGQGLEWMGGIIPISGTANYAQKFQGRVTITADESTSTAYMELSSLRSEDTAVYYCARDHSSSSYDYQYGLAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 1362 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgttttctgactacgctatctcttgggtgcgccaggccccgggccagggcctcgagtggatgggcggtatcatcccgatctctggcactgcgaactacgcccagaaatttcagggccgggtgaccattaccgccgatgaaagcaccagcaccgcctatatggaactgagcagcctgcgcagcgaagatacggccgtgtattattgcgcgcgtgaccattcttcttcttcttacgactaccagtacggtctggctgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 40 - protein, 11 aa, Artificial Sequence
  source: /note=“Description of Artificial Sequence Synthetic peptide”
SGDKLGDYYVH

SEQ ID NO: 41 - protein, 7 aa, Artificial Sequence
  source: /note=“Description of Artificial Sequence Synthetic peptide”
QDSKRPS

SEQ ID NO: 42 - protein, 11 aa, Artificial Sequence
  source: /note=“Description of Artificial Sequence Synthetic peptide”
GATDLSPWSIV

SEQ ID NO: 43 - protein, 11 aa, Artificial Sequence
  source: /note=“Description of Artificial Sequence Synthetic peptide”
SGDKLGDYYVH

SEQ ID NO: 44 - protein, 7 aa, Artificial Sequence
  source: /note=“Description of Artificial Sequence Synthetic peptide”
QDSKRPS

SEQ ID NO: 45 - protein, 11 aa, Artificial Sequence
  source: /note=“Description of Artificial Sequence Synthetic peptide”
GATDLSPWSIV

SEQ ID NO: 46 - protein, 7 aa, Artificial Sequence
  source: /note=“Description of Artificial Sequence Synthetic peptide”
DKLGDYY

SEQ ID NO: 47 - protein, 3 aa, Artificial Sequence
  source: /note=“Description of Artificial Sequence Synthetic peptide”
QDS

SEQ ID NO: 48 - protein, 8 aa, Artificial Sequence
  source: /note=“Description of Artificial Sequence Synthetic peptide”
TDLSPWSI

SEQ ID NO: 49 - protein, 108 aa, Artificial Sequence; identical to SEQ ID NO: 23
  source: /note=“Description of Artificial Sequence Synthetic polypeptide”

SEQ ID NO: 50 - DNA, 324 bp, Artificial Sequence
  source: /note=“Description of Artificial Sequence Synthetic polynucleotide”
gatatcgaactgacccagccgccgagcgtgagcgtgagcccgggcgagaccgcgagcattacctgtagcggcgataaactgggtgactactacgttcattggtaccagcagaaaccgggccaggcgccggtgctggtgatctaccaggactctaaacgtccgagcggcatcccggaacgttttagcggatccaacagcggcaacaccgcgaccctgaccattagcggcacccaggcggaagacgaagcggattattactgcggtgctactgacctgtctccgtggtctatcgtgtttggcggcggcacgaagttaaccgtccta

SEQ ID NO: 51 - protein, 214 aa, Artificial Sequence; identical to SEQ ID NO: 25
  source: /note=“Description of Artificial Sequence Synthetic polypeptide”

SEQ ID NO: 52 - DNA, 642 bp, Artificial Sequence
  source: /note=“Description of Artificial Sequence Synthetic polynucleotide”
gatatcgaactgacccagccgccgagcgtgagcgtgagcccgggcgagaccgcgagcattacctgtagcggcgataaactgggtgactactacgttcattggtaccagcagaaaccgggccaggcgccggtgctggtgatctaccaggactctaaacgtccgagcggcatcccggaacgttttagcggatccaacagcggcaacaccgcgaccctgaccattagcggcacccaggcggaagacgaagcggattattactgcggtgctactgacctgtctccgtggtctatcgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 53 - protein, 10 aa, Artificial Sequence
  source: /note=“Description of Artificial Sequence Synthetic peptide”
GGTFSSYAIS

SEQ ID NO: 54 - protein, 17 aa, Artificial Sequence
  source: /note=“Description of Artificial Sequence Synthetic peptide”
GIIPVLGTANYAQKFQG

SEQ ID NO: 55 - protein, 8 aa, Artificial Sequence
  source: /note=“Description of Artificial Sequence Synthetic peptide”
VPTDYFDY

SEQ ID NO: 56 - protein, 5 aa, Artificial Sequence
  source: /note=“Description of Artificial Sequence Synthetic peptide”
SYAIS

SEQ ID NO: 57 - protein, 17 aa, Artificial Sequence
  source: /note=“Description of Artificial Sequence Synthetic peptide”
GIIPVLGTANYAQKFQG

SEQ ID NO: 58 - protein, 8 aa, Artificial Sequence
  source: /note=“Description of Artificial Sequence Synthetic peptide”
VPTDYFDY

SEQ ID NO: 59 - protein, 7 aa, Artificial Sequence
  source: /note=“Description of Artificial Sequence Synthetic peptide”
GGTFSSY

SEQ ID NO: 60 - protein, 6 aa, Artificial Sequence
  source: /note=“Description of Artificial Sequence Synthetic peptide”
IPVLGT

SEQ ID NO: 61 - protein, 8 aa, Artificial Sequence
  source: /note=“Description of Artificial Sequence Synthetic peptide”
VPTDYFDY

SEQ ID NO: 62 - protein, 117 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPVLGTANYAQKFQGRVTITADESTSTAYMELSSLRSEDTAVYYCARVPTDYFDYWGQGTLVTVSS

SEQ ID NO: 63 - DNA, 351 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcaccttctctagctacgctattagctgggtcagacaggccccaggtcagggcctggagtggatgggcggaattatccccgtgctgggcaccgctaactacgctcagaaatttcagggtagagtgactatcaccgccgacgagtctactagcaccgcctatatggaactgtctagcctgagatcagaggacaccgccgtctactactgcgctagagtgcctaccgactacttcgactactggggtcagggcaccctggtcaccgtgtctagc

SEQ ID NO: 64 - protein, 447 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPVLGTANYAQKFQGRVTITADESTSTAYMELSSLRSEDTAVYYCARVPTDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1341 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcaccttctctagctacgctattagctgggtcagacaggccccaggtcagggcctggagtggatgggcggaattatccccgtgctgggcaccgctaactacgctcagaaatttcagggtagagtgactatcaccgccgacgagtctactagcaccgcctatatggaactgtctagcctgagatcagaggacaccgccgtctactactgcgctagagtgcctaccgactacttcgactactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 66 - protein, 11 aa, Artificial Sequence
  source: /note=“Description of Artificial Sequence Synthetic peptide”
SGDNLGSKYVH

SEQ ID NO: 67 - protein, 7 aa, Artificial Sequence
  source: /note=“Description of Artificial Sequence Synthetic peptide”
DDNKRPS

SEQ ID NO: 68 - protein, 9 aa, Artificial Sequence
  source: /note=“Description of Artificial Sequence Synthetic peptide”
QSWTLGNWV

SEQ ID NO: 69 - protein, 11 aa, Artificial Sequence
  source: /note=“Description of Artificial Sequence Synthetic peptide”
SGDNLGSKYVH

SEQ ID NO: 70 - protein, 7 aa, Artificial Sequence
  source: /note=“Description of Artificial Sequence Synthetic peptide”
DDNKRPS

SEQ ID NO: 71 - protein, 9 aa, Artificial Sequence
  source: /note=“Description of Artificial Sequence Synthetic peptide”
QSWTLGNWV

SEQ ID NO: 72 - protein, 7 aa, Artificial Sequence
  source: /note=“Description of Artificial Sequence Synthetic peptide”
DNLGSKY

SEQ ID NO: 73 - protein, 3 aa, Artificial Sequence
  source: /note=“Description of Artificial Sequence Synthetic peptide”
DDN

SEQ ID NO: 74 - protein, 6 aa, Artificial Sequence
  source: /note=“Description of Artificial Sequence Synthetic peptide”
WTLGNW

SEQ ID NO: 75 - protein, 106 aa, Artificial Sequence
  source: /note=“Description of Artificial Sequence Synthetic polypeptide”
DIELTQPPSVSVSPGQTASITCSGDNLGSKYVHWYQQKPGQAPVLVIYDDNKRPSGIPERFSGSNSGNTATLTISGTQAEDEADYYCQSWTLGNWVFGGGTKLTVL

SEQ ID NO: 76 - DNA, 318 bp, Artificial Sequence
  source: /note=“Description of Artificial Sequence Synthetic polynucleotide”
gatatcgagctgactcagccccctagcgtcagcgtcagccctggtcagaccgcctctatcacctgtagcggcgataacctgggctctaaatacgtgcactggtatcagcagaagcccggtcaggcccccgtgctggtgatctacgacgataacaagcggcctagcggaatccccgagcggtttagcggctctaatagcggtaacaccgctaccctgactattagcggcactcaggccgaggacgaggccgactactactgtcagtcctggaccctgggcaactgggtgttcggcggaggcactaagctgaccgtgctg

SEQ ID NO: 77 - protein, 212 aa, Artificial Sequence
  source: /note=“Description of Artificial Sequence Synthetic polypeptide”
DIELTQPPSVSVSPGQTASITCSGDNLGSKYVHWYQQKPGQAPVLVIYDDNKRPSGIPERFSGSNSGNTATLTISGTQAEDEADYYCQSWTLGNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 78 - DNA, 636 bp, Artificial Sequence
  source: /note=“Description of Artificial Sequence Synthetic polynucleotide”
gatatcgagctgactcagccccctagcgtcagcgtcagccctggtcagaccgcctctatcacctgtagcggcgataacctgggctctaaatacgtgcactggtatcagcagaagcccggtcaggcccccgtgctggtgatctacgacgataacaagcggcctagcggaatccccgagcggtttagcggctctaatagcggtaacaccgctaccctgactattagcggcactcaggccgaggacgaggccgactactactgtcagtcctggaccctgggcaactgggtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 79 - protein, 10 aa, Artificial Sequence
  source: /note=“Description of Artificial Sequence Synthetic peptide”
GGTFSSYAIS

SEQ ID NO: 80 - protein, 17 aa, Artificial Sequence
  source: /note=“Description of Artificial Sequence Synthetic peptide”
GIIPVLGTANYAQKFQG

SEQ ID NO: 81 - protein, 8 aa, Artificial Sequence
  source: /note=“Description of Artificial Sequence Synthetic peptide”
VPTDYFDY

SEQ ID NO: 82 - protein, 5 aa, Artificial Sequence
  source: /note=“Description of Artificial Sequence Synthetic peptide”
SYAIS

SEQ ID NO: 83 - protein, 17 aa, Artificial Sequence
  source: /note=“Description of Artificial Sequence Synthetic peptide”
GIIPVLGTANYAQKFQG

SEQ ID NO: 84 - protein, 8 aa, Artificial Sequence
  source: /note=“Description of Artificial Sequence Synthetic peptide”
VPTDYFDY

SEQ ID NO: 85 - protein, 7 aa, Artificial Sequence
  source: /note=“Description of Artificial Sequence Synthetic peptide”
GGTFSSY

SEQ ID NO: 86 - protein, 6 aa, Artificial Sequence
  source: /note=“Description of Artificial Sequence Synthetic peptide”
IPVLGT

SEQ ID NO: 87 - protein, 8 aa, Artificial Sequence
  source: /note=“Description of Artificial Sequence Synthetic peptide”
VPTDYFDY

SEQ ID NO: 88 - protein, 117 aa, Artificial Sequence; identical to SEQ ID NO: 62
  source: /note=“Description of Artificial Sequence Synthetic polypeptide”

SEQ ID NO: 89 - DNA, 351 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgttttcttcttacgctatctcttgggtgcgccaggccccgggccagggcctcgagtggatgggcggtatcatcccggttctgggcactgcgaactacgcccagaaatttcagggccgggtgaccattaccgccgatgaaagcaccagcaccgcctatatggaactgagcagcctgcgcagcgaagatacggccgtgtattattgcgcgcgtgttccgactgactacttcgattactggggccaaggcaccctggtgactgttagctca

SEQ ID NO: 90 - protein, 447 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPVLGTANYAQKFQGRVTITADESTSTAYMELSSLRSEDTAVYYCARVPTDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1341 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgttttcttcttacgctatctcttgggtgcgccaggccccgggccagggcctcgagtggatgggcggtatcatcccggttctgggcactgcgaactacgcccagaaatttcagggccgggtgaccattaccgccgatgaaagcaccagcaccgcctatatggaactgagcagcctgcgcagcgaagatacggccgtgtattattgcgcgcgtgttccgactgactacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 92 - protein, 11 aa, Artificial Sequence
  source: /note=“Description of Artificial Sequence Synthetic peptide”
SGDNLGSKYVH

SEQ ID NO: 93 - protein, 7 aa, Artificial Sequence
  source: /note=“Description of Artificial Sequence Synthetic peptide”
DDNKRPS

SEQ ID NO: 94 - protein, 9 aa, Artificial Sequence
  source: /note=“Description of Artificial Sequence Synthetic peptide”
QSWTLGNWV

SEQ ID NO: 95 - protein, 11 aa, Artificial Sequence
  source: /note=“Description of Artificial Sequence Synthetic peptide”
SGDNLGSKYVH

SEQ ID NO: 96 - protein, 7 aa, Artificial Sequence
  source: /note=“Description of Artificial Sequence Synthetic peptide”
DDNKRPS

SEQ ID NO: 97 - protein, 9 aa, Artificial Sequence
  source: /note=“Description of Artificial Sequence Synthetic peptide”
QSWTLGNWV

SEQ ID NO: 98 - protein, 7 aa, Artificial Sequence
  source: /note=“Description of Artificial Sequence Synthetic peptide”
DNLGSKY

SEQ ID NO: 99 - protein, 3 aa, Artificial Sequence
  source: /note=“Description of Artificial Sequence Synthetic peptide”
DDN

SEQ ID NO: 100 - protein, 6 aa, Artificial Sequence
  source: /note=“Description of Artificial Sequence Synthetic peptide”
WTLGNW

SEQ ID NO: 101 - protein, 106 aa, Artificial Sequence; identical to SEQ ID NO: 75
  source: /note=“Description of Artificial Sequence Synthetic polypeptide”

SEQ ID NO: 102 - DNA, 318 bp, Artificial Sequence
  source: /note=“Description of Artificial Sequence Synthetic polynucleotide”
gatatcgaactgacccagccgccgagcgtgagcgtgagcccgggccagaccgcgagcattacctgtagcggcgataacctgggttctaaatacgttcattggtaccagcagaaaccgggccaggcgccggtgctggtgatctacgacgacaacaaacgtccgagcggcatcccggaacgttttagcggatccaacagcggcaacaccgcgaccctgaccattagcggcacccaggcggaagacgaagcggattattactgccagtcttggactctgggtaactgggtgtttggcggcggcacgaagttaaccgtccta

SEQ ID NO: 103 - protein, 212 aa, Artificial Sequence; identical to SEQ ID NO: 77
  source: /note=“Description of Artificial Sequence Synthetic polypeptide”

SEQ ID NO: 104 - DNA, 636 bp, Artificial Sequence
  source: /note=“Description of Artificial Sequence Synthetic polynucleotide”
gatatcgaactgacccagccgccgagcgtgagcgtgagcccgggccagaccgcgagcattacctgtagcggcgataacctgggttctaaatacgttcattggtaccagcagaaaccgggccaggcgccggtgctggtgatctacgacgacaacaaacgtccgagcggcatcccggaacgttttagcggatccaacagcggcaacaccgcgaccctgaccattagcggcacccaggcggaagacgaagcggattattactgccagtcttggactctgggtaactgggtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 105 - protein, 10 aa, Artificial Sequence
  source: /note=“Description of Artificial Sequence Synthetic peptide”
GGTFSDNAIS

SEQ ID NO: 106 - protein, 17 aa, Artificial Sequence
  source: /note=“Description of Artificial Sequence Synthetic peptide”
GINPDFGWANYAQKFQG

SEQ ID NO: 107 - protein, 7 aa, Artificial Sequence
  source: /note=“Description of Artificial Sequence Synthetic peptide”
DSSGMGY

SEQ ID NO: 108 - protein, 5 aa, Artificial Sequence
  source: /note=“Description of Artificial Sequence Synthetic peptide”
DNAIS

SEQ ID NO: 109 - protein, 17 aa, Artificial Sequence
  source: /note=“Description of Artificial Sequence Synthetic peptide”
GINPDFGWANYAQKFQG

SEQ ID NO: 110 - protein, 7 aa, Artificial Sequence
  source: /note=“Description of Artificial Sequence Synthetic peptide”
DSSGMGY

SEQ ID NO: 111 - protein, 7 aa, Artificial Sequence
  source: /note=“Description of Artificial Sequence Synthetic peptide”
GGTFSDN

SEQ ID NO: 112 - protein, 6 aa, Artificial Sequence
  source: /note=“Description of Artificial Sequence Synthetic peptide”
NPDFGW

SEQ ID NO: 113 - protein, 7 aa, Artificial Sequence
  source: /note=“Description of Artificial Sequence Synthetic peptide”
DSSGMGY

SEQ ID NO: 114 - protein, 116 aa, Artificial Sequence
  source: /note=“Description of Artificial Sequence Synthetic polypeptide”
QVQLVQSGAEVKKPGSSVKVSCKASGGTFSDNAISWVRQAPGQGLEWMGGINPDFGWANYAQKFQGRVTITADESTSTAYMELSSLRSEDTAVYYCARDSSGMGYWGQGTLVTVSS

SEQ ID NO: 115 - DNA, 348 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagcgataacgctattagctgggtcagacaggccccaggtcagggcctggagtggatgggcgggattaaccccgacttcggctgggctaactacgctcagaaatttcagggtagagtgactatcaccgccgacgagtctactagcaccgcctatatggaactgtctagcctgagatcagaggacaccgccgtctactactgcgctagggactctagcggaatgggctactggggtcagggcaccctggtcaccgtgtctagc

SEQ ID NO: 116 - protein, 446 aa, Artificial Sequence
  source: /note=“Description of Artificial Sequence Synthetic polypeptide”
QVQLVQSGAEVKKPGSSVKVSCKASGGTFSDNAISWVRQAPGQGLEWMGGINPDFGWANYAQKFQGRVTITADESTSTAYMELSSLRSEDTAVYYCARDSSGMGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - DNA, 1338 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagcgataacgctattagctgggtcagacaggccccaggtcagggcctggagtggatgggcgggattaaccccgacttcggctgggctaactacgctcagaaatttcagggtagagtgactatcaccgccgacgagtctactagcaccgcctatatggaactgtctagcctgagatcagaggacaccgccgtctactactgcgctagggactctagcggaatgggctactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 118 - protein, 11 aa, Artificial Sequence
  source: /note=“Description of Artificial Sequence Synthetic peptide”
RASQDISSYLN

SEQ ID NO: 119 - protein, 7 aa, Artificial Sequence
  source: /note=“Description of Artificial Sequence Synthetic peptide”
DASTLQS

SEQ ID NO: 120 - protein, 10 aa, Artificial Sequence
  source: /note=“Description of Artificial Sequence Synthetic peptide”
QQSGHWLSKT

SEQ ID NO: 121 - protein, 11 aa, Artificial Sequence
  source: /note=“Description of Artificial Sequence Synthetic peptide”
RASQDISSYLN

SEQ ID NO: 122 - protein, 7 aa, Artificial Sequence
  source: /note=“Description of Artificial Sequence Synthetic peptide”
DASTLQS

SEQ ID NO: 123 - protein, 10 aa, Artificial Sequence
  source: /note=“Description of Artificial Sequence Synthetic peptide”
QQSGHWLSKT

SEQ ID NO: 124 - protein, 7 aa, Artificial Sequence
  source: /note=“Description of Artificial Sequence Synthetic peptide”
SQDISSY

SEQ ID NO: 125 - protein, 3 aa, Artificial Sequence
  source: /note=“Description of Artificial Sequence Synthetic peptide”
DAS

SEQ ID NO: 126 - protein, 7 aa, Artificial Sequence
  source: /note=“Description of Artificial Sequence Synthetic peptide”
SGHWLSK

SEQ ID NO: 127 - protein, 108 aa, Artificial Sequence
  source: /note=“Description of Artificial Sequence Synthetic polypeptide”
DIQMTQSPSSLSASVGDRVTITCRASQDISSYLNWYQQKPGKAPKLLIYDASTLQSGVPSRFSGSGSGTDFTLTISSLQPEDFATYYCQQSGHWLSKTFGQGTKVEIK

SEQ ID NO: 128 - DNA, 324 bp, Artificial Sequence
  source: /note=“Description of Artificial Sequence Synthetic polynucleotide”
gatattcagatgactcagtcacctagtagcctgagcgctagtgtgggcgatagagtgactatcacctgtagagcctctcaggatatctctagctacctgaactggtatcagcagaagcccggtaaagcccctaagctgctgatctacgacgcctctaccctgcagtcaggcgtgccctctaggtttagcggtagcggtagtggcaccgacttcaccctgactatctctagcctgcagcccgaggacttcgctacctactactgtcagcagtcaggccactggctgtctaagaccttcggtcagggcactaaggtcgagattaag

SEQ ID NO: 129 - protein, 215 aa, Artificial Sequence
  source: /note=“Description of Artificial Sequence Synthetic polypeptide”
DIQMTQSPSSLSASVGDRVTITCRASQDISSYLNWYQQKPGKAPKLLIYDASTLQSGVPSRFSGSGSGTDFTLTISSLQPEDFATYYCQQSGHWLSKTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - DNA, 645 bp, Artificial Sequence
  source: /note=“Description of Artificial Sequence Synthetic polynucleotide”
gatattcagatgactcagtcacctagtagcctgagcgctagtgtgggcgatagagtgactatcacctgtagagcctctcaggatatctctagctacctgaactggtatcagcagaagcccggtaaagcccctaagctgctgatctacgacgcctctaccctgcagtcaggcgtgccctctaggtttagcggtagcggtagtggcaccgacttcaccctgactatctctagcctgcagcccgaggacttcgctacctactactgtcagcagtcaggccactggctgtctaagaccttcggtcagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 131 - protein, 10 aa, Artificial Sequence
  source: /note=“Description of Artificial Sequence Synthetic peptide”
GGTFSDNAIS

SEQ ID NO: 132 - protein, 17 aa, Artificial Sequence
  source: /note=“Description of Artificial Sequence Synthetic peptide”
GINPDFGWANYAQKFQG

SEQ ID NO: 133 - protein, 7 aa, Artificial Sequence
  source: /note=“Description of Artificial Sequence Synthetic peptide”
DSSGMGY

SEQ ID NO: 134 - protein, 5 aa, Artificial Sequence
  source: /note=“Description of Artificial Sequence Synthetic peptide”
DNAIS

SEQ ID NO: 135 - protein, 17 aa, Artificial Sequence
  source: /note=“Description of Artificial Sequence Synthetic peptide”
GINPDFGWANYAQKFQG

SEQ ID NO: 136 - protein, 7 aa, Artificial Sequence
  source: /note=“Description of Artificial Sequence Synthetic peptide”
DSSGMGY

SEQ ID NO: 137 - protein, 7 aa, Artificial Sequence
  source: /note=“Description of Artificial Sequence Synthetic peptide”
GGTFSDN

SEQ ID NO: 138 - protein, 6 aa, Artificial Sequence
  source: /note=“Description of Artificial Sequence Synthetic peptide”
NPDFGW

SEQ ID NO: 139 - protein, 7 aa, Artificial Sequence
  source: /note=“Description of Artificial Sequence Synthetic peptide”
DSSGMGY

SEQ ID NO: 140 - protein, 116 aa, Artificial Sequence; identical to SEQ ID NO: 114
  source: /note=“Description of Artificial Sequence Synthetic polypeptide”

SEQ ID NO: 141 - DNA, 348 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgttttctgacaacgctatctcttgggtgcgccaggccccgggccagggcctcgagtggatgggcggtatcaacccggacttcggctgggcgaactacgcccagaaatttcagggccgggtgaccattaccgccgatgaaagcaccagcaccgcctatatggaactgagcagcctgcgcagcgaagatacggccgtgtattattgcgcgcgtgactcttctggtatgggttactggggccaaggcaccctggtgactgttagctca

SEQ ID NO: 142 - protein, 446 aa, Artificial Sequence
  source: /note=“Description of Artificial Sequence Synthetic polypeptide”
QVQLVQSGAEVKKPGSSVKVSCKASGGTFSDNAISWVRQAPGQGLEWMGGINPDFGWANYAQKFQGRVTITADESTSTAYMELSSLRSEDTAVYYCARDSSGMGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - DNA, 1338 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgttttctgacaacgctatctcttgggtgcgccaggccccgggccagggcctcgagtggatgggcggtatcaacccggacttcggctgggcgaactacgcccagaaatttcagggccgggtgaccattaccgccgatgaaagcaccagcaccgcctatatggaactgagcagcctgcgcagcgaagatacggccgtgtattattgcgcgcgtgactcttctggtatgggttactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 144 - protein, 11 aa, Artificial Sequence
  source: /note=“Description of Artificial Sequence Synthetic peptide”
RASQDISSYLN

SEQ ID NO: 145 - protein, 7 aa, Artificial Sequence
  source: /note=“Description of Artificial Sequence Synthetic peptide”
DASTLQS

SEQ ID NO: 146 - protein, 10 aa, Artificial Sequence
  source: /note=“Description of Artificial Sequence Synthetic peptide”
QQSGHWLSKT

SEQ ID NO: 147 - protein, 11 aa, Artificial Sequence
  source: /note=“Description of Artificial Sequence Synthetic peptide”
RASQDISSYLN

SEQ ID NO: 148 - protein, 7 aa, Artificial Sequence
  source: /note=“Description of Artificial Sequence Synthetic peptide”
DASTLQS

SEQ ID NO: 149 - protein, 10 aa, Artificial Sequence
  source: /note=“Description of Artificial Sequence Synthetic peptide”
QQSGHWLSKT

SEQ ID NO: 150 - protein, 7 aa, Artificial Sequence
  source: /note=“Description of Artificial Sequence Synthetic peptide”
SQDISSY

SEQ ID NO: 151 - protein, 3 aa, Artificial Sequence
  source: /note=“Description of Artificial Sequence Synthetic peptide”
DAS

SEQ ID NO: 152 - protein, 7 aa, Artificial Sequence
  source: /note=“Description of Artificial Sequence Synthetic peptide”
SGHWLSK

SEQ ID NO: 153 - protein, 108 aa, Artificial Sequence; identical to SEQ ID NO: 127
  source: /note=“Description of Artificial Sequence Synthetic polypeptide”

SEQ ID NO: 154 - DNA, 324 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccaggacatttcttcttacctgaactggtaccagcagaaaccgggcaaagcgccgaaactattaatctacgacgcttctactctgcaaagcggcgtgccgagccgctttagcggcagcggatccggcaccgatttcaccctgaccattagctctctgcaaccggaagactttgcgacctattattgccagcagtctggtcattggctgtctaaaacctttggccagggcacgaaagttgaaattaaa

SEQ ID NO: 155 - protein, 215 aa, Artificial Sequence; identical to SEQ ID NO: 129
  source: /note=“Description of Artificial Sequence Synthetic polypeptide”

SEQ ID NO: 156 - DNA, 645 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccaggacatttcttcttacctgaactggtaccagcagaaaccgggcaaagcgccgaaactattaatctacgacgcttctactctgcaaagcggcgtgccgagccgctttagcggcagcggatccggcaccgatttcaccctgaccattagctctctgcaaccggaagactttgcgacctattattgccagcagtctggtcattggctgtctaaaacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 157 - protein, 10 aa, Artificial Sequence
  source: /note=“Description of Artificial Sequence Synthetic peptide”
GGTFRDYAIS

SEQ ID NO: 158 - protein, 17 aa, Artificial Sequence
  source: /note=“Description of Artificial Sequence Synthetic peptide”
GIIPAFGTANYAQKFQG

SEQ ID NO: 159 - protein, 18 aa, Artificial Sequence
  source: /note=“Description of Artificial Sequence Synthetic peptide”
EQDPEYGYGGYPYEAMDV

SEQ ID NO: 160 - protein, 5 aa, Artificial Sequence
  source: /note=“Description of Artificial Sequence Synthetic peptide”
DYAIS

SEQ ID NO: 161 - protein, 17 aa, Artificial Sequence
  source: /note=“Description of Artificial Sequence Synthetic peptide”
GIIPAFGTANYAQKFQG

SEQ ID NO: 162 - protein, 18 aa, Artificial Sequence
  source: /note=“Description of Artificial Sequence Synthetic peptide”
EQDPEYGYGGYPYEAMDV

SEQ ID NO: 163 - protein, 7 aa, Artificial Sequence
  source: /note=“Description of Artificial Sequence Synthetic peptide”
GGTFRDY

SEQ ID NO: 164 - protein, 6 aa, Artificial Sequence
  source: /note=“Description of Artificial Sequence Synthetic peptide”
IPAFGT

SEQ ID NO: 165 - protein, 18 aa, Artificial Sequence
  source: /note=“Description of Artificial Sequence Synthetic peptide”
EQDPEYGYGGYPYEAMDV

SEQ ID NO: 166 - protein, 127 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGYGGYPYEAMDVWGQGTLVTVSS

SEQ ID NO: 167 - DNA, 381 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagtacggctacggcggctacccctacgaggctatggacgtgtggggtcagggcaccctggtcaccgtgtctagc

SEQ ID NO: 168 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - DNA, 1371 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagtacggctacggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 170 - protein, 11 aa, Artificial Sequence
  source: /note=“Description of Artificial Sequence Synthetic peptide”
SGDNIPQHSVH

SEQ ID NO: 171 - protein, 7 aa, Artificial Sequence
  source: /note=“Description of Artificial Sequence Synthetic peptide”
DDTERPS

SEQ ID NO: 172 - protein, 11 aa, Artificial Sequence
  source: /note=“Description of Artificial Sequence Synthetic peptide”
SSWDSSMDSVV

SEQ ID NO: 173 - protein, 11 aa, Artificial Sequence
  source: /note=“Description of Artificial Sequence Synthetic peptide”
SGDNIPQHSVH

SEQ ID NO: 174 - protein, 7 aa, Artificial Sequence
  source: /note=“Description of Artificial Sequence Synthetic peptide”
DDTERPS

SEQ ID NO: 175 - protein, 11 aa, Artificial Sequence
  source: /note=“Description of Artificial Sequence Synthetic peptide”
SSWDSSMDSVV

SEQ ID NO: 176 - protein, 7 aa, Artificial Sequence
  source: /note=“Description of Artificial Sequence Synthetic peptide”
DNIPQHS

SEQ ID NO: 177 - protein, 3 aa, Artificial Sequence
  source: /note=“Description of Artificial Sequence Synthetic peptide”
DDT

SEQ ID NO: 178 - protein, 8 aa, Artificial Sequence
  source: /note=“Description of Artificial Sequence Synthetic peptide”
WDSSMDSV

SEQ ID NO: 179 - protein, 108 aa, Artificial Sequence
  source: /note=“Description of Artificial Sequence Synthetic polypeptide”
SYELTQPLSVSVALGQTARITCSGDNIPQHSVHWYQQKPGQAPVLVIYDDTERPSGIPERFSGSNSGNTATLTISRAQAGDEADYYCSSWDSSMDSVVFGGGTKLTVL

SEQ ID NO: 180 - DNA, 324 bp, Artificial Sequence
  source: /note=“Description of Artificial Sequence Synthetic polynucleotide”
agctacgagctgactcagcccctgagcgtcagcgtggccctgggtcagaccgctagaatcacctgtagcggcgataatatccctcagcactcagtgcactggtatcagcagaagcccggtcaggcccccgtgctggtgatctacgacgacaccgagcggcctagcggaatccccgagcggtttagcggctctaatagcggtaacaccgctaccctgactatctctagggctcaggccggcgacgaggccgactactactgctctagctgggatagctctatggatagcgtggtgttcggcggaggcactaagctgaccgtgctg

SEQ ID NO: 181 - protein, 214 aa, Artificial Sequence
  source: /note=“Description of Artificial Sequence Synthetic polypeptide”
SYELTQPLSVSVALGQTARITCSGDNIPQHSVHWYQQKPGQAPVLVIYDDTERPSGIPERFSGSNSGNTATLTISRAQAGDEADYYCSSWDSSMDSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 182 - DNA, 642 bp, Artificial Sequence
  source: /note=“Description of Artificial Sequence Synthetic polynucleotide”
agctacgagctgactcagcccctgagcgtcagcgtggccctgggtcagaccgctagaatcacctgtagcggcgataatatccctcagcactcagtgcactggtatcagcagaagcccggtcaggcccccgtgctggtgatctacgacgacaccgagcggcctagcggaatccccgagcggtttagcggctctaatagcggtaacaccgctaccctgactatctctagggctcaggccggcgacgaggccgactactactgctctagctgggatagctctatggatagcgtggtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 183 - protein, 10 aa, Artificial Sequence
  source: /note=“Description of Artificial Sequence Synthetic peptide”
GGTFRDYAIS

SEQ ID NO: 184 - protein, 17 aa, Artificial Sequence
  source: /note=“Description of Artificial Sequence Synthetic peptide”
GIIPAFGTANYAQKFQG

SEQ ID NO: 185 - protein, 18 aa, Artificial Sequence
  source: /note=“Description of Artificial Sequence Synthetic peptide”
EQDPEYGYGGYPYEAMDV

SEQ ID NO: 186 - protein, 5 aa, Artificial Sequence
  source: /note=“Description of Artificial Sequence Synthetic peptide”
DYAIS

SEQ ID NO: 187 - protein, 17 aa, Artificial Sequence
  source: /note=“Description of Artificial Sequence Synthetic peptide”
GIIPAFGTANYAQKFQG

SEQ ID NO: 188 - protein, 18 aa, Artificial Sequence
  source: /note=“Description of Artificial Sequence Synthetic peptide”
EQDPEYGYGGYPYEAMDV

SEQ ID NO: 189 - protein, 7 aa, Artificial Sequence
  source: /note=“Description of Artificial Sequence Synthetic peptide”
GGTFRDY

SEQ ID NO: 190 - protein, 6 aa, Artificial Sequence
  source: /note=“Description of Artificial Sequence Synthetic peptide”
IPAFGT

SEQ ID NO: 191 - protein, 18 aa, Artificial Sequence
  source: /note=“Description of Artificial Sequence Synthetic peptide”
EQDPEYGYGGYPYEAMDV

SEQ ID NO: 192 - protein, 127 aa, Artificial Sequence; identical to SEQ ID NO: 166
  source: /note=“Description of Artificial Sequence Synthetic polypeptide”

SEQ ID NO: 193 - DNA, 381 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggttacccgtatgaagctatggatgtttggggccaaggcaccctggtgactgttagctca

SEQ ID NO: 194 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - DNA, 1371 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 196 - protein, 11 aa, Artificial Sequence
  source: /note=“Description of Artificial Sequence Synthetic peptide”
SGDNIPQHSVH

SEQ ID NO: 197 - protein, 7 aa, Artificial Sequence
  source: /note=“Description of Artificial Sequence Synthetic peptide”
DDTERPS

SEQ ID NO: 198 - protein, 11 aa, Artificial Sequence
  source: /note=“Description of Artificial Sequence Synthetic peptide”
SSWDSSMDSVV

SEQ ID NO: 199 - protein, 11 aa, Artificial Sequence
  source: /note=“Description of Artificial Sequence Synthetic peptide”
SGDNIPQHSVH

SEQ ID NO: 200 - protein, 7 aa, Artificial Sequence
  source: /note=“Description of Artificial Sequence Synthetic peptide”
DDTERPS

SEQ ID NO: 201 - protein, 11 aa, Artificial Sequence
  source: /note=“Description of Artificial Sequence Synthetic peptide”
SSWDSSMDSVV

SEQ ID NO: 202 - protein, 7 aa, Artificial Sequence
  source: /note=“Description of Artificial Sequence Synthetic peptide”
DNIPQHS

SEQ ID NO: 203 - protein, 3 aa, Artificial Sequence
  source: /note=“Description of Artificial Sequence Synthetic peptide”
DDT

SEQ ID NO: 204 - protein, 8 aa, Artificial Sequence
  source: /note=“Description of Artificial Sequence Synthetic peptide”
WDSSMDSV

SEQ ID NO: 205 - protein, 108 aa, Artificial Sequence; identical to SEQ ID NO: 179
  source: /note=“Description of Artificial Sequence Synthetic polypeptide”

SEQ ID NO: 206 - DNA, 324 bp, Artificial Sequence
  source: /note=“Description of Artificial Sequence Synthetic polynucleotide”
agctacgaactgacccagccgctgagcgtgagcgtggccctgggccagaccgcgaggattacctgtagcggcgataacatcccgcagcattctgttcattggtaccagcagaaaccgggccaggcgccggtgctggtgatctacgacgacactgaacgtccgagcggcatcccggaacgttttagcggatccaacagcggcaacaccgcgaccctgaccattagcagggcccaggcgggcgacgaagcggattattactgctcttcttgggactcttctatggactctgttgtgtttggcggcggcacgaagttaaccgtccta

SEQ ID NO: 207 - protein, 214 aa, Artificial Sequence; identical to SEQ ID NO: 181
  source: /note=“Description of Artificial Sequence Synthetic polypeptide”

SEQ ID NO: 208 - DNA, 642 bp, Artificial Sequence
  source: /note=“Description of Artificial Sequence Synthetic polynucleotide”
agctacgaactgacccagccgctgagcgtgagcgtggccctgggccagaccgcgaggattacctgtagcggcgataacatcccgcagcattctgttcattggtaccagcagaaaccgggccaggcgccggtgctggtgatctacgacgacactgaacgtccgagcggcatcccggaacgttttagcggatccaacagcggcaacaccgcgaccctgaccattagcagggcccaggcgggcgacgaagcggattattactgctcttcttgggactcttctatggactctgttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 209 - protein, 10 aa, Artificial Sequence
  source: /note=“Description of Artificial Sequence Synthetic peptide”
GFTFPTHGLH

SEQ ID NO: 210 - protein, 17 aa, Artificial Sequence
  source: /note=“Description of Artificial Sequence Synthetic peptide”
AISYDASETNYADSVKG

SEQ ID NO: 211 - protein, 9 aa, Artificial Sequence
  source: /note=“Description of Artificial Sequence Synthetic peptide”
ESIGGYFDY

SEQ ID NO: 212 - protein, 5 aa, Artificial Sequence
  source: /note=“Description of Artificial Sequence Synthetic peptide”
THGLH

SEQ ID NO: 213 - protein, 17 aa, Artificial Sequence
  source: /note=“Description of Artificial Sequence Synthetic peptide”
AISYDASETNYADSVKG

SEQ ID NO: 214 - protein, 9 aa, Artificial Sequence
  source: /note=“Description of Artificial Sequence Synthetic peptide”
ESIGGYFDY

SEQ ID NO: 215 - protein, 7 aa, Artificial Sequence
  source: /note=“Description of Artificial Sequence Synthetic peptide”
GFTFPTH

SEQ ID NO: 216 - protein, 6 aa, Artificial Sequence
  source: /note=“Description of Artificial Sequence Synthetic peptide”
SYDASE

SEQ ID NO: 217 - protein, 9 aa, Artificial Sequence
  source: /note=“Description of Artificial Sequence Synthetic peptide”
ESIGGYFDY

SEQ ID NO: 218 - protein, 118 aa, Artificial Sequence
  source: /note=“Description of Artificial Sequence Synthetic polypeptide”
QVQLLESGGGLVQPGGSLRLSCAASGFTFPTHGLHWVRQAPGKGLEWVSAISYDASETNYADSVKGRFTISRDNSKNTLYLQMNSLRAEDTAVYYCARESIGGYFDYWGQGTLVTVSS

SEQ ID NO: 219 - DNA, 354 bp, Artificial Sequence
  source: /note=“Description of Artificial Sequence Synthetic polynucleotide”
caggtgcagctgctggaatcaggcggcggactggtgcagcctggcggtagcctgagactgagctgcgctgctagtggcttcaccttccctactcacggcctgcactgggtcagacaggcccctggtaaaggcctggagtgggtcagcgctattagctacgacgctagtgaaactaactacgccgatagcgtgaagggccggttcactatctctagggataactctaagaacaccctgtacctgcagatgaatagcctgagagccgaggacaccgccgtctactactgcgctagagagtctatcggcggctacttcgactactggggtcagggcaccctggtcaccgtgtctagc

SEQ ID NO: 220 - protein, 448 aa, Artificial Sequence
  source: /note=“Description of Artificial Sequence Synthetic polypeptide”
QVQLLESGGGLVQPGGSLRLSCAASGFTFPTHGLHWVRQAPGKGLEWVSAISYDASETNYADSVKGRFTISRDNSKNTLYLQMNSLRAEDTAVYYCARESIGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - DNA, 1344 bp, Artificial Sequence
  source: /note=“Description of Artificial Sequence Synthetic polynucleotide”
caggtgcagctgctggaatcaggcggcggactggtgcagcctggcggtagcctgagactgagctgcgctgctagtggcttcaccttccctactcacggcctgcactgggtcagacaggcccctggtaaaggcctggagtgggtcagcgctattagctacgacgctagtgaaactaactacgccgatagcgtgaagggccggttcactatctctagggataactctaagaacaccctgtacctgcagatgaatagcctgagagccgaggacaccgccgtctactactgcgctagagagtctatcggcggctacttcgactactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 222 - protein, 11 aa, Artificial Sequence
  source: /note=“Description of Artificial Sequence Synthetic peptide”
SGDALGKNTVS

SEQ ID NO: 223 - protein, 7 aa, Artificial Sequence
  source: /note=“Description of Artificial Sequence Synthetic peptide”
DDTDRPS

SEQ ID NO: 224 - protein, 9 aa, Artificial Sequence
  source: /note=“Description of Artificial Sequence Synthetic peptide”
SSTDLSTVV

SEQ ID NO: 225 - protein, 11 aa, Artificial Sequence
  source: /note=“Description of Artificial Sequence Synthetic peptide”
SGDALGKNTVS

SEQ ID NO: 226 - protein, 7 aa, Artificial Sequence
  source: /note=“Description of Artificial Sequence Synthetic peptide”
DDTDRPS

SEQ ID NO: 227 - protein, 9 aa, Artificial Sequence
  source: /note=“Description of Artificial Sequence Synthetic peptide”
SSTDLSTVV

SEQ ID NO: 228 - protein, 7 aa, Artificial Sequence
  source: /note=“Description of Artificial Sequence Synthetic peptide”
DALGKNT

SEQ ID NO: 229 - protein, 3 aa, Artificial Sequence
  source: /note=“Description of Artificial Sequence Synthetic peptide”
DDT

SEQ ID NO: 230 - protein, 6 aa, Artificial Sequence
  source: /note=“Description of Artificial Sequence Synthetic peptide”
TDLSTV

SEQ ID NO: 231 - protein, 106 aa, Artificial Sequence
  source: /note=“Description of Artificial Sequence Synthetic polypeptide”
SYELTQPLSVSVALGQTARITCSGDALGKNTVSWYQQKPGQAPVLVIYDDTDRPSGIPERFSGSNSGNTATLTISRAQAGDEADYYCSSTDLSTVVFGGGTKLTVL

SEQ ID NO: 232 - DNA, 318 bp, Artificial Sequence
  source: /note=“Description of Artificial Sequence Synthetic polynucleotide”
agctacgagctgactcagcccctgagcgtcagcgtggccctgggtcagaccgctagaatcacctgtagcggcgacgccctgggtaaaaacaccgtcagctggtatcagcagaagcccggtcaggcccccgtgctggtgatctacgacgacaccgatagacctagcggaatccccgagcggtttagcggctctaatagcggtaacaccgctaccctgactatctctagggctcaggccggcgacgaggccgactactactgctctagcaccgacctgagcaccgtggtgttcggcggaggcactaagctgaccgtgctg

SEQ ID NO: 233 - protein, 212 aa, Artificial Sequence
  source: /note=“Description of Artificial Sequence Synthetic polypeptide”
SYELTQPLSVSVALGQTARITCSGDALGKNTVSWYQQKPGQAPVLVIYDDTDRPSGIPERFSGSNSGNTATLTISRAQAGDEADYYCSSTDLSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 234 - DNA, 636 bp, Artificial Sequence
  source: /note=“Description of Artificial Sequence Synthetic polynucleotide”
agctacgagctgactcagcccctgagcgtcagcgtggccctgggtcagaccgctagaatcacctgtagcggcgacgccctgggtaaaaacaccgtcagctggtatcagcagaagcccggtcaggcccccgtgctggtgatctacgacgacaccgatagacctagcggaatccccgagcggtttagcggctctaatagcggtaacaccgctaccctgactatctctagggctcaggccggcgacgaggccgactactactgctctagcaccgacctgagcaccgtggtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 235 - protein, 10 aa, Artificial Sequence
  source: /note=“Description of Artificial Sequence Synthetic peptide”
GFTFPTHGLH

SEQ ID NO: 236 - protein, 17 aa, Artificial Sequence
  source: /note=“Description of Artificial Sequence Synthetic peptide”
AISYDASETNYADSVKG

SEQ ID NO: 237 - protein, 9 aa, Artificial Sequence
  source: /note=“Description of Artificial Sequence Synthetic peptide”
ESIGGYFDY

SEQ ID NO: 238 - protein, 5 aa, Artificial Sequence
  source: /note=“Description of Artificial Sequence Synthetic peptide”
THGLH

SEQ ID NO: 239 - protein, 17 aa, Artificial Sequence
  source: /note=“Description of Artificial Sequence Synthetic peptide”
AISYDASETNYADSVKG

SEQ ID NO: 240 - protein, 9 aa, Artificial Sequence
  source: /note=“Description of Artificial Sequence Synthetic peptide”
ESIGGYFDY

SEQ ID NO: 241 - protein, 7 aa, Artificial Sequence
  source: /note=“Description of Artificial Sequence Synthetic peptide”
GFTFPTH

SEQ ID NO: 242 - protein, 6 aa, Artificial Sequence
  source: /note=“Description of Artificial Sequence Synthetic peptide”
SYDASE

SEQ ID NO: 243 - protein, 9 aa, Artificial Sequence
  source: /note=“Description of Artificial Sequence Synthetic peptide”
ESIGGYFDY

SEQ ID NO: 244 - protein, 118 aa, Artificial Sequence; identical to SEQ ID NO: 218
  source: /note=“Description of Artificial Sequence Synthetic polypeptide”

SEQ ID NO: 245 - DNA, 354 bp, Artificial Sequence
  source: /note=“Description of Artificial Sequence Synthetic polynucleotide”
caggtgcaattgctggaaagcggcggtggcctggtgcagccgggtggcagcctgcgtctgagctgcgcggcgtccggattcacctttcctactcatggtctgcattgggtgcgccaggccccgggcaaaggtctcgagtgggtttccgctatctcttacgacgcctctgaaaccaactatgcggatagcgtgaaaggccgctttaccatcagccgcgataattcgaaaaacaccctgtatctgcaaatgaacagcctgcgtgcggaagatacggccgtgtattattgcgcgcgtgaatctatcggtggttacttcgattactggggccaaggcaccctggtgactgttagctca

SEQ ID NO: 246 - protein, 448 aa, Artificial Sequence
  source: /note=“Description of Artificial Sequence Synthetic polypeptide”
QVQLLESGGGLVQPGGSLRLSCAASGFTFPTHGLHWVRQAPGKGLEWVSAISYDASETNYADSVKGRFTISRDNSKNTLYLQMNSLRAEDTAVYYCARESIGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - DNA, 1344 bp, Artificial Sequence
  source: /note=“Description of Artificial Sequence Synthetic polynucleotide”
caggtgcaattgctggaaagcggcggtggcctggtgcagccgggtggcagcctgcgtctgagctgcgcggcgtccggattcacctttcctactcatggtctgcattgggtgcgccaggccccgggcaaaggtctcgagtgggtttccgctatctcttacgacgcctctgaaaccaactatgcggatagcgtgaaaggccgctttaccatcagccgcgataattcgaaaaacaccctgtatctgcaaatgaacagcctgcgtgcggaagatacggccgtgtattattgcgcgcgtgaatctatcggtggttacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 248 - protein, 11 aa, Artificial Sequence
  source: /note=“Description of Artificial Sequence Synthetic peptide”
SGDALGKNTVS

SEQ ID NO: 249 - protein, 7 aa, Artificial Sequence
  source: /note=“Description of Artificial Sequence Synthetic peptide”
DDTDRPS

SEQ ID NO: 250 - protein, 9 aa, Artificial Sequence
  source: /note=“Description of Artificial Sequence Synthetic peptide”
SSTDLSTVV

SEQ ID NO: 251 - protein, 11 aa, Artificial Sequence
  source: /note=“Description of Artificial Sequence Synthetic peptide”
SGDALGKNTVS

SEQ ID NO: 252 - protein, 7 aa, Artificial Sequence
  source: /note=“Description of Artificial Sequence Synthetic peptide”
DDTDRPS

SEQ ID NO: 253 - protein, 9 aa, Artificial Sequence
  source: /note=“Description of Artificial Sequence Synthetic peptide”
SSTDLSTVV

SEQ ID NO: 254 - protein, 7 aa, Artificial Sequence
  source: /note=“Description of Artificial Sequence Synthetic peptide”
DALGKNT

SEQ ID NO: 255 - protein, 3 aa, Artificial Sequence
  source: /note=“Description of Artificial Sequence Synthetic peptide”
DDT

SEQ ID NO: 256 - protein, 6 aa, Artificial Sequence
  source: /note=“Description of Artificial Sequence Synthetic peptide”
TDLSTV

SEQ ID NO: 257 - protein, 106 aa, Artificial Sequence; identical to SEQ ID NO: 231
  source: /note=“Description of Artificial Sequence Synthetic polypeptide”

SEQ ID NO: 258 - DNA, 318 bp, Artificial Sequence
  source: /note=“Description of Artificial Sequence Synthetic polynucleotide”
agctacgaactgacccagccgctgagcgtgagcgtggccctgggccagaccgcgaggattacctgtagcggcgatgctctgggtaaaaacactgtttcttggtaccagcagaaaccgggccaggcgccggtgctggtgatctacgacgacactgaccgtccgagcggcatcccggaacgttttagcggatccaacagcggcaacaccgcgaccctgaccattagcagggcccaggcgggcgacgaagcggattattactgctcttctactgacctgtctactgttgtgtttggcggcggcacgaagttaaccgtccta

SEQ ID NO: 259 - protein, 212 aa, Artificial Sequence; identical to SEQ ID NO: 233
  source: /note=“Description of Artificial Sequence Synthetic polypeptide”

SEQ ID NO: 260 - DNA, 636 bp, Artificial Sequence
  source: /note=“Description of Artificial Sequence Synthetic polynucleotide”
agctacgaactgacccagccgctgagcgtgagcgtggccctgggccagaccgcgaggattacctgtagcggcgatgctctgggtaaaaacactgtttcttggtaccagcagaaaccgggccaggcgccggtgctggtgatctacgacgacactgaccgtccgagcggcatcccggaacgttttagcggatccaacagcggcaacaccgcgaccctgaccattagcagggcccaggcgggcgacgaagcggattattactgctcttctactgacctgtctactgttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 261 - protein, 10 aa, Artificial Sequence
  source: /note=“Description of Artificial Sequence Synthetic peptide”
GFTFPTHGLH

SEQ ID NO: 262 - protein, 17 aa, Artificial Sequence
  source: /note=“Description of Artificial Sequence Synthetic peptide”
AISYEGSETNYADSVKG

SEQ ID NO: 263 - protein, 9 aa, Artificial Sequence
  source: /note=“Description of Artificial Sequence Synthetic peptide”
ESIGGYFDY

SEQ ID NO: 264 - protein, 5 aa, Artificial Sequence
  source: /note=“Description of Artificial Sequence Synthetic peptide”
THGLH

SEQ ID NO: 265 - protein, 17 aa, Artificial Sequence
  source: /note=“Description of Artificial Sequence Synthetic peptide”
AISYEGSETNYADSVKG

SEQ ID NO: 266 - protein, 9 aa, Artificial Sequence
  source: /note=“Description of Artificial Sequence Synthetic peptide”
ESIGGYFDY

SEQ ID NO: 267 - protein, 7 aa, Artificial Sequence
  source: /note=“Description of Artificial Sequence Synthetic peptide”
GFTFPTH

SEQ ID NO: 268 - protein, 6 aa, Artificial Sequence
  source: /note=“Description of Artificial Sequence Synthetic peptide”
SYEGSE

SEQ ID NO: 269 - protein, 9 aa, Artificial Sequence
  source: /note=“Description of Artificial Sequence Synthetic peptide”
ESIGGYFDY

SEQ ID NO: 270 - protein, 118 aa, Artificial Sequence
  source: /note=“Description of Artificial Sequence Synthetic polypeptide”
QVQLLESGGGLVQPGGSLRLSCAASGFTFPTHGLHWVRQAPGKGLEWVSAISYEGSETNYADSVKGRFTISRDNSKNTLYLQMNSLRAEDTAVYYCARESIGGYFDYWGQGTLVTVSS

SEQ ID NO: 271 - DNA, 354 bp, Artificial Sequence
  source: /note=“Description of Artificial Sequence Synthetic polynucleotide”
caggtgcagctgctggaatcaggcggcggactggtgcagcctggcggtagcctgagactgagctgcgctgctagtggcttcaccttccctactcacggcctgcactgggtcagacaggcccctggtaaaggcctggagtgggtcagcgctattagctacgagggtagcgagactaactacgccgatagcgtgaagggccggttcactatctctagggataactctaagaacaccctgtacctgcagatgaatagcctgagagccgaggacaccgccgtctactactgcgctagagagtctatcggcggctacttcgactactggggtcagggcaccctggtcaccgtgtctagc

SEQ ID NO: 272 - protein, 448 aa, Artificial Sequence
  source: /note=“Description of Artificial Sequence Synthetic polypeptide”
QVQLLESGGGLVQPGGSLRLSCAASGFTFPTHGLHWVRQAPGKGLEWVSAISYEGSETNYADSVKGRFTISRDNSKNTLYLQMNSLRAEDTAVYYCARESIGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 273 - DNA, 1344 bp, Artificial Sequence
  source: /note=“Description of Artificial Sequence Synthetic polynucleotide”
caggtgcagctgctggaatcaggcggcggactggtgcagcctggcggtagcctgagactgagctgcgctgctagtggcttcaccttccctactcacggcctgcactgggtcagacaggcccctggtaaaggcctggagtgggtcagcgctattagctacgagggtagcgagactaactacgccgatagcgtgaagggccggttcactatctctagggataactctaagaacaccctgtacctgcagatgaatagcctgagagccgaggacaccgccgtctactactgcgctagagagtctatcggcggctacttcgactactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 274 - protein, 11 aa, Artificial Sequence
  source: /note=“Description of Artificial Sequence Synthetic peptide”
SGDALGKNTVS

SEQ ID NO: 275 - protein, 7 aa, Artificial Sequence
  source: /note=“Description of Artificial Sequence Synthetic peptide”
DDTDRPS

SEQ ID NO: 276 - protein, 9 aa, Artificial Sequence
  source: /note=“Description of Artificial Sequence Synthetic peptide”
SSTDLSTVV

SEQ ID NO: 277 - protein, 11 aa, Artificial Sequence
  source: /note=“Description of Artificial Sequence Synthetic peptide”
SGDALGKNTVS

SEQ ID NO: 278 - protein, 7 aa, Artificial Sequence
  source: /note=“Description of Artificial Sequence Synthetic peptide”
DDTDRPS

SEQ ID NO: 279 - protein, 9 aa, Artificial Sequence
  source: /note=“Description of Artificial Sequence Synthetic peptide”
SSTDLSTVV

SEQ ID NO: 280 - protein, 7 aa, Artificial Sequence
  source: /note=“Description of Artificial Sequence Synthetic peptide”
DALGKNT

SEQ ID NO: 281 - protein, 3 aa, Artificial Sequence
  source: /note=“Description of Artificial Sequence Synthetic peptide”
DDT

SEQ ID NO: 282 - protein, 6 aa, Artificial Sequence
  source: /note=“Description of Artificial Sequence Synthetic peptide”
TDLSTV

SEQ ID NO: 283 - protein, 106 aa, Artificial Sequence; identical to SEQ ID NO: 231
  source: /note=“Description of Artificial Sequence Synthetic polypeptide”

SEQ ID NO: 284 - DNA, 318 bp, Artificial Sequence; identical to SEQ ID NO: 232
  source: /note=“Description of Artificial Sequence Synthetic polynucleotide”

SEQ ID NO: 285 - protein, 212 aa, Artificial Sequence; identical to SEQ ID NO: 233
  source: /note=“Description of Artificial Sequence Synthetic polypeptide”

SEQ ID NO: 286 - DNA, 636 bp, Artificial Sequence; identical to SEQ ID NO: 234
  source: /note=“Description of Artificial Sequence Synthetic polynucleotide”

SEQ ID NO: 287 - protein, 10 aa, Artificial Sequence
  source: /note=“Description of Artificial Sequence Synthetic peptide”
GFTFPTHGLH

SEQ ID NO: 288 - protein, 17 aa, Artificial Sequence
  source: /note=“Description of Artificial Sequence Synthetic peptide”
AISYEGSETNYADSVKG

SEQ ID NO: 289 - protein, 9 aa, Artificial Sequence
  source: /note=“Description of Artificial Sequence Synthetic peptide”
ESIGGYFDY

SEQ ID NO: 290 - protein, 5 aa, Artificial Sequence
  source: /note=“Description of Artificial Sequence Synthetic peptide”
THGLH

SEQ ID NO: 291 - protein, 17 aa, Artificial Sequence
  source: /note=“Description of Artificial Sequence Synthetic peptide”
AISYEGSETNYADSVKG

SEQ ID NO: 292 - protein, 9 aa, Artificial Sequence
  source: /note=“Description of Artificial Sequence Synthetic peptide”
ESIGGYFDY

SEQ ID NO: 293 - protein, 7 aa, Artificial Sequence
  source: /note=“Description of Artificial Sequence Synthetic peptide”
GFTFPTH

SEQ ID NO: 294 - protein, 6 aa, Artificial Sequence
  source: /note=“Description of Artificial Sequence Synthetic peptide”
SYEGSE

SEQ ID NO: 295 - protein, 9 aa, Artificial Sequence
  source: /note=“Description of Artificial Sequence Synthetic peptide”
ESIGGYFDY

SEQ ID NO: 296 - protein, 118 aa, Artificial Sequence; identical to SEQ ID NO: 270
  source: /note=“Description of Artificial Sequence Synthetic polypeptide”

SEQ ID NO: 297 - DNA, 354 bp, Artificial Sequence
  source: /note=“Description of Artificial Sequence Synthetic polynucleotide”
caggtgcaattgctggaaagcggcggtggcctggtgcagccgggtggcagcctgcgtctgagctgcgcggcgtccggattcacctttcctactcatggtctgcattgggtgcgccaggccccgggcaaaggtctcgagtgggtttccgctatctcttacgagggttctgaaaccaactatgcggatagcgtgaaaggccgctttaccatcagccgcgataattcgaaaaacaccctgtatctgcaaatgaacagcctgcgtgcggaagatacggccgtgtattattgcgcgcgtgaatctatcggtggttacttcgattactggggccaaggcaccctggtgactgttagctca

SEQ ID NO: 298 - protein, 448 aa, Artificial Sequence
  source: /note=“Description of Artificial Sequence Synthetic polypeptide”
QVQLLESGGGLVQPGGSLRLSCAASGFTFPTHGLHWVRQAPGKGLEWVSAISYEGSETNYADSVKGRFTISRDNSKNTLYLQMNSLRAEDTAVYYCARESIGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - DNA, 1344 bp, Artificial Sequence
  source: /note=“Description of Artificial Sequence Synthetic polynucleotide”
caggtgcaattgctggaaagcggcggtggcctggtgcagccgggtggcagcctgcgtctgagctgcgcggcgtccggattcacctttcctactcatggtctgcattgggtgcgccaggccccgggcaaaggtctcgagtgggtttccgctatctcttacgagggttctgaaaccaactatgcggatagcgtgaaaggccgctttaccatcagccgcgataattcgaaaaacaccctgtatctgcaaatgaacagcctgcgtgcggaagatacggccgtgtattattgcgcgcgtgaatctatcggtggttacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 300 - protein, 11 aa, Artificial Sequence
  source: /note=“Description of Artificial Sequence Synthetic peptide”
SGDALGKNTVS

SEQ ID NO: 301 - protein, 7 aa, Artificial Sequence
  source: /note=“Description of Artificial Sequence Synthetic peptide”
DDTDRPS

SEQ ID NO: 302 - protein, 9 aa, Artificial Sequence
  source: /note=“Description of Artificial Sequence Synthetic peptide”
SSTDLSTVV

SEQ ID NO: 303 - protein, 11 aa, Artificial Sequence
  source: /note=“Description of Artificial Sequence Synthetic peptide”
SGDALGKNTVS

SEQ ID NO: 304 - protein, 7 aa, Artificial Sequence
  source: /note=“Description of Artificial Sequence Synthetic peptide”
DDTDRPS

SEQ ID NO: 305 - protein, 9 aa, Artificial Sequence
  source: /note=“Description of Artificial Sequence Synthetic peptide”
SSTDLSTVV

SEQ ID NO: 306 - protein, 7 aa, Artificial Sequence
  source: /note=“Description of Artificial Sequence Synthetic peptide”
DALGKNT

SEQ ID NO: 307 - protein, 3 aa, Artificial Sequence
  source: /note=“Description of Artificial Sequence Synthetic peptide”
DDT

SEQ ID NO: 308 - protein, 6 aa, Artificial Sequence
  source: /note=“Description of Artificial Sequence Synthetic peptide”
TDLSTV

SEQ ID NO: 309 - protein, 106 aa, Artificial Sequence; identical to SEQ ID NO: 231
  source: /note=“Description of Artificial Sequence Synthetic polypeptide”

SEQ ID NO: 310 - DNA, 318 bp, Artificial Sequence; identical to SEQ ID NO: 258
  source: /note=“Description of Artificial Sequence Synthetic polynucleotide”

SEQ ID NO: 311 - protein, 212 aa, Artificial Sequence; identical to SEQ ID NO: 233
  source: /note=“Description of Artificial Sequence Synthetic polypeptide”

SEQ ID NO: 312 - DNA, 636 bp, Artificial Sequence; identical to SEQ ID NO: 260
  source: /note=“Description of Artificial Sequence Synthetic polynucleotide”

SEQ ID NO: 313 - protein, 10 aa, Artificial Sequence
  source: /note=“Description of Artificial Sequence Synthetic peptide”
GGTFRDYAIS

SEQ ID NO: 314 - protein, 17 aa, Artificial Sequence
  source: /note=“Description of Artificial Sequence Synthetic peptide”
GIIPAFGTANYAQKFQG

SEQ ID NO: 315 - protein, 18 aa, Artificial Sequence
  source: /note=“Description of Artificial Sequence Synthetic peptide”
EQDPEFGYGGYPYEAMDV

SEQ ID NO: 316 - protein, 5 aa, Artificial Sequence
  source: /note=“Description of Artificial Sequence Synthetic peptide”
DYAIS

SEQ ID NO: 317 - protein, 17 aa, Artificial Sequence
  source: /note=“Description of Artificial Sequence Synthetic peptide”
GIIPAFGTANYAQKFQG

SEQ ID NO: 318 - protein, 18 aa, Artificial Sequence
  source: /note=“Description of Artificial Sequence Synthetic peptide”
EQDPEFGYGGYPYEAMDV

SEQ ID NO: 319 - protein, 7 aa, Artificial Sequence
  source: /note=“Description of Artificial Sequence Synthetic peptide”
GGTFRDY

SEQ ID NO: 320 - protein, 6 aa, Artificial Sequence
  source: /note=“Description of Artificial Sequence Synthetic peptide”
IPAFGT

SEQ ID NO: 321 - protein, 18 aa, Artificial Sequence
  source: /note=“Description of Artificial Sequence Synthetic peptide”
EQDPEFGYGGYPYEAMDV

SEQ ID NO: 322 - protein, 127 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEFGYGGYPYEAMDVWGQGTLVTVSS

SEQ ID NO: 323 - DNA, 381 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagttcggctacggcggctacccctacgaggctatggacgtgtggggtcagggcaccctggtcaccgtgtctagc

SEQ ID NO: 324 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEFGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - DNA, 1371 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagttcggctacggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 326 - protein, 11 aa, Artificial Sequence
  source: /note=“Description of Artificial Sequence Synthetic peptide”
SGDNIPQHSVH

SEQ ID NO: 327 - protein, 7 aa, Artificial Sequence
  source: /note=“Description of Artificial Sequence Synthetic peptide”
DDTERPS

SEQ ID NO: 328 - protein, 11 aa, Artificial Sequence
  source: /note=“Description of Artificial Sequence Synthetic peptide”
SSWDSSMDSVV

SEQ ID NO: 329 - protein, 11 aa, Artificial Sequence
  source: /note=“Description of Artificial Sequence Synthetic peptide”
SGDNIPQHSVH

SEQ ID NO: 330 - protein, 7 aa, Artificial Sequence
  source: /note=“Description of Artificial Sequence Synthetic peptide”
DDTERPS

SEQ ID NO: 331 - protein, 11 aa, Artificial Sequence
  source: /note=“Description of Artificial Sequence Synthetic peptide”
SSWDSSMDSVV

SEQ ID NO: 332 - protein, 7 aa, Artificial Sequence
  source: /note=“Description of Artificial Sequence Synthetic peptide”
DNIPQHS

SEQ ID NO: 333 - protein, 3 aa, Artificial Sequence
  source: /note=“Description of Artificial Sequence Synthetic peptide”
DDT

SEQ ID NO: 334 - protein, 8 aa, Artificial Sequence
  source: /note=“Description of Artificial Sequence Synthetic peptide”
WDSSMDSV

SEQ ID NO: 335 - protein, 108 aa, Artificial Sequence; identical to SEQ ID NO: 179
  source: /note=“Description of Artificial Sequence Synthetic polypeptide”

SEQ ID NO: 336 - DNA, 324 bp, Artificial Sequence; identical to SEQ ID NO: 180
  source: /note=“Description of Artificial Sequence Synthetic polynucleotide”

SEQ ID NO: 337 - protein, 214 aa, Artificial Sequence; identical to SEQ ID NO: 181
  source: /note=“Description of Artificial Sequence Synthetic polypeptide”

SEQ ID NO: 338 - DNA, 642 bp, Artificial Sequence; identical to SEQ ID NO: 182
  source: /note=“Description of Artificial Sequence Synthetic polynucleotide”

SEQ ID NO: 339 - protein, 10 aa, Artificial Sequence
  source: /note=“Description of Artificial Sequence Synthetic peptide”
GGTFRDYAIS

SEQ ID NO: 340 - protein, 17 aa, Artificial Sequence
  source: /note=“Description of Artificial Sequence Synthetic peptide”
GIIPAFGTANYAQKFQG

SEQ ID NO: 341 - protein, 18 aa, Artificial Sequence
  source: /note=“Description of Artificial Sequence Synthetic peptide”
EQDPEFGYGGYPYEAMDV

SEQ ID NO: 342 - protein, 5 aa, Artificial Sequence
  source: /note=“Description of Artificial Sequence Synthetic peptide”
DYAIS

SEQ ID NO: 343 - protein, 17 aa, Artificial Sequence
  source: /note=“Description of Artificial Sequence Synthetic peptide”
GIIPAFGTANYAQKFQG

SEQ ID NO: 344 - protein, 18 aa, Artificial Sequence
  source: /note=“Description of Artificial Sequence Synthetic peptide”
EQDPEFGYGGYPYEAMDV

SEQ ID NO: 345 - protein, 7 aa, Artificial Sequence
  source: /note=“Description of Artificial Sequence Synthetic peptide”
GGTFRDY

SEQ ID NO: 346 - protein, 6 aa, Artificial Sequence
  source: /note=“Description of Artificial Sequence Synthetic peptide”
IPAFGT

SEQ ID NO: 347 - protein, 18 aa, Artificial Sequence
  source: /note=“Description of Artificial Sequence Synthetic peptide”
EQDPEFGYGGYPYEAMDV

SEQ ID NO: 348 - protein, 127 aa, Artificial Sequence; identical to SEQ ID NO: 322
  source: /note=“Description of Artificial Sequence Synthetic polypeptide”

SEQ ID NO: 349 - DNA, 381 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaattcggttacggtggttacccgtatgaagctatggatgtttggggccaaggcaccctggtgactgttagctca

SEQ ID NO: 350 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEFGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - DNA, 1371 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaattcggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 352 - protein, 11 aa, Artificial Sequence
  source: /note=“Description of Artificial Sequence Synthetic peptide”
SGDNIPQHSVH

SEQ ID NO: 353 - protein, 7 aa, Artificial Sequence
  source: /note=“Description of Artificial Sequence Synthetic peptide”
DDTERPS

SEQ ID NO: 354 - protein, 11 aa, Artificial Sequence
  source: /note=“Description of Artificial Sequence Synthetic peptide”
SSWDSSMDSVV

SEQ ID NO: 355 - protein, 11 aa, Artificial Sequence
  source: /note=“Description of Artificial Sequence Synthetic peptide”
SGDNIPQHSVH

SEQ ID NO: 356 - protein, 7 aa, Artificial Sequence
  source: /note=“Description of Artificial Sequence Synthetic peptide”
DDTERPS

SEQ ID NO: 357 - protein, 11 aa, Artificial Sequence
  source: /note=“Description of Artificial Sequence Synthetic peptide”
SSWDSSMDSVV

SEQ ID NO: 358 - protein, 7 aa, Artificial Sequence
  source: /note=“Description of Artificial Sequence Synthetic peptide”
DNIPQHS

SEQ ID NO: 359 - protein, 3 aa, Artificial Sequence
  source: /note=“Description of Artificial Sequence Synthetic peptide”
DDT

SEQ ID NO: 360 - protein, 8 aa, Artificial Sequence
  source: /note=“Description of Artificial Sequence Synthetic peptide”
WDSSMDSV

SEQ ID NO: 361 - protein, 108 aa, Artificial Sequence; identical to SEQ ID NO: 179
  source: /note=“Description of Artificial Sequence Synthetic polypeptide”

SEQ ID NO: 362 - DNA, 324 bp, Artificial Sequence; identical to SEQ ID NO: 206
  source: /note=“Description of Artificial Sequence Synthetic polynucleotide”

SEQ ID NO: 363 - protein, 214 aa, Artificial Sequence; identical to SEQ ID NO: 181
  source: /note=“Description of Artificial Sequence Synthetic polypeptide”

SEQ ID NO: 364 - DNA, 642 bp, Artificial Sequence; identical to SEQ ID NO: 208
  source: /note=“Description of Artificial Sequence Synthetic polynucleotide”

SEQ ID NO: 365 - protein, 10 aa, Artificial Sequence
  source: /note=“Description of Artificial Sequence Synthetic peptide”
GGTFRDYAIS

SEQ ID NO: 366 - protein, 17 aa, Artificial Sequence
  source: /note=“Description of Artificial Sequence Synthetic peptide”
GIIPAFGTANYAQKFQG

SEQ ID NO: 367 - protein, 18 aa, Artificial Sequence
  source: /note=“Description of Artificial Sequence Synthetic peptide”
EQDPEAGYGGYPYEAMDV

SEQ ID NO: 368 - protein, 5 aa, Artificial Sequence
  source: /note=“Description of Artificial Sequence Synthetic peptide”
DYAIS

SEQ ID NO: 369 - protein, 17 aa, Artificial Sequence
  source: /note=“Description of Artificial Sequence Synthetic peptide”
GIIPAFGTANYAQKFQG

SEQ ID NO: 370 - protein, 18 aa, Artificial Sequence
  source: /note=“Description of Artificial Sequence Synthetic peptide”
EQDPEAGYGGYPYEAMDV

SEQ ID NO: 371 - protein, 7 aa, Artificial Sequence
  source: /note=“Description of Artificial Sequence Synthetic peptide”
GGTFRDY

SEQ ID NO: 372 - protein, 6 aa, Artificial Sequence
  source: /note=“Description of Artificial Sequence Synthetic peptide”
IPAFGT

SEQ ID NO: 373 - protein, 18 aa, Artificial Sequence
  source: /note=“Description of Artificial Sequence Synthetic peptide”
EQDPEAGYGGYPYEAMDV

SEQ ID NO: 374 - protein, 127 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEAGYGGYPYEAMDVWGQGTLVTVSS

SEQ ID NO: 375 - DNA, 381 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgaggccggctacggcggctacccctacgaggctatggacgtgtggggtcagggcaccctggtcaccgtgtctagc

SEQ ID NO: 376 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEAGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - DNA, 1371 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgaggccggctacggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 378 - protein, 11 aa, Artificial Sequence
  source: /note=“Description of Artificial Sequence Synthetic peptide”
SGDNIPQHSVH

SEQ ID NO: 379 - protein, 7 aa, Artificial Sequence
  source: /note=“Description of Artificial Sequence Synthetic peptide”
DDTERPS

SEQ ID NO: 380 - protein, 11 aa, Artificial Sequence
  source: /note=“Description of Artificial Sequence Synthetic peptide”
SSWDSSMDSVV

SEQ ID NO: 381 - protein, 11 aa, Artificial Sequence
  source: /note=“Description of Artificial Sequence Synthetic peptide”
SGDNIPQHSVH

SEQ ID NO: 382 - protein, 7 aa, Artificial Sequence
  source: /note=“Description of Artificial Sequence Synthetic peptide”
DDTERPS

SEQ ID NO: 383 - protein, 11 aa, Artificial Sequence
  source: /note=“Description of Artificial Sequence Synthetic peptide”
SSWDSSMDSVV

SEQ ID NO: 384 - protein, 7 aa, Artificial Sequence
  source: /note=“Description of Artificial Sequence Synthetic peptide”
DNIPQHS

SEQ ID NO: 385 - protein, 3 aa, Artificial Sequence
  source: /note=“Description of Artificial Sequence Synthetic peptide”
DDT

SEQ ID NO: 386 - protein, 8 aa, Artificial Sequence
  source: /note=“Description of Artificial Sequence Synthetic peptide”
WDSSMDSV

SEQ ID NO: 387 - protein, 108 aa, Artificial Sequence; identical to SEQ ID NO: 179
  source: /note=“Description of Artificial Sequence Synthetic polypeptide”

SEQ ID NO: 388 - DNA, 324 bp, Artificial Sequence; identical to SEQ ID NO: 180
  source: /note=“Description of Artificial Sequence Synthetic polynucleotide”

SEQ ID NO: 389 - protein, 214 aa, Artificial Sequence; identical to SEQ ID NO: 181
  source: /note=“Description of Artificial Sequence Synthetic polypeptide”

SEQ ID NO: 390 - DNA, 642 bp, Artificial Sequence; identical to SEQ ID NO: 182
  source: /note=“Description of Artificial Sequence Synthetic polynucleotide”

SEQ ID NO: 391 - protein, 10 aa, Artificial Sequence
  source: /note=“Description of Artificial Sequence Synthetic peptide”
GGTFRDYAIS

SEQ ID NO: 392 - protein, 17 aa, Artificial Sequence
  source: /note=“Description of Artificial Sequence Synthetic peptide”
GIIPAFGTANYAQKFQG

SEQ ID NO: 393 - protein, 18 aa, Artificial Sequence
  source: /note=“Description of Artificial Sequence Synthetic peptide”
EQDPEAGYGGYPYEAMDV

SEQ ID NO: 394 - protein, 5 aa, Artificial Sequence
  source: /note=“Description of Artificial Sequence Synthetic peptide”
DYAIS

SEQ ID NO: 395 - protein, 17 aa, Artificial Sequence
  source: /note=“Description of Artificial Sequence Synthetic peptide”
GIIPAFGTANYAQKFQG

SEQ ID NO: 396 - protein, 18 aa, Artificial Sequence
  source: /note=“Description of Artificial Sequence Synthetic peptide”
EQDPEAGYGGYPYEAMDV

SEQ ID NO: 397 - protein, 7 aa, Artificial Sequence
  source: /note=“Description of Artificial Sequence Synthetic peptide”
GGTFRDY

SEQ ID NO: 398 - protein, 6 aa, Artificial Sequence
  source: /note=“Description of Artificial Sequence Synthetic peptide”
IPAFGT

SEQ ID NO: 399 - protein, 18 aa, Artificial Sequence
  source: /note=“Description of Artificial Sequence Synthetic peptide”
EQDPEAGYGGYPYEAMDV

SEQ ID NO: 400 - protein, 127 aa, Artificial Sequence; identical to SEQ ID NO: 374
  source: /note=“Description of Artificial Sequence Synthetic polypeptide”

SEQ ID NO: 401 - DNA, 381 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaagccggttacggtggttacccgtatgaagctatggatgtttggggccaaggcaccctggtgactgttagctca

SEQ ID NO: 402 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEAGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 403 - DNA, 1371 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaagccggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 404 - protein, 11 aa, Artificial Sequence
  source: /note=“Description of Artificial Sequence Synthetic peptide”
SGDNIPQHSVH

SEQ ID NO: 405 - protein, 7 aa, Artificial Sequence
  source: /note=“Description of Artificial Sequence Synthetic peptide”
DDTERPS

SEQ ID NO: 406 - protein, 11 aa, Artificial Sequence
  source: /note=“Description of Artificial Sequence Synthetic peptide”
SSWDSSMDSVV

SEQ ID NO: 407 - protein, 11 aa, Artificial Sequence
  source: /note=“Description of Artificial Sequence Synthetic peptide”
SGDNIPQHSVH

SEQ ID NO: 408 - protein, 7 aa, Artificial Sequence
  source: /note=“Description of Artificial Sequence Synthetic peptide”
DDTERPS

SEQ ID NO: 409 - protein, 11 aa, Artificial Sequence
  source: /note=“Description of Artificial Sequence Synthetic peptide”
SSWDSSMDSVV

SEQ ID NO: 410 - protein, 7 aa, Artificial Sequence
  source: /note=“Description of Artificial Sequence Synthetic peptide”
DNIPQHS

SEQ ID NO: 411 - protein, 3 aa, Artificial Sequence
  source: /note=“Description of Artificial Sequence Synthetic peptide”
DDT

SEQ ID NO: 412 - protein, 8 aa, Artificial Sequence
  source: /note=“Description of Artificial Sequence Synthetic peptide”
WDSSMDSV

SEQ ID NO: 413 - protein, 108 aa, Artificial Sequence; identical to SEQ ID NO: 179
  source: /note=“Description of Artificial Sequence Synthetic polypeptide”

SEQ ID NO: 414 - DNA, 324 bp, Artificial Sequence; identical to SEQ ID NO: 206
  source: /note=“Description of Artificial Sequence Synthetic polynucleotide”

SEQ ID NO: 415 - protein, 214 aa, Artificial Sequence; identical to SEQ ID NO: 181
  source: /note=“Description of Artificial Sequence Synthetic polypeptide”

SEQ ID NO: 416 - DNA, 642 bp, Artificial Sequence; identical to SEQ ID NO: 208
  source: /note=“Description of Artificial Sequence Synthetic polynucleotide”

SEQ ID NO: 417 - protein, 10 aa, Artificial Sequence
  source: /note=“Description of Artificial Sequence Synthetic peptide”
GGTFRDYAIS

SEQ ID NO: 418 - protein, 17 aa, Artificial Sequence
  source: /note=“Description of Artificial Sequence Synthetic peptide”
GIIPAFGTANYAQKFQG

SEQ ID NO: 419 - protein, 18 aa, Artificial Sequence
  source: /note=“Description of Artificial Sequence Synthetic peptide”
EQDPESGYGGYPYEAMDV

SEQ ID NO: 420 - protein, 5 aa, Artificial Sequence
  source: /note=“Description of Artificial Sequence Synthetic peptide”
DYAIS

SEQ ID NO: 421 - protein, 17 aa, Artificial Sequence
  source: /note=“Description of Artificial Sequence Synthetic peptide”
GIIPAFGTANYAQKFQG

SEQ ID NO: 422 - protein, 18 aa, Artificial Sequence
  source: /note=“Description of Artificial Sequence Synthetic peptide”
EQDPESGYGGYPYEAMDV

SEQ ID NO: 423 - protein, 7 aa, Artificial Sequence
  source: /note=“Description of Artificial Sequence Synthetic peptide”
GGTFRDY

SEQ ID NO: 424 - protein, 6 aa, Artificial Sequence
  source: /note=“Description of Artificial Sequence Synthetic peptide”
IPAFGT

SEQ ID NO: 425 - protein, 18 aa, Artificial Sequence
  source: /note=“Description of Artificial Sequence Synthetic peptide”
EQDPESGYGGYPYEAMDV

SEQ ID NO: 426 - protein, 127 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPESGYGGYPYEAMDVWGQGTLVTVSS

SEQ ID NO: 427 - DNA, 381 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagtccggctacggcggctacccctacgaggctatggacgtgtggggtcagggcaccctggtcaccgtgtctagc

SEQ ID NO: 428 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPESGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 429 - DNA, 1371 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagtccggctacggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 430 - protein, 11 aa, Artificial Sequence
  source: /note=“Description of Artificial Sequence Synthetic peptide”
SGDNIPQHSVH

SEQ ID NO: 431 - protein, 7 aa, Artificial Sequence
  source: /note=“Description of Artificial Sequence Synthetic peptide”
DDTERPS

SEQ ID NO: 432 - protein, 11 aa, Artificial Sequence
  source: /note=“Description of Artificial Sequence Synthetic peptide”
SSWDSSMDSVV

SEQ ID NO: 433 - protein, 11 aa, Artificial Sequence
  source: /note=“Description of Artificial Sequence Synthetic peptide”
SGDNIPQHSVH

SEQ ID NO: 434 - protein, 7 aa, Artificial Sequence
  source: /note=“Description of Artificial Sequence Synthetic peptide”
DDTERPS

SEQ ID NO: 435 - protein, 11 aa, Artificial Sequence
  source: /note=“Description of Artificial Sequence Synthetic peptide”
SSWDSSMDSVV

SEQ ID NO: 436 - protein, 7 aa, Artificial Sequence
  source: /note=“Description of Artificial Sequence Synthetic peptide”
DNIPQHS

SEQ ID NO: 437 - protein, 3 aa, Artificial Sequence
  source: /note=“Description of Artificial Sequence Synthetic peptide”
DDT

SEQ ID NO: 438 - protein, 8 aa, Artificial Sequence
  source: /note=“Description of Artificial Sequence Synthetic peptide”
WDSSMDSV

SEQ ID NO: 439 - protein, 108 aa, Artificial Sequence; identical to SEQ ID NO: 179
  source: /note=“Description of Artificial Sequence Synthetic polypeptide”

SEQ ID NO: 440 - DNA, 324 bp, Artificial Sequence; identical to SEQ ID NO: 180
  source: /note=“Description of Artificial Sequence Synthetic polynucleotide”

SEQ ID NO: 441 - protein, 214 aa, Artificial Sequence; identical to SEQ ID NO: 181
  source: /note=“Description of Artificial Sequence Synthetic polypeptide”

SEQ ID NO: 442 - DNA, 642 bp, Artificial Sequence; identical to SEQ ID NO: 182
  source: /note=“Description of Artificial Sequence Synthetic polynucleotide”

SEQ ID NO: 443 - protein, 10 aa, Artificial Sequence
  source: /note=“Description of Artificial Sequence Synthetic peptide”
GGTFRDYAIS

SEQ ID NO: 444 - protein, 17 aa, Artificial Sequence
  source: /note=“Description of Artificial Sequence Synthetic peptide”
GIIPAFGTANYAQKFQG

SEQ ID NO: 445 - protein, 18 aa, Artificial Sequence
  source: /note=“Description of Artificial Sequence Synthetic peptide”
EQDPESGYGGYPYEAMDV

SEQ ID NO: 446 - protein, 5 aa, Artificial Sequence
  source: /note=“Description of Artificial Sequence Synthetic peptide”
DYAIS

SEQ ID NO: 447 - protein, 17 aa, Artificial Sequence
  source: /note=“Description of Artificial Sequence Synthetic peptide”
GIIPAFGTANYAQKFQG

SEQ ID NO: 448 - protein, 18 aa, Artificial Sequence
  source: /note=“Description of Artificial Sequence Synthetic peptide”
EQDPESGYGGYPYEAMDV

SEQ ID NO: 449 - protein, 7 aa, Artificial Sequence
  source: /note=“Description of Artificial Sequence Synthetic peptide”
GGTFRDY

SEQ ID NO: 450 - protein, 6 aa, Artificial Sequence
  source: /note=“Description of Artificial Sequence Synthetic peptide”
IPAFGT

SEQ ID NO: 451 - protein, 18 aa, Artificial Sequence
  source: /note=“Description of Artificial Sequence Synthetic peptide”
EQDPESGYGGYPYEAMDV

SEQ ID NO: 452 - protein, 127 aa, Artificial Sequence; identical to SEQ ID NO: 426
  source: /note=“Description of Artificial Sequence Synthetic polypeptide”

SEQ ID NO: 453 - DNA, 381 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaaagcggttacggtggttacccgtatgaagctatggatgtttggggccaaggcaccctggtgactgttagctca

SEQ ID NO: 454 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPESGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 455 - DNA, 1371 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaaagcggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 456 - protein, 11 aa, Artificial Sequence
  source: /note=“Description of Artificial Sequence Synthetic peptide”
SGDNIPQHSVH

SEQ ID NO: 457 - protein, 7 aa, Artificial Sequence
  source: /note=“Description of Artificial Sequence Synthetic peptide”
DDTERPS

SEQ ID NO: 458 - protein, 11 aa, Artificial Sequence
  source: /note=“Description of Artificial Sequence Synthetic peptide”
SSWDSSMDSVV

SEQ ID NO: 459 - protein, 11 aa, Artificial Sequence
  source: /note=“Description of Artificial Sequence Synthetic peptide”
SGDNIPQHSVH

SEQ ID NO: 460 - protein, 7 aa, Artificial Sequence
  source: /note=“Description of Artificial Sequence Synthetic peptide”
DDTERPS

SEQ ID NO: 461 - protein, 11 aa, Artificial Sequence
  source: /note=“Description of Artificial Sequence Synthetic peptide”
SSWDSSMDSVV

SEQ ID NO: 462 - protein, 7 aa, Artificial Sequence
  source: /note=“Description of Artificial Sequence Synthetic peptide”
DNIPQHS

SEQ ID NO: 463 - protein, 3 aa, Artificial Sequence
  source: /note=“Description of Artificial Sequence Synthetic peptide”
DDT

SEQ ID NO: 464 - protein, 8 aa, Artificial Sequence
  source: /note=“Description of Artificial Sequence Synthetic peptide”
WDSSMDSV

SEQ ID NO: 465 - protein, 108 aa, Artificial Sequence; identical to SEQ ID NO: 179
  source: /note=“Description of Artificial Sequence Synthetic polypeptide”

SEQ ID NO: 466 - DNA, 324 bp, Artificial Sequence; identical to SEQ ID NO: 206
  source: /note=“Description of Artificial Sequence Synthetic polynucleotide”

SEQ ID NO: 467 - protein, 214 aa, Artificial Sequence; identical to SEQ ID NO: 181
  source: /note=“Description of Artificial Sequence Synthetic polypeptide”

SEQ ID NO: 468 - DNA, 642 bp, Artificial Sequence; identical to SEQ ID NO: 208
  source: /note=“Description of Artificial Sequence Synthetic polynucleotide”

SEQ ID NO: 469 - protein, 10 aa, Artificial Sequence
  source: /note=“Description of Artificial Sequence Synthetic peptide”
GGTFRDYAIS

SEQ ID NO: 470 - protein, 17 aa, Artificial Sequence
  source: /note=“Description of Artificial Sequence Synthetic peptide”
GIIPAFGTANYAQKFQG

SEQ ID NO: 471 - protein, 18 aa, Artificial Sequence
  source: /note=“Description of Artificial Sequence Synthetic peptide”
EQDPEYGFGGYPYEAMDV

SEQ ID NO: 472 - protein, 5 aa, Artificial Sequence
  source: /note=“Description of Artificial Sequence Synthetic peptide”
DYAIS

SEQ ID NO: 473 - protein, 17 aa, Artificial Sequence
  source: /note=“Description of Artificial Sequence Synthetic peptide”
GIIPAFGTANYAQKFQG

SEQ ID NO: 474 - protein, 18 aa, Artificial Sequence
  source: /note=“Description of Artificial Sequence Synthetic peptide”
EQDPEYGFGGYPYEAMDV

SEQ ID NO: 475 - protein, 7 aa, Artificial Sequence
  source: /note=“Description of Artificial Sequence Synthetic peptide”
GGTFRDY

SEQ ID NO: 476 - protein, 6 aa, Artificial Sequence
  source: /note=“Description of Artificial Sequence Synthetic peptide”
IPAFGT

SEQ ID NO: 477 - protein, 18 aa, Artificial Sequence
  source: /note=“Description of Artificial Sequence Synthetic peptide”
EQDPEYGFGGYPYEAMDV

SEQ ID NO: 478 - protein, 127 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGFGGYPYEAMDVWGQGTLVTVSS

SEQ ID NO: 479 - DNA, 381 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagtacggcttcggcggctacccctacgaggctatggacgtgtggggtcagggcaccctggtcaccgtgtctagc

SEQ ID NO: 480 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGFGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 481 - DNA, 1371 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagtacggcttcggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 482 - protein, 11 aa, Artificial Sequence
  source: /note=“Description of Artificial Sequence Synthetic peptide”
SGDNIPQHSVH

SEQ ID NO: 483 - protein, 7 aa, Artificial Sequence
  source: /note=“Description of Artificial Sequence Synthetic peptide”
DDTERPS

SEQ ID NO: 484 - protein, 11 aa, Artificial Sequence
  source: /note=“Description of Artificial Sequence Synthetic peptide”
SSWDSSMDSVV

SEQ ID NO: 485 - protein, 11 aa, Artificial Sequence
  source: /note=“Description of Artificial Sequence Synthetic peptide”
SGDNIPQHSVH

SEQ ID NO: 486 - protein, 7 aa, Artificial Sequence
  source: /note=“Description of Artificial Sequence Synthetic peptide”
DDTERPS

SEQ ID NO: 487 - protein, 11 aa, Artificial Sequence
  source: /note=“Description of Artificial Sequence Synthetic peptide”
SSWDSSMDSVV

SEQ ID NO: 488 - protein, 7 aa, Artificial Sequence
  source: /note=“Description of Artificial Sequence Synthetic peptide”
DNIPQHS

SEQ ID NO: 489 - protein, 3 aa, Artificial Sequence
  source: /note=“Description of Artificial Sequence Synthetic peptide”
DDT

SEQ ID NO: 490 - protein, 8 aa, Artificial Sequence
  source: /note=“Description of Artificial Sequence Synthetic peptide”
WDSSMDSV

SEQ ID NO: 491 - protein, 108 aa, Artificial Sequence; identical to SEQ ID NO: 179
  source: /note=“Description of Artificial Sequence Synthetic polypeptide”

SEQ ID NO: 492 - DNA, 324 bp, Artificial Sequence; identical to SEQ ID NO: 180
  source: /note=“Description of Artificial Sequence Synthetic polynucleotide”

SEQ ID NO: 493 - protein, 214 aa, Artificial Sequence; identical to SEQ ID NO: 181
  source: /note=“Description of Artificial Sequence Synthetic polypeptide”

SEQ ID NO: 494 - DNA, 642 bp, Artificial Sequence; identical to SEQ ID NO: 182
  source: /note=“Description of Artificial Sequence Synthetic polynucleotide”

SEQ ID NO: 495 - protein, 10 aa, Artificial Sequence
  source: /note=“Description of Artificial Sequence Synthetic peptide”
GGTFRDYAIS

SEQ ID NO: 496 - protein, 17 aa, Artificial Sequence
  source: /note=“Description of Artificial Sequence Synthetic peptide”
GIIPAFGTANYAQKFQG

SEQ ID NO: 497 - protein, 18 aa, Artificial Sequence
  source: /note=“Description of Artificial Sequence Synthetic peptide”
EQDPEYGFGGYPYEAMDV

SEQ ID NO: 498 - protein, 5 aa, Artificial Sequence
  source: /note=“Description of Artificial Sequence Synthetic peptide”
DYAIS

SEQ ID NO: 499 - protein, 17 aa, Artificial Sequence
  source: /note=“Description of Artificial Sequence Synthetic peptide”
GIIPAFGTANYAQKFQG

SEQ ID NO: 500 - protein, 18 aa, Artificial Sequence
  source: /note=“Description of Artificial Sequence Synthetic peptide”
EQDPEYGFGGYPYEAMDV

SEQ ID NO: 501 - protein, 7 aa, Artificial Sequence
  source: /note=“Description of Artificial Sequence Synthetic peptide”
GGTFRDY

SEQ ID NO: 502 - protein, 6 aa, Artificial Sequence
  source: /note=“Description of Artificial Sequence Synthetic peptide”
IPAFGT

SEQ ID NO: 503 - protein, 18 aa, Artificial Sequence
  source: /note=“Description of Artificial Sequence Synthetic peptide”
EQDPEYGFGGYPYEAMDV

SEQ ID NO: 504 - protein, 127 aa, Artificial Sequence; identical to SEQ ID NO: 478
  source: /note=“Description of Artificial Sequence Synthetic polypeptide”

SEQ ID NO: 505 - DNA, 381 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggtttcggtggttacccgtatgaagctatggatgtttggggccaaggcaccctggtgactgttagctca

SEQ ID NO: 506 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGFGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 507 - DNA, 1371 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggtttcggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 508 - protein, 11 aa, Artificial Sequence
  source: /note=“Description of Artificial Sequence Synthetic peptide”
SGDNIPQHSVH

SEQ ID NO: 509 - protein, 7 aa, Artificial Sequence
  source: /note=“Description of Artificial Sequence Synthetic peptide”
DDTERPS

SEQ ID NO: 510 - protein, 11 aa, Artificial Sequence
  source: /note=“Description of Artificial Sequence Synthetic peptide”
SSWDSSMDSVV

SEQ ID NO: 511 - protein, 11 aa, Artificial Sequence
  source: /note=“Description of Artificial Sequence Synthetic peptide”
SGDNIPQHSVH

SEQ ID NO: 512 - protein, 7 aa, Artificial Sequence
  source: /note=“Description of Artificial Sequence Synthetic peptide”
DDTERPS

SEQ ID NO: 513 - protein, 11 aa, Artificial Sequence
  source: /note=“Description of Artificial Sequence Synthetic peptide”
SSWDSSMDSVV

SEQ ID NO: 514 - protein, 7 aa, Artificial Sequence
  source: /note=“Description of Artificial Sequence Synthetic peptide”
DNIPQHS

SEQ ID NO: 515 - protein, 3 aa, Artificial Sequence
  source: /note=“Description of Artificial Sequence Synthetic peptide”
DDT

SEQ ID NO: 516 - protein, 8 aa, Artificial Sequence
  source: /note=“Description of Artificial Sequence Synthetic peptide”
WDSSMDSV

SEQ ID NO: 517 - protein, 108 aa, Artificial Sequence; identical to SEQ ID NO: 179
  source: /note=“Description of Artificial Sequence Synthetic polypeptide”

SEQ ID NO: 518 - DNA, 324 bp, Artificial Sequence; identical to SEQ ID NO: 206
  source: /note=“Description of Artificial Sequence Synthetic polynucleotide”

SEQ ID NO: 519 - protein, 214 aa, Artificial Sequence; identical to SEQ ID NO: 181
  source: /note=“Description of Artificial Sequence Synthetic polypeptide”

SEQ ID NO: 520 - DNA, 642 bp, Artificial Sequence; identical to SEQ ID NO: 208
  source: /note=“Description of Artificial Sequence Synthetic polynucleotide”

SEQ ID NO: 521 - protein, 10 aa, Artificial Sequence
  source: /note=“Description of Artificial Sequence Synthetic peptide”
GGTFRDYAIS

SEQ ID NO: 522 - protein, 17 aa, Artificial Sequence
  source: /note=“Description of Artificial Sequence Synthetic peptide”
GIIPAFGTANYAQKFQG

SEQ ID NO: 523 - protein, 18 aa, Artificial Sequence
  source: /note=“Description of Artificial Sequence Synthetic peptide”
EQDPEYGYGGFPYEAMDV

SEQ ID NO: 524 - protein, 5 aa, Artificial Sequence
  source: /note=“Description of Artificial Sequence Synthetic peptide”
DYAIS

SEQ ID NO: 525 - protein, 17 aa, Artificial Sequence
  source: /note=“Description of Artificial Sequence Synthetic peptide”
GIIPAFGTANYAQKFQG

SEQ ID NO: 526 - protein, 18 aa, Artificial Sequence
  source: /note=“Description of Artificial Sequence Synthetic peptide”
EQDPEYGYGGFPYEAMDV

SEQ ID NO: 527 - protein, 7 aa, Artificial Sequence
  source: /note=“Description of Artificial Sequence Synthetic peptide”
GGTFRDY

SEQ ID NO: 528 - protein, 6 aa, Artificial Sequence
  source: /note=“Description of Artificial Sequence Synthetic peptide”
IPAFGT

SEQ ID NO: 529 - protein, 18 aa, Artificial Sequence
  source: /note=“Description of Artificial Sequence Synthetic peptide”
EQDPEYGYGGFPYEAMDV

SEQ ID NO: 530 - protein, 127 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGYGGFPYEAMDVWGQGTLVTVSS

SEQ ID NO: 531 - DNA, 381 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggtttcccgtatgaagctatggatgtttggggccaaggcaccctggtgactgttagctca

SEQ ID NO: 532 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGYGGFPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 533 - DNA, 1371 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggtttcccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 534 - protein, 11 aa, Artificial Sequence
  source: /note=“Description of Artificial Sequence Synthetic peptide”
SGDNIPQHSVH

SEQ ID NO: 535 - protein, 7 aa, Artificial Sequence
  source: /note=“Description of Artificial Sequence Synthetic peptide”
DDTERPS

SEQ ID NO: 536 - protein, 11 aa, Artificial Sequence
  source: /note=“Description of Artificial Sequence Synthetic peptide”
SSWDSSMDSVV

SEQ ID NO: 537 - protein, 11 aa, Artificial Sequence
  source: /note=“Description of Artificial Sequence Synthetic peptide”
SGDNIPQHSVH

SEQ ID NO: 538 - protein, 7 aa, Artificial Sequence
  source: /note=“Description of Artificial Sequence Synthetic peptide”
DDTERPS

SEQ ID NO: 539 - protein, 11 aa, Artificial Sequence
  source: /note=“Description of Artificial Sequence Synthetic peptide”
SSWDSSMDSVV

SEQ ID NO: 540 - protein, 7 aa, Artificial Sequence
  source: /note=“Description of Artificial Sequence Synthetic peptide”
DNIPQHS

SEQ ID NO: 541 - protein, 3 aa, Artificial Sequence
  source: /note=“Description of Artificial Sequence Synthetic peptide”
DDT

SEQ ID NO: 542 - protein, 8 aa, Artificial Sequence
  source: /note=“Description of Artificial Sequence Synthetic peptide”
WDSSMDSV

SEQ ID NO: 543 - protein, 108 aa, Artificial Sequence; identical to SEQ ID NO: 179
  source: /note=“Description of Artificial Sequence Synthetic polypeptide”

SEQ ID NO: 544 - DNA, 324 bp, Artificial Sequence; identical to SEQ ID NO: 206
  source: /note=“Description of Artificial Sequence Synthetic polynucleotide”

SEQ ID NO: 545 - protein, 214 aa, Artificial Sequence; identical to SEQ ID NO: 181
  source: /note=“Description of Artificial Sequence Synthetic polypeptide”

SEQ ID NO: 546 - DNA, 642 bp, Artificial Sequence; identical to SEQ ID NO: 208
  source: /note=“Description of Artificial Sequence Synthetic polynucleotide”

SEQ ID NO: 547 - protein, 10 aa, Artificial Sequence
  source: /note=“Description of Artificial Sequence Synthetic peptide”
GGTFRDYAIS

SEQ ID NO: 548 - protein, 17 aa, Artificial Sequence
  source: /note=“Description of Artificial Sequence Synthetic peptide”
GIIPAFGTANYAQKFQG

SEQ ID NO: 549 - protein, 18 aa, Artificial Sequence
  source: /note=“Description of Artificial Sequence Synthetic peptide”
EQDPEYGYGGYPFEAMDV

SEQ ID NO: 550 - protein, 5 aa, Artificial Sequence
  source: /note=“Description of Artificial Sequence Synthetic peptide”
DYAIS

SEQ ID NO: 551 - protein, 17 aa, Artificial Sequence
  source: /note=“Description of Artificial Sequence Synthetic peptide”
GIIPAFGTANYAQKFQG

SEQ ID NO: 552 - protein, 18 aa, Artificial Sequence
  source: /note=“Description of Artificial Sequence Synthetic peptide”
EQDPEYGYGGYPFEAMDV

SEQ ID NO: 553 - protein, 7 aa, Artificial Sequence
  source: /note=“Description of Artificial Sequence Synthetic peptide”
GGTFRDY

SEQ ID NO: 554 - protein, 6 aa, Artificial Sequence
  source: /note=“Description of Artificial Sequence Synthetic peptide”
IPAFGT

SEQ ID NO: 555 - protein, 18 aa, Artificial Sequence
  source: /note=“Description of Artificial Sequence Synthetic peptide”
EQDPEYGYGGYPFEAMDV

SEQ ID NO: 556 - protein, 127 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGYGGYPFEAMDVWGQGTLVTVSS

SEQ ID NO: 557 - DNA, 381 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggttacccgttcgaagctatggatgtttggggccaaggcaccctggtgactgttagctca

SEQ ID NO: 558 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGYGGYPFEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 559 - DNA, 1371 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggttacccgttcgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 560 - protein, 11 aa, Artificial Sequence
  source: /note=“Description of Artificial Sequence Synthetic peptide”
SGDNIPQHSVH

SEQ ID NO: 561 - protein, 7 aa, Artificial Sequence
  source: /note=“Description of Artificial Sequence Synthetic peptide”
DDTERPS

SEQ ID NO: 562 - protein, 11 aa, Artificial Sequence
  source: /note=“Description of Artificial Sequence Synthetic peptide”
SSWDSSMDSVV

SEQ ID NO: 563 - protein, 11 aa, Artificial Sequence
  source: /note=“Description of Artificial Sequence Synthetic peptide”
SGDNIPQHSVH

SEQ ID NO: 564 - protein, 7 aa, Artificial Sequence
  source: /note=“Description of Artificial Sequence Synthetic peptide”
DDTERPS

SEQ ID NO: 565 - protein, 11 aa, Artificial Sequence
  source: /note=“Description of Artificial Sequence Synthetic peptide”
SSWDSSMDSVV

SEQ ID NO: 566 - protein, 7 aa, Artificial Sequence
  source: /note=“Description of Artificial Sequence Synthetic peptide”
DNIPQHS

SEQ ID NO: 567 - protein, 3 aa, Artificial Sequence
  source: /note=“Description of Artificial Sequence Synthetic peptide”
DDT

SEQ ID NO: 568 - protein, 8 aa, Artificial Sequence
  source: /note=“Description of Artificial Sequence Synthetic peptide”
WDSSMDSV

SEQ ID NO: 569 - protein, 108 aa, Artificial Sequence; identical to SEQ ID NO: 179
  source: /note=“Description of Artificial Sequence Synthetic polypeptide”

SEQ ID NO: 570 - DNA, 324 bp, Artificial Sequence; identical to SEQ ID NO: 206
  source: /note=“Description of Artificial Sequence Synthetic polynucleotide”

SEQ ID NO: 571 - protein, 214 aa, Artificial Sequence; identical to SEQ ID NO: 181
  source: /note=“Description of Artificial Sequence Synthetic polypeptide”

SEQ ID NO: 572 - DNA, 642 bp, Artificial Sequence; identical to SEQ ID NO: 208
  source: /note=“Description of Artificial Sequence Synthetic polynucleotide”

SEQ ID NO: 573 - protein, 10 aa, Artificial Sequence
  source: /note=“Description of Artificial Sequence Synthetic peptide”
GGTFRDYAIS

SEQ ID NO: 574 - protein, 17 aa, Artificial Sequence
  source: /note=“Description of Artificial Sequence Synthetic peptide”
GIIPAFGTANYAQKFQG

SEQ ID NO: 575 - protein, 18 aa, Artificial Sequence
  source: /note=“Description of Artificial Sequence Synthetic peptide”
EQDPSYGYGGYPYEAMDV

SEQ ID NO: 576 - protein, 5 aa, Artificial Sequence
  source: /note=“Description of Artificial Sequence Synthetic peptide”
DYAIS

SEQ ID NO: 577 - protein, 17 aa, Artificial Sequence
  source: /note=“Description of Artificial Sequence Synthetic peptide”
GIIPAFGTANYAQKFQG

SEQ ID NO: 578 - protein, 18 aa, Artificial Sequence
  source: /note=“Description of Artificial Sequence Synthetic peptide”
EQDPSYGYGGYPYEAMDV

SEQ ID NO: 579 - protein, 7 aa, Artificial Sequence
  source: /note=“Description of Artificial Sequence Synthetic peptide”
GGTFRDY

SEQ ID NO: 580 - protein, 6 aa, Artificial Sequence
  source: /note=“Description of Artificial Sequence Synthetic peptide”
IPAFGT

SEQ ID NO: 581 - protein, 18 aa, Artificial Sequence
  source: /note=“Description of Artificial Sequence Synthetic peptide”
EQDPSYGYGGYPYEAMDV

SEQ ID NO: 582 - protein, 127 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPSYGYGGYPYEAMDVWGQGTLVTVSS

SEQ ID NO: 583 - DNA, 381 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggacccctcctacggctacggcggctacccctacgaggctatggacgtgtggggtcagggcaccctggtcaccgtgtctagc

SEQ ID NO: 584 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPSYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 585 - DNA, 1371 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggacccctcctacggctacggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 586 - protein, 11 aa, Artificial Sequence
  source: /note=“Description of Artificial Sequence Synthetic peptide”
SGDNIPQHSVH

SEQ ID NO: 587 - protein, 7 aa, Artificial Sequence
  source: /note=“Description of Artificial Sequence Synthetic peptide”
DDTERPS

SEQ ID NO: 588 - protein, 11 aa, Artificial Sequence
  source: /note=“Description of Artificial Sequence Synthetic peptide”
SSWDSSMDSVV

SEQ ID NO: 589 - protein, 11 aa, Artificial Sequence
  source: /note=“Description of Artificial Sequence Synthetic peptide”
SGDNIPQHSVH

SEQ ID NO: 590 - protein, 7 aa, Artificial Sequence
  source: /note=“Description of Artificial Sequence Synthetic peptide”
DDTERPS

SEQ ID NO: 591 - protein, 11 aa, Artificial Sequence
  source: /note=“Description of Artificial Sequence Synthetic peptide”
SSWDSSMDSVV

SEQ ID NO: 592 - protein, 7 aa, Artificial Sequence
  source: /note=“Description of Artificial Sequence Synthetic peptide”
DNIPQHS

SEQ ID NO: 593 - protein, 3 aa, Artificial Sequence
  source: /note=“Description of Artificial Sequence Synthetic peptide”
DDT

SEQ ID NO: 594 - protein, 8 aa, Artificial Sequence
  source: /note=“Description of Artificial Sequence Synthetic peptide”
WDSSMDSV

SEQ ID NO: 595 - protein, 108 aa, Artificial Sequence; identical to SEQ ID NO: 179
  source: /note=“Description of Artificial Sequence Synthetic polypeptide”

SEQ ID NO: 596 - DNA, 324 bp, Artificial Sequence; identical to SEQ ID NO: 180
  source: /note=“Description of Artificial Sequence Synthetic polynucleotide”

SEQ ID NO: 597 - protein, 214 aa, Artificial Sequence; identical to SEQ ID NO: 181
  source: /note=“Description of Artificial Sequence Synthetic polypeptide”

SEQ ID NO: 598 - DNA, 642 bp, Artificial Sequence; identical to SEQ ID NO: 182
  source: /note=“Description of Artificial Sequence Synthetic polynucleotide”

SEQ ID NO: 599 - protein, 10 aa, Artificial Sequence
  source: /note=“Description of Artificial Sequence Synthetic peptide”
GGTFRDYAIS

SEQ ID NO: 600 - protein, 17 aa, Artificial Sequence
  source: /note=“Description of Artificial Sequence Synthetic peptide”
GIIPAFGTANYAQKFQG

SEQ ID NO: 601 - protein, 18 aa, Artificial Sequence
  source: /note=“Description of Artificial Sequence Synthetic peptide”
EQDPSYGYGGYPYEAMDV

SEQ ID NO: 602 - protein, 5 aa, Artificial Sequence
  source: /note=“Description of Artificial Sequence Synthetic peptide”
DYAIS

SEQ ID NO: 603 - protein, 17 aa, Artificial Sequence
  source: /note=“Description of Artificial Sequence Synthetic peptide”
GIIPAFGTANYAQKFQG

SEQ ID NO: 604 - protein, 18 aa, Artificial Sequence
  source: /note=“Description of Artificial Sequence Synthetic peptide”
EQDPSYGYGGYPYEAMDV

SEQ ID NO: 605 - protein, 7 aa, Artificial Sequence
  source: /note=“Description of Artificial Sequence Synthetic peptide”
GGTFRDY

SEQ ID NO: 606 - protein, 6 aa, Artificial Sequence
  source: /note=“Description of Artificial Sequence Synthetic peptide”
IPAFGT

SEQ ID NO: 607 - protein, 18 aa, Artificial Sequence
  source: /note=“Description of Artificial Sequence Synthetic peptide”
EQDPSYGYGGYPYEAMDV

SEQ ID NO: 608 - protein, 127 aa, Artificial Sequence; identical to SEQ ID NO: 582
  source: /note=“Description of Artificial Sequence Synthetic polypeptide”

SEQ ID NO: 609 - DNA, 381 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccgagctacggttacggtggttacccgtatgaagctatggatgtttggggccaaggcaccctggtgactgttagctca

SEQ ID NO: 610 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPSYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 611 - DNA, 1371 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccgagctacggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 612 - protein, 11 aa, Artificial Sequence
  source: /note=“Description of Artificial Sequence Synthetic peptide”
SGDNIPQHSVH

SEQ ID NO: 613 - protein, 7 aa, Artificial Sequence
  source: /note=“Description of Artificial Sequence Synthetic peptide”
DDTERPS

SEQ ID NO: 614 - protein, 11 aa, Artificial Sequence
  source: /note=“Description of Artificial Sequence Synthetic peptide”
SSWDSSMDSVV

SEQ ID NO: 615 - protein, 11 aa, Artificial Sequence
  source: /note=“Description of Artificial Sequence Synthetic peptide”
SGDNIPQHSVH

SEQ ID NO: 616 - protein, 7 aa, Artificial Sequence
  source: /note=“Description of Artificial Sequence Synthetic peptide”
DDTERPS

SEQ ID NO: 617 - protein, 11 aa, Artificial Sequence
  source: /note=“Description of Artificial Sequence Synthetic peptide”
SSWDSSMDSVV

SEQ ID NO: 618 - protein, 7 aa, Artificial Sequence
  source: /note=“Description of Artificial Sequence Synthetic peptide”
DNIPQHS

SEQ ID NO: 619 - protein, 3 aa, Artificial Sequence
  source: /note=“Description of Artificial Sequence Synthetic peptide”
DDT

SEQ ID NO: 620 - protein, 8 aa, Artificial Sequence
  source: /note=“Description of Artificial Sequence Synthetic peptide”
WDSSMDSV

SEQ ID NO: 621 - protein, 108 aa, Artificial Sequence; identical to SEQ ID NO: 179
  source: /note=“Description of Artificial Sequence Synthetic polypeptide”

SEQ ID NO: 622 - DNA, 324 bp, Artificial Sequence; identical to SEQ ID NO: 206
  source: /note=“Description of Artificial Sequence Synthetic polynucleotide”

SEQ ID NO: 623 - protein, 214 aa, Artificial Sequence; identical to SEQ ID NO: 181
  source: /note=“Description of Artificial Sequence Synthetic polypeptide”

SEQ ID NO: 624 - DNA, 642 bp, Artificial Sequence; identical to SEQ ID NO: 208
  source: /note=“Description of Artificial Sequence Synthetic polynucleotide”

SEQ ID NO: 625 - protein, 10 aa, Artificial Sequence
  source: /note=“Description of Artificial Sequence Synthetic peptide”
GGTFRDYAIS

SEQ ID NO: 626 - protein, 17 aa, Artificial Sequence
  source: /note=“Description of Artificial Sequence Synthetic peptide”
GIIPAFGTANYAQKFQG

SEQ ID NO: 627 - protein, 18 aa, Artificial Sequence
  source: /note=“Description of Artificial Sequence Synthetic peptide”
EQSPEYGYGGYPYEAMDV

SEQ ID NO: 628 - protein, 5 aa, Artificial Sequence
  source: /note=“Description of Artificial Sequence Synthetic peptide”
DYAIS

SEQ ID NO: 629 - protein, 17 aa, Artificial Sequence
  source: /note=“Description of Artificial Sequence Synthetic peptide”
GIIPAFGTANYAQKFQG

SEQ ID NO: 630 - protein, 18 aa, Artificial Sequence
  source: /note=“Description of Artificial Sequence Synthetic peptide”
EQSPEYGYGGYPYEAMDV

SEQ ID NO: 631 - protein, 7 aa, Artificial Sequence
  source: /note=“Description of Artificial Sequence Synthetic peptide”
GGTFRDY

SEQ ID NO: 632 - protein, 6 aa, Artificial Sequence
  source: /note=“Description of Artificial Sequence Synthetic peptide”
IPAFGT

SEQ ID NO: 633 - protein, 18 aa, Artificial Sequence
  source: /note=“Description of Artificial Sequence Synthetic peptide”
EQSPEYGYGGYPYEAMDV

SEQ ID NO: 634 - protein, 127 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQSPEYGYGGYPYEAMDVWGQGTLVTVSS

SEQ ID NO: 635 - DNA, 381 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcagtcccccgagtacggctacggcggctacccctacgaggctatggacgtgtggggtcagggcaccctggtcaccgtgtctagc

SEQ ID NO: 636 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQSPEYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 637 - DNA, 1371 bp, Artificial Sequence
  source: /note=“Description of Artificial Sequence Synthetic polynucleotide”
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcagtcccccgagtacggctacggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 638 - protein, 11 aa, Artificial Sequence
  source: /note=“Description of Artificial Sequence Synthetic peptide”
SGDNIPQHSVH

SEQ ID NO: 639 - protein, 7 aa, Artificial Sequence
  source: /note=“Description of Artificial Sequence Synthetic peptide”
DDTERPS

SEQ ID NO: 640 - protein, 11 aa, Artificial Sequence
  source: /note=“Description of Artificial Sequence Synthetic peptide”
SSWDSSMDSVV

SEQ ID NO: 641 - protein, 11 aa, Artificial Sequence
  source: /note=“Description of Artificial Sequence Synthetic peptide”
SGDNIPQHSVH

SEQ ID NO: 642 - protein, 7 aa, Artificial Sequence
  source: /note=“Description of Artificial Sequence Synthetic peptide”
DDTERPS

SEQ ID NO: 643 - protein, 11 aa, Artificial Sequence
  source: /note=“Description of Artificial Sequence Synthetic peptide”
SSWDSSMDSVV

SEQ ID NO: 644 - protein, 7 aa, Artificial Sequence
  source: /note=“Description of Artificial Sequence Synthetic peptide”
DNIPQHS

SEQ ID NO: 645 - protein, 3 aa, Artificial Sequence
  source: /note=“Description of Artificial Sequence Synthetic peptide”
DDT

SEQ ID NO: 646 - protein, 8 aa, Artificial Sequence
  source: /note=“Description of Artificial Sequence Synthetic peptide”
WDSSMDSV

SEQ ID NO: 647 - protein, 108 aa, Artificial Sequence; identical to SEQ ID NO: 179
  source: /note=“Description of Artificial Sequence Synthetic polypeptide”

SEQ ID NO: 648 - DNA, 324 bp, Artificial Sequence; identical to SEQ ID NO: 180
  source: /note=“Description of Artificial Sequence Synthetic polynucleotide”

SEQ ID NO: 649 - protein, 214 aa, Artificial Sequence; identical to SEQ ID NO: 181
  source: /note=“Description of Artificial Sequence Synthetic polypeptide”

SEQ ID NO: 650 - DNA, 642 bp, Artificial Sequence; identical to SEQ ID NO: 182
  source: /note=“Description of Artificial Sequence Synthetic polynucleotide”

SEQ ID NO: 651 - protein, 10 aa, Artificial Sequence
  source: /note=“Description of Artificial Sequence Synthetic peptide”
GGTFRDYAIS

SEQ ID NO: 652 - protein, 17 aa, Artificial Sequence
  source: /note=“Description of Artificial Sequence Synthetic peptide”
GIIPAFGTANYAQKFQG

SEQ ID NO: 653 - protein, 18 aa, Artificial Sequence
  source: /note=“Description of Artificial Sequence Synthetic peptide”
EQSPEYGYGGYPYEAMDV

SEQ ID NO: 654 - protein, 5 aa, Artificial Sequence
  source: /note=“Description of Artificial Sequence Synthetic peptide”
DYAIS

SEQ ID NO: 655 - protein, 17 aa, Artificial Sequence
  source: /note=“Description of Artificial Sequence Synthetic peptide”
GIIPAFGTANYAQKFQG

SEQ ID NO: 656 - protein, 18 aa, Artificial Sequence
  source: /note=“Description of Artificial Sequence Synthetic peptide”
EQSPEYGYGGYPYEAMDV

SEQ ID NO: 657 - protein, 7 aa, Artificial Sequence
  source: /note=“Description of Artificial Sequence Synthetic peptide”
GGTFRDY

SEQ ID NO: 658 - protein, 6 aa, Artificial Sequence
  source: /note=“Description of Artificial Sequence Synthetic peptide”
IPAFGT

SEQ ID NO: 659 - protein, 18 aa, Artificial Sequence
  source: /note=“Description of Artificial Sequence Synthetic peptide”
EQSPEYGYGGYPYEAMDV

SEQ ID NO: 660 - protein, 127 aa, Artificial Sequence; identical to SEQ ID NO: 634
  source: /note=“Description of Artificial Sequence Synthetic polypeptide”

SEQ ID NO: 661 - DNA, 381 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacagagcccggaatacggttacggtggttacccgtatgaagctatggatgtttggggccaaggcaccctggtgactgttagctca

SEQ ID NO: 662 - protein, 457 aa, Artificial Sequence
  source: /note=“Description of Artificial Sequence Synthetic polypeptide”
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQSPEYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 663 - DNA, 1371 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacagagcccggaatacggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 664 - protein, 11 aa, Artificial Sequence
  source: /note=“Description of Artificial Sequence Synthetic peptide”
SGDNIPQHSVH

SEQ ID NO: 665 - protein, 7 aa, Artificial Sequence
  source: /note=“Description of Artificial Sequence Synthetic peptide”
DDTERPS

SEQ ID NO: 666 - protein, 11 aa, Artificial Sequence
  source: /note=“Description of Artificial Sequence Synthetic peptide”
SSWDSSMDSVV

SEQ ID NO: 667 - protein, 11 aa, Artificial Sequence
  source: /note=“Description of Artificial Sequence Synthetic peptide”
SGDNIPQHSVH

SEQ ID NO: 668 - protein, 7 aa, Artificial Sequence
  source: /note=“Description of Artificial Sequence Synthetic peptide”
DDTERPS

SEQ ID NO: 669 - protein, 11 aa, Artificial Sequence
  source: /note=“Description of Artificial Sequence Synthetic peptide”
SSWDSSMDSVV

SEQ ID NO: 670 - protein, 7 aa, Artificial Sequence
  source: /note=“Description of Artificial Sequence Synthetic peptide”
DNIPQHS

SEQ ID NO: 671 - protein, 3 aa, Artificial Sequence
  source: /note=“Description of Artificial Sequence Synthetic peptide”
DDT

SEQ ID NO: 672 - protein, 8 aa, Artificial Sequence
  source: /note=“Description of Artificial Sequence Synthetic peptide”
WDSSMDSV

SEQ ID NO: 673 - protein, 108 aa, Artificial Sequence; identical to SEQ ID NO: 179
  source: /note=“Description of Artificial Sequence Synthetic polypeptide”

SEQ ID NO: 674 - DNA, 324 bp, Artificial Sequence; identical to SEQ ID NO: 206
  source: /note=“Description of Artificial Sequence Synthetic polynucleotide”

SEQ ID NO: 675 - protein, 214 aa, Artificial Sequence; identical to SEQ ID NO: 181
  source: /note=“Description of Artificial Sequence Synthetic polypeptide”

SEQ ID NO: 676 - DNA, 642 bp, Artificial Sequence; identical to SEQ ID NO: 208
  source: /note=“Description of Artificial Sequence Synthetic polynucleotide”

SEQ ID NO: 677 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 678 - protein, 310 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 679 - protein, 291 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 680 - protein, 180 aa, Homo sapiens
TPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPM

SEQ ID NO: 681 - protein, 185 aa, Homo sapiens
QAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGI

SEQ ID NO: 682 - protein, 175 aa, Homo sapiens
TPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAP

SEQ ID NO: 683 - protein, 18 aa, Artificial Sequence
  source: /note=“Description of Artificial Sequence Synthetic peptide”
EQDPEYGYGGFPYEAMDV

SEQ ID NO: 684 - protein, 6 aa, Artificial Sequence
  source: /note=“Description of Artificial Sequence Synthetic 6xHis tag”
HHHHHH

SEQ ID NO: 685 - protein, 17 aa, Homo sapiens
VLRCHSWKDKPLVKVTF

SEQ ID NO: 686 - protein, 8 aa, Homo sapiens
SWKDKPLV

SEQ ID NO: 687 - protein, 7 aa, Homo sapiens
SRSDPNF